SEQUENCE LISTING (190 sequences)

SEQ ID NO: 1 - protein, 326 aa, synthetic construct
  REGION 1..326: note = amino acid sequence of human PVRIG
MRTEAQVPALQPPEPGLEGAMGHRTLVLPWVLLTLCVTAGTPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLAGILGVSGVLLFGCVYLLHLLRRHKHRPAPRLQPSRTSPQAPRARAWAPSQASQAALHVPYATINTSCRPATLDTAHPHGGPSWWASLPTHAAHRPQGPAAWASTPIPARGSFVSVENGLYAQAGERPPHTGPGLTLFPDPRGPRAMEGPLGVR

SEQ ID NO: 2 - protein, 322 aa, synthetic construct
  REGION 1..322: note = Amino acid sequence of Cynomolgus Monkey ( Cyno ) PVRIG
MRTEAQVLALQSPEPGLEGAMGRRTLALPWVLLTLCVTAGTPEVWVQVQMEATELSSFTVHCGFLGPGSISLVTVSWGGPDGAGGTKLAVLHPELGTRQWAPARQARWETQSSISLALEDSGASSPFANTTFCCKFASFPEGSWESCGSLPPSSDPGLSAPPTPVPILRADLAGILGLSGVLLFGCGYLLHLLRRQKHRPTPRLQPSHTNSQALTAQAWAPSQASQAALHDPYATINTSFCPATLDTAHPNGWASLPTHAAHQPQGPAASASTPILARGSFVSVENGLYTQAGERPPHTGPGLTLFPDCRGPRAVEGRFGVR

SEQ ID NO: 3 - protein, 538 aa, synthetic construct
  REGION 1..538: note = Amino acid sequence of HveB or Nectin-2
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 4 - protein, 5 aa, synthetic construct
  REGION 1..5: note = BGA38 HCDR1 (Kabat)
DYWMY

SEQ ID NO: 5 - protein, 17 aa, synthetic construct
  REGION 1..17: note = BGA38 HCDR2 (Kabat)
TIDTSDHKTSYNQKFRG

SEQ ID NO: 6 - protein, 13 aa, synthetic construct
  REGION 1..13: note = BGA38 HCDR3 (Kabat)
DFIITVNYPVVDY

SEQ ID NO: 7 - protein, 10 aa, synthetic construct
  REGION 1..10: note = BGA38 LCDR1 (Kabat)
SAGSSVNYMH

SEQ ID NO: 8 - protein, 7 aa, synthetic construct
  REGION 1..7: note = BGA38 LCDR2 (Kabat)
STSNLAS

SEQ ID NO: 9 - protein, 9 aa, synthetic construct
  REGION 1..9: note = BGA38 LCDR3 (Kabat)
QQRSSLPFT

SEQ ID NO: 10 - protein, 122 aa, synthetic construct
  REGION 1..122: note = BGA38 VH AA
EVQLQESGAEHVMPGASVKMSCKAAGYTFTDYWMYWVKQRPGQGLEWIGTIDTSDHKTSYNQKFRGKATLTVDESSSTAYMQLSSLTSEDSAVYYCARDFIITVNYPVVDYWGQGTSVTVSS

SEQ ID NO: 11 - protein, 106 aa, synthetic construct
  REGION 1..106: note = BGA38 VL AA
QNVLTQSPAIMSASPGEKVTITCSAGSSVNYMHWFQQKPGTSPKLWIYSTSNLASGVPARFSGSGSGTSYSLTISRMEAEDAATYYCQQRSSLPFTFGSGTKLEIK

SEQ ID NO: 12 - DNA, 366 bp, Synthetic construct
  misc_feature 1..366: note = BGA38 VH DNA
  source 1..366: mol_type = other DNA
gaggtgcagctgcaagagagcggcgccgagcacgtgatgcccggcgcctccgtgaagatgagctgcaaggccgccggctacaccttcaccgactactggatgtactgggtgaagcagagacccggccaaggcctggagtggatcggcaccatcgacacaagcgaccacaagacaagctacaatcagaagttcagaggcaaggccaccctgaccgtggacgagagcagcagcaccgcctacatgcagctgagcagcctgacaagcgaggacagcgccgtgtactactgcgctagagacttcatcatcaccgtgaactaccccgtggtggactactggggccaaggcacaagcgtgaccgtgagcagc

SEQ ID NO: 13 - DNA, 318 bp, Synthetic construct
  misc_feature 1..318: note = BGA38 VL DNA
  source 1..318: mol_type = other DNA
cagaacgtgctgacacagagccccgccatcatgagcgcctcccccggcgagaaggtgaccatcacctgcagcgccggcagcagcgtgaactacatgcactggtttcagcagaagcccggcacaagccccaagctgtggatctacagcacaagcaacctggcctccggcgtgcccgctagattcagcggcagcggcagcgggacaagctacagcctgaccatcagcagaatggaggccgaggacgccgccacctactactgtcagcagagaagcagcctgcccttcaccttcggcagcggcaccaagctggagatcaag

SEQ ID NO: 14 - protein, 5 aa, synthetic construct
  REGION 1..5: note = BGA381 HCDR1 (Kabat)
DYWIY

SEQ ID NO: 15 - protein, 17 aa, synthetic construct
  REGION 1..17: note = BGA381 HCDR2 (Kabat)
TIDTSDHKTSYNQKFRG

SEQ ID NO: 16 - protein, 13 aa, synthetic construct
  REGION 1..13: note = BGA381 HCDR3 (Kabat)
DFIITVNYPVVDY

SEQ ID NO: 17 - protein, 10 aa, synthetic construct
  REGION 1..10: note = BGA381 LCDR1 (Kabat)
SAGSSVNYMH

SEQ ID NO: 18 - protein, 7 aa, synthetic construct
  REGION 1..7: note = BGA381 LCDR2 (Kabat)
STSNLAS

SEQ ID NO: 19 - protein, 9 aa, synthetic construct
  REGION 1..9: note = BGA381 LCDR3 (Kabat)
QQRSSLPFT

SEQ ID NO: 20 - protein, 122 aa, synthetic construct
  REGION 1..122: note = BGA381 VH AA
EVQLVQSGAEVKKPGASVKVSCKASGYTFTDYWIYWVRQAPGQGLEWMGTIDTSDHKTSYNQKFRGRVTMTRDTSTSTVYMELSSLRSEDTAVYYCARDFIITVNYPVVDYWGQGTLVTVSS

SEQ ID NO: 21 - protein, 106 aa, synthetic construct
  REGION 1..106: note = BGA381 VL AA
QIVLTQSPATLSLSPGERATLSCSAGSSVNYMHWFQQKPGQAPRLLIYSTSNLASGVPARFSGSGSGTDYTLTISSLEPEDFAVYYCQQRSSLPFTFGQGTKLEIK

SEQ ID NO: 22 - DNA, 366 bp, Synthetic construct
  misc_feature 1..366: note = BGA381 VH DNA
  source 1..366: mol_type = other DNA
gaggtgcagctggtgcagagcggcgccgaggtgaagaagcccggcgctagcgtgaaggtgagctgcaaggctagcggctacaccttcaccgactactggatctactgggtgagacaagcccccggccaaggcctggagtggatgggcaccatcgacacaagcgaccacaagacaagctacaatcagaagttcagaggcagagtgaccatgacaagagacacaagcacaagcaccgtgtacatggagctgagcagcctgagaagcgaggacaccgccgtgtactactgcgctagagacttcatcatcaccgtgaactaccccgtggtggactactggggccaaggcaccctggtgaccgtgagcagc

SEQ ID NO: 23 - DNA, 318 bp, Synthetic construct
  misc_feature 1..318: note = BGA381 VL DNA
  source 1..318: mol_type = other DNA
cagatcgtgctgacacagagccccgccaccctgagcctgagccccggcgaaagagccaccctgagctgcagcgccggcagcagcgtgaactacatgcactggtttcagcagaagcccggccaagcccctagactgctgatctacagcacaagcaacctggctagcggcgtgcccgctagattcagcggcagcggcagcggcaccgactacaccctgaccatcagcagcctggagcccgaggacttcgccgtgtactactgtcagcagagaagcagcctgcccttcaccttcggccaaggcaccaagctggagatcaag

SEQ ID NO: 24 - protein, 5 aa, synthetic construct
  REGION 1..5: note = BGA382 HCDR1 (Kabat)
DYWIY

SEQ ID NO: 25 - protein, 17 aa, synthetic construct
  REGION 1..17: note = BGA382 HCDR2 (Kabat)
TIDTSDHKTSYNQKFRG

SEQ ID NO: 26 - protein, 13 aa, synthetic construct
  REGION 1..13: note = BGA382 HCDR3 (Kabat)
DFIITVNYPVVDY

SEQ ID NO: 27 - protein, 10 aa, synthetic construct
  REGION 1..10: note = BGA382 LCDR1 (Kabat)
SAGSSVNYLH

SEQ ID NO: 28 - protein, 7 aa, synthetic construct
  REGION 1..7: note = BGA382 LCDR2 (Kabat)
STSNLAS

SEQ ID NO: 29 - protein, 9 aa, synthetic construct
  REGION 1..9: note = BGA382 LCDR3 (Kabat)
QQRSSLPFT

SEQ ID NO: 30 - protein, 122 aa, synthetic construct; identical to SEQ ID NO: 20
  REGION 1..122: note = BGA382 VH AA

SEQ ID NO: 31 - protein, 106 aa, synthetic construct
  REGION 1..106: note = BGA382 VL AA
QNVLTQSPATLSLSPGERATLSCSAGSSVNYLHWFQQKPGQAPRLLIYSTSNLASGVPARFSGSGSGTDYTLTISSLEPEDFAVYYCQQRSSLPFTFGQGTKLEIK

SEQ ID NO: 32 - DNA, 366 bp, Synthetic construct; identical to SEQ ID NO: 22
  misc_feature 1..366: note = BGA382 VH DNA
  source 1..366: mol_type = other DNA

SEQ ID NO: 33 - DNA, 318 bp, Synthetic construct
  misc_feature 1..318: note = BGA382 VL DNA
  source 1..318: mol_type = other DNA
cagaacgtgctgacacagagccccgccaccctgagcctgagccccggcgaaagagccaccctgagctgcagcgccggcagcagcgtgaactacctgcactggtttcagcagaagcccggccaagcccctagactgctgatctacagcacaagcaacctggctagcggcgtgcccgctagattcagcggcagcggcagcggcaccgactacaccctgaccatcagcagcctggagcccgaggacttcgccgtgtactactgtcagcagagaagcagcctgcccttcaccttcggccaaggcaccaagctggagatcaag

SEQ ID NO: 34 - protein, 5 aa, synthetic construct
  REGION 1..5: note = BGA383 HCDR1 (Kabat)
DYWIY

SEQ ID NO: 35 - protein, 17 aa, synthetic construct
  REGION 1..17: note = BGA383 HCDR2 (Kabat)
TIDTSDAKTSYNQKFRG

SEQ ID NO: 36 - protein, 13 aa, synthetic construct
  REGION 1..13: note = BGA383 HCDR3 (Kabat)
DFIITVNYPVVDY

SEQ ID NO: 37 - protein, 10 aa, synthetic construct
  REGION 1..10: note = BGA383 LCDR1 (Kabat)
SAGSSVNYMH

SEQ ID NO: 38 - protein, 7 aa, synthetic construct
  REGION 1..7: note = BGA383 LCDR2 (Kabat)
STSNLAS

SEQ ID NO: 39 - protein, 9 aa, synthetic construct
  REGION 1..9: note = BGA383 LCDR3 (Kabat)
QQRSSLPFT

SEQ ID NO: 40 - protein, 122 aa, synthetic construct
  REGION 1..122: note = BGA383 VH AA
EVQLVQSGAEVKKPGASVKVSCKASGYTFTDYWIYWVRQAPGQGLEWMGTIDTSDAKTSYNQKFRGRVTMTRDTSTSTVYMELSSLRSEDTAVYYCARDFIITVNYPVVDYWGQGTLVTVSS

SEQ ID NO: 41 - protein, 106 aa, synthetic construct; identical to SEQ ID NO: 21
  REGION 1..106: note = BGA383 VL AA

SEQ ID NO: 42 - DNA, 366 bp, Synthetic construct
  misc_feature 1..366: note = BGA383 VH DNA
  source 1..366: mol_type = other DNA
gaggtgcagctggtgcagagcggcgccgaggtgaagaagcccggcgctagcgtgaaggtgagctgcaaggctagcggctacaccttcaccgactactggatctactgggtgagacaagcccccggccaaggcctggagtggatgggcaccatcgacacaagcgacgccaagacaagctacaatcagaagttcagaggcagagtgaccatgacaagagacacaagcacaagcaccgtgtacatggagctgagcagcctgagaagcgaggacaccgccgtgtactactgcgctagagacttcatcatcaccgtgaactaccccgtggtggactactggggccaaggcaccctggtgaccgtgagcagc

SEQ ID NO: 43 - DNA, 318 bp, Synthetic construct; identical to SEQ ID NO: 33
  misc_feature 1..318: note = BGA383 VL DNA
  source 1..318: mol_type = other DNA

SEQ ID NO: 44 - protein, 5 aa, synthetic construct
  REGION 1..5: note = BGA384 HCDR1 (Kabat)
DYWIY

SEQ ID NO: 45 - protein, 17 aa, synthetic construct
  REGION 1..17: note = BGA384 HCDR2 (Kabat)
TIDTSEHKTSYNQKFRG

SEQ ID NO: 46 - protein, 13 aa, synthetic construct
  REGION 1..13: note = BGA384 HCDR3 (Kabat/ Chothia)
DFIITVNYPVVDY

SEQ ID NO: 47 - protein, 10 aa, synthetic construct
  REGION 1..10: note = BGA384 LCDR1 (Kabat/ Chothia)
SAGSSVNYMH

SEQ ID NO: 48 - protein, 7 aa, synthetic construct
  REGION 1..7: note = BGA384 LCDR2 (Kabat/ Chothia)
STSNLAS

SEQ ID NO: 49 - protein, 9 aa, synthetic construct
  REGION 1..9: note = BGA384 LCDR3 (Kabat/ Chothia)
QQRSSLPFT

SEQ ID NO: 50 - protein, 122 aa, synthetic construct
  REGION 1..122: note = BGA384 VH AA
EVQLVQSGAEVKKPGASVKVSCKASGYTFTDYWIYWVRQAPGQGLEWMGTIDTSEHKTSYNQKFRGRVTMTRDTSTSTVYMELSSLRSEDTAVYYCARDFIITVNYPVVDYWGQGTLVTVSS

SEQ ID NO: 51 - protein, 106 aa, synthetic construct; identical to SEQ ID NO: 21
  REGION 1..106: note = BGA384 VL AA

SEQ ID NO: 52 - DNA, 366 bp, Synthetic construct
  misc_feature 1..366: note = BGA384 VH DNA
  source 1..366: mol_type = other DNA
gaggtgcagctggtgcagagcggcgccgaggtgaagaagcccggcgctagcgtgaaggtgagctgcaaggctagcggctacaccttcaccgactactggatctactgggtgagacaagcccccggccaaggcctggagtggatgggcaccatcgacacaagcgagcacaagacaagctacaatcagaagttcagaggcagagtgaccatgacaagagacacaagcacaagcaccgtgtacatggagctgagcagcctgagaagcgaggacaccgccgtgtactactgcgctagagacttcatcatcaccgtgaactaccccgtggtggactactggggccaaggcaccctggtgaccgtgagcagc

SEQ ID NO: 53 - DNA, 318 bp, Synthetic construct; identical to SEQ ID NO: 33
  misc_feature 1..318: note = BGA384 VL DNA
  source 1..318: mol_type = other DNA

SEQ ID NO: 54 - protein, 5 aa, synthetic construct
  REGION 1..5: note = BGA86 HCDR1 (Kabat)
SYGMS

SEQ ID NO: 55 - protein, 17 aa, synthetic construct
  REGION 1..17: note = BGA86 HCDR2 (Kabat)
EISSGGTYTYYPDTVTG

SEQ ID NO: 56 - protein, 6 aa, synthetic construct
  REGION 1..6: note = BGA86 HCDR3 (Kabat)
GDPFGY

SEQ ID NO: 57 - protein, 11 aa, synthetic construct
  REGION 1..11: note = BGA86 LCDR1 (Kabat)
KASQDVMTAVA

SEQ ID NO: 58 - protein, 7 aa, synthetic construct
  REGION 1..7: note = BGA86 LCDR2 (Kabat)
STSYRFT

SEQ ID NO: 59 - protein, 9 aa, synthetic construct
  REGION 1..9: note = BGA86 LCDR3 (Kabat)
QQHYSTPRT

SEQ ID NO: 60 - protein, 115 aa, synthetic construct
  REGION 1..115: note = BGA86 VH AA
EVQLQESGGGSVKPGGSLQLSCAASGFTFSSYGMSWIRQSPEKKLEWVAEISSGGTYTYYPDTVTGRFTISRDNAKNTLYLEMSSLRSEDTAMYYCARGDPFGYWGQGTLVTVSA

SEQ ID NO: 61 - protein, 107 aa, synthetic construct
  REGION 1..107: note = BGA86 VL AA
DVVMTQSHKFVSTSVGGRVSITCKASQDVMTAVAWYQQKPGQSPKLLIYSTSYRFTGVPDRFTGSGSGTDFTLTINGVQAEDLAVYSCQQHYSTPRTFGGGTKLEIK

SEQ ID NO: 62 - DNA, 345 bp, Synthetic construct
  misc_feature 1..345: note = BGA86 VH DNA
  source 1..345: mol_type = other DNA
gaggtgcagctgcaagagtccggcgggggcagcgtgaagcctgggggctccctgcagctgagctgcgccgcctccggcttcaccttcagcagctacggcatgagctggatcagacagagccccgagaagaagctggagtgggtggccgagatcagcagcggcggcacctacacctactaccccgacaccgtgaccggcagattcaccatcagcagagacaacgccaagaacaccctgtacctggagatgagcagcctgagaagcgaggacaccgccatgtactactgcgctagaggcgaccccttcggctactggggccaaggcaccctggtgaccgtgagcgcc

SEQ ID NO: 63 - DNA, 321 bp, Synthetic construct
  misc_feature 1..321: note = BGA86 VL DNA
  source 1..321: mol_type = other DNA
gacgtggtgatgacacagagccacaagttcgtgagcacaagcgtgggcggcagagtgagcatcacctgcaaggcctcccaagacgtgatgaccgccgtggcctggtatcagcagaagcccgggcagagccccaagctgctgatctacagcacaagctacagattcaccggcgtgcccgaccggttcaccggcagcggcagcggcaccgacttcaccctgaccatcaacggcgtgcaagccgaggacctggccgtgtacagctgtcagcagcactacagcacccctagaaccttcggcgggggcaccaagctggagatcaag

SEQ ID NO: 64 - protein, 5 aa, synthetic construct
  REGION 1..5: note = BGA861 HCDR1 (Kabat)
SYGIS

SEQ ID NO: 65 - protein, 17 aa, synthetic construct
  REGION 1..17: note = BGA861 HCDR2 (Kabat)
EISSGGTYTYYPDTVTG

SEQ ID NO: 66 - protein, 6 aa, synthetic construct
  REGION 1..6: note = BGA861 HCDR3 (Kabat)
GDPFGY

SEQ ID NO: 67 - protein, 11 aa, synthetic construct
  REGION 1..11: note = BGA861 LCDR1 (Kabat)
KASQDVMTAVA

SEQ ID NO: 68 - protein, 7 aa, synthetic construct
  REGION 1..7: note = BGA861 LCDR2 (Kabat)
STSYRFT

SEQ ID NO: 69 - protein, 9 aa, synthetic construct
  REGION 1..9: note = BGA861 LCDR3 (Kabat)
QQHYSTPRT

SEQ ID NO: 70 - protein, 115 aa, synthetic construct
  REGION 1..115: note = BGA861 VH AA
EVQLVESGGGLVKPGGSLRLSCAASGFTFSSYGISWVRQAPGKGLEWVSEISSGGTYTYYPDTVTGRFTISRDNAKNTLYLQMSSLRAEDTAVYYCARGDPFGYWGQGTLVTVSS

SEQ ID NO: 71 - protein, 107 aa, synthetic construct
  REGION 1..107: note = BGA861 VL AA
DIQMTQSPSSLSASVGDRVTITCKASQDVMTAVAWYQQKPGKAPKLLIYSTSYRFTGVPSRFSGSGSGTDFTLTISSLQPEDFATYYCQQHYSTPRTFGGGTKVEIK

SEQ ID NO: 72 - DNA, 345 bp, Synthetic construct
  misc_feature 1..345: note = BGA861 VH DNA
  source 1..345: mol_type = other DNA
gaggtgcagctggtggaatccggcgggggcctggtgaaacccggcggcagcctgagactgagctgcgccgctagcggcttcaccttcagcagctacggcatcagctgggtgagacaagcccccggcaagggcctggagtgggtgagcgagatcagcagcggcggcacctacacctactaccccgacaccgtgaccggcagattcaccatcagcagagacaacgccaagaacaccctgtacctgcagatgagcagcctgagagccgaggacaccgccgtgtactactgcgctagaggcgaccccttcggctactggggccaaggcaccctggtgaccgtgagcagc

SEQ ID NO: 73 - DNA, 321 bp, Synthetic construct
  misc_feature 1..321: note = BGA861 VL DNA
  source 1..321: mol_type = other DNA
gacattcagatgacacagagccctagcagcctgtccgctagcgtgggcgacagagtgaccatcacctgcaaggctagccaagacgtgatgaccgccgtggcctggtatcagcagaagcccggcaaggcccccaagctgctgatctacagcacaagctacagattcaccggcgtgcctagcagattcagcggcagcggcagcggcaccgacttcaccctgaccatcagcagcctgcagcccgaggacttcgccacctactactgtcagcagcactacagcacccctagaaccttcggcgggggcaccaaggtggagatcaag

SEQ ID NO: 74 - protein, 5 aa, synthetic construct
  REGION 1..5: note = BGA862 HCDR1 (Kabat)
SYGLS

SEQ ID NO: 75 - protein, 17 aa, synthetic construct
  REGION 1..17: note = BGA862 HCDR2 (Kabat)
EISSGGTYTYYPDTVTG

SEQ ID NO: 76 - protein, 6 aa, synthetic construct
  REGION 1..6: note = BGA862 HCDR3 (Kabat)
GDPFGY

SEQ ID NO: 77 - protein, 11 aa, synthetic construct
  REGION 1..11: note = BGA862 LCDR1 (Kabat)
KASQDVMTAVA

SEQ ID NO: 78 - protein, 7 aa, synthetic construct
  REGION 1..7: note = BGA862 LCDR2 (Kabat)
STSYRFT

SEQ ID NO: 79 - protein, 9 aa, synthetic construct
  REGION 1..9: note = BGA862 LCDR3 (Kabat)
QQHYSTPRT

SEQ ID NO: 80 - protein, 115 aa, synthetic construct
  REGION 1..115: note = BGA862 VH AA
EVQLVESGGGLVKPGGSLRLSCAASGFTFSSYGLSWVRQAPGKGLEWVSEISSGGTYTYYPDTVTGRFTISRDNAKNTLYLQMSSLRAEDTAVYYCARGDPFGYWGQGTLVTVSS

SEQ ID NO: 81 - protein, 107 aa, synthetic construct; identical to SEQ ID NO: 71
  REGION 1..107: note = BGA862 VL AA

SEQ ID NO: 82 - DNA, 345 bp, Synthetic construct
  misc_feature 1..345: note = BGA862 VH DNA
  source 1..345: mol_type = other DNA
gaggtgcagctggtggaatccggcgggggcctggtgaaacccggcggcagcctgagactgagctgcgccgctagcggcttcaccttcagcagctacggcctcagctgggtgagacaagcccccggcaagggcctggagtgggtgagcgagatcagcagcggcggcacctacacctactaccccgacaccgtgaccggcagattcaccatcagcagagacaacgccaagaacaccctgtacctgcagatgagcagcctgagagccgaggacaccgccgtgtactactgcgctagaggcgaccccttcggctactggggccaaggcaccctggtgaccgtgagcagc

SEQ ID NO: 83 - DNA, 321 bp, Synthetic construct; identical to SEQ ID NO: 73
  misc_feature 1..321: note = BGA862 VL DNA
  source 1..321: mol_type = other DNA

SEQ ID NO: 84 - protein, 5 aa, synthetic construct
  REGION 1..5: note = BGA12 HCDR1 (Kabat)
DYAMH

SEQ ID NO: 85 - protein, 17 aa, synthetic construct
  REGION 1..17: note = BGA12 HCDR2 (Kabat)
VVLTHNDNTNYNQKFKA

SEQ ID NO: 86 - protein, 16 aa, synthetic construct
  REGION 1..16: note = BGA12 HCDR3 (Kabat)
EVYYDFDDGNYFPMDY

SEQ ID NO: 87 - protein, 17 aa, synthetic construct
  REGION 1..17: note = BGA12 LCDR1 (Kabat)
KSSQSLLYSDNQKNYLA

SEQ ID NO: 88 - protein, 7 aa, synthetic construct
  REGION 1..7: note = BGA12 LCDR2 (Kabat)
WASTRES

SEQ ID NO: 89 - protein, 8 aa, synthetic construct
  REGION 1..8: note = BGA12 LCDR3 (Kabat)
QQYYSYHT

SEQ ID NO: 90 - protein, 125 aa, synthetic construct
  REGION 1..125: note = BGA12 VH AA
QVQLQQSGPEVVRPGVSVKISCKGSGYTFTDYAMHWVKQSHGKSLEWIGVVLTHNDNTNYNQKFKAKATMTVDRSSSTAYMELARLTSEDSAIYYCAREVYYDFDDGNYFPMDYWGQGTSVTVSS

SEQ ID NO: 91 - protein, 112 aa, synthetic construct
  REGION 1..112: note = BGA12 VL AA
DIVMSQSPSSLAVSVGEKVTMSCKSSQSLLYSDNQKNYLAWYQQKPGQSPKLLIYWASTRESGVPVRFTGSGSGTDFTLTISSVKAEDLAVYYCQQYYSYHTFGGGTKLEIK

SEQ ID NO: 92 - DNA, 444 bp, Synthetic construct
  misc_feature 1..444: note = BGA12 VH DNA
  source 1..444: mol_type = other DNA
ggatccgccaccatggagtttgggctgagctggctttttcttgtcgcgattcttaagggtgtccagtgccaagtgcagctgcagcagagcggccccgaggtggtgagacccggcgtgagcgtgaagatcagctgcaagggcagcggctacaccttcaccgactacgccatgcactgggtgaagcagagccacggcaagagcctggagtggatcggcgtggtgctgacccacaacgacaacaccaactacaatcagaagttcaaggccaaggccaccatgaccgtggacagaagcagcagcaccgcctacatggagctggctagactgacaagcgaggacagcgccatctactactgcgctagagaggtgtactacgacttcgacgacggcaactacttccccatggactactggggccaaggcacaagcgtgaccgtgagcagc

SEQ ID NO: 93 - DNA, 336 bp, Synthetic construct
  misc_feature 1..336: note = BGA12 VL DNA
  source 1..336: mol_type = other DNA
gacatcgtgatgagccaaagcccctcctccctggccgtgagcgtgggcgagaaggtgaccatgagctgcaagagcagccaaagcctgctgtacagcgacaatcagaagaactacctggcctggtatcagcagaagcccgggcagagccccaaactcctgatctactgggctagcacaagagagagcggcgtgcccgtgagattcaccggcagcggcagcggcaccgacttcaccctgaccatcagcagcgtgaaggccgaggacctggccgtgtactactgtcagcagtactacagctaccacaccttcggcgggggcaccaagctggagatcaag

SEQ ID NO: 94 - protein, 5 aa, synthetic construct
  REGION 1..5: note = BGA121 HCDR1 (Kabat)
DYAMH

SEQ ID NO: 95 - protein, 17 aa, synthetic construct
  REGION 1..17: note = BGA121 HCDR2 (Kabat)
VVLTHNDNTNYNQKFKA

SEQ ID NO: 96 - protein, 16 aa, synthetic construct
  REGION 1..16: note = BGA121 HCDR3 (Kabat)
EVYYDFDDGNYFPMDY

SEQ ID NO: 97 - protein, 17 aa, synthetic construct
  REGION 1..17: note = BGA121 LCDR1 (Kabat)
KSSQSLLYSDNQKNYLA

SEQ ID NO: 98 - protein, 7 aa, synthetic construct
  REGION 1..7: note = BGA121 LCDR2 (Kabat)
WASTRES

SEQ ID NO: 99 - protein, 8 aa, synthetic construct
  REGION 1..8: note = BGA121 LCDR3 (Kabat)
QQYYSYHT

SEQ ID NO: 100 - protein, 125 aa, synthetic construct
  REGION 1..125: note = BGA121 VH AA
QVQLVQSGAEVKKPGASVKVSCKASGYTFTDYAMHWVRQAPGQSLEWMGVVLTHNDNTNYNQKFKARVTMTVDTSASTAYMELSSLRSEDTAVYYCAREVYYDFDDGNYFPMDYWGQGTLVTVSS

SEQ ID NO: 101 - protein, 112 aa, synthetic construct
  REGION 1..112: note = BGA121 VL AA
DIVMTQSPDSLAVSLGERATINCKSSQSLLYSDNQKNYLAWYQQKPGQPPKLLIYWASTRESGVPDRFSGSGSGTDFTLTISSLQAEDVAVYYCQQYYSYHTFGGGTKLEIK

SEQ ID NO: 102 - DNA, 375 bp, Synthetic construct
  misc_feature 1..375: note = BGA121 VH DNA
  source 1..375: mol_type = other DNA
caagtgcagctggtgcagagcggcgccgaggtgaagaagcccggcgctagcgtgaaggtgagctgcaaggctagcggctacaccttcaccgactacgccatgcactgggtgagacaagcccccgggcagagcctggagtggatgggcgtggtgctgacccacaacgacaacaccaactacaatcagaagttcaaggctagagtgaccatgaccgtggacacatccgccagcaccgcctacatggagctgagcagcctgagaagcgaggacaccgccgtgtactactgcgctagagaggtgtactacgacttcgacgacggcaactacttccccatggactactggggccaaggcaccctggtgaccgtgagcagc

SEQ ID NO: 103 - DNA, 336 bp, Synthetic construct
  misc_feature 1..336: note = BGA121 VL DNA
  source 1..336: mol_type = other DNA
gacatcgtgatgacacagagccccgacagcctggccgtgagcctgggcgagagagccaccatcaactgcaagagcagccaaagcctgctgtacagcgacaatcagaagaactacctggcctggtatcagcagaagcccgggcagccccccaagctgctgatctactgggctagcacaagagagagcggcgtgcccgacagattcagcggcagcggcagcggcaccgacttcaccctgaccatcagcagcctgcaagccgaggacgtggccgtgtactactgtcagcagtactacagctaccacaccttcggcgggggcaccaagctggagatcaag

SEQ ID NO: 104 - protein, 5 aa, synthetic construct
  REGION 1..5: note = BGA122 HCDR1 (Kabat)
DYAMH

SEQ ID NO: 105 - protein, 17 aa, synthetic construct
  REGION 1..17: note = BGA122 HCDR2 (Kabat)
VVLTHNDNTNYNQKFKA

SEQ ID NO: 106 - protein, 16 aa, synthetic construct
  REGION 1..16: note = BGA122 HCDR3 (Kabat)
EVYYDFDDGNYFPMDY

SEQ ID NO: 107 - protein, 17 aa, synthetic construct
  REGION 1..17: note = BGA122 LCDR1 (Kabat)
KSSQSLLYSDNQKNYLA

SEQ ID NO: 108 - protein, 7 aa, synthetic construct
  REGION 1..7: note = BGA122 LCDR2 (Kabat)
WASTRES

SEQ ID NO: 109 - protein, 8 aa, synthetic construct
  REGION 1..8: note = BGA122 LCDR3 (Kabat)
QQYYSYHT

SEQ ID NO: 110 - protein, 125 aa, synthetic construct; identical to SEQ ID NO: 100
  REGION 1..125: note = BGA122 VH AA

SEQ ID NO: 111 - protein, 112 aa, synthetic construct
  REGION 1..112: note = BGA122 VL AA
DIVMTQSPDSLAVSLGERATINCKSSQSLLYSDNQKNYLAWYQQKPGQSPKLLIYWASTRESGVPDRFSGSGSGTDFTLTISSLQAEDVAVYYCQQYYSYHTFGGGTKVEIK

SEQ ID NO: 112 - DNA, 375 bp, Synthetic construct; identical to SEQ ID NO: 102
  misc_feature 1..375: note = BGA122 VH DNA
  source 1..375: mol_type = other DNA

SEQ ID NO: 113 - DNA, 337 bp, Synthetic construct
  misc_feature 1..337: note = BGA122 VL DNA
  source 1..337: mol_type = other DNA
gacatcgtgatgacacagagccccgacagcctggccgtgagcctgggcgagagagccaccatcaactgcaagagcagccaaagcctgctgtacagcgacaatcagaagaactacctggcctggtatcagcagaagcccgggcagagccccaagctgctgatctactgggctagcacaagagagagcggcgtgcccgacagattcagcggcagcggcagcggcaccgacttcaccctgaccatcagcagcctgcaagccgaggacgtggccgtgtactactgtcagcagtactacagctaccacaccttcggcgggggcaccaaggtggagatcaagc

SEQ ID NO: 114 - protein, 5 aa, synthetic construct
  REGION 1..5: note = BGA50 HCDR1 (Kabat)
GYFLN

SEQ ID NO: 115 - protein, 17 aa, synthetic construct
  REGION 1..17: note = BGA50 HCDR2 (Kabat)
RISPYNGDTFYNQKFKG

SEQ ID NO: 116 - protein, 14 aa, synthetic construct
  REGION 1..14: note = BGA50 HCDR3 (Kabat)
GRSGSFTLFYAMDY

SEQ ID NO: 117 - protein, 17 aa, synthetic construct
  REGION 1..17: note = BGA50 LCDR1 (Kabat)
KSSQSLFHSGNQKNYLA

SEQ ID NO: 118 - protein, 7 aa, synthetic construct
  REGION 1..7: note = BGA50 LCDR2 (Kabat)
WASTRES

SEQ ID NO: 119 - protein, 9 aa, synthetic construct
  REGION 1..9: note = BGA50 LCDR3 (Kabat)
QQYYSYPIT

SEQ ID NO: 120 - protein, 121 aa, synthetic construct
  REGION 1..121: note = BGA50 VH AA
EVQLQQSGPEPVKPGASVKISCKASGYSFTGYFLNWVKQSHGKSLEWIGRISPYNGDTFYNQKFKGKATLTVDKSSSTAHMELLSLTSEDSAVYYCGRSGSFTLFYAMDYWGQGTSVTVSS

SEQ ID NO: 121 - protein, 113 aa, synthetic construct
  REGION 1..113: note = BGA50 VL AA
DIVMSQSPSSLAVSVGENITMTCKSSQSLFHSGNQKNYLAWYQQKPGQSPKLLIYWASTRESGVPDRFTGSGSETDFTLTISSVKAEDLAVYFCQQYYSYPITFGAGTKLELK

SEQ ID NO: 122 - DNA, 363 bp, Synthetic construct
  misc_feature 1..363: note = BGA50 VH DNA
  source 1..363: mol_type = other DNA
gaggttcagctgcagcagtctggacctgaaccggtgaagcctggggcttcagtgaagatatcctgcaaggcttctggttactcatttactggctactttttgaactgggtgaagcagagccatggaaagagccttgagtggattggacgtattagtccttacaatggtgatactttctacaaccagaagttcaagggcaaggccacattgactgtagacaaatcctctagcacagcccacatggagctcctgagcctgacatctgaggactctgcagtctattattgtggaagatcgggttccttcactcttttctatgctatggactactggggtcaaggaacctcagtcaccgtctcctca

SEQ ID NO: 123 - DNA, 339 bp, Synthetic construct
  misc_feature 1..339: note = BGA50 VL DNA
  source 1..339: mol_type = other DNA
gacattgtgatgtcacagtctccatcctccctagctgtgtcagttggagagaatattactatgacctgcaagtccagtcagagccttttccatagtggcaatcaaaagaactacttggcctggtaccagcagaaaccagggcagtctcctaaactgctgatttactgggcatccactagggaatctggggtccctgatcgcttcacaggcagtggatctgagacagatttcactctcaccatcagcagtgtgaaggctgaagacctggcagtttatttctgtcagcaatattatagctatcctatcacgttcggtgctgggaccaagctggagctgaaa

SEQ ID NO: 124 - protein, 5 aa, synthetic construct
  REGION 1..5: note = BGA501 HCDR1 (Kabat)
GYFLN

SEQ ID NO: 125 - protein, 17 aa, synthetic construct
  REGION 1..17: note = BGA501 HCDR2 (Kabat)
RISPYQGDTFYNQKFKG

SEQ ID NO: 126 - protein, 14 aa, synthetic construct
  REGION 1..14: note = BGA501 HCDR3 (Kabat)
GRSGSFTLFYAIDY

SEQ ID NO: 127 - protein, 17 aa, synthetic construct
  REGION 1..17: note = BGA501 LCDR1 (Kabat)
KSSQSLFHSGNQKNYLA

SEQ ID NO: 128 - protein, 7 aa, synthetic construct
  REGION 1..7: note = BGA501 LCDR2 (Kabat)
WASTRES

SEQ ID NO: 129 - protein, 9 aa, synthetic construct
  REGION 1..9: note = BGA501 LCDR3 (Kabat)
QQYYSYPIT

SEQ ID NO: 130 - protein, 121 aa, synthetic construct
  REGION 1..121: note = BGA501 VH AA
QVQLVQSGAEVKKPGASVKVSCKASGYSFTGYFLNWVRQAPGQGLEWIGRISPYQGDTFYNQKFKGRVTMTTDKSTSTAYMELRSLRSDDTAVYYCGRSGSFTLFYAIDYWGQGTLVTVSS

SEQ ID NO: 131 - protein, 113 aa, synthetic construct
  REGION 1..113: note = BGA501 VL AA
DIVMTQSPSSLAVSLGERATINCKSSQSLFHSGNQKNYLAWYQQKPGQSPKLLIYWASTRESGVPDRFSGSGSGTDFTLTISSLQAEDVAVYYCQQYYSYPITFGQGTKLEIK

SEQ ID NO: 132 - DNA, 361 bp, Synthetic construct
  misc_feature 1..361: note = BGA501 VH DNA
  source 1..361: mol_type = other DNA
caagtgcagctggtgcagagcggcgccgaggtgaagaagcccggcgctagcgtgaaggtgagctgcaaggctagcggctacagcttcaccggctacttcctgaactgggtgagacaagcccccggccaaggcctggagtggatcggcagaatcagcccctaccaaggcgacaccttctacaatcagaagttcaagggcagagtgaccatgaccaccgacaagagcacaagcaccgcctacatggagctgagaagcctgagaagcgacgacaccgccgtgtactactgcggcagaagcggcagcttcaccctgttctacgccatcgactactggggccaaggcaccctggtgaccgtgagca

SEQ ID NO: 133 - DNA, 339 bp, Synthetic construct
  misc_feature 1..339: note = BGA501 VL DNA
  source 1..339: mol_type = other DNA
gacatcgtgatgacacagagccctagcagcctggccgtgagcctgggcgagagagccaccatcaactgcaagagcagccaaagcctgttccacagcggcaatcagaagaactacctggcctggtatcagcagaagcccgggcagagccccaagctgctgatctactgggctagcacaagagagagcggcgtgcccgacagattcagcggcagcggcagcggcaccgacttcaccctgaccatcagcagcctgcaagccgaggacgtggccgtgtactactgtcagcagtactacagctaccccatcaccttcggccaaggcaccaagctggagatcaag

SEQ ID NO: 134 - protein, 5 aa, synthetic construct
  REGION 1..5: note = BGA502 HCDR1 (Kabat)
GYFLN

SEQ ID NO: 135 - protein, 17 aa, synthetic construct
  REGION 1..17: note = BGA502 HCDR2 (Kabat)
RISPYQGDTFYNQKFKG

SEQ ID NO: 136 - protein, 14 aa, synthetic construct
  REGION 1..14: note = BGA502 HCDR3 (Kabat)
GRSGSFTLFYALDY

SEQ ID NO: 137 - protein, 17 aa, synthetic construct
  REGION 1..17: note = BGA502 LCDR1 (Kabat)
KSSQSLFHSGNQKNYLA

SEQ ID NO: 138 - protein, 7 aa, synthetic construct
  REGION 1..7: note = BGA502 LCDR2 (Kabat)
WASTRES

SEQ ID NO: 139 - protein, 9 aa, synthetic construct
  REGION 1..9: note = BGA502 LCDR3 (Kabat)
QQYYSYPIT

SEQ ID NO: 140 - protein, 121 aa, synthetic construct
  REGION 1..121: note = BGA502 VH AA
QVQLVQSGAEVKKPGASVKVSCKASGYSFTGYFLNWVRQAPGQGLEWIGRISPYQGDTFYNQKFKGRVTMTTDKSTSTAYMELRSLRSDDTAVYYCGRSGSFTLFYALDYWGQGTLVTVSS

SEQ ID NO: 141 - protein, 113 aa, synthetic construct; identical to SEQ ID NO: 131
  REGION 1..113: note = BGA502 VL AA

SEQ ID NO: 142 - DNA, 363 bp, Synthetic construct
  misc_feature 1..363: note = BGA502 VH DNA
  source 1..363: mol_type = other DNA
caagtgcagctggtgcagagcggcgccgaggtgaagaagcccggcgctagcgtgaaggtgagctgcaaggctagcggctacagcttcaccggctacttcctgaactgggtgagacaagcccccggccaaggcctggagtggatcggcagaatcagcccctaccaaggcgacaccttctacaatcagaagttcaagggcagagtgaccatgaccaccgacaagagcacaagcaccgcctacatggagctgagaagcctgagaagcgacgacaccgccgtgtactactgcggcagaagcggcagcttcaccctgttctacgccatcgactactggggccaaggcaccctggtgaccgtgagcagc

SEQ ID NO: 143 - DNA, 339 bp, Synthetic construct; identical to SEQ ID NO: 133
  misc_feature 1..339: note = BGA502 VL DNA
  source 1..339: mol_type = other DNA

SEQ ID NO: 144 - protein, 5 aa, synthetic construct
  REGION 1..5: note = BGA503 HCDR1 (Kabat)
GYFLN

SEQ ID NO: 145 - protein, 17 aa, synthetic construct
  REGION 1..17: note = BGA503 HCDR2 (Kabat)
RISPYQGDTFYNQKFKG

SEQ ID NO: 146 - protein, 14 aa, synthetic construct
  REGION 1..14: note = BGA503 HCDR3 (Kabat)
GRSGSFTLFYALDY

SEQ ID NO: 147 - protein, 17 aa, synthetic construct
  REGION 1..17: note = BGA503 LCDR1 (Kabat)
KSSQSLFHSGNQKNYLA

SEQ ID NO: 148 - protein, 7 aa, synthetic construct
  REGION 1..7: note = BGA503 LCDR2 (Kabat)
WASTRES

SEQ ID NO: 149 - protein, 9 aa, synthetic construct
  REGION 1..9: note = BGA503 LCDR3 (Kabat)
QQYYSYPIT

SEQ ID NO: 150 - protein, 121 aa, synthetic construct
  REGION 1..121: note = BGA503 VH AA
QVQLVQSGAEVKKPGASVKVSCKASGYSFTGYFLNWVRQAPGQGLEWIGRISPYQGDTFYNQKFKGRVTMTVDKSTSTAYMELRSLRSDDTAVYYCGRSGSFTLFYALDYWGQGTLVTVSS

SEQ ID NO: 151 - protein, 113 aa, synthetic construct; identical to SEQ ID NO: 131
  REGION 1..113: note = BGA503 VL AA

SEQ ID NO: 152 - DNA, 363 bp, Synthetic construct
  misc_feature 1..363: note = BGA503 VH DNA
  source 1..363: mol_type = other DNA
caagtgcagctggtgcagagcggcgccgaggtgaagaagcccggcgctagcgtgaaggtgagctgcaaggctagcggctacagcttcaccggctacttcctgaactgggtgagacaagcccccggccaaggcctggagtggatcggcagaatcagcccctaccaaggcgacaccttctacaatcagaagttcaagggcagagtgaccatgaccgtggacaagagcacaagcaccgcctacatggagctgagaagcctgagaagcgacgacaccgccgtgtactactgcggcagaagcggcagcttcaccctgttctacgccctggactactggggccaaggcaccctggtgaccgtgagcagc

SEQ ID NO: 153 - DNA, 339 bp, Synthetic construct; identical to SEQ ID NO: 133
  misc_feature 1..339: note = BGA503 VL DNA
  source 1..339: mol_type = other DNA

SEQ ID NO: 154 - protein, 131 aa, synthetic construct
  REGION 1..131: note = human PVRIG from amino acid 41 to amino acid 171
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRAD

SEQ ID NO: 155 - protein, 98 aa, synthetic construct
  REGION 1..98: note = BGA38 Human framework (Human IGVH1-46)
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 156 - protein, 95 aa, synthetic construct
  REGION 1..95: note = BGA38 Human framework (Human IGVK3-11)
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 157 - protein, 98 aa, synthetic construct
  REGION 1..98: note = BGA86 Human framework (Human IGVH3-21)
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 158 - protein, 95 aa, synthetic construct
  REGION 1..95: note = BGA86 Human framework (Human IGVH1-39)
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 159 - protein, 98 aa, synthetic construct
  REGION 1..98: note = BGA12 Human framework (Human IGVH1-3)
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 160 - protein, 101 aa, synthetic construct
  REGION 1..101: note = BGA12 Human framework (Human IGVK4-1)
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 161 - protein, 98 aa, synthetic construct
  REGION 1..98: note = BGA50 Human framework (Human IGVH1-18)
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 162 - protein, 101 aa, synthetic construct; identical to SEQ ID NO: 160
  REGION 1..101: note = BGA50 Human framework (Human IGVK4-1)

SEQ ID NO: 163 - protein, 10 aa, synthetic construct
  REGION 1..10: note = Tislelizumab HCDR1
GFSLTSYGVH

SEQ ID NO: 164 - protein, 16 aa, synthetic construct
  REGION 1..16: note = Tislelizumab HCDR1
VIYADGSTNYNPSLKS

SEQ ID NO: 165 - protein, 12 aa, synthetic construct
  REGION 1..12: note = Tislelizumab HCDR3
ARAYGNYWYIDV

SEQ ID NO: 166 - protein, 11 aa, synthetic construct
  REGION 1..11: note = Tislelizumab LCDR1
KSSESVSNDVA

SEQ ID NO: 167 - protein, 7 aa, synthetic construct
  REGION 1..7: note = Tislelizumab LCDR2
YAFHRFT

SEQ ID NO: 168 - protein, 9 aa, synthetic construct
  REGION 1..9: note = Tislelizumab LCDR3
HQAYSSPYT

SEQ ID NO: 169 - protein, 118 aa, synthetic construct
  REGION 1..118: note = Tislelizumab VH
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 170 - protein, 107 aa, synthetic construct
  REGION 1..107: note = Tislelizumab VL
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 171 - protein, 327 aa, synthetic construct
  REGION 1..327: note = Tislelizumab IgG4 constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 172 - protein, 445 aa, synthetic construct
  REGION 1..445: note = Tislelizumab Heavy Chain
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 173 - protein, 214 aa, synthetic construct
  REGION 1..214: note = Tislelizumab Light chain
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 5 aa, synthetic construct
  REGION 1..5: note = Ociperlimab HCDR1
DYYMY

SEQ ID NO: 175 - protein, 17 aa, synthetic construct
  REGION 1..17: note = Ociperlimab HCDR2
YITKGGGSTYYPDSVKG

SEQ ID NO: 176 - protein, 10 aa, synthetic construct
  REGION 1..10: note = Ociperlimab HCDR3
QTNYDFTMDY

SEQ ID NO: 177 - protein, 11 aa, synthetic construct
  REGION 1..11: note = Ociperlimab LCDR1
KASQDVGTSVA

SEQ ID NO: 178 - protein, 7 aa, synthetic construct
  REGION 1..7: note = Ociperlimab LCDR2
WASARHT

SEQ ID NO: 179 - protein, 9 aa, synthetic construct
  REGION 1..9: note = Ociperlimab LCDR3
QQYSSYPLT

SEQ ID NO: 180 - protein, 119 aa, synthetic construct
  REGION 1..119: note = Ociperlimab VH
EVQLVESGGGLVQPGGSLRLSCAASGFTFSDYYMYWVRQAPGKGLEWVAYITKGGGSTYYPDSVKGRFTISRDNAKNTLYLQMNSLRAEDTAVYYCARQTNYDFTMDYWGQGTLVTVSS

SEQ ID NO: 181 - protein, 107 aa, synthetic construct
  REGION 1..107: note = Ociperlimab VL
EIVMTQSPATLSVSPGERATLSCKASQDVGTSVAWYQQKPGQAPRLLIYWASARHTGIPARFSGSGSGTEFTLTISSLQSEDFAVYYCQQYSSYPLTFGGGTKVEIK

SEQ ID NO: 182 - protein, 449 aa, synthetic construct
  REGION 1..449: note = Ociperlimab Heavy chain
EVQLVESGGGLVQPGGSLRLSCAASGFTFSDYYMYWVRQAPGKGLEWVAYITKGGGSTYYPDSVKGRFTISRDNAKNTLYLQMNSLRAEDTAVYYCARQTNYDFTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 214 aa, synthetic construct
  REGION 1..214: note = Ociperlimab Light chain
EIVMTQSPATLSVSPGERATLSCKASQDVGTSVAWYQQKPGQAPRLLIYWASARHTGIPARFSGSGSGTEFTLTISSLQSEDFAVYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 330 aa, synthetic construct
  REGION 1..330: note = Ociperlimab IgG1 WT
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 9 aa, synthetic construct
  REGION 1..9: note = CMV pp65 peptide
NLVPMVATV

SEQ ID NO: 186 - protein, 5 aa, synthetic construct
  REGION 1..5: note = a single G4S segment
GGGGS

SEQ ID NO: 187 - protein, 7 aa, synthetic construct
  REGION 1..7: note = BGA384 HCDR1 (Chothia)
GYTFTDY

SEQ ID NO: 188 - protein, 6 aa, synthetic construct
  REGION 1..6: note = BGA384 HCDR2 (Chothia)
DTSEHK

SEQ ID NO: 189 - protein, 452 aa, synthetic construct
  REGION 1..452: note = BGA384 HC AA
EVQLVQSGAEVKKPGASVKVSCKASGYTFTDYWIYWVRQAPGQGLEWMGTIDTSEHKTSYNQKFRGRVTMTRDTSTSTVYMELSSLRSEDTAVYYCARDFIITVNYPVVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 213 aa, synthetic construct
  REGION 1..213: note = BGA384 LC AA
QIVLTQSPATLSLSPGERATLSCSAGSSVNYMHWFQQKPGQAPRLLIYSTSNLASGVPARFSGSGSGTDYTLTISSLEPEDFAVYYCQQRSSLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0326842 A1
(43) Pub. Date: Oct. 23, 2025

(54) ANTI-PVRIG ANTIBODIES AND METHODS OF USE

(71) Applicant: BeiGene Switzerland GmbH, Basel (CH)

(72) Inventors: Xiaoyan TANG, Beijing (CN); Dan LI, Shanghai (CN); Mengran QIAN, Shanghai (CN); Ruyue Ji, Shanghai (CN); Jie PAN, Shanghai (CN); Xin CHEN, Beijing (CN); Lei JIANG, Beijing (CN); Xiao DING, Beijing (CN); Hui LI, Beijing (CN); Jing SONG, Beijing (CN); Jian Sun, Beijing (CN); Meiqi YI, Beijing (CN)

(73) Assignee: BeiGene Switzerland GmbH, Basel (CH)

(21) Appl. No.: 19/048,629

(22) Filed: Feb. 7, 2025

Related U.S. Application Data
(63) Continuation of application No. PCT/CN2023/112161, filed on Aug. 10, 2023.

(30) Foreign Application Priority Data
Aug. 10, 2022 (WO) PCT/CN2022/111326
May 22, 2023 (WO) PCT/CN2023/095564

(51) Int. Cl. C07K 16/28; A61K 39/00; A61P 35/00; A61P 37/04

(52) U.S. Cl. CPC C07K 16/2803; A61P 35/00; A61P 37/04; A61K 2039/505; C07K 2317/24; C07K 2317/41; C07K 2317/52; C07K 2317/565; C07K 2317/92

(57) ABSTRACT

Anti-PVRIG antibodies and antigen-binding fragments thereof, are provided, as are related compositions and methods. The antibodies of the disclosure bind to human PVRIG receptor and can block the interaction between the PVRL2 ligand on one cell with PVRIG on an immune cell.

### CROSS-REFERENCE TO RELATED APPLICATIONS

This application is a continuation of International Patent Application No. PCT/CN2023/112161, filed Aug. 10, 2023, which claims priority from International Patent Application No. PCT/CN2022/111326, filed Aug. 10, 2022, and International Patent Application No. PCT/CN2023/095564, filed May 22, 2023. The contents of these applications is incorporated herein by reference in their entirety.

### SEQUENCE LISTING

The instant application contains a sequence listing which has been submitted electronically in xml format, and is hereby incorporated by reference in its entirety. Said xml file was created on Feb. 6, 2025, is 251 bytes in size, and named 138881_1201_Sequence_Listing.xml.

### FIELD OF THE DISCLOSURE

Disclosed herein are compositions comprising anti-PVRIG antibodies, or antigen-binding fragments thereof, and related methods for treating cancer and other disorders responsive to PVRIG/PVRL2 antagonism.

Humanized, mouse or chimeric anti-PVRIG monoclonal antibodies are provided. The antibodies bind to human glycosylated and a glycosylated PVRIG with optimized binding kinetics, they disrupt the human PVRIG-PVRL2 interaction, and find use in various therapeutic and preventive methods. The present disclosure includes isolated antibodies and derivatives and fragments thereof, pharmaceutical formulations comprising one or more of the humanized or chimeric anti-PVRIG monoclonal antibodies; and cell lines that produce these monoclonal antibodies. Also provided are amino acid and nucleotide sequences of the antibodies.

### BACKGROUND

Immune checkpoint inhibitors that block inhibitory checkpoint proteins such as CTLA4 or PD-1/PD-L1 have made a significant impact on the development of anticancer therapies. In the clinic, several immune checkpoint inhibitors targeting either PD-1 or PD-L1 have been approved and have met with success, for example, nivolumab and pembrolizumab. However, there remains an unmet need for additional checkpoint inhibitors as clinical results have shown that responses can vary between patients and that resistance is possible.

Poliovirus receptor-related immunoglobulin domain-containing protein (PVRIG, also known as CD112R) was named for the homology observed between its second exon and the variable immunoglobulin domain of the polio virus receptor (PVR/CD155). PVRIG is a coinhibitory receptor expressed on the surface of T and natural killer (NK) cells. The receptor protein includes a single extracellular IgV domain, one transmembrane domain, and a long intracellular domain with an ITIM-like motif. PVRIG is a high affinity receptor for the ligand PVRL2, which is also known as Poliovirus receptor-related 2 (PVRL2), Nectin-2 and CD112. PVRL2 is up-regulated on antigen-presenting cells (APCs) and tumor cells, and is a high-affinity ligand for PVRIG (Zhu et al., 213(2), 167-176).

PVRIG plays an important role in immune cell activation because the interaction between PVRIG and its ligand inhibits the strength with which NK and T cells respond to cancer. PVRL2 binds the PVRIG receptor, which transmits an inhibitory signal to immune cells. Lead clinical candidates interfere with this inhibitory signal to reinstall the immune system's capability to fight cancer. The proposed indication for a fully human anti-PVRIG therapeutic antibody is as a checkpoint inhibitor in cancerous tissue where PVRL2 expression levels are high. This includes lung, kidney, ovarian, prostate and endometrial cancers, among others. (Whelan et al., 2019 Immunol Res. 2019; 7:257-68; Turnis et al., 2015 Eur J Immunol. 2015; 45:1892-905).

As the regulation of immunity is complex, multiple immune checkpoint inhibitors can prove to be beneficial. It was demonstrated that antibodies to PVRIG and TIGIT either as a single agent or in combination sensitized NK cell response (Xu et al., 2017 Cancer Immunol Immunother 66:1367-75). It was also reported that a triple combination of anti-PVRIG, anti-TIGIT and anti-PD1 antibodies resulted in a greater increase in INF-γ production and the increase in CD8⁺ T cell function. (Whelan et al., 2019 Immunol Res. 2019; 7:257-68).

The present disclosure provides anti-PVRIG antibodies showing various desirable characteristics for the treatment of a disease such as cancer in a subject, e.g., a human.

### SUMMARY OF THE DISCLOSURE

The present disclosure encompasses the following embodiments.

The present disclosure is directed to anti-PVRIG antibodies and antigen-binding fragments thereof that specifically bind PVRIG.

In certain embodiments, the present disclosure is directed to an antibody or antigen-binding fragment thereof, which specifically binds to human PVRIG from amino acid 41 to amino acid 171 (SEQ ID NO:154).

In certain embodiments, the present disclosure is directed to an antibody, or antigen-binding fragment thereof, which binds to human PVRIG comprising:
(i)
three heavy chain CDRs:
HCDR1 comprising an amino acid sequence as set forth in SEQ ID NO:44,
HCDR2 comprising an amino acid sequence as set forth in SEQ ID NO:45,
HCDR3 comprising an amino acid sequence as set forth in SEQ ID NO:46, and
three light chain CDRs:
LCDR1 comprising an amino acid sequence as set forth in SEQ ID NO:47,
LCDR2 comprising an amino acid sequence as set forth in SEQ ID NO:48,
LCDR3 comprising an amino acid sequence as set forth in SEQ ID NO:49;
(ii)
three heavy chain CDRs:
HCDR1 comprising an amino acid sequence as set forth in SEQ ID NO:34,
HCDR2 comprising an amino acid sequence as set forth in SEQ ID NO:35,
HCDR3 comprising an amino acid sequence as set forth in SEQ ID NO:36, and
three light chain CDRs:
LCDR1 comprising an amino acid sequence as set forth in SEQ ID NO:37,
LCDR2 comprising an amino acid sequence as set forth in SEQ ID NO:38,
LCDR3 comprising an amino acid sequence as set forth in SEQ ID NO:39;
(iii)
three heavy chain CDRs:
HCDR1 comprising an amino acid sequence as set forth in SEQ ID NO:14,
HCDR2 comprising an amino acid sequence as set forth in SEQ ID NO:15,
HCDR3 comprising an amino acid sequence as set forth in SEQ ID NO:16, and
three light chain CDRs:
LCDR1 comprising an amino acid sequence as set forth in SEQ ID NO:17,
LCDR2 comprising an amino acid sequence as set forth in SEQ ID NO:18,
LCDR3 comprising an amino acid sequence as set forth in SEQ ID NO:19;
(iv)
three heavy chain CDRs:
HCDR1 comprising an amino acid sequence as set forth in SEQ ID NO:4,
HCDR2 comprising an amino acid sequence as set forth in SEQ ID NO:5,
HCDR3 comprising an amino acid sequence as set forth in SEQ ID NO:6, and
three light chain CDRs:
LCDR1 comprising an amino acid sequence as set forth in SEQ ID NO:7,
LCDR2 comprising an amino acid sequence as set forth in SEQ ID NO:8,
LCDR3 comprising an amino acid sequence as set forth in SEQ ID NO:9;
(v)
three heavy chain CDRs:
HCDR1 comprising an amino acid sequence as set forth in SEQ ID NO:24,
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:29;
(vi)
three heavy chain CDRs:
HCDR1 comprising an amino acid sequence as set forth in SEQ ID NO:54,
HCDR2 comprising an amino acid sequence as set forth in SEQ ID NO:55,
HCDR3 comprising an amino acid sequence as set forth in SEQ ID NO:56, and
three light chain CDRs:
LCDR1 comprising an amino acid sequence as set forth in SEQ ID NO:57,
LCDR2 comprising an amino acid sequence as set forth in SEQ ID NO:58,
LCDR3 comprising an amino acid sequence as set forth in SEQ ID NO:59;
(vii)
three heavy chain CDRs:
HCDR1 comprising an amino acid sequence as set forth in SEQ ID NO:64,
HCDR2 comprising an amino acid sequence as set forth in SEQ ID NO:65,
HCDR3 comprising an amino acid sequence as set forth in SEQ ID NO:66, and
three light chain CDRs:
LCDR1 comprising an amino acid sequence as set forth in SEQ ID NO:67,
LCDR2 comprising an amino acid sequence as set forth in SEQ ID NO:68,
LCDR3 comprising an amino acid sequence as set forth in SEQ ID NO:69;
(viii)
three heavy chain CDRs:
HCDR1 comprising an amino acid sequence as set forth in SEQ ID NO:74,
HCDR2 comprising an amino acid sequence as set forth in SEQ ID NO:75,
HCDR3 comprising an amino acid sequence as set forth in SEQ ID NO:76, and
three light chain CDRs:
LCDR1 comprising an amino acid sequence as set forth in SEQ ID NO:77,
LCDR2 comprising an amino acid sequence as set forth in SEQ ID NO:78,
LCDR3 comprising an amino acid sequence as set forth in SEQ ID NO:79;
(ix)
three heavy chain CDRs:
HCDR1 comprising an amino acid sequence as set forth in SEQ ID NO:84,
HCDR2 comprising an amino acid sequence as set forth in SEQ ID NO:85,
HCDR3 comprising an amino acid sequence as set forth in SEQ ID NO:86, and
three light chain CDRs:
LCDR1 comprising an amino acid sequence as set forth in SEQ ID NO:87,
LCDR2 comprising an amino acid sequence as set forth in SEQ ID NO:88,
LCDR3 comprising an amino acid sequence as set forth in SEQ ID NO:89;
(x)
three heavy chain CDRs:
HCDR1 comprising an amino acid sequence as set forth in SEQ ID NO:94,
HCDR2 comprising an amino acid sequence as set forth in SEQ ID NO:95,
HCDR3 comprising an amino acid sequence as set forth in SEQ ID NO:96, and
three light chain CDRs:
LCDR1 comprising an amino acid sequence as set forth in SEQ ID NO:97,
LCDR2 comprising an amino acid sequence as set forth in SEQ ID NO:98,
LCDR3 comprising an amino acid sequence as set forth in SEQ ID NO:99;
(xi)
three heavy chain CDRs:
HCDR1 comprising an amino acid sequence as set forth in SEQ ID NO:104,
HCDR2 comprising an amino acid sequence as set forth in SEQ ID NO:105,
HCDR3 comprising an amino acid sequence as set forth in SEQ ID NO:106, and
three light chain CDRs:
LCDR1 comprising an amino acid sequence as set forth in SEQ ID NO:107,
LCDR2 comprising an amino acid sequence as set forth in SEQ ID NO:108,
LCDR3 comprising an amino acid sequence as set forth in SEQ ID NO:109;
(xii)
three heavy chain CDRs:
HCDR1 comprising an amino acid sequence as set forth in SEQ ID NO:114,
HCDR2 comprising an amino acid sequence as set forth in SEQ ID NO:115,
HCDR3 comprising an amino acid sequence as set forth in SEQ ID NO:116, and
three light chain CDRs:
LCDR1 comprising an amino acid sequence as set forth in SEQ ID NO:117,
LCDR2 comprising an amino acid sequence as set forth in SEQ ID NO:118,
LCDR3 comprising an amino acid sequence as set forth in SEQ ID NO:119;
(xiii)
three heavy chain CDRs:
HCDR1 comprising an amino acid sequence as set forth in SEQ ID NO:124,
HCDR2 comprising an amino acid sequence as set forth in SEQ ID NO:125,
HCDR3 comprising an amino acid sequence as set forth in SEQ ID NO:126, and
three light chain CDRs:
LCDR1 comprising an amino acid sequence as set forth in SEQ ID NO:127,
LCDR2 comprising an amino acid sequence as set forth in SEQ ID NO:128,
LCDR3 comprising an amino acid sequence as set forth in SEQ ID NO:129;
(xiv)
three heavy chain CDRs:
HCDR1 comprising an amino acid sequence as set forth in SEQ ID NO:134,
HCDR2 comprising an amino acid sequence as set forth in SEQ ID NO:135,
HCDR3 comprising an amino acid sequence as set forth in SEQ ID NO:136, and
three light chain CDRs:
LCDR1 comprising an amino acid sequence as set forth in SEQ ID NO:137,
LCDR2 comprising an amino acid sequence as set forth in SEQ ID NO:138,
LCDR3 comprising an amino acid sequence as set forth in SEQ ID NO:139;
(xv)
three heavy chain CDRs:
HCDR1 comprising an amino acid sequence as set forth in SEQ ID NO:144,
HCDR2 comprising an amino acid sequence as set forth in SEQ ID NO:145,
HCDR3 comprising an amino acid sequence as set forth in SEQ ID NO:146, and
three light chain CDRs:
LCDR1 comprising an amino acid sequence as set forth in SEQ ID NO:147,
LCDR2 comprising an amino acid sequence as set forth in SEQ ID NO:148,
LCDR3 comprising an amino acid sequence as set forth in SEQ ID NO:149; or
(xvi)
three heavy chain CDRs:
HCDR1 comprising an amino acid sequence as set forth in SEQ ID NO:187,
HCDR2 comprising an amino acid sequence as set forth in SEQ ID NO:188,
HCDR3 comprising an amino acid sequence as set forth in SEQ ID NO:46, and
three light chain CDRs:
LCDR1 comprising an amino acid sequence as set forth in SEQ ID NO:47,
LCDR2 comprising an amino acid sequence as set forth in SEQ ID NO:48,
LCDR3 comprising an amino acid sequence as set forth in SEQ ID NO:49.

In certain embodiments, the present disclosure is directed to an antibody or antigen-binding fragment comprising:
(i) a heavy chain variable region (VH) comprising an amino acid sequence at least 90, 91, 92, 93, 94, 95, 96, 97, 98 or 99% identical to SEQ ID NO:50, and a light chain variable region (VL) comprising an amino acid sequence at least 90, 91, 92, 93, 94, 95, 96, 97, 98 or 99% identical to SEQ ID NO:51;
(ii) a heavy chain variable region (VH) comprising an amino acid sequence at least 90, 91, 92, 93, 94, 95, 96, 97, 98 or 99% identical to SEQ ID NO:40, and a light chain variable region (VL) comprising an amino acid sequence at least 90, 91, 92, 93, 94, 95, 96, 97, 98 or 99% identical to SEQ ID NO:41;
(iii) a heavy chain variable region (VH) comprising an amino acid sequence at least 90, 91, 92, 93, 94, 95, 96, 97, 98 or 99% identical to SEQ ID NO:20, and a light chain variable region (VL) comprising an amino acid sequence at least 90, 91, 92, 93, 94, 95, 96, 97, 98 or 99% identical to SEQ ID NO:21;
(iv) a heavy chain variable region (VH) comprising an amino acid sequence at least 90, 91, 92, 93, 94, 95, 96, 97, 98 or 99% identical to SEQ ID NO:10, and a light chain variable region (VL) comprising an amino acid sequence at least 90, 91, 92, 93, 94, 95, 96, 97, 98 or 99% identical to SEQ ID NO:11;
(v) a heavy chain variable region (VH) comprising an amino acid sequence at least 90, 91, 92, 93, 94, 95, 96, 97, 98 or 99% identical to SEQ ID NO:30, and a light chain variable region (VL) comprising an amino acid sequence at least 90, 91, 92, 93, 94, 95, 96, 97, 98 or 99% identical to SEQ ID NO:31;
(vi) a heavy chain variable region (VH) comprising an amino acid sequence at least 90, 91, 92, 93, 94, 95, 96, 97, 98 or 99% identical to SEQ ID NO:60, and a light chain variable region (VL) comprising an amino acid sequence at least 90, 91, 92, 93, 94, 95, 96, 97, 98 or 99% identical to SEQ ID NO:61;
(vii) a heavy chain variable region (VH) comprising an amino acid sequence at least 90, 91, 92, 93, 94, 95, 96, 97, 98 or 99% identical to SEQ ID NO:70, and a light chain variable region (VL) comprising an amino acid sequence at least 90, 91, 92, 93, 94, 95, 96, 97, 98 or 99% identical to SEQ ID NO:71;
(viii) a heavy chain variable region (VH) comprising an amino acid sequence at least 90, 91, 92, 93, 94, 95, 96, 97, 98 or 99% identical to SEQ ID NO:80, and a light chain variable region (VL) comprising an amino acid sequence at least 90, 91, 92, 93, 94, 95, 96, 97, 98 or 99% identical to SEQ ID NO:81;
(ix) a heavy chain variable region (VH) comprising an amino acid sequence at least 90, 91, 92, 93, 94, 95, 96, 97, 98 or 99% identical to SEQ ID NO:90, and a light chain variable region (VL) comprising an amino acid sequence at least 90, 91, 92, 93, 94, 95, 96, 97, 98 or 99% identical to SEQ ID NO:91;
(x) a heavy chain variable region (VH) comprising an amino acid sequence at least 90, 91, 92, 93, 94, 95, 96, 97, 98 or 99% identical to SEQ ID NO:100, and a light chain variable region (VL) comprising an amino acid sequence at least 90, 91, 92, 93, 94, 95, 96, 97, 98 or 99% identical to SEQ ID NO:101;
(xi) a heavy chain variable region (VH) comprising an amino acid sequence at least 90, 91, 92, 93, 94, 95, 96, 97, 98 or 99% identical to SEQ ID NO:110, and a light chain variable region (VL) comprising an amino acid sequence at least 90, 91, 92, 93, 94, 95, 96, 97, 98 or 99% identical to SEQ ID NO:111;
(xii) a heavy chain variable region (VH) comprising an amino acid sequence at least 90, 91, 92, 93, 94, 95, 96, 97, 98 or 99% identical to SEQ ID NO:120, and a light chain variable region (VL) comprising an amino acid sequence at least 90, 91, 92, 93, 94, 95, 96, 97, 98 or 99% identical to SEQ ID NO:121;
(xiii) a heavy chain variable region (VH) comprising an amino acid sequence at least 90, 91, 92, 93, 94, 95, 96, 97, 98 or 99% identical to SEQ ID NO:130, and a light chain variable region (VL) comprising an amino acid sequence at least 90, 91, 92, 93, 94, 95, 96, 97, 98 or 99% identical to SEQ ID NO:131;
(xiv) a heavy chain variable region (VH) comprising an amino acid sequence at least 90, 91, 92, 93, 94, 95, 96, 97, 98 or 99% identical to SEQ ID NO:140, and a light chain variable region (VL) comprising an amino acid sequence at least 90, 91, 92, 93, 94, 95, 96, 97, 98 or 99% identical to SEQ ID NO:141; or
(xv) a heavy chain variable region (VH) comprising an amino acid sequence at least 90, 91, 92, 93, 94, 95, 96, 97, 98 or 99% identical to SEQ ID NO:150, and a light chain variable region (VL) comprising an amino acid sequence at least 90, 91, 92, 93, 94, 95, 96, 97, 98 or 99% identical to SEQ ID NO:151.

In certain embodiments, the present disclosure is directed to an antibody or antigen-binding fragment thereof, wherein one, two, three, four, five, six, seven, eight, nine, or ten amino acids within SEQ ID Nos: 50 and 51, SEQ ID Nos: 40 and 41, SEQ ID Nos: 20 and 21, SEQ ID Nos: 10 and 11, SEQ ID Nos: 30 and 31, SEQ ID Nos: 60 and 61, SEQ ID Nos: 70 and 71, SEQ ID Nos: 80 and 81, SEQ ID Nos: 90 and 91, SEQ ID Nos: 100 and 101, SEQ ID Nos: 110 and 111, SEQ ID Nos: 120 and 121, SEQ ID Nos: 130 and 131, SEQ ID Nos: 140 and 141, or SEQ ID Nos: 150 and 151, have been inserted, deleted or substituted.

In certain embodiments, the present disclosure is directed to an antibody or antigen-binding fragment thereof, which comprises:
(i) a heavy chain variable region (VH) comprising an amino acid sequence as set forth in SEQ ID NO:50, and a light chain variable region (VL) comprising an amino acid sequence as set forth in SEQ ID NO:51;
(ii) a heavy chain variable region (VH) comprising an amino acid sequence as set forth in SEQ ID NO:40, and a light chain variable region (VL) comprising an amino acid sequence as set forth in SEQ ID NO:41;
(iii) a heavy chain variable region (VH) comprising an amino acid sequence as set forth in SEQ ID NO:20, and a light chain variable region (VL) comprising an amino acid sequence as set forth in SEQ ID NO:21;
(iv) a heavy chain variable region (VH) comprising an amino acid sequence as set forth in SEQ ID NO:10, and a light chain variable region (VL) comprising an amino acid sequence as set forth in SEQ ID NO:11;
(v) a heavy chain variable region (VH) comprising an amino acid sequence as set forth in SEQ ID NO:30, and a light chain variable region (VL) comprising an amino acid sequence as set forth in SEQ ID NO:31;
(vi) a heavy chain variable region (VH) comprising an amino acid sequence as set forth in SEQ ID NO:60, and a light chain variable region (VL) comprising an amino acid sequence as set forth in SEQ ID NO:61;
(vii) a heavy chain variable region (VH) comprising an amino acid sequence as set forth in SEQ ID NO:50, and a light chain variable region (VL) comprising an amino acid sequence as set forth in SEQ ID NO:51;
(viii) a heavy chain variable region (VH) comprising an amino acid sequence as set forth in SEQ ID NO:70, and a light chain variable region (VL) comprising an amino acid sequence as set forth in SEQ ID NO:71;
(ix) a heavy chain variable region (VH) comprising an amino acid sequence as set forth in SEQ ID NO:80, and a light chain variable region (VL) comprising an amino acid sequence as set forth in SEQ ID NO:81;
(x) a heavy chain variable region (VH) comprising an amino acid sequence as set forth in SEQ ID NO:90, and a light chain variable region (VL) comprising an amino acid sequence as set forth in SEQ ID NO:91;
(xi) a heavy chain variable region (VH) comprising an amino acid sequence as set forth in SEQ ID NO:100, and a light chain variable region (VL) comprising an amino acid sequence as set forth in SEQ ID NO:101;
(xii) a heavy chain variable region (VH) comprising an amino acid sequence as set forth in SEQ ID NO:110, and a light chain variable region (VL) comprising an amino acid sequence as set forth in SEQ ID NO:111;
(xiii) a heavy chain variable region (VH) comprising an amino acid sequence as set forth in SEQ ID NO:120, and a light chain variable region (VL) comprising an amino acid sequence as set forth in SEQ ID NO:121;
(xiv) a heavy chain variable region (VH) comprising an amino acid sequence as set forth in SEQ ID NO:140, and a light chain variable region (VL) comprising an amino acid sequence as set forth in SEQ ID NO:141;
(xv) a heavy chain variable region (VH) comprising an amino acid sequence as set forth in SEQ ID NO:150, and a light chain variable region (VL) comprising an amino acid sequence as set forth in SEQ ID NO:151.

In certain embodiments, the present disclosure is directed to an antibody or antigen-binding fragment thereof, comprising an antigen binding domain that specifically binds to human PVRIG at an epitope comprising S27, S31, L32, H52, F99, K95, P100, E101, S103 and E105 of SEQ ID NO:154. In certain embodiments, the present disclosure is directed to an antibody or antigen-binding fragment thereof, comprising an antigen binding domain that specifically binds to human PVRIG at an epitope comprising F99 of SEQ ID NO:154.

In certain embodiments, the present disclosure is directed to an antibody or antigen-binding fragment thereof, which comprises: a heavy chain comprising an amino acid sequence as set forth in SEQ ID NO:189, and a light chain comprising an amino acid sequence as set forth in SEQ ID NO:190.

In certain embodiments, the present disclosure is directed to an antibody or antigen-binding fragment thereof, wherein the antibody or antigen-binding portion thereof cross-competes for binding to PVRIG with a reference antibody, or a reference antigen-binding portion thereof, comprising any of the antibodies or antigen-binding fragments disclosed herein.

In certain embodiments, the present disclosure is directed to an antibody or antigen-binding fragment thereof, wherein the antibody binds to a discontinuous epitope on PVRIG.

In certain embodiments, the present disclosure is directed to an antibody or antigen-binding fragment thereof, wherein the antibody binds to a discontinuous epitope on PVRIG, wherein the discontinuous epitope comprises amino acid residues S27, S31 and L32 of human PVRIG comprising SEQ ID NO:154.

In certain embodiments, the present disclosure is directed to an antibody or antigen-binding fragment thereof, wherein the discontinuous epitope further comprises amino acid residues H52, K95, F99, P100, E101, S103 and E105 of human PVRIG comprising SEQ ID NO:154.

In certain embodiments, the present disclosure is directed to an antibody or antigen-binding fragment thereof, wherein the antibody is a monoclonal antibody, a chimeric antibody, a humanized antibody, a human engineered antibody, a single chain antibody (scFv), a Fab fragment, a Fab′ fragment, or a F(ab′)₂ fragment.

In certain embodiments, the present disclosure is directed to an antibody or antigen-binding fragment thereof, wherein the antibody or antigen-binding fragment thereof has reduced glycosylation or no glycosylation or is hypofucosylated. In some embodiments, the antibody or antigen-binding fragment thereof has reduced glycosylation or no glycosylation or is hypofucosylated relative to an unmodified antibody or antigen-binding fragment thereof.

In certain embodiments, the present disclosure is directed to an antibody or antigen-binding fragment thereof, wherein the antibody or antigen-binding fragment thereof comprises increased bisecting GlcNac structures. In some embodiments, the antibody or antigen-binding fragment thereof has increased bisecting GlcNac structures relative to an unmodified antibody or antigen-binding fragment thereof.

In certain embodiments, the present disclosure is directed to an antibody or antigen-binding fragment thereof, wherein the Fc domain is of an IgG1.

In certain embodiments, the present disclosure is directed to a pharmaceutical composition comprising an antibody or antigen-binding fragment thereof, as disclosed herein, and a pharmaceutically acceptable carrier.

In certain embodiments, the present disclosure is directed to an isolated nucleic acid that encodes an antibody or antigen-binding fragment as disclosed herein.

In certain embodiments, the present disclosure is directed to a vector comprising said nucleic acid disclosed herein.

In certain embodiments, the present disclosure is directed to a host cell comprising said nucleic acid disclosed herein or said vector disclosed herein.

In certain embodiments, the present disclosure is directed to a process for producing an antibody or antigen-binding fragment thereof, comprising cultivating said host cell in culture media and recovering the antibody or antigen-binding fragment thereof, from said culture media.

In certain embodiments, the present disclosure is directed to a purified composition comprising an anti-human PVRIG antibody or antigen binding fragment thereof, produced by said process.

In certain embodiments, the present disclosure is directed to a kit comprising an antibody or antigen-binding fragment thereof disclosed herein and instructions for using the antibody or antigen-binding fragment thereof disclosed herein (e.g., an antibody selected from BGA38, BGA381, BGA382, BGA383 and BGA384 or an antigen-binding fragment thereof).

In certain embodiments, the present disclosure is directed to a kit wherein the antibody or antigen-binding fragment thereof forms a complex with PVRIG that is detected by an assay comprising an enzyme linked immunosorbent assay (ELISA), radioimmune assay (RIA), and/or Western blot.

In certain embodiments, the present disclosure is directed to a method of treating cancer comprising administering to a patient in need thereof an effective amount of said pharmaceutical composition described herein or said purified composition described herein.

In certain embodiments, the present disclosure is directed to said method of treating cancer, wherein the cancer is selected from the group consisting of PVRL2-expressing cancers, PVRL2-accumulating cancers, neoplasms of the central nervous system, mesothelioma, lymphoma, leukemia, myeloma, sarcoma, and cancers and metastases thereof of the bladder, stomach, uterus/cervix, prostate, testes, esophagus, intestine, colon, pancreas, breast, head and neck, kidney, liver, lung, germ cell, bone, liver, thyroid, ovary and skin.

In certain embodiments, the antibody or antigen-binding fragment is administered in combination with another therapeutic agent.

In certain embodiments, the therapeutic agent is a chemotherapeutic agent.

In certain embodiments, the therapeutic agent is an immune checkpoint inhibitor.

In certain embodiments, the immune checkpoint inhibitor is an anti-PD1 antibody.

In certain embodiments, the anti-PD1 antibody is BGB-A317.

In certain embodiments, the immune checkpoint inhibitor is an anti-TIGIT antibody.

In certain embodiments, the anti-TIGIT antibody is BGB-A1217.

In certain embodiments, the immune checkpoint inhibitor comprises a combination of BGB-A317 and A1217.

In certain embodiments, the present disclosure is directed to a method of stimulating an immune response in a subject comprising administering to a subject said pharmaceutical composition or said purified composition.

In certain embodiments, the step of administering increases antibody-dependent trogocytosis or effector T cell response in the subject. In some embodiments, a step of administering an antibody or antigen-binding fragment thereof described herein results in an increase in antibody-dependent trogocytosis or effector T cell response in the subject relative to administering the antibody or antigen-binding fragment thereof comprising one or more mutations in the Fc domain.

In certain embodiments, the present disclosure is directed to a method of inducing trogocytosis of PVRIG from a donor cell to an acceptor cell, comprising contacting the donor cell with an antibody or antigen-binding fragment as disclosed herein, for a time sufficient to induce trogocytosis from the donor cell to the acceptor cell.

In certain embodiments, the present disclosure is directed to a method of activating NK-cells comprising administering to a patient in need thereof an effective amount of said pharmaceutical composition or said purified composition.

In certain embodiments of said method, the patient has cancer and an immune response against said cancer is stimulated.

In certain further embodiments, the cancer is selected from the group consisting of PVRL2-expressing cancers, PVRL2-accumulating cancers, neoplasms of the central nervous system, mesothelioma, lymphoma, leukemia, myeloma, sarcoma, and cancers and metastases thereof of the bladder, stomach, uterus/cervix, prostate, testes, esophagus, intestine, colon, pancreas, breast, head and neck, kidney, liver, lung, germ cell, bone, liver, thyroid, ovary and skin.

### BRIEF DESCRIPTION OF THE DRAWINGS

FIG. 1 shows BGA12 and engineered versions BGA121 and BGA122 binding to cells expressing human PVRIG.

FIG. 2 demonstrates that BGA12, BGA121 and BGA 122 antibodies greatly reduced PVRIG binding to PVRL2 in a dose dependent manner.

FIGS. 3A-B indicate that humanized variants of BGA38 showed comparable binding to hPVRIG-Jurkat.

FIGS. 4A-B shows that humanized variants of BGA38 show comparable binding to Cyno PVRIG-293T.

FIGS. 5A-B indicate that humanized variants of BGA38 showed comparable reduction in PVRL2 binding.

FIG. 6 shows that humanized variants of BGA86 showed comparable binding to hPVRIG-Jurkat.

FIG. 7 indicates that humanized variants of BGA86 showed comparable binding to Cyno PVRIG-293T cells.

FIG. 8 shows that humanized variants of BGA86 showed comparable reduction in PVRL2 binding.

FIG. 9 shows that humanized variants of BGA50 showed comparable binding to hPVRIG-Jurkat.

FIG. 10 shows that humanized variants of BGA50 showed comparable binding to Cyno PVRIG-293T.

FIG. 11 shows that humanized variants of BGA50 showed comparable reduction in PVRL2 binding.

FIGS. 12A-C, 12D-F and 12G show the results of a Jurkat/NFAT and A549 co-culture assay with anti-PVRIG antibodies.

FIGS. 13A-C show the results of a Jurkat/NFAT/hPVRIG⁺THP1/OS8 low assay with anti-PVRIG antibodies.

FIGS. 13D-G show the results of a Jurkat/NFAT/hPVRIG⁺THP1/OS8 low assay with humanized anti-PVRIG antibodies.

FIGS. 14A-B show the activation of human T cells by anti-PVRIG parental antibodies.

FIGS. 14C-D show the activation of human T cells by anti-PVRIG humanized antibodies.

FIGS. 15A-B and 15C-D demonstrate that anti-PVRIG antibodies can be used in combination with other immune checkpoint antibodies.

FIGS. 16A-E show that human NK cells can be activated by anti-PVRIG antibodies.

FIGS. 17A-B show the change in trogocytosis of cells when treated with anti-PVRIG antibodies.

FIG. 18 shows the crystal structure of A538-EH Fab bound to human PVRIG. Panel (A) is the crystal structure of A538-EH Fab bound to human PVRIG, with A538-EH heavy chain (HC) and light chain (LC) regions colored in black and gray cartoon while PVIRG in white surface. Panel (B) shows the atomic interactions on the binding surface of A538-EH/PVRIG complex, identifying certain key residues of A538-EH (paratope residues shown in line) and PVRIG (epitope residues shown in stick). For clarify, non-CDR region of A538-EH are removed with PVRIG colored in white transparent surface. The underlined residues were identified as functionally important epitopic residues by alanine scanning mutagenesis. Panel (C) shows the surrounding residues of F99 PVRIG and P100 PVRIG within A538-EH is shown to emphasize the interaction among these residues which is critical for PVRIG binding. F99PVRIG and P100PVRIG are highlighted with underlined larger text.

FIG. 19 is an alignment of heavy chain variable regions suitable for use in practicing the claims. The coverage (cov) and percent identities (pid) are shown according to Brown et al. (1998). The scheme highlights residues by identity and properties using known amino acid physicochemical classes (Taylor, 1986). Residues are arranged by color if identical to their counterpart in the stacked alignment. A structural consensus of which CDRs, showing which subset of residues in the combination of CDRs, is essential for the recited function of specifically binding to human PVRIG is shown in the alignment. (See MView: A Web compatible database search or multiple alignment viewer. Bioinformatics. 14 (4):380-381).

FIG. 20 is a stacked alignment showing structural similarities and differences among the primary amino acid sequences of variable heavy chain regions suitable for practicing the claims. The alignments show the common structural features that are critical to carrying out the claimed function of specifically binding to human PVRIG.

FIG. 21 shows the amino acid sequence of the heavy chain (A) and light chain (B) of BGA-384. The variable region is presented in capital letters, and constant region is in lower case. Complementarity determining regions are highlighted by bold face (Kabat and Wu 1971) and/or underline (Chothia and Lesk 1987; Chothia and Lesk 1987).

FIGS. 22A-B show binding of BGA-384 to recombinant PVRIG as determined by ELISA.

FIGS. 23A-B show BGA-384 binding to recombinant PVRIG as determined by FACS.

FIGS. 24A-B show a non-specific binding test of BGA-384 as determined by FACS.

FIG. 25 shows an amino acid sequence alignment of human, cynomolgus monkey, and murine PVRIG extracellular domains.

FIGS. 26A-C show sensorgrams of BGA-384 binding to human, cynomolgus, and mouse PVRIG.

FIG. 27 shows an analysis of the epitopes in PVRIG involved in BGA384 binding.

FIGS. 28A-B shows BGA-384 blockade activity against PVRIG-PVRL2 binding as performed by ELISA assay.

FIGS. 29A-B shows BGA-384 blockade activity against PVRIG-PVRL2 binding in a cell-based assay.

FIG. 30 shows an analysis of BGA-384 activity in PVRIG+ Jurkat T cells.

FIGS. 31A-C show IFN-γ secretion by CMV-specific T cells in response to BGA-384 in co-culture.

FIGS. 32A-C show IFN-γ secretion by CMV-specific T cells treated with BGA-384 alone or in combination with one or both of BGB-A317 or BGB-A1217.

FIG. 33 shows an analysis of BGA384 binding to C1q by ELISA.

FIG. 34 shows ADCC activity against primary human PBMC cells treated with BGA384.

FIGS. 35A-C show CDC activity with PBMCs of BGA384 and a control antibody.

FIG. 36 shows activation of Jurkat cells by BGA384 to produce NFAT-RE-mediated luminescence.

FIGS. 37A-D shows the percentage of CD107a+ cells in primary NK cells co-cultured with SK-BR-3, a PVRL2+ human breast cancer cell line, in the presence of BGA384.

FIG. 38 shows tumor growth inhibition (TGI) during weekly intraperitoneal administration of 1, 5, and 15 mg/kg of the anti-mouse PVRIG surrogate antibody of BGA384 (AB-407 WT).

FIG. 39 shows animal body weight during weekly intraperitoneal administration of 1, 5, and 15 mg/kg of AB-407 WT.

FIG. 40 shows cytokine release assays of GM-CSF, IFN-γ, IL-10, IL-12p70, IL-13, and IL-17A in PBMCs exposed to BGA384 at 10 μg/well and 100 μg/well relative to huIgG and OKT3.

FIG. 41 shows cytokine release assays of IL-10, IL-2, IL-4, IL-6, IL-8, and IP-10/CXCL10 in PBMCs exposed to BGA384 at 10 μg/well and 100 μg/well relative to huIgG and OKT3.

FIG. 42 shows cytokine release assays of MCP-1/CCL2, MIP-1a/CCL3, and TNF-α in PBMCs exposed to BGA384 at 10 μg/well and 100 μg/well relative to huIgG and OKT3.

FIG. 43 shows simulated human PK profiles of BGA384 at different dose levels.

### ABBREVIATIONS

As used throughout the specification and appended claims, the following abbreviations apply:
API active pharmaceutical ingredient
BOR best overall response
BID one dose twice daily
CDR clinical benefit rate
CDR complementarity determining region in the immunoglobulin
CHO Chinese hamster ovary
CR complete response
DCR disease control rate
DFS disease free survival
DLT dose limiting toxicity
DOR duration of response
DS drug substance
DSDR durable stable disease rate
CI confidence interval
EC50 concentration resulting in 50% efficacy or binding
ELISA enzyme-linked immunosorbent assay
FFPE formalin-fixed, paraffin-embedded
FR framework region
HC heavy chain
HNSCC head and neck squamous cell carcinoma
HP-HIC high performance hydrophobic interaction chromatography
HP-IEX high performance ion-exchange chromatography
HP-SEC high performance size exclusion chromatography
IC50 concentration resulting in 50% inhibition
IgG immunoglobulin G
IV intravenous
IHC immunohistochemistry or immunohistochemical
MTD maximum tolerated dose
mAb monoclonal antibody
NCBI National Center for Biotechnology Information
NSCLC non-small cell lung cancer
NCI National Cancer Institute
ORR objective response rate
OS overall survival
PCR polymerase chain reaction
PD progressive disease
PD-1 programmed death 1
PD-L1 programmed death 1 ligand 1
PR partial response
RECIST Response Evaluation Criteria in Solid Tumors
SD stable disease
TPI toxicity probability interval
SWFI sterile water for injection
TNBC triple negative breast cancer
VH immunoglobulin heavy chain variable region
VK immunoglobulin kappa light chain variable region
VL immunoglobulin light chain variable region
v/v volume per volume
w/v weight per volume

### Definitions

In order for the present disclosure to be more readily understood, certain terms are first defined below. Additional definitions for the following terms and other terms are set forth throughout the Specification.

While various embodiments and aspects of the present disclosure are shown and described herein, it will be obvious to those skilled in the art that such embodiments and aspects are provided by way of example only. Numerous variations, changes, and substitutions will occur to those skilled in the art without departing from what is disclosed herein. It should be understood that various alternatives to the embodiments disclosed or described herein can be employed in practicing the instant disclosure.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the same meaning as commonly understood by one of ordinary skill in the art to which this disclosure is related. For example, Juo, The Concise Dictionary of Biomedicine and Molecular Biology, 2nd ed., (2001), CRC Press; The Dictionary of Cell & Molecular Biology, 5th ed., (2013), Academic Press; and “The Oxford Dictionary of Biochemistry and Molecular Biology,” Cammack et al. eds., 2nd ed, (2006), Oxford University Press, provide those of skill in the art with a general dictionary for many of the terms used in this disclosure. Additional definitions of common terms in molecular biology may be found in Benjamin Lewin, Genes V, published by Oxford University Press, 1994 (ISBN 0-19 854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

The term “Poliovirus receptor-related immunoglobulin domain-containing protein” or “PVRIG” or “CD112R” refers a cell receptor. The amino acid sequence of human PVRIG, (SEQ ID NO:1) can also be found at accession number Q6DKI7 (PVRIG_HUMAN) or NM_024070. The amino acid sequence of Cynomolgus Monkey (“Cyno”) PVRIG, (SEQ ID NO:2) can also be found at accession number A0A2K5WVV8_MACFA or XM_005549224.

(SEQ ID NO: 1)
MRTEAQVPALQPPEPGLEGAMGHRTLVLPWVLLTLCVTAGTPEVWVQVRM
EATELSSFTIRCGFLGSGSISLVTVSWGGPNGAGGTTLAVLHPERGIRQW
APARQARWETQSSISLILEGSGASSPCANTTFCCKFASFPEGSWEACGSL
PPSSDPGLSAPPTPAPILRADLAGILGVSGVLLFGCVYLLHLLRRHKHRP
APRLQPSRTSPQAPRARAWAPSQASQAALHVPYATINTSCRPATLDTAHP
HGGPSWWASLPTHAAHRPQGPAAWASTPIPARGSFVSVENGLYAQAGERP
PHTGPGLTLFPDPRGPRAMEGPLGVR
(SEQ ID NO: 2)
MRTEAQVLALQSPEPGLEGAMGRRTLALPWVLLTLCVTAGTPEVWVQVQM
EATELSSFTVHCGFLGPGSISLVTVSWGGPDGAGGTKLAVLHPELGTRQW
APARQARWETQSSISLALEDSGASSPFANTTFCCKFASFPEGSWESCGSL
PPSSDPGLSAPPTPVPILRADLAGILGLSGVLLFGCGYLLHLLRRQKHRP
TPRLQPSHTNSQALTAQAWAPSQASQAALHDPYATINTSFCPATLDTAHP
NGWASLPTHAAHQPQGPAASASTPILARGSFVSVENGLYTQAGERPPHTG
PGLTLFPDCRGPRAVEGRFGVR

A ligand for PVRIG is known as “Poliovirus receptor-related protein 2” (PVRL2), “Herpes virus entry mediator B” (HveB), or “Nectin-2” and the amino acid sequence can be found at NP_002847 (SEQ ID NO:3).

(SEQ ID NO: 3)
MARAAALLPSRSPPTPLLWPLLLLLLLETGAQDVRVQVLPEVRGQLGGTV
ELPCHLLPPVPGLYISLVTWQRPDAPANHQNVAAFHPKMGPSFPSPKPGS
ERLSFVSAKQSTGQDTEAELQDATLALHGLTVEDEGNYTCEFATFPKGSV
RGMTWLRVIAKPKNQAEAQKVTFSQDPTTVALCISKEGRPPARISWLSSL
DWEAKETQVSGTLAGTVTVTSRFTLVPSGRADGVTVTCKVEHESFEEPAL
IPVTLSVRYPPEVSISGYDDNWYLGRTDATLSCDVRSNPEPTGYDWSTTS
GTFPTSAVAQGSQLVIHAVDSLFNTTFVCTVTNAVGMGRAEQVIFVRETP
NTAGAGATGGIIGGIIAAIIATAVAATGILICRQQRKEQTLQGAEEDEDL
EGPPSYKPPTPKAKLEAQEMPSQLFTLGASEHSPLKTPYFDAGASCTEQE
MPRYHELPTLEERSGPLHPGATSLGSPIPVPPGPPAVEDVSLDLEDEEGE
EEEEYLDKINPIYDALSYSSPSDSYQGKGFVMSRAMYV

As used herein, including the appended claims, the singular forms of words such as “a,” “an,” and “the,” include their corresponding plural references unless the context clearly dictates otherwise. The term “or” is used to mean, and is used interchangeably with, the term “and/or” unless the context clearly dictates otherwise. The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” as used herein, are used merely by way of example, without limitation intended, and should not be construed as referring only those items explicitly enumerated in the specification.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

Ranges can be expressed herein as from “about” one particular value, and/or to “about” another particular value. When such a range is expressed, another case includes from the one particular value and/or to the other particular value. Similarly, when values are expressed as approximations, by use of the antecedent “about,” it will be understood that the particular value forms another case. It will be further understood that the endpoints of each of the ranges are significant both in relation to the other endpoint, and independently of the other endpoint. As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systeme International de Unites (SI) accepted form. Numeric ranges are inclusive of the numbers defining the range.

The terms “at least”, “more than”, “or more”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between. The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “subject” or “patient” refers to any single subject for which therapy is desired or that is participating in a clinical trial, epidemiological study or used as a control, and includes any organism, preferably an animal, more preferably a mammal (e.g., rat, mouse, dog, cat, horse, cattle), and most preferably a human.

Exemplary routes of administration for the formulations disclosed herein include intravenous, intramuscular, subcutaneous, intraperitoneal, spinal or other parenteral routes of administration, for example by injection, infusion or by way of a syringe infusion system. The phrase “parenteral administration” means modes of administration other than enteral and topical administration, usually by injection, and includes, without limitation, intravenous, intramuscular, intraarterial, intrathecal, intralymphatic, intralesional, intracapsular, infraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “affinity” as used herein refers to the strength of interaction between antibody and antigen. The variable regions of an antibody interact through non-covalent forces with an antigen at numerous sites. In general, the more interactions, the stronger the affinity.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “agent” may refer to a molecule or entity of any class comprising, or a plurality of molecules or entities, any of which may be, for example, a polypeptide, nucleic acid, saccharide, lipid, small molecule, metal, cell, or organism (for example, a fraction or extract thereof) or component thereof. In some embodiments, an agent may be utilized in isolated or pure form. In some embodiments, an agent may be utilized in a crude or impure form. In some embodiments, an agent may be provided as a population, collection, or library, for example that may be screened to identify or characterize members present therein.

The term “allogeneic” refers to any material derived from one individual which is then introduced to another individual of the same species, e.g., allogeneic NK or T cell transplantation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof can be replaced with an amino acid residue with a similar side chain.

Exemplary amino acid categorizations are summarized in Tables 1 and 2 below:

TABLE 1

| | | | | | Hydropathy |
|---|---|---|---|---|---|
| Amino Acid | 3-Letter | 1-Letter | Property | Property | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 2

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Such conservatively modified variants are in addition to and do not exclude polymorphic variants, interspecies homologs, and alleles.

The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptophan/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine. The following eight groups each contain exemplary amino acids that may be considered conservative substitutions for one another (see, e.g., Creighton, Proteins (1984)).
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

TABLE 3
Individual Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr(T) | Ser |
| Trp (W) | Tyr; Phe |
| Tyr (Y) | Trp; Phe |
| Val (V) | Ile; Leu |

In some embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions. In some embodiments, there may be 1, 2, 3, 4, 5, 6, 7, 8, 9, 10, 15, 20, 25, 30, 35, or 40 conservative substitutions. Thus, a “conservative amino acid substitution” refers to substitutions of amino acids in a protein with other amino acids having similar characteristics (e.g., charge, side-chain size, hydrophobicity/hydrophilicity, backbone conformation and rigidity, etc.) such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents, including cellular therapies.

Antibodies are large, complex molecules (molecular weight of ˜150,000 or about 1320 amino acids) with intricate internal structure. The term “antibody” (Ab) includes, without limitation, a glycoprotein immunoglobulin which binds specifically to an antigen. In general, antibodies are large, complex molecules (molecular weight of ˜150,000 or about 1320 amino acids) with an intricate internal structure. An antibody can comprise at least two heavy (H) chains and two light (L) chains interconnected by disulfide bonds, or an antigen-binding molecule thereof. Each H chain comprises a heavy chain variable region (abbreviated herein as HCVR or VH) and a heavy chain constant region. The heavy chain constant region comprises three constant domains, CH1, CH2 and CH3. Each light chain comprises a light chain variable region (abbreviated herein as LCVR or VL) and a light chain constant region. The term “antibody” as used herein refers to a polypeptide of the immunoglobulin family that can bind a corresponding antigen non-covalently, reversibly, and in a specific manner.

The light and heavy chain variable regions come together in 3-dimensional space to form a variable region that binds the antigen (for example, a receptor on the surface of a cell). The variable regions of each light/heavy chain (VL/VH) pair form the antibody binding site. In general, an intact antibody has two binding sites. Except in bifunctional or bispecific antibodies, the two binding sites are, generally, the same in primary sequence.

As will be presented in more detail below, within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The terms “LCDR1,” “LCDR2” and “LCDR3” as provided herein refer to the complementarity determining regions (CDR) 1, 2, and 3 of the variable light (L) chain of an antibody. In embodiments, the variable light chain provided herein includes in N-terminal to C-terminal direction a LCDR1, a LCDR2 and a LCDR3. Likewise, the terms “HCDR1,” “HCDR2” and “HCDR3” as provided herein refer to the complementarity determining regions (CDR) 1, 2, and 3 of the variable heavy (H) chain of an antibody. In certain embodiments, the variable heavy chain provided herein includes in N-terminal to C-terminal direction a HCDR1, a HCDR2 and a HCDR3. As detailed further below, the position and length of the CDRs have been precisely defined by Kabat, E. et al., Sequences of Proteins of Immunological Interest, U.S. Department of Health and Human Services, 1983, 1987. Naturally-produced antibodies are also glycosylated, e.g., on the CH2 domain.

The part of a variable region not contained in the CDRs is called the framework (“FR”), which forms the environment for the CDRs. Thus, the complementarity determining regions (CDRs) are interspersed with regions that are more conserved, i.e., the framework regions. Accordingly, each VH and VL comprises three CDRs and four FRs, arranged from amino-terminus to carboxy-terminus in the following order: FR1, CDR1, FR2, CDR2, FR3, CDR3, and FR4. While the variable regions of the heavy and light chains contain the binding domain that interacts with an antigen, the constant regions of the antibody may mediate the binding of the immunoglobulin to host tissues or factors, including various cells of the immune system (e.g., effector cells) and the first component (C1q) of the classical complement system.

TABLE 4
CDR Numbering

| Loop | Kabat | AbM | Chothia | Contact |
|---|---|---|---|---|
| L1 | L24--L34 | L24--L34 | L24--L34 | L30--L36 |
| L2 | L50--L56 | L50--L56 | L50--L56 | L46--L55 |
| L3 | L89--L97 | L89--L97 | L89--L97 | L89--L96 |
| H1 | H31--H35B | H26--H35B | H26--H32 . . . 34 | H30--H35B |
| | (Kabat | | | |
| | Numbering) | | | |
| H1 | H31--H35 | H26--H35 | H26--H32 | H30--H35 |
| | (Chothia | | | |
| | Numbering) | | | |
| H2 | H50--H65 | H50--H58 | H52--H56 | H47--H58 |
| H3 | H95--H102 | H95--H102 | H95--H102 | H93--H101 |

In certain aspects, the CDRs of an antibody can be determined according to the Chothia numbering scheme, which refers to the location of immunoglobulin structural loops (see, e.g., Chothia C. & Lesk A. M., (1987), J Mol Biol 196: 901-917; Al-Lazikani B. et al., (1997) J Mol Biol 273: 927-948; Chothia C. et al., (1992) J Mol Biol 227: 799-817; Tramontano A. et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). In a specific embodiment, the CDRs of the antibodies described herein have been determined according to the Chothia numbering scheme.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M. P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT, the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGapAlign.

When an antibody is said to comprise CDRs by a certain definition of CDRs (e.g., Kabat) that definition specifies the minimum number of CDR residues present in the antibody (i.e., the Kabat CDRs). It does not exclude the fact that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which HCDR1 is a composite Chothia-Kabat HCDR1 and other CDRs contain Kabat CDR residues and no additional CDR residues based on other definitions.

In some instances, a CDR is substantially identical to one found in a reference antibody (e.g., an antibody of the present disclosure) and/or the sequence of a CDR provided in the present disclosure. In some embodiments, a CDR is substantially identical to a reference CDR (e.g., a CDR provided in the present disclosure) in that it is either identical in sequence or contains between 1, 2, 3, 4, or 5 (e.g. 1-5) amino acid substitutions as compared with the reference CDR. In some embodiments a CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In some embodiments a CDR is substantially identical to a reference CDR in that it shows at least 96%, 96%, 97%, 98%, 99%, or 100% sequence identity with the reference CDR. In some embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g. 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody.

The terms “anti-PVRIG antibody” and “an antibody that binds to PVRIG” refer to an antibody that is capable of binding PVRIG with sufficient affinity such that the antibody is useful as a diagnostic and/or therapeutic agent in targeting PVRIG. In one embodiment, the extent of binding of an anti-PVRIG antibody to an unrelated, non-PVRIG protein is less than about 10% of the binding of the antibody to PVRIG as measured, e.g., by a radioimmunoassay (RIA). In certain embodiments, an antibody that binds to PVRIG has a dissociation constant (K_{D}) of 1 M, ≤100 nM, ≤10 nM, ≤5 nm, ≤4 nM, ≤3 nM, ≤2 nM, ≤1 nM, ≤0.1 nM, ≤0.01 nM, or ≤0.001 nM (e.g., 10⁻⁸ M or less, e.g. from 10⁻⁸ M to 10⁻¹³ M, e.g., from 10⁻⁹ M to 10⁻¹³ M).

The term “antibody” further includes, but is not limited to, monoclonal antibodies, human antibodies, humanized antibodies, chimeric antibodies, and anti-idiotypic (anti-Id) antibodies. Antibodies can include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen binding fragments of any of the above. In certain embodiments, antibodies described herein refer to polyclonal antibody populations. Antibodies may also comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof), camelid antibodies, single chain or Tandem diabodies (TandAb®), Anticalins®, Nanobodies® minibodies, BiTE®s, ankyrin repeat proteins or DARPINs®, Avimers®, DARTs, TCR-like antibodies, Adnectins®, Affilins®, Trans-Bodies®, Affibodies®, TrimerX®, MicroProteins, Fynomers®, Centyrins®, and KALBITOR®s.

An immunoglobulin may derive from any of the commonly known isotypes, including but not limited to IgA, secretory IgA, IgG, IgE, IgM, IgD, IgA and IgY. IgG subclasses are also well known to those in the art and include but are not limited to human IgG1, IgG2, IgG3, IgG4, IgA1 and IgA2. The term “antibody” and “immunoglobulin” or “Ig” may be used interchangeably herein.

The term “antibody” includes intact antibodies and binding fragments thereof. Typically, fragments compete with the intact antibody from which they were derived for specific binding to the target. Fragments include separate heavy chains, light chain Fab, Fab′, F(ab′)₂, Fv fragments and single domain antibodies. Single (variable) domain antibodies include VH regions separated from their VL partners (or vice versa) in conventional antibodies (Ward et al., 1989, Nature 341: 544-546) as well as VH regions (sometimes known as VHH) from species such as Camelidae or cartilaginous fish (e.g., a nurse shark) in which VH regions are not associated with VL regions (see, e.g., WO 9404678). For example, natural single variable region antibodies from Camelidae include a VHH variable region, and CH2 and CH3 constant regions. Single domain antibodies can be subject to humanization by analogous approaches to conventional antibodies. The Dabs type of antibodies are usually obtained from antibodies of human origin. Nanobody types of antibodies are of Camelidae or shark origin and can be subject to humanization. Fragments can be produced by recombinant DNA techniques, or by enzymatic or chemical separation of intact immunoglobulins. The term “antibody” also includes bi-, tri- and multi-specific antibodies. For example, a bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)).

Antibodies can exist, e.g., as intact immunoglobulins or as a number of well-characterized fragments produced by digestion with various peptidases. For example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al. (1990)).

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens. In one particular embodiment, the antigen is all or a fragment of PVRIG.

An “antigen binding molecule,” “antigen binding portion,” or “antibody fragment” refers to any molecule that comprises the antigen binding parts (e.g., CDRs) of the antibody from which the molecule is derived. Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In certain embodiments, the antigen binding molecule binds to PVRIG. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv).

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multi-specific antibodies formed from antibody fragments. In some embodiments, the antigen binding molecule binds to an antigen on a tumor cell. In certain embodiments, the antigen binding molecule binds to PVRIG.

An antigen binding fragment may be produced by any means. For example, in some embodiments, an antigen binding fragment may be enzymatically or chemically produced by fragmentation of an intact antibody. In some embodiments, an antigen binding fragment may be recombinantly produced (i.e., by expression of an engineered nucleic acid sequence. In some embodiments, an antigen binding fragment may be wholly or partially synthetically produced. In some embodiments, an antigen binding fragment may have a length of at least about 50, 60, 70, 80, 90, 100, 110, 120, 130, 140, 150, 160, 170, 180, 190 amino acids or more; in some embodiments at least about 200 amino acids (e.g., 50-100, 50-150, 50-200, or 100-200 amino acids).

“Antigen presenting cell” or “APC” refers to cells that process and present antigens to T-cells. Exemplary APCs comprise dendritic cells, macrophages, B cells, certain activated epithelial cells, and other cell types capable of TCR stimulation and appropriate T cell co-stimulation.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor.

“Anti-tumor response” when referring to a cancer patient treated with a therapeutic regimen, such as a therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression-free survival. Positive therapeutic effects in cancer can be measured in a number of ways (See, e.g., Weber, W. A. “Assessing tumor response to therapy.” Journal of nuclear medicine 50.Suppl 1 (2009): 1S-10S); Eisenhauer et al., supra). In some embodiments, an anti-tumor response to a therapy described herein is assessed using RECIST 1.1 criteria, bidimentional irRC or unidimensional irRC. In some embodiments, an anti-tumor response is any of SD, PR, CR, PFS, or DFS.

Two events or entities are “associated” with one another if the presence, level, and/or form of one is correlated with that of the other. For example, an entity (e.g., polypeptide, genetic signature, metabolite, microbe, etc.) is considered to be associated with a disease, disorder, or condition, if its presence, level, and/or form correlates with incidence of and/or susceptibility to the disease, disorder, or condition (e.g., across a relevant population). For example, two or more entities are physically “associated” with one another if they interact, directly or indirectly, so that they are and/or remain in physical proximity with one another (e.g., bind). In additional examples, two or more entities that are physically associated with one another are covalently linked or connected to one another, or non-covalently associated, for example by means of hydrogen bonds, van der Waals interaction, hydrophobic interactions, magnetism, and combinations thereof.

B cells play a principal role in humoral immunity (with antibody involvement). B cells make antibodies and perform the role of antigen-presenting cells (APCs), turning into memory B cells after activation by antigen interaction. In mammals, immature B cells are formed in the bone marrow, from which the name is derived.

The term “binding” generally refers to a non-covalent association between or among two or more entities. Direct binding involves physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

“Binding affinity” generally refers to the strength of the sum total of non-covalent interactions between a single binding site of a molecule (e.g., an antibody) and its binding partner (e.g., an antigen). Binding affinity can be measured and/or expressed in a number of ways known in the art, including, but not limited to, equilibrium dissociation constant (K_{D}), and equilibrium association constant (KA). Generally, the affinity of a molecule X for its partner Y can be represented by the equilibrium dissociation constant (K_{D}).

The term “buffer” encompasses those agents which maintain the solution pH of the compositions of the present disclosure in an acceptable range.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

Generally, a “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream.

A “cancer” or “cancer tissue” can include a tumor. Examples of cancers that can be treated by the methods of the present disclosure include, but are not limited to, cancers of the immune system including lymphoma, leukemia, myeloma, and other leukocyte malignancies. In some embodiments, the methods of the present disclosure can be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. In one particular embodiment, the cancer is multiple myeloma. The particular cancer can be responsive to chemo- or radiation therapy or the cancer can be refractory. A refractory cancer refers to a cancer that is not amendable to surgical intervention and the cancer is either initially unresponsive to chemo- or radiation therapy or the cancer becomes unresponsive over time. Cancer further includes relapsed or refractory large B-cell lymphoma after two or more lines of systemic therapy, including diffuse large B-cell lymphoma (DLBCL) not otherwise specified, primary mediastinal large B-cell lymphoma after two or more lines of systemic therapy, high grade B-cell lymphoma, and DLBCL arising from follicular lymphoma.

“Biotherapeutic agent” means a biological molecule, such as an antibody or fusion protein, that blocks ligand/receptor signaling in any biological pathway that supports tumor maintenance and/or growth or suppresses the anti-tumor immune response.

“Chemotherapeutic agent” refers to a chemical or biological substance that can cause death of cancer cells, or interfere with growth, division, repair, and/or function of cancer cells. Classes of chemotherapeutic agents include, but are not limited to: microtubule inhibitors, e.g., taxanes (paclitaxel, docetaxel), and vinca alkaloids (vincristine, vinblastine, vinorelbine); antimetabolites (5-fluorouracil, capecitabine, gemcitabine), and antifolates (methotrexate, pemetrexed); kinase inhibitors (crizotinib, erlotinib, sunitinib); topoisomerase inhibitors (deruxtecan, topotecan, irinotecan, anthracyclines, etoposide); cell-cycle independent drugs, e.g., platinum compounds and analogues (oxaliplatin), triazenes, alkylating agents (cyclophosphamide), spindle poison plant alkaloids (doxorubicin), cytotoxic/antitumor antibiotics (bleomycin, mithramycin, mitomycin), photosensitizers, anti-estrogens and selective estrogen receptor modulators (SERMs), anti-progesterones, estrogen receptor down-regulators (ERDs), estrogen receptor antagonists, luteinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Current standard of care (SOC) treatments for certain cancers, such as colon, in the early-line setting include chemotherapy based on fluoropyrimidine, oxaliplatin, and irinotecan used in combination or sequentially, with option for monoclonal antibodies targeting vascular endothelial growth factor (VEGF) (e.g., bevacizumab, ziv-aflibercept) or its receptors (e.g., ramucirumab), and in patients with Ras wild type tumors, monoclonal antibodies targeting the epidermal growth factor (EGF) receptor (e.g., cetuximab, panitumumab). Treatment options for heavily pre-treated patients beyond the second-line setting are especially limited and associated toxicities can be severe.

“Co-administration” as used herein for agents such as the disclosed anti-PVRIG antibodies, and the anti-PD-1 antibody, Tislelizumab, means that the agents are administered so as to have overlapping therapeutic activities, and not necessarily that the agents are administered simultaneously to the subject. The agents may or may not be in physical combination prior to administration. In an embodiment, the agents are administered to a subject simultaneously or at about the same time. For example, the anti-PVRIG antibody and anti-PD-1 antibody may be contained in separate vials, when in liquid solution, may be mixed into the same intravenous infusion bag or injection device, and administered simultaneously to the patient.

“Co-formulated” or “co-formulation” or “coformulation” or “coformulated” as used herein refers to at least two different antibodies or antigen binding fragments thereof that are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

“Combination therapy” refers to those situations in which a subject is simultaneously exposed to two or more therapeutic regimens (e.g., two or more therapeutic moieties). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity). Such administration also encompasses co-administration in multiple, or in separate containers (e.g., capsules, powders, and liquids) for each active ingredient. Powders and/or liquids can be reconstituted or diluted to a desired dose prior to administration. Thus, the phrase “in combination with” means that an anti-PVRIG antibody is administered to a subject at the same time as, just before, or just after administration of an additional therapeutic agent. In certain embodiments, an anti-PVRIG antibody is administered as a co-formulation with an additional therapeutic agent.

The term “comparison window”, as used herein, includes reference to a segment of any one of the number of contiguous or non-contiguous positions which may range from 10 to 600 positions. In some cases, a comparison window may comprise any subset of the total alignment, either contiguous positions in primary sequence, adjacent positions in tertiary space but discontinuous in the primary sequence, or any other subset of 1 up to all residues in the alignment. In some cases, the comparison window may comprise at least 10, 20, 50, 100, 200, 300, 400, 500, or 600 positions. In some cases, the comparison window may comprise at most 10, 20, 50, 100, 200, 300, 400, 500, or 600 positions. In some cases, the comparison window may comprise at least 50 to 200 positions, or at least 100 to at least 150 positions in which a sequence may be compared to a reference sequence of the same number of contiguous or non-contiguous positions after the two sequences are optimally aligned.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

The terms “constant region” and “constant domain” are interchangeable and have a meaning common in the art. The constant region is an antibody portion, e.g., a carboxyl terminal portion of a light and/or heavy chain which is not directly involved in binding of an antibody to an antigen but which can exhibit various effector functions, such as interaction with the Fc receptor. The constant region of an immunoglobulin molecule generally has a more conserved amino acid sequence relative to an immunoglobulin variable domain.

“Chothia” as used herein means an antibody numbering system described in Al-Lazikani et al., JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.) such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth below in Table 3.

“Contacting” is used in accordance with its plain ordinary meaning and refers to the process of allowing at least two distinct species (e.g., chemical compounds including biomolecules or cells) to become sufficiently proximal to react, interact or physically touch. Thus, the term “contacting” may include allowing two species to react, interact, or physically touch, wherein the two species may be, for example, an anti-PVRIG antibody as described herein and a PVRIG antigen. In certain embodiments, contacting includes, for example, allowing an anti-PVRIG antibody as described herein to interact with a PVRIG antigen.

“Corresponding to” may be used to designate the position/identity of a structural element in a molecule or composition through comparison with an appropriate reference molecule or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, for purposes of simplicity, residues in a polypeptide may be designated using a canonical numbering system based on a reference related polypeptide, so that an amino acid “corresponding to” a residue at position 100, for example, need not actually be the 100th amino acid in an amino acid chain provided it corresponds to the residue found at position 100 in the reference polypeptide. Various sequence alignment strategies are available, comprising software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that may be utilized, for example, to identify “corresponding” residues in polypeptides and/or nucleic acids in accordance with the present disclosure.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “cross-competes” refers to the ability of antibodies, or antigen-binding fragments thereof, to cross-compete for binding to an antigen indicates that such antibodies, or antigen-binding fragments thereof, bind to the same epitope region on the antigen and sterically hinder the binding of other cross-competing antibodies, or antigen-binding fragments thereof, to that particular epitope region. Such cross-competing antibodies, or antigen-binding fragments thereof, are expected to have functional properties very similar to those of the anti-PVRIG antibodies disclosed herein, by virtue of their binding to the same epitope region on PVRIG. Cross-competing antibodies, or antigen-binding fragments thereof, can be readily identified based on their ability to cross-compete with the anti-PVRIG antibodies disclosed herein in standard binding assays such as BIAcore analysis (Acro Biosystems), ELISA assays or flow cytometry.

An antigen binding molecule, an antibody, or an antigen binding molecule thereof “cross-competes” with a reference antibody or an antigen binding molecule thereof if the interaction between an antigen and the first binding molecule, an antibody, or an antigen binding molecule thereof blocks, limits, inhibits, or otherwise reduces the ability of the reference binding molecule, reference antibody, or an antigen binding molecule thereof to interact with the antigen. Cross competition can be complete, e.g., binding of the binding molecule to the antigen completely blocks the ability of the reference binding molecule to bind the antigen, or it can be partial, e.g., binding of the binding molecule to the antigen reduces the ability of the reference binding molecule to bind the antigen. In certain embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope as the reference antigen binding molecule. Numerous types of competitive binding assays can be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin IL-7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) γ. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

The term “domain” refers to a portion of an entity. In some embodiments, a “domain” is associated with a structural and/or functional feature of the entity, e.g., so that, when the domain is physically separated from the rest of its parent entity, it substantially or entirely retains the structural and/or functional feature. In some embodiments, a domain may comprise a portion of an entity that, when separated from that (parent) entity and linked or connected with a different (recipient) entity, substantially retains and/or imparts on the recipient entity one or more structural and/or functional features, e.g., that characterized it in the parent entity. In some embodiments, a domain is a portion of a molecule (e.g., a small molecule, carbohydrate, lipid, nucleic acid, or polypeptide). In some embodiments, a domain is a section of a polypeptide; in some such embodiments, a domain is characterized by a structural element (e.g., an amino acid sequence or sequence motif, α-helix character, β-sheet character, coiled-coil character, random coil character, etc.), and/or by a functional feature (e.g., binding activity, enzymatic activity, folding activity, signaling activity, etc.).

“DCR” or “Disease Control Rate” means CR+PR+SD.

The level of PVRL2 mRNA expression may be compared to the mRNA expression levels of one or more reference genes that are frequently used in quantitative RT-PCR. In some embodiments, a level of PVRL2 expression (protein and/or mRNA) by malignant cells and/or by infiltrating immune cells within a tumor is determined to be “overexpressed” or “elevated” based on comparison with the level of PVRL2 expression (protein and/or mRNA) by an appropriate control. For example, a control PVRL2 protein or mRNA expression level may be the level quantified in nonmalignant cells of the same type or in a section from a matched normal tissue. In some preferred embodiments, PVRL2 expression in a tumor sample is determined to be elevated if PVRL2 protein (and/or PVRL2 mRNA) in the sample is at least 10%, 20%, or 30% greater than in the control.

The term “dosage form” may be used to refer to a physically discrete unit of an active agent (e.g., an antigen binding system or antibody) for administration to a subject. Generally, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population. The total amount of a therapeutic composition or agent administered to a subject is determined by one or more medical practitioners and may involve administration of more than one dosage forms.

The term “dosing regimen” may be used to refer to a set of one or more unit doses that are administered individually to a subject. In some embodiments, a given therapeutic agent has a recommended dosing regimen, which may involve one or more doses. In some embodiments, a dosing regimen comprises a plurality of doses each of which is separated in time from other doses. In some embodiments, a dosing regimen comprises a plurality of doses and consecutive doses are separated from one another by time periods of equal length; in some embodiments, a dosing regimen comprises a plurality of doses and consecutive doses are separated from one another by time periods of at least two different lengths. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen is periodically adjusted to achieve a desired or beneficial outcome.

A “Durable Stable Disease Rate” means stable disease (SD) for −23 weeks, where SD is cancer that is neither decreasing nor increasing in extent or severity.

EC50 is a measure of an antibody's potency. The units of EC50 are given as a concentration, expressed in molar units (M), where 1 M is equivalent to 1 mol/L. Thus, EC50 is the molar concentration of antibody, or antigen-binding fragment thereof, required to obtain 50% binding saturation.

“Effector cell” refers to a cell of the immune system that expresses one or more Fc receptors and mediates one or more effector functions. In some embodiments, effector cells may comprise, without limitation, one or more of monocytes, macrophages, neutrophils, dendritic cells, eosinophils, mast cells, platelets, large granular lymphocytes, Langerhans' cells, natural killer (NK) cells, T-lymphocytes, and B-lymphocytes. Effector cells may be of any organism comprising, without limitation, humans, mice, rats, rabbits, and monkeys.

As used herein, the “elution solution” refers to the solution used to elute the antibody or antigen-binding fragment of interest from the stationary phase. The elution solution can comprise one or more of a salt, or buffering species. The presence of one or more of salt, buffering species, pH or conductivity of the elution solution is/are such that the antibody or antigen-binding fragment is eluted from the stationary phase.

“Effector function” refers to a biological result of interaction of an antibody Fc region with an Fc receptor or ligand. Effector functions comprise, without limitation, antibody-dependent cell-mediated cytotoxicity (ADCC), antibody-dependent cell-mediated phagocytosis (ADCP), and complement-mediated cytotoxicity (CMC). An effector function may be antigen binding dependent, antigen binding independent, or both. ADCC refers to lysis of antibody-bound target cells by immune effector cells. Without wishing to be bound by any theory, ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, eosinophils.

An “epitope” is an antigenic determinant. These are particular chemical groups or peptide sequences on a molecule that are antigenic, i.e., that elicit a specific immune response. An antibody specifically binds a particular antigenic epitope on a polypeptide such as PVRIG. Thus, the epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

As used in this disclosure, the term “epitope” is meant to refer to any antigenic determinant on an immunogen, e.g., any primary immunogen, to which an antibody binds through an antigenic binding site. Determinants or antigenic determinants on an antigen usually consist of chemically active surface groupings of molecules such as amino acids or sugar side chains and usually have specific three-dimensional structural characteristics, as well as specific charge characteristics. In some cases, an epitope may be an area of surface exposed residues and/or carbohydrate moieties on an antigen. In some cases, the area ranges from 100 Å-1500 Å.

Thus, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D. Biol Crystallogr 50 (Pt 4): 339-350; McPherson A. (1990) Eur J Biochem 189: 1-23; Chayen N. E. (1997) Structure 5: 1269-1274; McPherson A. (1976) J Biol Chem 251: 6300-6303). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H. W. et al.; U.S. 2004/0014194), and BUSTER (Bricogne G. (1993) Acta Crystallogr D Biol Crystallogr 49 (Pt 1): 37-60; Bricogne G. (1997) Meth Enzymol 276A: 361-423, ed Carter C. W; Roversi P. et al., (2000) Acta Crystallogr D Biol Crystallogr 56 (Pt 10): 1316-1323). Mutagenesis mapping studies may be accomplished using any method known to one of skill in the art. See, e.g., Champe M. et al., (1995) J Biol Chem 270: 1388-1394 and Cunningham B C & Wells J. A. (1989) Science 244: 1081-1085 for a description of mutagenesis techniques, including alanine scanning mutagenesis techniques.

The term “equilibrium dissociation constant (K_{D})” refers to the dissociation rate constant (k_{off} (kd), time⁻¹) divided by the association rate constant (kₒₙ(ka), time⁻¹, M⁻¹). The kₒₙ and k_{off} can be determined by techniques known to one of ordinary skill in the art, such as BIACORE® or KinExA. Thus, the dissociation constant (K_{D}) is the concentration of a given antibody, such as an anti-PVRIG antibody, or an antigen-binding fragment thereof, at which half of the available binding sites of PVRIG target epitopes are occupied in the system at equilibrium. The smaller the K_{D} in Molar concentration, the greater the affinity an antibody exhibits against its target antigen. Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

In a specific embodiment, provided herein is an antibody, or an antigen-binding fragment thereof, that binds to a target human antigen, e.g., human PVRIG, with higher affinity than to another species of the target antigen, e.g., a non-human PVRIG. In some embodiments, provided herein is an antibody, or an antigen-binding fragment thereof, that binds to human PVRIG, with higher affinity than to another species of one or both target antigens, e.g., a non-human PVRIG, non-human PVRIG, or both. In certain embodiments, provided herein is an antibody, or an antigen-binding fragment thereof, that binds to the target human antigen, e.g., human PVRIG, with a 5%, 10%, 15%, 20%, 25%, 30%, 35%, 40%, 45%, 50%, 55%, 60%, 65%, 70% or higher affinity than to another species of the target antigen as measured by, e.g., a radioimmunoassay, surface plasmon resonance, or kinetic exclusion assay. In certain embodiments, provided herein is an antibody, or an antigen-binding fragment thereof, that binds to human PVRIG, with a 5%, 10%, 15%, 20%, 25%, 30%, 35%, 40%, 45%, 50%, 55%, 60%, 65%, 70% or higher affinity than to another species of one or both target antigens as measured by, e.g., a radioimmunoassay, surface plasmon resonance, or kinetic exclusion assay. In a specific embodiment, an antibody, or an antigen-binding fragment thereof, described herein, which binds to a target human antigen, will bind to another species of the target antigen with less than 10%, 15%, or 20% of the binding of the antibody, or an antigen-binding fragment thereof, to the human antigen as measured by, e.g., a radioimmunoassay, surface plasmon resonance, or kinetic exclusion assay.

In a specific embodiment, molecules that specifically bind to an antigen bind to the antigen with a KA that is at least 2 logs, 2.5 logs, 3 logs, 4 logs or greater than the KA when the molecules bind to another antigen. Binding may comprise preferential association of a binding motif, antibody, or antigen binding system with a target of the binding motif, antibody, or antigen binding system as compared to association of the binding motif, antibody, or an antigen-binding fragment thereof, with an entity that is not the target (i.e., non-target).

In further embodiments, a binding motif, antibody, antigen-binding fragment thereof, or antigen binding system selectively binds a target if binding between the binding motif, antibody, or an antigen-binding fragment thereof, and the target is greater than 2-fold, greater than 5-fold, greater than 10-fold, 20-fold, 30-fold, 40-fold, 50-fold, 60-fold, 70-fold, 80-fold, 90-fold, or greater than 100-fold as compared with binding of the binding motif, antibody, or an antigen-binding fragment thereof, and a non-target. In some embodiments, a binding motif, antibody, or an antigen-binding fragment thereof, selectively binds a target if the binding affinity is less than about 10⁻⁵ M, less than about 10⁻⁶ M, less than about 10⁻⁷ M, less than about 10⁻⁷ M, or less than about 10⁻⁹ M.

In another embodiment, molecules that specifically bind to an antigen bind with a dissociation constant (K_{D}) of about 1×10⁻⁷M. In some embodiments, the antigen binding molecule specifically binds an antigen with “high affinity” when the K_{D} is about 1×10⁻⁹ M to about 5×10⁻⁹ M. In some embodiments, the antigen binding molecule specifically binds an antigen with “very high affinity” when the K_{D} is 1×10⁻¹⁰ M to about 5×10⁻¹⁰ M. In one embodiment, the antigen binding molecule has a K_{D} of 10⁻⁹ M. In one embodiment, the off-rate is less than about 1×10⁻⁵. In other embodiments, the antigen binding molecule binds human PVRIG with a K_{D} of between about 1×10⁻⁷ M and about 1×10⁻¹³ M. In yet another embodiment, the antigen binding molecule binds human PVRIG with a K_{D} of about 1×10⁻¹⁰ M to about 5×10⁻¹⁰ M. In some embodiments, the antigen binding molecule binds human PVRIG with a K_{D} of between about 1×10⁻⁷ M and about 1×10⁻³ M. In yet another embodiment, the antigen binding molecule binds human PVRIG with a K_{D} of about 1×10⁻¹⁰ M to about 5×10⁻¹⁰ M.

The term “excipient” refers to an agent that may be comprised in a composition, for example to provide or contribute to a desired consistency or stabilizing effect. In some embodiments, a suitable excipient may comprise, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

“Extracellular domain” (or “ECD”) refers to a portion of a polypeptide that, when the polypeptide is present in a cell membrane, is understood to reside outside of the cell membrane, in the extracellular space.

The term “fed-batch culture”, as used herein, refers to a method of culturing cells in which additional nutrients are provided to the culture during the cultivation process. A fed-batch culture is typically stopped at some point and the cells and/or components in the medium are harvested. The product accumulates and remains in the bioreactor until the end of the run.

As used herein, “harvesting” an antibody or antigen-binding fragment involves separating it from particulate matter that can include host cells, cell aggregates, and/or lysed cell fragments, into a cell-free fraction that is substantially free of host cells and cellular debris, i.e., a cell-free “permeate.” Such cells and cellular debris are removed from the cell culture broth, for example, by centrifugation, depth filtration and/or microfiltration. For example, to make the cell-free permeate, one can employ hollow fiber membranes or a series of filtration steps such as depth filtration.

A “fragment” or “portion” of a material or entity as described herein has a structure that comprises a discrete portion of the whole, e.g., of a physical entity or abstract entity. In some embodiments, a fragment lacks one or more moieties found in the whole. In some embodiments, a fragment consists of or comprises a characteristic structural element, domain or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer (e.g., 85-90%, 85-95%, 85-100%, 90-95%, 90-100%, or 95-100%). The whole material or entity may in some embodiments be referred to as the “parent” of the fragment.

A “Fab” refers to the “fragment antigen-binding region” which is a region on an antibody that binds to an antigen. It is composed of one constant and one variable domain from each of the heavy and the light chains. The variable domain contains the paratope (the antigen-binding site), comprising the set of six (6) complementarity-determining regions, at the amino terminal end of the monomer. Each arm of the Y-shaped structure binds an epitope on the antigen. The enzyme papain can be used to cleave an immunoglobulin monomer into two Fab fragments and an Fc fragment. Conversely, the enzyme pepsin cleaves below the hinge region, resulting instead in a F(ab′)₂ fragment and a pFc′ fragment (Janeway, C. A., Jr. et al. 2001).

The term “framework” or “framework region” or “FR” refers to amino acid sequences interposed between CDRs. The framework regions serve to align the CDRs for specific binding to an epitope of an antigen (i.e., hold the CDRs in an appropriate orientation for antigen binding). Thus, the phrase “framework region residues” means those variable domain residues other than the hypervariable region residues defined herein as CDR residues. The framework region includes variable light and variable heavy framework regions.

The term “fusion polypeptide” or “fusion protein” generally refers to a polypeptide comprising at least two segments. Generally, a polypeptide containing at least two such segments is considered to be a fusion polypeptide if the two segments are moieties that (1) are not comprised in nature in the same peptide, and/or (2) have not previously been linked or connected to one another in a single polypeptide, and/or (3) have been linked or connected to one another through action of the hand of man.

The term “gene product” or “expression product” generally refers to an RNA transcribed from the gene (pre- and/or post-processing) or a polypeptide (pre- and/or post-modification) encoded by an RNA transcribed from the gene.

The term “heavy chain” when used in reference to an antibody can refer to any distinct type, e.g., alpha (α), delta (δ), epsilon (ε), γ (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “heterologous” means from any source other than naturally occurring sequences. For example, a heterologous nucleotide sequence refers to a nucleotide sequence other than that of the wildtype human costimulatory protein-encoding sequence.

As used herein, a “host cell” refers to any cell of any organism that is used for the purpose of producing a recombinant protein encoded by an expression vector or propagating the expression vector introduced into the host cell. A “mammalian recombinant host cell” refers to a mammalian host cell that comprises a heterologous expression vector, which may or may not be integrated into the host cell chromosome. A “bacterial recombinant host cell” refers to a bacterial host cell that comprises a heterologous expression vector, which may or may not be integrated into the host cell chromosome.

The term “human antibody” herein means an antibody that comprises human immunoglobulin protein sequences only. The variable and constant domain sequences generated may be assembled, or derived from human immunoglobulin sequences, or sequences indistinguishable therefrom. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation).

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fe), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons. In some embodiments, a “humanized” antibody comprises one or more framework domains having substantially the amino acid sequence of a human framework domain, and one or more complementary determining regions having substantially the amino acid sequence as that of a non-human antibody. In some embodiments, a humanized antibody comprises at least a portion of an immunoglobulin constant region (Fe), generally that of a human immunoglobulin constant domain. In some embodiments, a humanized antibody may comprise a CH1, hinge, CH2, CH3, and, optionally, a CH4 region of a human heavy chain constant domain.

A “hybridoma” producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

The term “hypervariable region” means the amino acid residues of an antibody that are responsible for antigen-binding. The hypervariable region comprises amino acid residues from a CDR (e.g., LCDR1, LCDR2 and LCDR3 in the light chain variable domain and HCDR1, HCDR2 and HCDR3 in the heavy chain variable domain).

IC50 is a measure of an antibody's potency. The units of IC50 are given as a concentration, expressed in molar units (M), where 1 M is equivalent to 1 mol/L. Thus, IC50 is the molar concentration of antibody, or antigen-binding fragment thereof, required to obtain 50% inhibition of a specific biological or biochemical function.

The term “identity” refers to the overall relatedness between polymeric molecules, e.g., between nucleic acid molecules (e.g., DNA molecules and/or RNA molecules) and/or between polypeptide molecules. The term “identical” or “percent identity” is a numeric score determined for a pair of aligned amino acid or nucleic acid sequences. Percent identity measures the number of identical residues (“identity”) between two sequences in relation to the length of the alignment across a “comparison window.” The number shows the % of amino acid residues or nucleotides that are the same between two sequences and indicates the degree of primary structure similarity.

Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally considering the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

To calculate percent identity, the sequences being compared are typically aligned in a way that gives the largest match between the sequences. For sequence comparison, generally one sequence acts as a reference sequence, to which test sequences may be compared. When using a sequence comparison algorithm, test and reference sequences may be entered into a computer, subsequent coordinates may be designated, if necessary, and sequence algorithm program parameters may be designated. A polypeptide may be considered to be identical or substantially identical to a second polypeptide, for example, where the two peptides differ only by conservative substitutions. Methods of alignment of sequences for comparison are well-known in the art.

One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm). In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm. Other algorithms are also available for comparison of amino acid or nucleic acid sequences, comprising those available in commercial computer programs such as BLASTN for nucleotide sequences and BLASTP, gapped BLAST, and PSI-BLAST for amino acid sequences. Exemplary such programs are described in Altschul et al., “Basic local alignment search tool,” J. Mol. Biol., 215(3): 403-410, 1990; Altschul et al., Methods in Enzymology; Altschul et al., “Gapped BLAST and PSI-BLAST: a new generation of protein database search programs,” Nucleic Acids Res. 25:3389-3402, 1997; Baxevanis et al., Bioinformatics: A Practical Guide to the Analysis of Genes and Proteins, Wiley, 1998; and Misener et al., (eds.), Bioinformatics Methods and Protocols (Methods in Molecular Biology, Vol. 132), Humana Press, 1999.

In addition to identifying similar sequences, the programs mentioned above generally provide an indication of the degree of similarity. In some embodiments, two sequences are considered to be substantially similar if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues (e.g., 85-90%, 85-95%, 85-100%, 90-95%, 90-100%, or 95-100%). In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. Sequences with substantial sequence similarity may be homologs of one another.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

Positive therapeutic effects in cancer can be measured in a number of ways (See, W. A Weber, J Nucl. Med. 50: I S-IOS (2009)). One common measure of efficacy is the T/C ratio, which is the ratio of tumor volume in control versus treated mice at a specified time, where T/C (%)=Median tumor volume of the treated/Median tumor volume of the control×100. With respect to tumor growth inhibition, according to NCI standards, a T/C≤42% is the minimum level of anti-tumor activity. A T/C<10% is considered a high anti-tumor activity level. In some embodiments, the treatment achieved by administration of a composition of the present disclosure is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the compositions, treatment methods, and uses of the present disclosure may not be effective in achieving a positive therapeutic effect in every patient, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, Id.), or other assays known in the art. Thus, under certain designated immunoassay conditions, the antibodies or antigen-binding fragments thereof specifically bind to a particular antigen at least two (2) fold when compared to the background level and do not specifically bind in a significant amount to other antigens present in the sample. In another aspect, under designated immunoassay conditions, the antibodies or antigen-binding fragments thereof, specifically bind to a particular antigen at least ten (10) fold when compared to the background level of binding and do not specifically bind in a significant amount to other antigens present in the sample.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T and NK cell therapies. T and NK cell therapies can include adoptive cell therapies, tumor-infiltrating lymphocyte (TIL) immunotherapies, autologous cell therapies, engineered autologous cell therapy (eACT™), and allogeneic T and NK cell transplantation.

The terms “improve,” “increase,” “inhibit,” and “reduce” indicate values that are relative to a baseline or other reference measurement. In some embodiments, an appropriate reference measurement may comprise a measurement in certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in comparable system known or expected to respond in a comparable way, in presence of the relevant agent or treatment.

The term “in vitro” refers to events occurring in an artificial environment, e.g., in a test tube, reaction vessel, cell culture, etc., rather than within a multi-cellular organism. The term “in vitro cell” refers to any cell which is cultured ex vivo. In particular, an in vitro cell can include a T cell. The term “in vivo” refers to events that occur within a multi-cellular organism, such as a human or a non-human animal.

The term “isolated” refers to a substance that (1) has been separated from at least some components with which it was associated at an earlier time or with which the substance would otherwise be associated, and/or (2) is present in a composition that comprises a limited or defined amount or concentration of one or more known or unknown contaminants. An isolated substance, in some embodiments, may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% (e.g., 85-90%, 85-95%, 85-100%, 90-95%, 90-100%, or 95-100%) of other non-substance components with which the substance was associated at an earlier time, e.g., other components or contaminants with which the substance was previously or otherwise would be associated. In certain instances, a substance is isolated if it is present in a composition that comprises a limited or reduced amount or concentration of molecules of a same or similar type. For instance, in certain instances, a nucleic acid, DNA, or RNA substance is isolated if it is present in a composition that comprises a limited or reduced amount or concentration of non-substance nucleic acid, DNA, or RNA molecules. For instance, in certain instances, a polypeptide substance is isolated if it is present in a composition that comprises a limited or reduced amount or concentration of non-substance polypeptide molecules. In certain embodiments, an amount may be, e.g., an amount measured relative to the amount of a desired substance present in a composition. In certain embodiments, a limited amount may be an amount that is no more than 100% of the amount of substance in a composition, e.g., no more than 1%, 5%, 10%, 20%, 30%, 40%, 50%, 60%, 70%, 80%, 90%, or 95% of the amount of substance in a composition (e.g., 85-90%, 85-95%, 85-100%, 90-95%, 90-100%, or 95-100%). In certain instances, a composition is pure or substantially pure with respect to a selected substance. In some embodiments, an isolated substance is about 80%, about 85%, about 90%, about 91%, about 92%, about 93%, about 94%, about 95%, about 96%, about 97%, about 98%, about 99%, or more than about 99% pure (e.g., 85-90%, 85-95%, 85-100%, 90-95%, 90-100%, or 95-100%).

“Isotype” refers to the antibody class or subclass (e.g., IgM or IgG1) that is encoded by the heavy chain constant region genes. Thus, the term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman Ab may be humanized by recombinant methods to reduce its immunogenicity in man. Where not expressly stated, and unless the context indicates otherwise, the term “antibody” also includes an antigen binding fragment or an antigen-binding portion of any of the aforementioned immunoglobulins, and includes a monovalent and a divalent fragment or portion, and a single chain Ab.

The term “Kabat numbering” and like terms are recognized in the art and refer to a system of numbering amino acid residues in the heavy and light chain variable regions of an antibody, or an antigen-binding molecule thereof. In certain aspects, the CDRs of an antibody can be determined according to the Kabat numbering system (see, e.g., Kabat E. A. & Wu T. T. (1971) Ann NY Acad Sci 190: 382-391 and Kabat E. A. et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

The term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

“Linker” (L) or “linker domain” or “linker region” refers to an oligo- or polypeptide region from about 1 to 100 amino acids in length, which links together any of the domains/regions of the antigen-binding fragments of the disclosure. Linkers may be composed of flexible residues like glycine and serine so that the adjacent protein domains are free to move relative to one another. Longer linkers may be used when it is desirable to ensure that two adjacent domains do not sterically interfere with one another. Two scFvs may be linked by (G4S)×3 (times three), where a single G4S segment is GGGGS (SEQ ID NO:186).

A “loading dose” is an initial higher dose of a drug that may be given at the beginning of a course of treatment before dropping down to a lower maintenance dose.

The term “lymphocyte” includes natural killer (NK) cells, T cells, or B cells. NK cells are a type of cytotoxic (cell toxic) lymphocyte that represent a major component of the inherent immune system. NK cells reject tumors and cells infected by viruses through the cytolytic process of apoptosis or programmed cell death. NK cells are termed “natural killers” because they do not require activation in order to kill cells.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies comprising different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art (see, e.g., Kohler et al., Nature 1975 256:495-497; U.S. Pat. No. 4,376,110; Ausubel et al., Current Protocols in Molecular Biology 1992; Harlow et al., Antibodies: A Laboratory Manual, Cold spring Harbor Laboratory 1988; and Colligan et al., Current Protocols in Immunology 1993).

Natural killer (NK) cells mediate effective cytotoxicity against tumor cells and unlike T cells, lack the potential to cause graft versus host disease (GVHD) in the allogeneic setting. NK cells may be made available as an off-the-shelf cellular therapy product for immediate clinical use (Daher et al., 2018).

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Thus, the term “nucleic acid” refers to any polymeric chain of nucleotides. A nucleic acid may be DNA, RNA, or a combination thereof. In some embodiments, a nucleic acid comprises one or more natural nucleic acid residues. In some embodiments, a nucleic acid comprises of one or more nucleic acid analogs. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long (e.g., 20 to 100, 20 to 500, 20 to 1000, 20 to 2000, or 20 to 5000 or more residues). In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

“Operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element. In the context of nucleic acids, the term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “peptidyl” and “peptidyl moiety” means a monovalent peptide.

“PVRL2 expression positive” or “PD-L1 expression positive” refers to a Tumor Proportion Score, Mononuclear Inflammatory Density Score or Combined Positive Score of at least 1%; or elevated level of PVRL2 expression or PD-L1 expression (protein and/or mRNA) by malignant cells and/or by infiltrating immune cells within a tumor compared to an appropriate control.

The term “pharmaceutical composition” refers to preparations with pharmaceutically acceptable excipients which are in such form as to permit the active ingredients to be effective, and which contains no additional components which are toxic to the subjects to which the composition would be administered. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some aspects, the present disclosure provides compositions, e.g., pharmaceutically acceptable compositions, which include an anti-PVRIG antibody described herein, formulated together with at least one pharmaceutically acceptable excipient.

The term “pharmaceutically acceptable” refers to a molecule or composition that, when administered to a recipient, is not deleterious to the recipient thereof, or that any deleterious effect is outweighed by a benefit to the recipient thereof.

Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; tale; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

A “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection. In some embodiments, the antibody is administered by way of a syringe infusion system.

As used herein, “purifying” an antibody or antigen-binding fragment of interest or “purified composition” refers to increasing the degree of purity of the antibody or antigen binding fragment in the composition by removing (completely or partially) at least one impurity from the composition. The impurity can be host cell components such as serum, proteins or nucleic acids, cellular debris, growth medium or antibody aggregates. The term is not intended to refer to a complete absence of such biological molecules or to an absence of water, buffers, or salts or to components of a pharmaceutical composition that includes the antibody or antigen binding fragment.

“RECIST 1.1 Response Criteria” as used herein means the definitions set forth in Eisenhauer et al., Eur. J Cancer 45:228-247 (2009) for target lesions or nontarget lesions, as appropriate based on the context in which response is being measured.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

“Regulatory T cells” (“Treg”, “Treg cells”, or “Tregs”) refer to a lineage of CD4⁺ T lymphocytes that participate in controlling certain immune activities, e.g., autoimmunity, allergy, and response to infection. Tregs may be identified by the expression of the biomarkers CD4, CD25 and Foxp3, and low expression of CD127. Naturally occurring Treg cells normally constitute about 5-10% of the peripheral CD4⁺ T lymphocytes. However, Treg cells within a tumor microenvironment (i.e., tumor-infiltrating Treg cells), may make up as much as 20-30% of the total CD4⁺ T lymphocyte population.

“Responder patient” when referring to a specific anti-tumor response to treatment with a therapy as described herein, means the patient exhibited the anti-tumor response.

The term “sample” generally refers to an aliquot of material obtained or derived from a source of interest. In some embodiments, a source of interest is a biological or environmental source. In some embodiments, a source of interest may comprise a cell or an organism, such as a cell population, tissue, or animal (e.g., a human). In some embodiments, a source of interest comprises biological tissue or fluid. In some embodiments, a biological tissue or fluid may comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may comprise an intracellular fluid, an extracellular fluid, an intravascular fluid (blood plasma), an interstitial fluid, a lymphatic fluid, and/or a transcellular fluid. In some embodiments, a biological fluid may comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

The term “stage of cancer” refers to a qualitative or quantitative assessment of the level of advancement of a cancer. In some embodiments, criteria used to determine the stage of a cancer may comprise, without limitation, one or more of where the cancer is located in a body, tumor size, whether the cancer has spread to lymph nodes, whether the cancer has spread to one or more different parts of the body, etc. In some embodiments, cancer may be staged using the so-called TNM System, according to which T refers to the size and extent of the main tumor, usually called the primary tumor; N refers to the number of nearby lymph nodes that have cancer; and M refers to whether the cancer has metastasized. In some embodiments, a cancer may be referred to as Stage 0 (abnormal cells are present without having spread to nearby tissue, also called carcinoma in situ, or CIS; CIS is not cancer, though could become cancer), Stage I-III (cancer is present; the higher the number, the larger the tumor and the more it has spread into nearby tissues), or Stage IV (the cancer has spread to visceral organs and/or distant parts of the body). In some embodiments, a cancer may be assigned to a stage selected from the group consisting of: in situ; localized (cancer is limited to the place where it started, with no sign that it has spread); regional (cancer has spread to nearby lymph nodes, tissues, or organs): distant (cancer has spread to distant parts of the body); and unknown (there is not enough information to determine the stage).

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex, that specifically binds with a cognate stimulatory ligand present on an antigen presenting cell. A “stimulatory ligand” is a ligand that when present on an antigen presenting cell (e.g., an APC, a dendritic cell, a B-cell, and the like) can specifically bind with a stimulatory molecule on an NK cell, a B cell or a T lymphocyte, thereby mediating a primary response by the immune cell, including, but not limited to, activation, initiation of an immune response, proliferation, and the like.

The term “subject” in the context of the present disclosure is a mammal, e.g., a primate, preferably a higher primate, e.g., a human (e.g., a patient having, or at risk of having, a disorder described herein).

In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution.

A “sustained response” means a sustained therapeutic effect after cessation of treatment with a therapeutic agent, or a combination of agents, as described herein. In some embodiments, the sustained response has a duration that is at least the same as the treatment duration, or at least 1.5, 2.0, 2.5 or 3 times longer than the treatment duration.

T cells play a major role in cell-mediated-immunity (no antibody involvement). The thymus, a specialized organ of the immune system, is primarily responsible for the T cell's maturation. There are six types of T cells, namely: Helper T-cells (e.g., CD4⁺ cells), Cytotoxic T cells (also known as TC, cytotoxic T lymphocyte, CTL, T-killer cell, cytolytic T cell, CD8⁺ T cells or killer T cell), Memory T-cells ((i) stem memory T_{SCM} cells, like naive cells, are CD45RO⁻, CCR7⁺, CD45RA⁺, CD62L⁺ (L-selectin), CD27⁺, CD28⁺ and IL-7Ra⁺, but they also express large amounts of CD95, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T-cells (Tregs, suppressor T cells, or CD4⁺CD25⁺ regulatory T cells), Natural Killer T-cells (NKT) and F Delta T-cells.

A “target” is any molecule bound by a binding motif, antigen binding system, or binding agent, e.g., an antibody. In some embodiments, a target is an antigen or epitope of the present disclosure.

The phrase “therapeutic agent” may refer to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms or human subjects. In some embodiments, an appropriate population may be defined by various criteria, such as a certain age group, gender, genetic background, preexisting clinical conditions, in accordance with presence or absence of a biomarker, etc. In some embodiments, a therapeutic agent is a substance that may be used to alleviate, ameliorate, relieve, inhibit, prevent, delay onset of, reduce severity of, and/or reduce incidence of one or more symptoms or features of a disease, disorder, and/or condition. In some embodiments, a therapeutic agent is an agent that has been or is required to be approved by a government agency before it may be marketed for administration to humans. In some embodiments, a therapeutic agent is an agent for which a medical prescription is required for administration to humans.

A “therapeutically effective amount,” “effective dose,” “effective amount,” or “therapeutically effective dosage” of a therapeutic agent, e.g., an antibody, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

“Tissue Section” refers to a single part or piece of a tissue sample, e.g., a thin slice of tissue cut from a sample of a normal tissue or of a tumor.

The terms “transduction” and “transduced” refer to the process whereby foreign DNA is introduced into a cell via viral vector (Jones et al., 1998, Genetics: principles and analysis, Boston: Jones & Bartlett Publ.). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Transformation” refers to any process by which exogenous DNA is introduced into a host cell. Transformation may occur under natural or artificial conditions using various methods. Transformation may be achieved using any known method for the insertion of foreign nucleic acid sequences into a prokaryotic or eukaryotic host cell. In some embodiments, some transformation methodology is selected based on the host cell being transformed and/or the nucleic acid to be inserted. Methods of transformation may comprise, yet are not limited to, viral infection, electroporation, and lipofection. In some embodiments, a “transformed” cell is stably transformed in that the inserted DNA is capable of replication either as an autonomously replicating plasmid or as part of the host chromosome. In some embodiments, a transformed cell may express introduced nucleic acid.

“Treat” or “treating” a cancer, as used herein, refers to the administration of a composition of the present disclosure to a subject having an immune condition or cancerous condition, or diagnosed with a cancer or pathogenic infection (e.g. viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an anti-tumor immune response, stimulating an immune response to a pathogen, toxin, and/or self-antigen, stimulating an immune response to a viral infection, decreasing the number of one or more tumor markers, halting or delaying the growth of a tumor or blood cancer or progression of disease associated with PVRIG binding to its ligand PVRL2 (“PVRIG-related disease”) such as cancer, stabilization of PVRIG-related disease, inhibiting the growth or survival of tumor cells, eliminating or reducing the size of one or more cancerous lesions or tumors, decreasing the level of one or more tumor markers, ameliorating, abrogating the clinical manifestations of PVRIG-related disease, reducing the severity or duration of the clinical symptoms of PVRIG-related disease such as cancer, prolonging the survival of a patient relative to the expected survival in a similar untreated patient, inducing complete or partial remission of a cancerous condition or other PVRIG-related disease.

The terms “treatment regimen”, “dosing protocol” and “dosing regimen” are used interchangeably to refer to the dose and timing of administration of each therapeutic agent in a combination of the invention.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and 30 lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC” refers to the set of criteria described in Nishino et al., Developing a Common Language for Tumor Response to Immunotherapy: Immune-related Response Criteria using Unidimensional measurements. Clin Cancer Res. 2013; 19(14):3936-3943. These criteria utilize the longest diameter (cm) of each lesion. The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Tumor Proportion Score (TPS)” refers to the percentage of tumor cells expressing PD-L1 or another tumor antigen, such as PVRL2, on the cell membrane at any intensity (weak, moderate or strong). Linear partial or complete cell membrane staining is interpreted as positive for PD-L1 or another antigen of interest, such as PVRL2.

The term “variable region” or “variable domain” as used herein means the segment of IgG chains which is variable in sequence between different antibodies. Typically, it extends to Kabat residue 109 in the light chain and 113 in the heavy chain. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

Term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference for any purpose.

The terms “VL”, LCVR” and “VL domain” are used interchangeably to refer to the light chain variable region of an antibody or an antigen-binding molecule thereof.

The terms “VH”, “HCVR” and “VH domain” are used interchangeably to refer to the heavy chain variable region of an antibody or an antigen-binding molecule thereof.

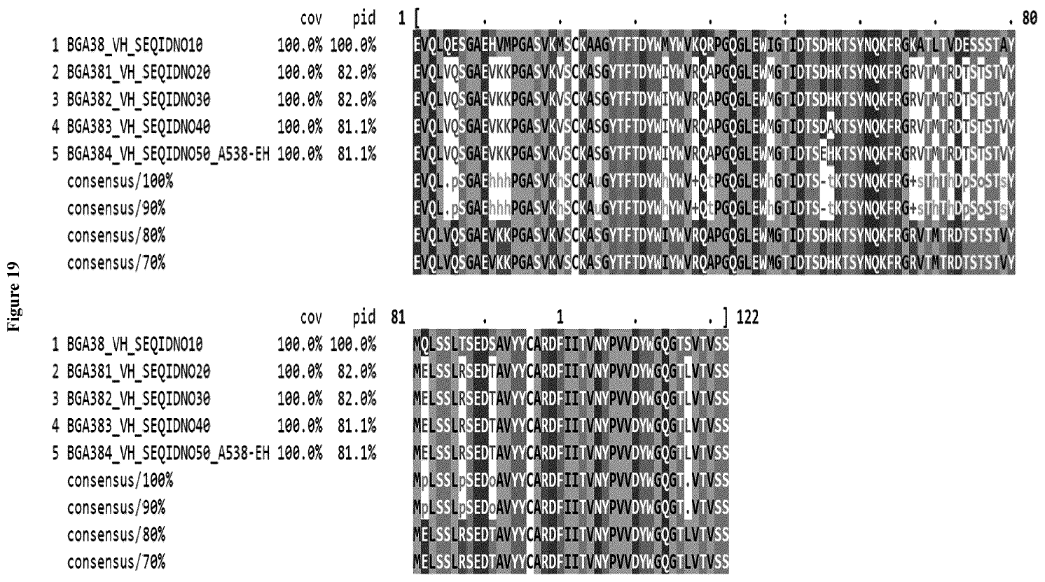

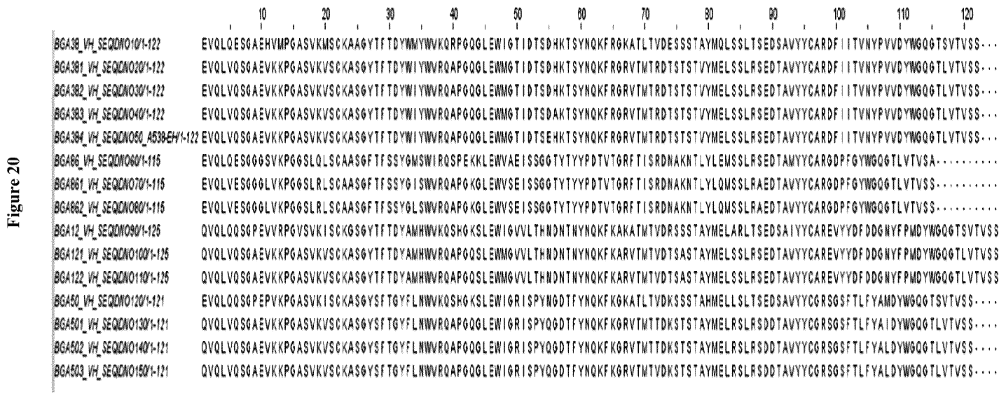

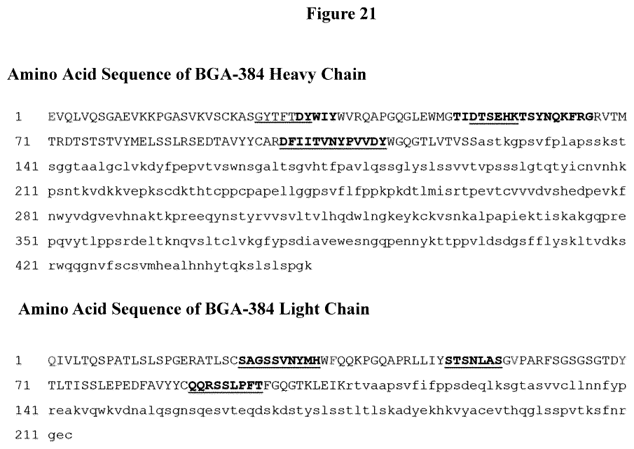

Disclosed herein are humanized monoclonal antibodies, and antigen-binding fragments thereof, that specifically bind human PVRIG. Certain common structural features are critical to carrying out the claimed function of specifically binding to human PVRIG. A structural consensus of which CDRs, showing which subset of residues in the combination of CDRs, is essential for the recited function of specifically binding to human PVRIG is shown in the alignments of FIGS. 19 and 20. Information from the alignments provides guidance to a skilled artisan as to how to identify other species that fall within the claimed functional genus. The disclosed detailed alignments of the claimed CDRs, as well as the full length variable heavy and light chain binding regions, reflect the full variety of the claimed genus.

In certain embodiments, a HCVR sequence having at least 82%, 83%, 84%, 85%, 86%, 87%, 88%, 89%, 90%, 91%, 92%, 93%, 94%, 95%, 96%, 97%, 98% or 99% amino acid sequence identity to the amino acid sequence of SEQ ID NO:20 or SEQ ID NO:50 contains substitutions (e.g., conservative substitutions), insertions, or deletions relative to the reference sequence, but an anti-PVRIG antibody or antigen-binding fragment thereof comprising that sequence retains the ability to specifically bind to PVRIG. In certain embodiments, a total of 1 to 10 amino acids have been substituted, inserted and/or deleted in SEQ ID NO:20 or SEQ ID NO:50. In certain embodiments, a total of 1 to 5 amino acids have been substituted, inserted and/or deleted in SEQ ID NO:20 or SEQ ID NO:50. In certain embodiments, the substitutions, insertions, or deletions occur in regions outside the CDRs (i.e., in the FRs).

In certain embodiments, a LCVR sequence having at least 83%, 84%, 85%, 86%, 87%, 88%, 89%, 90%, 91%, 92%, 93%, 94%, 95%, 96%, 97%, 98% or 99% amino acid sequence identity to the amino acid sequence of SEQ ID NO:21 or SEQ ID NO:51 contains substitutions (e.g., conservative substitutions), insertions, or deletions relative to the reference sequence, but an anti-PVRIG antibody or antigen-binding fragment thereof comprising that sequence retains the ability to specifically bind to PVRIG. In certain embodiments, a total of 1 to 10 amino acids have been substituted, inserted and/or deleted in SEQ ID NO:21 or SEQ ID NO:51. In certain embodiments, a total of 1 to 5 amino acids have been substituted, inserted and/or deleted in SEQ ID NO:21 or SEQ ID NO:51. In certain embodiments, the substitutions, insertions, or deletions occur in regions outside the CDRs (i.e., in the FRs).

The structural consensus sequences disclosed herein are intended to guide those skilled in the art as to how to predict other species that fall within the claimed structural/functional genus of specifically binding to PVRIG. The inventors have disclosed common structural features of a genus of antibodies, or antigen-binding fragments thereof, that correlate with a recited function. The common structural features are disclosed as species which are representative of the full variety of the claimed genus.

As used herein, a “variant of BGA384” means a monoclonal antibody that comprises heavy chain and light chain sequences that are substantially identical to those in BGA384, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, BGA384 and a BGA384 variant comprise identical CDR sequences, but differ from each other due to having a conservative amino acid substitution at no more than three or six other positions in their full-length light and heavy chain sequences, respectively. A BGA384 variant is substantially the same as BGA384 with respect to the following properties: binding affinity to PVRIG and ability to block the binding of PVRIG to PVRL2.

### DETAILED DESCRIPTION

The present disclosure provides for antibodies, antigen-binding fragments, that specifically bind human PVRIG. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders. In some embodiments, anti-PVRIG antibodies or antigen-binding fragments thereof described herein enhance T cell activation, e.g., relative to a reference anti-PVRIG antibody or antigen-binding fragment thereof (e.g., AB-407 is a chimeric monoclonal antibody against the mouse PVRIG). In some embodiments, anti-PVRIG antibodies or antigen-binding fragments thereof described herein enhance NK cell activation, e.g., relative to a reference anti-PVRIG antibody or antigen-binding fragment thereof (e.g., AB-407). In some embodiments, anti-PVRIG antibodies or antigen-binding fragments thereof described herein enhance IFN-γ production by immune cells described herein, e.g., relative to a reference anti-PVRIG antibody or antigen-binding fragment thereof (e.g., AB-407). In some embodiments, anti-PVRIG antibodies or antigen-binding fragments thereof described herein in combination with one or more immune checkpoint inhibitors described herein (e.g., one or both of an anti-PD-1 antibody or antigen-binding fragment thereof or an anti-TIGIT antibody or antigen-binding fragment thereof) decrease exhaustion of immune effector cells, e.g., relative to each agent alone (e.g., AB-407).

### Anti-PVRIG Antibodies

The present disclosure provides for antibodies or antigen-binding fragments thereof that specifically bind to PVRIG. Antibodies or antigen-binding fragments of the present disclosure include, but are not limited to, the antibodies or antigen-binding fragments thereof, generated as described, below.

The present disclosure provides antibodies or antigen-binding fragments that specifically bind to PVRIG, wherein said antibodies or antibody fragments (e.g., antigen-binding fragments) comprise a VH domain comprising an amino acid sequence of SEQ ID NO:10, SEQ ID NO:20, SEQ ID NO:30, SEQ ID NO:40, SEQ ID NO:50, SEQ ID NO:60, SEQ ID NO:70, SEQ ID NO:80, SEQ ID NO:90, SEQ ID NO:100, SEQ ID NO:110, SEQ ID NO:120, SEQ ID NO:130, SEQ ID NO:140 or SEQ ID NO:150 (Table 5). The present disclosure also provides antibodies or antigen-binding fragments that specifically bind PVRIG, wherein said antibodies or antigen-binding fragments comprise a HCDR comprising an amino acid sequence of any one of the HCDRs listed in Table 5. In one aspect, the present disclosure provides antibodies or antigen-binding fragments that specifically bind to PVRIG, wherein said antibodies comprise (or alternatively, consist of) one, two, three, or more HCDRs comprising an amino acid sequence of any of the HCDRs listed in Table 5.

The present disclosure provides for antibodies or antigen-binding fragments that specifically bind to PVRIG, wherein said antibodies or antigen-binding fragments comprise a VL domain comprising an amino acid sequence of SEQ ID NO:11, SEQ ID NO:21, SEQ ID NO:31, SEQ ID NO:41, SEQ ID NO:51, SEQ ID NO:61, SEQ ID NO:71, SEQ ID NO:81, SEQ ID NO:91, SEQ ID NO:101, SEQ ID NO:111, SEQ ID NO:121, SEQ ID NO:131, SEQ ID NO:141 or SEQ ID NO:151 (Table 5). The present disclosure also provides antibodies or antigen-binding fragments that specifically bind to PVRIG, wherein said antibodies or antigen-binding fragments comprise a LCDR comprising an amino acid sequence of any one of the LCDRs listed in Table 5. In particular, the disclosure provides for antibodies or antigen-binding fragments that specifically bind to PVRIG, said antibodies or antigen-binding fragments comprise (or alternatively, consist of) one, two, three or more LCDRs comprising an amino acid sequence of any of the LCDRs listed in Table 5.

Because specific binding was achieved with heavy and light chain variable regions having as little as 82% sequence identity to each other, there is structural support for claims directed to a genus of antibodies having a heavy chain variable region (HCVR) comprising an amino acid sequence having at least 82%, 83%, 84%, 85%, 86%, 87%, 88%, 89%, 90%, 91%, 92%, 93%, 94%, 95%, 96%, 97%, 98% or 99% sequence identity to SEQ ID NO:20 or SEQ ID NO:50, and a light chain variable region (LCVR) comprising an amino sequence having at least 82%, 83%, 84%, 85%, 86%, 87%, 88%, 89%, 90%, 91%, 92%, 93%, 94%, 95%, 96%, 97%, 98% or 99% sequence identity to SEQ ID NO:21 or SEQ ID NO:51.

The present disclosure also provides nucleic acid sequences that encode VH, VL, the full length heavy chain, and the full length light chain of the antibodies that specifically bind to PVRIG. Such nucleic acid sequences can be optimized for expression in mammalian cells.

TABLE 5
Anti-PVRIG Antibody Sequences

| Antibody | SEQ ID NO | | SEQUENCE |
|---|---|---|---|
| BGA38 | SEQ ID NO: 4 | HCDR1 | DYWMY |
| | | (Kabat) | |
| | SEQ ID NO: 5 | HCDR2 | TIDTSDHKTSYNQKFRG |
| | | (Kabat) | |
| | SEQ ID NO: 6 | HCDR3 | DFIITVNYPVVDY |
| | | (Kabat) | |
| | SEQ ID NO: 7 | LCDR1 | SAGSSVNYMH |
| | | (Kabat) | |
| | SEQ ID NO: 8 | LCDR2 | STSNLAS |
| | | (Kabat) | |
| | SEQ ID NO: 9 | LCDR3 | QQRSSLPFT |
| | | (Kabat) | |
| | SEQ ID NO: 10 | VH AA | EVQLQESGAEHVMPGASVKMSCKAAGYTF |
| | | | TDYWMYWVKQRPGQGLEWIGTIDTSDHKT |
| | | | SYNQKFRGKATLTVDESSSTAYMQLSSLTSE |
| | | | DSAVYYCARDFIITVNYPVVDYWGQGTSVT |
| | | | VSS |
| | SEQ ID NO: 11 | VL AA | QNVLTQSPAIMSASPGEKVTITCSAGSSVNY |
| | | | MHWFQQKPGTSPKLWIYSTSNLASGVPARF |
| | | | SGSGSGTSYSLTISRMEAEDAATYYCQQRSS |
| | | | LPFTFGSGTKLEIK |
| | SEQ ID NO: 12 | VH DNA | GAGGTGCAGCTGCAAGAGAGCGGCGCCG |
| | | | AGCACGTGATGCCCGGCGCCTCCGTGAAG |
| | | | ATGAGCTGCAAGGCCGCCGGCTACACCTT |
| | | | CACCGACTACTGGATGTACTGGGTGAAGC |
| | | | AGAGACCCGGCCAAGGCCTGGAGTGGATC |
| | | | GGCACCATCGACACAAGCGACCACAAGAC |
| | | | AAGCTACAATCAGAAGTTCAGAGGCAAGG |
| | | | CCACCCTGACCGTGGACGAGAGCAGCAGC |
| | | | ACCGCCTACATGCAGCTGAGCAGCCTGAC |
| | | | AAGCGAGGACAGCGCCGTGTACTACTGCG |
| | | | CTAGAGACTTCATCATCACCGTGAACTACC |
| | | | CCGTGGTGGACTACTGGGGCCAAGGCACA |
| | | | AGCGTGACCGTGAGCAGC |
| | SEQ ID NO: 13 | VL DNA | CAGAACGTGCTGACACAGAGCCCCGCCAT |
| | | | CATGAGCGCCTCCCCCGGCGAGAAGGTGA |
| | | | CCATCACCTGCAGCGCCGGCAGCAGCGTG |
| | | | AACTACATGCACTGGTTTCAGCAGAAGCC |
| | | | CGGCACAAGCCCCAAGCTGTGGATCTACA |
| | | | GCACAAGCAACCTGGCCTCCGGCGTGCCC |
| | | | GCTAGATTCAGCGGCAGCGGCAGCGGGAC |
| | | | AAGCTACAGCCTGACCATCAGCAGAATGG |
| | | | AGGCCGAGGACGCCGCCACCTACTACTGT |
| | | | CAGCAGAGAAGCAGCCTGCCCTTCACCTT |
| | | | CGGCAGCGGCACCAAGCTGGAGATCAAG |
| BGA381 | SEQ ID NO: 14 | HCDR1 | DYWIY |
| | | (Kabat) | |
| | SEQ ID NO: 15 | HCDR2 | TIDTSDHKTSYNQKFRG |
| | | (Kabat) | |
| | SEQ ID NO: 16 | HCDR3 | DFIITVNYPVVDY |
| | | (Kabat) | |
| | SEQ ID NO: 17 | LCDR1 | SAGSSVNYMH |
| | | (Kabat) | |
| | SEQ ID NO: 18 | LCDR2 | STSNLAS |
| | | (Kabat) | |
| | SEQ ID NO: 19 | LCDR3 | QQRSSLPFT |
| | | (Kabat) | |
| | SEQ ID NO: 20 | VH AA | EVQLVQSGAEVKKPGASVKVSCKASGYTFT |
| | | | DYWIYWVRQAPGQGLEWMGTIDTSDHKTS |
| | | | YNQKFRGRVTMTRDTSTSTVYMELSSLRSE |
| | | | DTAVYYCARDFIITVNYPVVDYWGQGTLVT |
| | | | VSS |
| | SEQ ID NO: 21 | VL AA | QIVLTQSPATLSLSPGERATLSCSAGSSVNYM |
| | | | HWFQQKPGQAPRLLIYSTSNLASGVPARFSG |
| | | | SGSGTDYTLTISSLEPEDFAVYYCQQRSSLPF |
| | | | TFGQGTKLEIK |
| | SEQ ID NO: 22 | VH DNA | GAGGTGCAGCTGGTGCAGAGCGGCGCCG |
| | | | AGGTGAAGAAGCCCGGCGCTAGCGTGAA |
| | | | GGTGAGCTGCAAGGCTAGCGGCTACACCT |
| | | | TCACCGACTACTGGATCTACTGGGTGAGAC |
| | | | AAGCCCCCGGCCAAGGCCTGGAGTGGATG |
| | | | GGCACCATCGACACAAGCGACCACAAGAC |
| | | | AAGCTACAATCAGAAGTTCAGAGGCAGAG |
| | | | TGACCATGACAAGAGACACAAGCACAAGC |
| | | | ACCGTGTACATGGAGCTGAGCAGCCTGAG |
| | | | AAGCGAGGACACCGCCGTGTACTACTGCG |
| | | | CTAGAGACTTCATCATCACCGTGAACTACC |
| | | | CCGTGGTGGACTACTGGGGCCAAGGCACC |
| | | | CTGGTGACCGTGAGCAGC |
| | SEQ ID NO: 23 | VL DNA | CAGATCGTGCTGACACAGAGCCCCGCCAC |
| | | | CCTGAGCCTGAGCCCCGGCGAAAGAGCCA |
| | | | CCCTGAGCTGCAGCGCCGGCAGCAGCGTG |
| | | | AACTACATGCACTGGTTTCAGCAGAAGCC |
| | | | CGGCCAAGCCCCTAGACTGCTGATCTACA |
| | | | GCACAAGCAACCTGGCTAGCGGCGTGCCC |
| | | | GCTAGATTCAGCGGCAGCGGCAGCGGCAC |
| | | | CGACTACACCCTGACCATCAGCAGCCTGG |
| | | | AGCCCGAGGACTTCGCCGTGTACTACTGTC |
| | | | AGCAGAGAAGCAGCCTGCCCTTCACCTTC |
| | | | GGCCAAGGCACCAAGCTGGAGATCAAG |
| BGA382 | SEQ ID NO: 24 | HCDR1 | DYWIY |
| | | (Kabat) | |
| | SEQ ID NO: 25 | HCDR2 | TIDTSDHKTSYNQKFRG |
| | | (Kabat) | |
| | SEQ ID NO: 26 | HCDR3 | DFIITVNYPVVDY |
| | | (Kabat) | |
| | SEQ ID NO: 27 | LCDR1 | SAGSSVNYLH |
| | | (Kabat) | |
| | SEQ ID NO: 28 | LCDR2 | STSNLAS |
| | | (Kabat) | |
| | SEQ ID NO: 29 | LCDR3 | QQRSSLPFT |
| | | (Kabat) | |
| | SEQ ID NO: 30 | VH | EVQLVQSGAEVKKPGASVKVSCKASGYTFT |
| | | AA | DYWIYWVRQAPGQGLEWMGTIDTSDHKTS |
| | | | YNQKFRGRVTMTRDTSTSTVYMELSSLRSE |
| | | | DTAVYYCARDFIITVNYPVVDYWGQGTLVT |
| | | | VSS |
| | SEQ ID NO: 31 | VL | QNVLTQSPATLSLSPGERATLSCSAGSSVNYL |
| | | AA | HWFQQKPGQAPRLLIYSTSNLASGVPARFSG |
| | | | SGSGTDYTLTISSLEPEDFAVYYCQQRSSLPF |
| | | | TFGQGTKLEIK |
| | SEQ ID NO: 32 | VH | GAGGTGCAGCTGGTGCAGAGCGGCGCCG |
| | | DNA | AGGTGAAGAAGCCCGGCGCTAGCGTGAA |
| | | | GGTGAGCTGCAAGGCTAGCGGCTACACCT |
| | | | TCACCGACTACTGGATCTACTGGGTGAGAC |
| | | | AAGCCCCCGGCCAAGGCCTGGAGTGGATG |
| | | | GGCACCATCGACACAAGCGACCACAAGAC |
| | | | AAGCTACAATCAGAAGTTCAGAGGCAGAG |
| | | | TGACCATGACAAGAGACACAAGCACAAGC |
| | | | ACCGTGTACATGGAGCTGAGCAGCCTGAG |
| | | | AAGCGAGGACACCGCCGTGTACTACTGCG |
| | | | CTAGAGACTTCATCATCACCGTGAACTACC |
| | | | CCGTGGTGGACTACTGGGGCCAAGGCACC |
| | | | CTGGTGACCGTGAGCAGC |
| | SEQ ID NO: 33 | VL | CAGAACGTGCTGACACAGAGCCCCGCCAC |
| | | DNA | CCTGAGCCTGAGCCCCGGCGAAAGAGCCA |
| | | | CCCTGAGCTGCAGCGCCGGCAGCAGCGTG |
| | | | AACTACCTGCACTGGTTTCAGCAGAAGCC |
| | | | CGGCCAAGCCCCTAGACTGCTGATCTACA |
| | | | GCACAAGCAACCTGGCTAGCGGCGTGCCC |
| | | | GCTAGATTCAGCGGCAGCGGCAGCGGCAC |
| | | | CGACTACACCCTGACCATCAGCAGCCTGG |
| | | | AGCCCGAGGACTTCGCCGTGTACTACTGTC |
| | | | AGCAGAGAAGCAGCCTGCCCTTCACCTTC |
| | | | GGCCAAGGCACCAAGCTGGAGATCAAG |
| BGA383 | SEQ ID NO: 34 | HCDR1 | DYWIY |
| | | (Kabat) | |
| | SEQ ID NO: 35 | HCDR2 | TIDTSDAKTSYNQKFRG |
| | | (Kabat) | |
| | SEQ ID NO: 36 | HCDR3 | DFIITVNYPVVDY |
| | | (Kabat) | |
| | SEQ ID NO: 37 | LCDR1 | SAGSSVNYMH |
| | | (Kabat) | |
| | SEQ ID NO: 38 | LCDR2 | STSNLAS |
| | | (Kabat) | |
| | SEQ ID NO: 39 | LCDR3 | QQRSSLPFT |
| | | (Kabat) | |
| | SEQ ID NO: 40 | VH | EVQLVQSGAEVKKPGASVKVSCKASGYTFT |
| | | AA | DYWIYWVRQAPGQGLEWMGTIDTSDAKTS |
| | | | YNQKFRGRVTMTRDTSTSTVYMELSSLRSE |
| | | | DTAVYYCARDFIITVNYPVVDYWGQGTLVT |
| | | | VSS |
| | SEQ ID NO: 41 | VL | QIVLTQSPATLSLSPGERATLSCSAGSSVNYM |
| | | AA | HWFQQKPGQAPRLLIYSTSNLASGVPARFSG |
| | | | SGSGTDYTLTISSLEPEDFAVYYCQQRSSLPF |
| | | | TFGQGTKLEIK |
| | SEQ ID NO: 42 | VH | GAGGTGCAGCTGGTGCAGAGCGGCGCCG |
| | | DNA | AGGTGAAGAAGCCCGGCGCTAGCGTGAA |
| | | | GGTGAGCTGCAAGGCTAGCGGCTACACCT |
| | | | TCACCGACTACTGGATCTACTGGGTGAGAC |
| | | | AAGCCCCCGGCCAAGGCCTGGAGTGGATG |
| | | | GGCACCATCGACACAAGCGACGCCAAGAC |
| | | | AAGCTACAATCAGAAGTTCAGAGGCAGAG |
| | | | TGACCATGACAAGAGACACAAGCACAAGC |
| | | | ACCGTGTACATGGAGCTGAGCAGCCTGAG |
| | | | AAGCGAGGACACCGCCGTGTACTACTGCG |
| | | | CTAGAGACTTCATCATCACCGTGAACTACC |
| | | | CCGTGGTGGACTACTGGGGCCAAGGCACC |
| | | | CTGGTGACCGTGAGCAGC |
| | SEQ ID NO: 43 | VL | CAGAACGTGCTGACACAGAGCCCCGCCAC |
| | | DNA | CCTGAGCCTGAGCCCCGGCGAAAGAGCCA |
| | | | CCCTGAGCTGCAGCGCCGGCAGCAGCGTG |
| | | | AACTACCTGCACTGGTTTCAGCAGAAGCC |
| | | | CGGCCAAGCCCCTAGACTGCTGATCTACA |
| | | | GCACAAGCAACCTGGCTAGCGGCGTGCCC |
| | | | GCTAGATTCAGCGGCAGCGGCAGCGGCAC |
| | | | CGACTACACCCTGACCATCAGCAGCCTGG |
| | | | AGCCCGAGGACTTCGCCGTGTACTACTGTC |
| | | | AGCAGAGAAGCAGCCTGCCCTTCACCTTC |
| | | | GGCCAAGGCACCAAGCTGGAGATCAAG |
| BGA384 | SEQ ID NO: 44 | HCDR1 | DYWIY |
| | | (Kabat) | |
| | SEQ ID NO: 187 | HCDR1 | GYTFTDY |
| | | (Chothia) | |
| | SEQ ID NO: 45 | HCDR2 | TIDTSEHKTSYNQKFRG |
| | | (Kabat) | |
| | SEQ ID NO: 188 | HCDR2 | DTSEHK |
| | | (Chothia) | |
| | SEQ ID NO: 46 | HCDR3 | DFIITVNYPVVDY |
| | | (Kabat/ | |
| | | Chothia) | |
| | SEQ ID NO: 47 | LCDR1 | SAGSSVNYMH |
| | | (Kabat/ | |
| | | Chothia) | |
| | SEQ ID NO: 48 | LCDR2 | STSNLAS |
| | | (Kabat/ | |
| | | Chothia) | |
| | SEQ ID NO: 49 | LCDR3 | QQRSSLPFT |
| | | (Kabat/ | |
| | | Chothia) | |
| | SEQ ID NO: 50 | VH | EVQLVQSGAEVKKPGASVKVSCKASGYTFT |
| | | AA | DYWIYWVRQAPGQGLEWMGTIDTSEHKTS |
| | | | YNQKFRGRVTMTRDTSTSTVYMELSSLRSE |
| | | | DTAVYYCARDFIITVNYPVVDYWGQGTLVT |
| | | | VSS |
| | SEQ ID NO: 51 | VL | QIVLTQSPATLSLSPGERATLSCSAGSSVNYM |
| | | AA | HWFQQKPGQAPRLLIYSTSNLASGVPARFSG |
| | | | SGSGTDYTLTISSLEPEDFAVYYCQQRSSLPF |
| | | | TFGQGTKLEIK |
| | SEQ ID NO: 189 | HC | EVQLVQSGAEVKKPGASVKVSCKASGYTFT |
| | | AA | DYWIYWVRQAPGQGLEWMGTIDTSEHKTS |
| | | | YNQKFRGRVTMTRDTSTSTVYMELSSLRSE |
| | | | DTAVYYCARDFIITVNYPVVDYWGQGTLVT |
| | | | VSSASTKGPSVFPLAPSSKSTSGGTAALGCL |
| | | | VKDYFPEPVTVSWNSGALTSGVHTFPAVLQS |
| | | | SGLYSLSSVVTVPSSSLGTQTYICNVNHKPS |
| | | | NTKVDKKVEPKSCDKTHTCPPCPAPELLGG |
| | | | PSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNK |
| | | | ALPAPIEKTISKAKGQPREPQVYTLPPSRDEL |
| | | | TKNQVSLTCLVKGFYPSDIAVEWESNGQPEN |
| | | | NYKTTPPVLDSDGSFFLYSKLTVDKSRWQQ |
| | | | GNVFSCSVMHEALHNHYTQKSLSLSPGK |
| | SEQ ID NO: 190 | LC | QIVLTQSPATLSLSPGERATLSCSAGSSVNYM |
| | | ACC | HWFQQKPGQAPRLLIYSTSNLASGVPARFSG |
| | | | SGSGTDYTLTISSLEPEDFAVYYCQQRSSLPF |
| | | | TFGQGTKLEIKRTVAAPSVFIFPPSDEQLKSG |
| | | | TASVVCLLNNFYPREAKVQWKVDNALQSG |
| | | | NSQESVTEQDSKDSTYSLSSTLTLSKADYEK |
| | | | HKVYACEVTHQGLSSPVTKSFNRGEC |
| | SEQ ID NO: 52 | VH | GAGGTGCAGCTGGTGCAGAGCGGCGCCG |
| | | DNA | AGGTGAAGAAGCCCGGCGCTAGCGTGAA |
| | | | GGTGAGCTGCAAGGCTAGCGGCTACACCT |
| | | | TCACCGACTACTGGATCTACTGGGTGAGAC |
| | | | AAGCCCCCGGCCAAGGCCTGGAGTGGATG |
| | | | GGCACCATCGACACAAGCGAGCACAAGAC |
| | | | AAGCTACAATCAGAAGTTCAGAGGCAGAG |
| | | | TGACCATGACAAGAGACACAAGCACAAGC |
| | | | ACCGTGTACATGGAGCTGAGCAGCCTGAG |
| | | | AAGCGAGGACACCGCCGTGTACTACTGCG |
| | | | CTAGAGACTTCATCATCACCGTGAACTACC |
| | | | CCGTGGTGGACTACTGGGGCCAAGGCACC |
| | | | CTGGTGACCGTGAGCAGC |
| | SEQ ID NO: 53 | VL | CAGAACGTGCTGACACAGAGCCCCGCCAC |
| | | DNA | CCTGAGCCTGAGCCCCGGCGAAAGAGCCA |
| | | | CCCTGAGCTGCAGCGCCGGCAGCAGCGTG |
| | | | AACTACCTGCACTGGTTTCAGCAGAAGCC |
| | | | CGGCCAAGCCCCTAGACTGCTGATCTACA |
| | | | GCACAAGCAACCTGGCTAGCGGCGTGCCC |
| | | | GCTAGATTCAGCGGCAGCGGCAGCGGCAC |
| | | | CGACTACACCCTGACCATCAGCAGCCTGG |
| | | | AGCCCGAGGACTTCGCCGTGTACTACTGTC |
| | | | AGCAGAGAAGCAGCCTGCCCTTCACCTTC |
| | | | GGCCAAGGCACCAAGCTGGAGATCAAG |
| BGA86 | SEQ ID NO: 54 | HCDR1 | SYGMS |
| | | (Kabat) | |
| | SEQ ID NO: 55 | HCDR2 | EISSGGTYTYYPDTVTG |
| | | (Kabat) | |
| | SEQ ID NO: 56 | HCDR3 | GDPFGY |
| | | (Kabat) | |
| | SEQ ID NO: 57 | LCDR1 | KASQDVMTAVA |
| | | (Kabat) | |
| | SEQ ID NO: 58 | LCDR2 | STSYRFT |
| | | (Kabat) | |
| | SEQ ID NO: 59 | LCDR3 | QQHYSTPRT |
| | | (Kabat) | |
| | SEQ ID NO: 60 | VH AA | EVQLQESGGGSVKPGGSLQLSCAASGFTFSS |
| | | | YGMSWIRQSPEKKLEWVAEISSGGTYTYYP |
| | | | DTVTGRFTISRDNAKNTLYLEMSSLRSEDTA |
| | | | MYYCARGDPFGYWGQGTLVTVSA |
| | SEQ ID NO: 61 | VL AA | DVVMTQSHKFVSTSVGGRVSITCKASQDVM |
| | | | TAVAWYQQKPGQSPKLLIYSTSYRFTGVPDR |
| | | | FTGSGSGTDFTLTINGVQAEDLAVYSCQQHY |
| | | | STPRTFGGGTKLEIK |
| | SEQ ID NO: 62 | VH DNA | GAGGTGCAGCTGCAAGAGTCCGGCGGGG |
| | | | GCAGCGTGAAGCCTGGGGGCTCCCTGCAG |
| | | | CTGAGCTGCGCCGCCTCCGGCTTCACCTTC |
| | | | AGCAGCTACGGCATGAGCTGGATCAGACA |
| | | | GAGCCCCGAGAAGAAGCTGGAGTGGGTG |
| | | | GCCGAGATCAGCAGCGGCGGCACCTACAC |
| | | | CTACTACCCCGACACCGTGACCGGCAGATT |
| | | | CACCATCAGCAGAGACAACGCCAAGAACA |
| | | | CCCTGTACCTGGAGATGAGCAGCCTGAGA |
| | | | AGCGAGGACACCGCCATGTACTACTGCGC |
| | | | TAGAGGCGACCCCTTCGGCTACTGGGGCC |
| | | | AAGGCACCCTGGTGACCGTGAGCGCC |
| | SEQ ID NO: 63 | VL DNA | GACGTGGTGATGACACAGAGCCACAAGTT |
| | | | CGTGAGCACAAGCGTGGGCGGCAGAGTG |
| | | | AGCATCACCTGCAAGGCCTCCCAAGACGT |
| | | | GATGACCGCCGTGGCCTGGTATCAGCAGA |
| | | | AGCCCGGGCAGAGCCCCAAGCTGCTGATC |
| | | | TACAGCACAAGCTACAGATTCACCGGCGT |
| | | | GCCCGACCGGTTCACCGGCAGCGGCAGCG |
| | | | GCACCGACTTCACCCTGACCATCAACGGC |
| | | | GTGCAAGCCGAGGACCTGGCCGTGTACAG |
| | | | CTGTCAGCAGCACTACAGCACCCCTAGAA |
| | | | CCTTCGGCGGGGGCACCAAGCTGGAGATC |
| | | | AAG |
| BGA861 | SEQ ID NO: 64 | HCDR1 | SYGIS |
| | | (Kabat) | |
| | SEQ ID NO: 65 | HCDR2 | EISSGGTYTYYPDTVTG |
| | | (Kabat) | |
| | SEQ ID NO: 66 | HCDR3 | GDPFGY |
| | | (Kabat) | |
| | SEQ ID NO: 67 | LCDR1 | KASQDVMTAVA |
| | | (Kabat) | |
| | SEQ ID NO: 68 | LCDR2 | STSYRFT |
| | | (Kabat) | |
| | SEQ ID NO: 69 | LCDR3 | QQHYSTPRT |
| | | (Kabat) | |
| | SEQ ID NO: 70 | VH AA | EVQLVESGGGLVKPGGSLRLSCAASGFTFSS |
| | | | YGISWVRQAPGKGLEWVSEISSGGTYTYYP |
| | | | DTVTGRFTISRDNAKNTLYLQMSSLRAEDTA |
| | | | VYYCARGDPFGYWGQGTLVTVSS |
| | SEQ ID NO: 71 | VL AA | DIQMTQSPSSLSASVGDRVTITCKASQDVMT |
| | | | AVAWYQQKPGKAPKLLIYSTSYRFTGVPSRF |
| | | | SGSGSGTDFTLTISSLQPEDFATYYCQQHYST |
| | | | PRTFGGGTKVEIK |
| | SEQ ID NO: 72 | VH DNA | GAGGTGCAGCTGGTGGAATCCGGCGGGGG |
| | | | CCTGGTGAAACCCGGCGGCAGCCTGAGAC |
| | | | TGAGCTGCGCCGCTAGCGGCTTCACCTTC |
| | | | AGCAGCTACGGCATCAGCTGGGTGAGACA |
| | | | AGCCCCCGGCAAGGGCCTGGAGTGGGTGA |
| | | | GCGAGATCAGCAGCGGCGGCACCTACACC |
| | | | TACTACCCCGACACCGTGACCGGCAGATTC |
| | | | ACCATCAGCAGAGACAACGCCAAGAACAC |
| | | | CCTGTACCTGCAGATGAGCAGCCTGAGAG |
| | | | CCGAGGACACCGCCGTGTACTACTGCGCT |
| | | | AGAGGCGACCCCTTCGGCTACTGGGGCCA |
| | | | AGGCACCCTGGTGACCGTGAGCAGC |
| | SEQ ID NO: 73 | VL DNA | GACATTCAGATGACACAGAGCCCTAGCAG |
| | | | CCTGTCCGCTAGCGTGGGCGACAGAGTGA |
| | | | CCATCACCTGCAAGGCTAGCCAAGACGTG |
| | | | ATGACCGCCGTGGCCTGGTATCAGCAGAA |
| | | | GCCCGGCAAGGCCCCCAAGCTGCTGATCT |
| | | | ACAGCACAAGCTACAGATTCACCGGCGTG |
| | | | CCTAGCAGATTCAGCGGCAGCGGCAGCGG |
| | | | CACCGACTTCACCCTGACCATCAGCAGCC |
| | | | TGCAGCCCGAGGACTTCGCCACCTACTACT |
| | | | GTCAGCAGCACTACAGCACCCCTAGAACC |
| | | | TTCGGCGGGGGCACCAAGGTGGAGATCAA |
| | | | G |
| BGA862 | SEQ ID NO: 74 | HCDR1 | SYGLS |
| | | (Kabat) | |
| | SEQ ID NO: 75 | HCDR2 | EISSGGTYTYYPDTVTG |
| | | (Kabat) | |
| | SEQ ID NO: 76 | HCDR3 | GDPFGY |
| | | (Kabat) | |
| | SEQ ID NO: 77 | LCDR1 | KASQDVMTAVA |
| | | (Kabat) | |
| | SEQ ID NO: 78 | LCDR2 | STSYRFT |
| | | (Kabat) | |
| | SEQ ID NO: 79 | LCDR3 | QQHYSTPRT |
| | | (Kabat) | |
| | SEQ ID NO: 80 | VH AA | EVQLVESGGGLVKPGGSLRLSCAASGFTFSS |
| | | | YGLSWVRQAPGKGLEWVSEISSGGTYTYYP |
| | | | DTVTGRFTISRDNAKNTLYLQMSSLRAEDTA |
| | | | VYYCARGDPFGYWGQGTLVTVSS |
| | SEQ ID NO: 81 | VL AA | DIQMTQSPSSLSASVGDRVTITCKASQDVMT |
| | | | AVAWYQQKPGKAPKLLIYSTSYRFTGVPSRF |
| | | | SGSGSGTDFTLTISSLQPEDFATYYCQQHYST |
| | | | PRTFGGGTKVEIK |
| | SEQ ID NO: 82 | VH DNA | GAGGTGCAGCTGGTGGAATCCGGCGGGGG |
| | | | CCTGGTGAAACCCGGCGGCAGCCTGAGAC |
| | | | TGAGCTGCGCCGCTAGCGGCTTCACCTTC |
| | | | AGCAGCTACGGCCTCAGCTGGGTGAGACA |
| | | | AGCCCCCGGCAAGGGCCTGGAGTGGGTGA |
| | | | GCGAGATCAGCAGCGGCGGCACCTACACC |
| | | | TACTACCCCGACACCGTGACCGGCAGATTC |
| | | | ACCATCAGCAGAGACAACGCCAAGAACAC |
| | | | CCTGTACCTGCAGATGAGCAGCCTGAGAG |
| | | | CCGAGGACACCGCCGTGTACTACTGCGCT |
| | | | AGAGGCGACCCCTTCGGCTACTGGGGCCA |
| | | | AGGCACCCTGGTGACCGTGAGCAGC |
| | SEQ ID NO: 83 | VL DNA | GACATTCAGATGACACAGAGCCCTAGCAG |
| | | | CCTGTCCGCTAGCGTGGGCGACAGAGTGA |
| | | | CCATCACCTGCAAGGCTAGCCAAGACGTG |
| | | | ATGACCGCCGTGGCCTGGTATCAGCAGAA |
| | | | GCCCGGCAAGGCCCCCAAGCTGCTGATCT |
| | | | ACAGCACAAGCTACAGATTCACCGGCGTG |
| | | | CCTAGCAGATTCAGCGGCAGCGGCAGCGG |
| | | | CACCGACTTCACCCTGACCATCAGCAGCC |
| | | | TGCAGCCCGAGGACTTCGCCACCTACTACT |
| | | | GTCAGCAGCACTACAGCACCCCTAGAACC |
| | | | TTCGGCGGGGGCACCAAGGTGGAGATCAA |
| | | | G |
| BGA12 | SEQ ID NO: 84 | HCDR1 | DYAMH |
| | | (Kabat) | |
| | SEQ ID NO: 85 | HCDR2 | VVLTHNDNTNYNQKFKA |
| | | (Kabat) | |
| | SEQ ID NO: 86 | HCDR3 | EVYYDFDDGNYFPMDY |
| | | (Kabat) | |
| | SEQ ID NO: 87 | LCDR1 | KSSQSLLYSDNQKNYLA |
| | | (Kabat) | |
| | SEQ ID NO: 88 | LCDR2 | WASTRES |
| | | (Kabat) | |
| | SEQ ID NO: 89 | LCDR3 | QQYYSYHT |
| | | (Kabat) | |
| | SEQ ID NO: 90 | VH AA | QVQLQQSGPEVVRPGVSVKISCKGSGYTFT |
| | | | DYAMHWVKQSHGKSLEWIGVVLTHNDNTN |
| | | | YNQKFKAKATMTVDRSSSTAYMELARLTSE |
| | | | DSAIYYCAREVYYDFDDGNYFPMDYWGQG |
| | | | TSVTVSS |
| | SEQ ID NO: 91 | VL AA | DIVMSQSPSSLAVSVGEKVTMSCKSSQSLLY |
| | | | SDNQKNYLAWYQQKPGQSPKLLIYWASTRE |
| | | | SGVPVRFTGSGSGTDFTLTISSVKAEDLAVY |
| | | | YCQQYYSYHTFGGGTKLEIK |
| | SEQ ID NO: 92 | VH DNA | GGATCCGCCACCATGGAGTTTGGGCTGAG |
| | | | CTGGCTTTTTCTTGTCGCGATTCTTAAGGG |
| | | | TGTCCAGTGCCAAGTGCAGCTGCAGCAGA |
| | | | GCGGCCCCGAGGTGGTGAGACCCGGCGTG |
| | | | AGCGTGAAGATCAGCTGCAAGGGCAGCGG |
| | | | CTACACCTTCACCGACTACGCCATGCACTG |
| | | | GGTGAAGCAGAGCCACGGCAAGAGCCTG |
| | | | GAGTGGATCGGCGTGGTGCTGACCCACAA |
| | | | CGACAACACCAACTACAATCAGAAGTTCA |
| | | | AGGCCAAGGCCACCATGACCGTGGACAGA |
| | | | AGCAGCAGCACCGCCTACATGGAGCTGGC |
| | | | TAGACTGACAAGCGAGGACAGCGCCATCT |
| | | | ACTACTGCGCTAGAGAGGTGTACTACGACT |
| | | | TCGACGACGGCAACTACTTCCCCATGGAC |
| | | | TACTGGGGCCAAGGCACAAGCGTGACCGT |
| | | | GAGCAGC |
| | SEQ ID NO: 93 | VL DNA | GACATCGTGATGAGCCAAAGCCCCTCCTC |
| | | | CCTGGCCGTGAGCGTGGGCGAGAAGGTGA |
| | | | CCATGAGCTGCAAGAGCAGCCAAAGCCTG |
| | | | CTGTACAGCGACAATCAGAAGAACTACCT |
| | | | GGCCTGGTATCAGCAGAAGCCCGGGCAGA |
| | | | GCCCCAAACTCCTGATCTACTGGGCTAGCA |
| | | | CAAGAGAGAGCGGCGTGCCCGTGAGATTC |
| | | | ACCGGCAGCGGCAGCGGCACCGACTTCAC |
| | | | CCTGACCATCAGCAGCGTGAAGGCCGAGG |
| | | | ACCTGGCCGTGTACTACTGTCAGCAGTACT |
| | | | ACAGCTACCACACCTTCGGCGGGGGCACC |
| | | | AAGCTGGAGATCAAG |
| BGA121 | SEQ ID NO: 94 | HCDR1 | DYAMH |
| | | (Kabat) | |
| | SEQ ID NO: 95 | HCDR2 | VVLTHNDNTNYNQKFKA |
| | | (Kabat) | |
| | SEQ ID NO: 96 | HCDR3 | EVYYDFDDGNYFPMDY |
| | | (Kabat) | |
| | SEQ ID NO: 97 | LCDR1 | KSSQSLLYSDNQKNYLA |
| | | (Kabat) | |
| | SEQ ID NO: 98 | LCDR2 | WASTRES |
| | | (Kabat) | |
| | SEQ ID NO: 99 | LCDR3 | QQYYSYHT |
| | | (Kabat) | |
| | SEQ ID NO: 100 | VH AA | QVQLVQSGAEVKKPGASVKVSCKASGYTFT |
| | | | DYAMHWVRQAPGQSLEWMGVVLTHNDNT |
| | | | NYNQKFKARVTMTVDTSASTAYMELSSLRS |
| | | | EDTAVYYCAREVYYDFDDGNYFPMDYWGQ |
| | | | GTLVTVSS |
| | SEQ ID NO: 101 | VL AA | DIVMTQSPDSLAVSLGERATINCKSSQSLLYS |
| | | | DNQKNYLAWYQQKPGQPPKLLIYWASTRES |
| | | | GVPDRFSGSGSGTDFTLTISSLQAEDVAVYY |
| | | | CQQYYSYHTFGGGTKLEIK |
| | SEQ ID NO: 102 | VH DNA | CAAGTGCAGCTGGTGCAGAGCGGCGCCGA |
| | | | GGTGAAGAAGCCCGGCGCTAGCGTGAAG |
| | | | GTGAGCTGCAAGGCTAGCGGCTACACCTT |
| | | | CACCGACTACGCCATGCACTGGGTGAGAC |
| | | | AAGCCCCCGGGCAGAGCCTGGAGTGGATG |
| | | | GGCGTGGTGCTGACCCACAACGACAACAC |
| | | | CAACTACAATCAGAAGTTCAAGGCTAGAG |
| | | | TGACCATGACCGTGGACACATCCGCCAGC |
| | | | ACCGCCTACATGGAGCTGAGCAGCCTGAG |
| | | | AAGCGAGGACACCGCCGTGTACTACTGCG |
| | | | CTAGAGAGGTGTACTACGACTTCGACGAC |
| | | | GGCAACTACTTCCCCATGGACTACTGGGGC |
| | | | CAAGGCACCCTGGTGACCGTGAGCAGC |
| | SEQ ID NO: 103 | VL DNA | GACATCGTGATGACACAGAGCCCCGACAG |
| | | | CCTGGCCGTGAGCCTGGGCGAGAGAGCCA |
| | | | CCATCAACTGCAAGAGCAGCCAAAGCCTG |
| | | | CTGTACAGCGACAATCAGAAGAACTACCT |
| | | | GGCCTGGTATCAGCAGAAGCCCGGGCAGC |
| | | | CCCCCAAGCTGCTGATCTACTGGGCTAGCA |
| | | | CAAGAGAGAGCGGCGTGCCCGACAGATTC |
| | | | AGCGGCAGCGGCAGCGGCACCGACTTCAC |
| | | | CCTGACCATCAGCAGCCTGCAAGCCGAGG |
| | | | ACGTGGCCGTGTACTACTGTCAGCAGTACT |
| | | | ACAGCTACCACACCTTCGGCGGGGGCACC |
| | | | AAGCTGGAGATCAAG |
| BGA122 | SEQ ID NO: 104 | HCDR1 | DYAMH |
| | | (Kabat) | |
| | SEQ ID NO: 105 | HCDR2 | VVLTHNDNTNYNQKFKA |
| | | (Kabat) | |
| | SEQ ID NO: 106 | HCDR3 | EVYYDFDDGNYFPMDY |
| | | (Kabat) | |
| | SEQ ID NO: 107 | LCDR1 | KSSQSLLYSDNQKNYLA |
| | | (Kabat) | |
| | SEQ ID NO: 108 | LCDR2 | WASTRES |
| | | (Kabat) | |
| | SEQ ID NO: 109 | LCDR3 | QQYYSYHT |
| | | (Kabat) | |
| | SEQ ID NO: 110 | VH AA | QVQLVQSGAEVKKPGASVKVSCKASGYTFT |
| | | | DYAMHWVRQAPGQSLEWMGVVLTHNDNT |
| | | | NYNQKFKARVTMTVDTSASTAYMELSSLRS |
| | | | EDTAVYYCAREVYYDFDDGNYFPMDYWGQ |
| | | | GTLVTVSS |
| | SEQ ID NO: 111 | VL AA | DIVMTQSPDSLAVSLGERATINCKSSQSLLYS |
| | | | DNQKNYLAWYQQKPGQSPKLLIYWASTRES |
| | | | GVPDRFSGSGSGTDFTLTISSLQAEDVAVYY |
| | | | CQQYYSYHTFGGGTKVEIK |
| | SEQ ID NO: 112 | VH DNA | CAAGTGCAGCTGGTGCAGAGCGGCGCCGA |
| | | | GGTGAAGAAGCCCGGCGCTAGCGTGAAG |
| | | | GTGAGCTGCAAGGCTAGCGGCTACACCTT |
| | | | CACCGACTACGCCATGCACTGGGTGAGAC |
| | | | AAGCCCCCGGGCAGAGCCTGGAGTGGATG |
| | | | GGCGTGGTGCTGACCCACAACGACAACAC |
| | | | CAACTACAATCAGAAGTTCAAGGCTAGAG |
| | | | TGACCATGACCGTGGACACATCCGCCAGC |
| | | | ACCGCCTACATGGAGCTGAGCAGCCTGAG |
| | | | AAGCGAGGACACCGCCGTGTACTACTGCG |
| | | | CTAGAGAGGTGTACTACGACTTCGACGAC |
| | | | GGCAACTACTTCCCCATGGACTACTGGGGC |
| | | | CAAGGCACCCTGGTGACCGTGAGCAGC |
| | SEQ ID NO: 113 | VL DNA | GACATCGTGATGACACAGAGCCCCGACAG |
| | | | CCTGGCCGTGAGCCTGGGCGAGAGAGCCA |
| | | | CCATCAACTGCAAGAGCAGCCAAAGCCTG |
| | | | CTGTACAGCGACAATCAGAAGAACTACCT |
| | | | GGCCTGGTATCAGCAGAAGCCCGGGCAGA |
| | | | GCCCCAAGCTGCTGATCTACTGGGCTAGCA |
| | | | CAAGAGAGAGCGGCGTGCCCGACAGATTC |
| | | | AGCGGCAGCGGCAGCGGCACCGACTTCAC |
| | | | CCTGACCATCAGCAGCCTGCAAGCCGAGG |
| | | | ACGTGGCCGTGTACTACTGTCAGCAGTACT |
| | | | ACAGCTACCACACCTTCGGCGGGGGCACC |
| | | | AAGGTGGAGATCAAGC |
| BGA50 | SEQ ID NO: 114 | HCDR1 | GYFLN |
| | | (Kabat) | |
| | SEQ ID NO: 115 | HCDR2 | RISPYNGDTFYNQKFKG |
| | | (Kabat) | |
| | SEQ ID NO: 116 | HCDR3 | GRSGSFTLFYAMDY |
| | | (Kabat) | |
| | SEQ ID NO: 117 | LCDR1 | KSSQSLFHSGNQKNYLA |
| | | (Kabat) | |
| | SEQ ID NO: 118 | LCDR2 | WASTRES |
| | | (Kabat) | |
| | SEQ ID NO: 119 | LCDR3 | QQYYSYPIT |
| | | (Kabat) | |
| | SEQ ID NO: 120 | VH AA | EVQLQQSGPEPVKPGASVKISCKASGYSFTG |
| | | | YFLNWVKQSHGKSLEWIGRISPYNGDTFYN |
| | | | QKFKGKATLTVDKSSSTAHMELLSLTSEDSA |
| | | | VYYCGRSGSFTLFYAMDYWGQGTSVTVSS |
| | SEQ ID NO: 121 | VL AA | DIVMSQSPSSLAVSVGENITMTCKSSQSLFHS |
| | | | GNQKNYLAWYQQKPGQSPKLLIYWASTRES |
| | | | GVPDRFTGSGSETDFTLTISSVKAEDLAVYFC |
| | | | QQYYSYPITFGAGTKLELK |
| | SEQ ID NO: 122 | VH DNA | GAGGTTCAGCTGCAGCAGTCTGGACCTGA |
| | | | ACCGGTGAAGCCTGGGGCTTCAGTGAAGA |
| | | | TATCCTGCAAGGCTTCTGGTTACTCATTTAC |
| | | | TGGCTACTTTTTGAACTGGGTGAAGCAGA |
| | | | GCCATGGAAAGAGCCTTGAGTGGATTGGA |
| | | | CGTATTAGTCCTTACAATGGTGATACTTTCT |
| | | | ACAACCAGAAGTTCAAGGGCAAGGCCAC |
| | | | ATTGACTGTAGACAAATCCTCTAGCACAGC |
| | | | CCACATGGAGCTCCTGAGCCTGACATCTG |
| | | | AGGACTCTGCAGTCTATTATTGTGGAAGAT |
| | | | CGGGTTCCTTCACTCTTTTCTATGCTATGGA |
| | | | CTACTGGGGTCAAGGAACCTCAGTCACCG |
| | | | TCTCCTCA |
| | SEQ ID NO: 123 | VL DNA | GACATTGTGATGTCACAGTCTCCATCCTCC |
| | | | CTAGCTGTGTCAGTTGGAGAGAATATTACT |
| | | | ATGACCTGCAAGTCCAGTCAGAGCCTTTTC |
| | | | CATAGTGGCAATCAAAAGAACTACTTGGC |
| | | | CTGGTACCAGCAGAAACCAGGGCAGTCTC |
| | | | CTAAACTGCTGATTTACTGGGCATCCACTA |
| | | | GGGAATCTGGGGTCCCTGATCGCTTCACA |
| | | | GGCAGTGGATCTGAGACAGATTTCACTCTC |
| | | | ACCATCAGCAGTGTGAAGGCTGAAGACCT |
| | | | GGCAGTTTATTTCTGTCAGCAATATTATAGC |
| | | | TATCCTATCACGTTCGGTGCTGGGACCAAG |
| | | | CTGGAGCTGAAA |
| BGA501 | SEQ ID NO: 124 | HCDR1 | GYFLN |
| | | (Kabat) | |
| | SEQ ID NO: 125 | HCDR2 | RISPYQGDTFYNQKFKG |
| | | (Kabat) | |
| | SEQ ID NO: 126 | HCDR3 | GRSGSFTLFYAIDY |
| | | (Kabat) | |
| | SEQ ID NO: 127 | LCDR1 | KSSQSLFHSGNQKNYLA |
| | | (Kabat) | |
| | SEQ ID NO: 128 | LCDR2 | WASTRES |
| | | (Kabat) | |
| | SEQ ID NO: 129 | LCDR3 | QQYYSYPIT |
| | | (Kabat) | |
| | SEQ ID NO: 130 | VH | QVQLVQSGAEVKKPGASVKVSCKASGYSFT |
| | | AA | GYFLNWVRQAPGQGLEWIGRISPYQGDTFY |
| | | | NQKFKGRVTMTTDKSTSTAYMELRSLRSDD |
| | | | TAVYYCGRSGSFTLFYAIDYWGQGTLVTVSS |
| | SEQ ID NO: 131 | VL | DIVMTQSPSSLAVSLGERATINCKSSQSLFHS |
| | | AA | GNQKNYLAWYQQKPGQSPKLLIYWASTRES |
| | | | GVPDRFSGSGSGTDFTLTISSLQAEDVAVYY |
| | | | CQQYYSYPITFGQGTKLEIK |
| | SEQ ID NO: 132 | VH | CAAGTGCAGCTGGTGCAGAGCGGCGCCGA |
| | | DNA | GGTGAAGAAGCCCGGCGCTAGCGTGAAG |
| | | | GTGAGCTGCAAGGCTAGCGGCTACAGCTT |
| | | | CACCGGCTACTTCCTGAACTGGGTGAGAC |
| | | | AAGCCCCCGGCCAAGGCCTGGAGTGGATC |
| | | | GGCAGAATCAGCCCCTACCAAGGCGACAC |
| | | | CTTCTACAATCAGAAGTTCAAGGGCAGAG |
| | | | TGACCATGACCACCGACAAGAGCACAAGC |
| | | | ACCGCCTACATGGAGCTGAGAAGCCTGAG |
| | | | AAGCGACGACACCGCCGTGTACTACTGCG |
| | | | GCAGAAGCGGCAGCTTCACCCTGTTCTAC |
| | | | GCCATCGACTACTGGGGCCAAGGCACCCT |
| | | | GGTGACCGTGAGCA |
| | SEQ ID NO: 133 | VL | GACATCGTGATGACACAGAGCCCTAGCAG |
| | | DNA | CCTGGCCGTGAGCCTGGGCGAGAGAGCCA |
| | | | CCATCAACTGCAAGAGCAGCCAAAGCCTG |
| | | | TTCCACAGCGGCAATCAGAAGAACTACCT |
| | | | GGCCTGGTATCAGCAGAAGCCCGGGCAGA |
| | | | GCCCCAAGCTGCTGATCTACTGGGCTAGCA |
| | | | CAAGAGAGAGCGGCGTGCCCGACAGATTC |
| | | | AGCGGCAGCGGCAGCGGCACCGACTTCAC |
| | | | CCTGACCATCAGCAGCCTGCAAGCCGAGG |
| | | | ACGTGGCCGTGTACTACTGTCAGCAGTACT |
| | | | ACAGCTACCCCATCACCTTCGGCCAAGGC |
| | | | ACCAAGCTGGAGATCAAG |
| BGA502 | SEQ ID NO: 134 | HCDR1 | GYFLN |
| | | (Kabat) | |
| | SEQ ID NO: 135 | HCDR2 | RISPYQGDTFYNQKFKG |
| | | (Kabat) | |
| | SEQ ID NO: 136 | HCDR3 | GRSGSFTLFYALDY |
| | | (Kabat) | |
| | SEQ ID NO: 137 | LCDR1 | KSSQSLFHSGNQKNYLA |
| | | (Kabat) | |
| | SEQ ID NO: 138 | LCDR2 | WASTRES |
| | | (Kabat) | |
| | SEQ ID NO: 139 | LCDR3 | QQYYSYPIT |
| | | (Kabat) | |
| | SEQ ID NO: 140 | VH | QVQLVQSGAEVKKPGASVKVSCKASGYSFT |
| | | AA | GYFLNWVRQAPGQGLEWIGRISPYQGDTFY |
| | | | NQKFKGRVTMTTDKSTSTAYMELRSLRSDD |
| | | | TAVYYCGRSGSFTLFYALDYWGQGTLVTVS |
| | | | S |
| | SEQ ID NO: 141 | VL | DIVMTQSPSSLAVSLGERATINCKSSQSLFHS |
| | | AA | GNQKNYLAWYQQKPGQSPKLLIYWASTRES |
| | | | GVPDRFSGSGSGTDFTLTISSLQAEDVAVYY |
| | | | CQQYYSYPITFGQGTKLEIK |
| | SEQ ID NO: 142 | VH | CAAGTGCAGCTGGTGCAGAGCGGCGCCGA |
| | | DNA | GGTGAAGAAGCCCGGCGCTAGCGTGAAG |
| | | | GTGAGCTGCAAGGCTAGCGGCTACAGCTT |
| | | | CACCGGCTACTTCCTGAACTGGGTGAGAC |
| | | | AAGCCCCCGGCCAAGGCCTGGAGTGGATC |
| | | | GGCAGAATCAGCCCCTACCAAGGCGACAC |
| | | | CTTCTACAATCAGAAGTTCAAGGGCAGAG |
| | | | TGACCATGACCACCGACAAGAGCACAAGC |
| | | | ACCGCCTACATGGAGCTGAGAAGCCTGAG |
| | | | AAGCGACGACACCGCCGTGTACTACTGCG |
| | | | GCAGAAGCGGCAGCTTCACCCTGTTCTAC |
| | | | GCCATCGACTACTGGGGCCAAGGCACCCT |
| | | | GGTGACCGTGAGCAGC |
| | SEQ ID NO: 143 | VL | GACATCGTGATGACACAGAGCCCTAGCAG |
| | | DNA | CCTGGCCGTGAGCCTGGGCGAGAGAGCCA |
| | | | CCATCAACTGCAAGAGCAGCCAAAGCCTG |
| | | | TTCCACAGCGGCAATCAGAAGAACTACCT |
| | | | GGCCTGGTATCAGCAGAAGCCCGGGCAGA |
| | | | GCCCCAAGCTGCTGATCTACTGGGCTAGCA |
| | | | CAAGAGAGAGCGGCGTGCCCGACAGATTC |
| | | | AGCGGCAGCGGCAGCGGCACCGACTTCAC |
| | | | CCTGACCATCAGCAGCCTGCAAGCCGAGG |
| | | | ACGTGGCCGTGTACTACTGTCAGCAGTACT |
| | | | ACAGCTACCCCATCACCTTCGGCCAAGGC |
| | | | ACCAAGCTGGAGATCAAG |
| BGA503 | SEQ ID NO: 144 | HCDR1 | GYFLN |
| | | (Kabat) | |
| | SEQ ID NO: 145 | HCDR2 | RISPYQGDTFYNQKFKG |
| | | (Kabat) | |
| | SEQ ID NO: 146 | HCDR3 | GRSGSFTLFYALDY |
| | | (Kabat) | |
| | SEQ ID NO: 147 | LCDR1 | KSSQSLFHSGNQKNYLA |
| | | (Kabat) | |
| | SEQ ID NO: 148 | LCDR2 | WASTRES |
| | | (Kabat) | |
| | SEQ ID NO: 149 | LCDR3 | QQYYSYPIT |
| | | (Kabat) | |
| | SEQ ID NO: 150 | VH | QVQLVQSGAEVKKPGASVKVSCKASGYSFT |
| | | AA | GYFLNWVRQAPGQGLEWIGRISPYQGDTFY |
| | | | NQKFKGRVTMTVDKSTSTAYMELRSLRSDD |
| | | | TAVYYCGRSGSFTLFYALDYWGQGTLVTVS |
| | | | S |
| | SEQ ID NO: 151 | VL | DIVMTQSPSSLAVSLGERATINCKSSQSLFHS |
| | | AA | GNQKNYLAWYQQKPGQSPKLLIYWASTRES |
| | | | GVPDRFSGSGSGTDFTLTISSLQAEDVAVYY |
| | | | CQQYYSYPITFGQGTKLEIK |
| | SEQ ID NO: 152 | VH | CAAGTGCAGCTGGTGCAGAGCGGCGCCGA |
| | | DNA | GGTGAAGAAGCCCGGCGCTAGCGTGAAG |
| | | | GTGAGCTGCAAGGCTAGCGGCTACAGCTT |
| | | | CACCGGCTACTTCCTGAACTGGGTGAGAC |
| | | | AAGCCCCCGGCCAAGGCCTGGAGTGGATC |
| | | | GGCAGAATCAGCCCCTACCAAGGCGACAC |
| | | | CTTCTACAATCAGAAGTTCAAGGGCAGAG |
| | | | TGACCATGACCGTGGACAAGAGCACAAGC |
| | | | ACCGCCTACATGGAGCTGAGAAGCCTGAG |
| | | | AAGCGACGACACCGCCGTGTACTACTGCG |
| | | | GCAGAAGCGGCAGCTTCACCCTGTTCTAC |
| | | | GCCCTGGACTACTGGGGCCAAGGCACCCT |
| | | | GGTGACCGTGAGCAGC |
| | SEQ ID NO: 153 | VL | GACATCGTGATGACACAGAGCCCTAGCAG |
| | | DNA | CCTGGCCGTGAGCCTGGGCGAGAGAGCCA |
| | | | CCATCAACTGCAAGAGCAGCCAAAGCCTG |
| | | | TTCCACAGCGGCAATCAGAAGAACTACCT |
| | | | GGCCTGGTATCAGCAGAAGCCCGGGCAGA |
| | | | GCCCCAAGCTGCTGATCTACTGGGCTAGCA |
| | | | CAAGAGAGAGCGGCGTGCCCGACAGATTC |
| | | | AGCGGCAGCGGCAGCGGCACCGACTTCAC |
| | | | CCTGACCATCAGCAGCCTGCAAGCCGAGG |
| | | | ACGTGGCCGTGTACTACTGTCAGCAGTACT |
| | | | ACAGCTACCCCATCACCTTCGGCCAAGGC |
| | | | ACCAAGCTGGAGATCAAG |

Identification of Epitopes and Antibodies that Bind to the Same Epitope

The present disclosure provides antibodies and antigen-binding fragments thereof that bind to an epitope of human PVRIG. In certain aspects the antibodies and antigen-binding fragments can bind to the same epitope of PVRIG.

The present disclosure also provides for antibodies and antigen-binding fragments thereof that bind to the same epitope as do the anti-PVRIG antibodies described in Table 5. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to PVRIG demonstrates that the test antibody can compete with that antibody or antigen-binding fragments thereof for binding to PVRIG. Such an antibody can, without being bound to any one theory, bind to the same or a related (e.g., a structurally similar or spatially proximal) epitope on PVRIG as the antibody or antigen-binding fragments thereof with which it competes. In a certain aspect, the antibody that binds to the same epitope on PVRIG as the antibodies or antigen-binding fragments thereof of the present disclosure is a human or humanized monoclonal antibody. Such human or humanized monoclonal antibodies can be prepared and isolated as described herein.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In still another aspect, the glycosylation of an antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al, 1998 Biochemistry, 37:9266-9273; Aalberse et al., 2002 Immunol, 105:9-19). Reduced ADCC can be achieved by operably linking the antibody to an IgG4 Fe engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate PVRIG antibodies with low ADCC and CDC but with good stability, it is possible to modify the hinge and Fc region of human IgG4 and introduce a number of alterations. These modified IgG4 Fe molecules can be found in SEQ ID NOs:83-88, U.S. Pat. No. 8,735,553 to Li et al.

### PVRIG Antibody Production

Anti-PVRIG antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

### Protein Expression

Generally, the methods of the disclosure for manufacturing the claimed anti-PVRIG antibodies involve culturing antibody-secreting mammalian cells. Such cultured mammalian cells are typically made by recombinant DNA technology involving transient or stable transfection. Pooled plasmid constructs (expression vectors) from this cloning step can be transfected into a plurality of host cells (e.g., mammalian, HEK 293 or CHO, bacterial, insect, yeast cells) for expression using a cationic lipid, polyethylenimine, Lipofectamine™, or ExpiFectamine™, or electroporation. The skilled practitioner is aware of numerous suitable means for transfecting to achieve expression of recombinant antibodies.

### Upstream Cell Culture Process

The host cells used to produce the antibodies of the disclosure can be cultured in a variety of types of cell culture media. Commercially available media such as CD-CHO liquid or CD-CHO AGT™ Powder (Life Technologies), Ham's FIO (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Patent Re. No. 30,985 may be used as culture media for the host cells. Any of these media may be supplemented with other ingredients as necessary at appropriate concentrations that would be known to those skilled in the art. For example, hormones and/or other growth factors (such as insulin, transferrin, or epidermal growth factor), salts (such as sodium chloride, sodium bicarbonate, calcium, iron, potassium, zinc, copper sulfate, manganese, magnesium, and phosphate), nucleotides (such as adenosine, adenine, thymidine, cytidine, guanosine, uridine, purine), any of the amino acids (tyrosine, cysteine, cystine, glutamic acid), vitamin or supplements (choline, inositol, thiamine, folic acid, biotin, calcium, niacinamide, paminobenzoic acid, pyridoxine, riboflavin, thymidine, cyanocobalamin, pyruvate, lipoic acid, linoleic acid, selenite, glycine, putrescine, ethanolamine), selection agents that confer resistance or survival to selectable markers such as antibiotics (such as geneticin, neomycin, hygromycin B, puromycin, zeocin, Gentamycin™), trace elements (defined as inorganic compounds usually present at final concentrations in the micromolar range), and glucose, galactose or an equivalent energy source, such that the physiological conditions of the cell in, or on, the medium promote expression of the protein of interest by the host cell. In one embodiment, non-ionic surfactants such as Kolliphor® P188 at 2-10 g/L can be added to maintain high cell density and viability. See Xu et al., Bioprocess Biosyst Eng (2017) 40: 1317-1326. In another embodiment, the copper concentration in the cell culture medium is 10-35 ppb. The culture medium is preferably serum-free. In some embodiments, the aqueous medium is liquid, such that the host cells are cultured in a cell suspension within the liquid medium.

The culture conditions, such as temperature (for mammalian cells, typically, about 37°±1° C.), pH (typically, but not necessarily, the cell culture medium is maintained within the range of about pH 6.5-7.5), oxygenation, and the like, will be apparent to the ordinarily skilled artisan. In one embodiment, the dissolved oxygen level is about 15-100%, and the pH is about 6.7-7.3.

There are likely to be small variations of the temperature, pH, and other culture conditions over time, and from location to location throughout the culture vessel (i.e., the bioreactor) such that there is an operating range for these parameters. (See, also, e.g., Oguchi et al., pH Condition in temperature shift cultivation enhances cell longevity and specific hMab productivity in CHO culture, Cytotechnology. 52(3): 199-207 (2006); Al-Fageeh et al., The cold-shock response in cultured mammalian cells: Harnessing the response for the improvement of recombinant protein production, Biotechnol. Bioeng. 93:829-835 (2006); Marchant, R. J. et al., Metabolic rates, growth phase, and mRNA levels influence cell-specific antibody production levels from in vitro cultured mammalian cells at sub-physiological temperatures, Mol. Biotechnol. 39:69-77 (2008)).

Upon culturing the transfected or transformed host cells, the anti-PVRIG antibodies of the present disclosures are directly secreted into the cell culture medium (by employing appropriate secretory-directing signal peptides) and are harvested therefrom.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO:12, SEQ ID NO:22, SEQ ID NO:32, SEQ ID NO:42, SEQ ID NO:52, SEQ ID NO:62, SEQ ID NO:72, SEQ ID NO:82, SEQ ID NO:92, SEQ ID NO:102, SEQ ID NO:112, SEQ ID NO:122, or SEQ ID NO:132. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting SEQ ID NO:13, SEQ ID NO:23, SEQ ID NO:33, SEQ ID NO:43, SEQ ID NO:53, SEQ ID NO:63, SEQ ID NO:73, SEQ ID NO:83, SEQ ID NO:93, SEQ ID NO:103, SEQ ID NO:113, SEQ ID NO:123, or SEQ ID NO:133.

The polynucleotides of the present disclosure can encode the variable region sequence of an anti-PVRIG antibody. They can also encode both a variable region and a constant region of the antibody. Some of the polynucleotide sequences encode a polypeptide that comprises variable regions of both the heavy chain and the light chain of one of the exemplified anti-PVRIG antibodies.

Also provided in the present disclosure are expression vectors and host cells for producing the anti-PVRIG antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-PVRIG antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-PVRIG antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-PVRIG antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-PVRIG polypeptides. Insect cells in combination with baculovirus vectors can also be used.

In other aspects, mammalian host cells are used to express and produce the anti-PVRIG polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Downstream Process

The compositions of the present disclosure from the upstream process may further undergo continuous or semi-continuous downstream purification, or a downstream batch purification process, to further purify the antibody or antigen-binding fragment composition.

During purification, a stationary phase is any surface onto which one or more ligands can be immobilized. The stationary phase may be a suspension, a discontinuous phase of discrete particles, a plate, sensor, chip, capsule, cartridge, resin, beads, monolith, gel, a membrane, or membrane adsorber, etc. Stationary phases may be packed into a purification column (e.g., packed with resin beads). Examples of materials for forming the stationary phase include mechanically stable matrices such as porous or non-porous beads, inorganic materials (e.g., porous silica, controlled pore glass (CPG) and hydroxyapatite), synthetic organic polymers (e.g., poly acrylamide, polymethylmethacrylate, poly(styrenedivinyl)benzene, polyacrylamide, ceramic particles and derivatives of any of the above) and polysaccharides (e.g., cellulose, agarose and dextran). See Jansson, J. C.; Ryden, L. Protein Purification; Wiley: New York, 1998.

Affinity chromatography separates molecules based on a highly specific interaction between the molecule of interest and the functional group of the resin, such as interaction between antigen and antibody, enzyme and substrate, receptor and ligand, or protein and nucleic acid, etc. Some commonly used affinity chromatographic resins include protein A or protein G resin to purify antibodies, avidin biotin resin to purify biotin/avidin and their derivatives, glutathione resin to purify GST-tagged recombinant proteins, heparin resin to separate plasma coagulation proteins, IMAC resin to purify proteins that specifically interact with the metal ions, etc. Operating conditions of each affinity chromatography depend on the mechanism of the interaction and factors that affect the interaction. Commercial affinity chromatographic resins include but are not limited to MabSelect Sure, UNOsphere SUPrA™, Affi-Gel®, and Affi-Prep®. In one embodiment, the affinity chromatography step is protein A chromatography performed in bind and elute mode. In one embodiment, the harvest cell culture fluid is purified with a Protein A affinity chromatography comprising the steps of:
a) binding the HCCF to a stationary phase; and
b) eluting the antibody or antigen binding fragment from the Protein A stationary phase with an elution solution.

In one embodiment, prior to step (a), equilibrating the stationary phase with an equilibration solution is performed. In one embodiment, one or more impurities are in the flowthrough of step a). In another aspect of the method, after step a) but prior to step b), the method further comprises the step of washing the stationary phase with one or more wash solutions. In one embodiment, one or more impurities are removed from the wash step. In one embodiment, the wash solution or elution solution comprises a salt, preferably a monovalent metal ion salt, such as NaCl or KCl. In one embodiment, the wash solution comprises about 400-600 mM NaCl or KCl. In one embodiment, the pH of the wash or elution solution is about 6-7. In one embodiment, the pH of the wash or elution solution is about 6.5. In another embodiment, the elution solution comprises about 5-50 mM Sodium Acetate. In another embodiment, the elution solution comprises about 5-30 mM Sodium Acetate. In another embodiment, the elution solution comprises about 20 mM Sodium Acetate. In another embodiment, the elution solution has a pH of about 3-4.

### Purified Compositions

The disclosure provides a composition comprising an anti-human PVRIG antibody or antigen-binding fragment thereof, wherein the anti-human PVRIG antibody or antigen binding fragment thereof comprises a light chain variable region comprising three light chain CDRs selected from the group set forth in Table 5, and a heavy chain variable region comprising three heavy chain CDRs selected from the group set forth in Table 5.

### Methods of Treatment

The antibodies or antigen-binding fragments of the present disclosure are useful in a variety of applications including, but not limited to, methods for the treatment of a PVRIG-associated disorder or disease. In one aspect, the PVRIG-associated disorder or disease is a cancer.

In one aspect, the present disclosure provides a method of treating cancer. In certain aspects, the method comprises administering to a patient in need an effective amount of an anti-PVRIG antibody or antigen-binding fragment. The cancer can include, without limitation, PVRL2-expressing cancers, PVRL2-accumulating cancers and cancers and metastases thereof of the colon and rectum (e.g., colon cancer and rectal cancer), anus, including anal canal and anorectum peritoneum and lower gastrointestinal; stomach (e.g., gastrointestinal stromal, adenocarcinoma and carcinoid), pleura, small and large intestine; prostate, including castration-resistant prostate cancer (CRPC); hepatocellular, including cancer of the liver and intrahepatic bile duct, gallbladder and other biliary cancers (e.g., cholangiosarcoma), pancreas (e.g., pancreatic adenocarcinoma), respiratory system, including larynx and esophageal (e.g., esophageal squamous cell carcinoma or esophageal adenocarninoma), bronchus and lung (mesothelioma, non-small cell lung cancer (NSCLC, e.g., lung squamous or lung adenocarcinoma), and other respiratory organs; skin (including basal and squamous), melanoma of the skin and non-epithelial skin; glandular (salviary and mucsinous); breast (e.g., hormone receptor positive breast cancer, estrogen receptor and progesterone receptor negative breast cancer, triple negative breast cancer (TNBC), and PRLR positive (PRLR⁺) breast cancers); genitourinary system, including uterine, cervix, uterine corpus, ovary (e.g., serous tumor, mucinous tumor, clear cell tumor, endometrioid tumor, transitional cell tumor, Brenner tumor, carcinosarcoma of the ovary, mixed epithelial tumor, borderline epithelial tumor, undifferentiated carcinoma tumor, tumor of the fallopian tube and peritoneum); cancer of the vulva, vagina, renal pelvis and other female and male genitalia, including penis, testis, ureter and other urinary organs; bladder cancer (e.g., bladder urothealia); cancer of the kidney (e.g., renal cell carcinoma and Wilms tumor); brain and nervous system including astrocytoma, glioblastoma, malignant gliomas and malignant peripheral nerve sheath tumors (MPNST); thyroid and other endocrine cancers (e.g., papillary, follicular, medullary, and anaplastic); cancer of the eye and orbit; head and neck (e.g., laryngeal, hypopharyngeal, lip and oral cavity), metastatic squamous neck cancer, nasopharyngeal, oropharyngeal, paranasal sinus and nasal cavity, salivary gland); bones and joints (e.g., osteosarcoma), soft tissue (including heart), e.g., Kaposi's sarcoma, rhabdomyosarcoma and undifferentiated pleiomorphic sarcoma, and other connective tissues (e.g., myxofibrosarcoma and fibromyxoid sarcoma); virus-related cancers, myeloma and B-cell cancers, including Hodgkin lymphoma and non-Hodgkin lymphoma (e.g., diffuse large B-cell lymphoma (DLBCL), follicular lymphoma, primary mediastinal B cell lymphoma, mantle cell lymphoma, post-transplant lymphoproliferative disorder, marginal zone lymphoma, Waldenstrom's macroglobulinemia and Burkitt lymphoma); T-cell cancers, including anaplastic large cell lymphoma, peripheral T cell lymphoma, angioimmunoblastic lymphoma, hepatosplenic T-cell lymphoma and extranodal NK/T-cell lymphoma; and leukemias including acute lymphocytic leukemia, chronic lymphocytic leukemia, acute myeloid leukemia, chronic myeloid leukemia.

In addition to anatomical location, cancers may be identified and treated by individual and/or combinations of cancer driver mutations, such as, for example BRAF, CTNNB1, EGFR HER2/ERBB2, FGFR2, GNA11, IDH1/2, KIT, KRAS, MAP2K, NRAS, PIK3CA, TP53 (Bailey et al., 2018. Comprehensive characterization of cancer driver genes and mutations. Cell, 173(2), pp. 371-385).

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g., every week or every three weeks (e.g., such that the patient receives from about two to about twenty, or e.g., about six doses of the antibody). An initial higher loading dose, followed by one or more lower doses can be administered. However, other dosage regimens can be useful. The progress of this therapy is easily monitored by conventional techniques and assays.

### Combination Therapy

In one aspect, PVRIG antibodies of the present disclosure can be used in combination with other therapeutic agents. Other therapeutic agents that can be used with the PVRIG antibodies of the present disclosure include, but are not limited to, a chemotherapeutic agent (e.g., paclitaxel or a paclitaxel agent; (e.g., Abraxane®), docetaxel; carboplatin; topotecan; deruxtecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, R05072759, LFB-R603), CD52 targeting agent (e.g., alemtuzumab), prednisolone, darbepoetin alfa, lenalidomide, Bel-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

Anti-PVRIG antibodies of the present disclosure can be used in combination with other therapeutics, for example, other immune checkpoint antibodies. Such immune checkpoint antibodies can include anti-PD1 antibodies. Anti-PD1 antibodies can include, without limitation, antibodies disclosed in U.S. Pat. No. 8,735,553. Pembrolizumab (formerly MK-3475), as disclosed by Merck, is a humanized IgG4-K immunoglobulin with a molecular weight of about 149 kDa, which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb is a fully human IgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

Other immune checkpoint antibodies for combination with anti-PVRIG antibodies can include anti-TIGIT antibodies. Such anti-TIGIT antibodies can include without limitation, anti-TIGIT antibodies disclosed in WO2019/129261.

### Pharmaceutical Compositions and Formulations

Also provided are compositions, including pharmaceutical formulations, comprising an anti-PVRIG antibody or antigen-binding fragment thereof, or polynucleotides comprising sequences encoding an anti-PVRIG antibody or antigen-binding fragment. In certain embodiments, compositions comprise one or more antibodies or antigen-binding fragments that bind to PVRIG, or one or more polynucleotides comprising sequences encoding one or more antibodies or antigen-binding fragments that bind to PVRIG. These compositions can further comprise suitable carriers, such as pharmaceutically acceptable excipients including buffers, which are well known in the art.

Pharmaceutical formulations of an anti-PVRIG antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Nos. U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. Sustained-release preparations can be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility can be readily accomplished, e.g., by filtration through sterile filtration membranes.

### Methods of Use

The disclosure also relates to a method of treating cancer in a subject, the method comprising administering an effective amount of any of the compositions of the disclosed present disclosures; i.e., any composition described herein, to the subject. In some embodiments of this method, the composition is administered to the subject by intravenous or subcutaneous administration.

In some embodiments, the cancer is a solid tumor with a high mutational burden. In one embodiment, the Tumor Mutation Burden (TMB) is ≥10 mutations/megabase as determined by an FDA approved test. In one embodiment, the tumor is metastatic or unresectable.

In some embodiments, the cancer is selected from the group consisting of: melanoma, non-small cell lung cancer, relapsed or refractory classical Hodgkin lymphoma, head and neck squamous cell carcinoma, urothelial cancer, esophageal cancer, gastric cancer, DLBCL and hepatocellular cancer.

In other embodiments of the above treatment methods, the cancer is a Heme malignancy. In certain embodiments, the Heme malignancy is acute lymphoblastic leukemia (ALL), acute myeloid leukemia (AML), chronic lymphocytic leukemia (CLL), chronic myeloid leukemia (CML), DLBCL, EBY-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mel-1), myelodysplastic syndrome (MDS), non-Hodgkin lymphoma (NHL), or small lymphocytic lymphoma (SLL).

Malignancies that demonstrate improved disease-free and overall survival in relation to the presence of tumor-infiltrating lymphocytes in biopsy or surgical material, e.g. melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

In some embodiments, the compositions of the present disclosure are administered to a subject having a cancer characterized by elevated expression of PVRL2 in tested tissue samples, including: ovarian, renal, colorectal, pancreatic, breast, liver, gastric, esophageal cancers and melanoma. Additional cancers that can benefit from treatment with anti-PVRIG antibodies may include those associated with persistent infection with viruses such as human immunodeficiency viruses, hepatitis viruses class A, B and C, Epstein Barr virus, human papilloma viruses that are known to be causally related to for instance Kaposi's sarcoma, liver cancer, nasopharyngeal cancer, lymphoma, cervical, vulval, anal, penile and oral cancers.

In one embodiment, the present disclosure comprises a method of treating cancer in a human patient comprising administering any composition disclosed herein to the patient. For example, in one embodiment, the present disclosure comprises a method of treating unresectable or metastatic melanoma in a human patient comprising administering any composition of the present disclosure to the patient. In another embodiment, the present disclosure comprises a method of treating metastatic non-small cell lung cancer (NSCLC) in a human patient comprising administering a composition of the present disclosure to the patient. In specific further embodiments, the patient has a tumor with high PVRL2 expression, for example, Tumor Proportion Score (TPS) 50%. In other embodiments, the patient has a tumor with PVRL2 expression (TPS 1%). In still other embodiments, the patient was or was not previously treated with platinum-containing chemotherapy. In specific embodiments, the patient had disease progression on or after receiving platinum-containing chemotherapy. In one embodiment, the cancer is metastatic or Stage III. In certain embodiments, the patient has a tumor with PVRL2 expression with a Combined Positive Score (CPS) 1%. In certain embodiments, the patient was previously treated with platinum-containing chemotherapy. In certain embodiments, the patient had disease progression on or after platinum-containing chemotherapy. In specific embodiments, the patient's tumor expresses PVRL2 with a CPS of 1%. In one embodiment, the treatment is in combination with platinum and FU.

Embodiments of the present disclosure also include one or more of the biological compositions described herein
(i) for use in,
(ii) for use as a medicament or composition for, or
(iii) for use in the preparation of a medicament for:
   a. therapy (e.g., of the human body);
   b. medicine;
   c. induction of or augmenting an anti-tumor immune response;
   d. decreasing the number of one or more tumor markers in a patient;
   e. halting or delaying the growth of a tumor or a blood cancer;
   f. halting or delaying the progression of PVRIG-related disease;
   g. halting or delaying the progression of cancer;
   h. stabilization of PVRIG-related disease;
   i. inhibiting the growth or survival of tumor cells;
   j. eliminating or reducing the size of one or more cancerous lesions or tumors;
   k. reduction of the progression, onset or severity of PVRIG-related disease;
   l. reducing the severity or duration of the clinical symptoms of PVRIG-related disease such as cancer;
   m. prolonging the survival of a patient relative to the expected survival in a similar untreated patient;
   n. inducing complete or partial remission of a cancerous condition or other PVRIG-related disease; or
   o. treatment of cancer.

### Kits

The present disclosure, among other things, provides kits comprising anti-PVRIG antibodies or antigen-binding fragments thereof, and instructions for use and/or administration. In some embodiments, a kit comprises at least one anti-PVRIG antibody or antigen-binding fragment thereof and a pharmaceutically acceptable carrier, and instructions for use and/or administration.

Also provided are kits for use in various methods disclosed herein. Instructions can comprise a description of administering of one or more pharmaceutical compositions described herein to a subject to achieve an intended activity in a subject. A kit may further comprise a description of selecting a human suitable for treatment based on identifying whether the human is in need of treatment. In some embodiments, instructions comprise a description of administering at least one anti-PVRIG antibody or antigen-binding fragment thereof to a subject who is in need of the treatment.

Instructions relating to administering one or more doses of at least one anti-PVRIG antibody or antigen-binding fragment thereof generally include information as to dosage, dosing schedule, and route of administration for an intended treatment. Containers may be unit doses, bulk packages (e.g., multi-dose packages) or sub-unit doses. Instructions supplied in kits of the disclosure are typically written instructions on a label or package insert. A label or package insert may indicate that one or more pharmaceutical compositions described herein are used for treating, delaying the onset, and/or alleviating a disease, disorder or condition in a subject.

In some embodiments, kits provided herein are in suitable packaging. Suitable packaging includes, but is not limited to, vials, bottles, jars, flexible packaging, and the like. Also contemplated are packages for use in combination with a specific device, such as an infusion device. A kit may have a sterile access port (for example, a container may be an intravenous solution bag or a vial having a stopper pierce able by a hypodermic injection needle). A container may also have a sterile access port.

Kits can include additional components such as buffers and interpretive information. A kit can comprise a container and a label or one or more package inserts on or associated with a container. In some embodiment, the disclosure provides articles of manufacture comprising contents of kits described above.

### EXAMPLES

### Example 1. Generation of Mouse Anti-PVRIG Antibodies

To generate Abs against PVRIG, cohorts of 25 BALB/C, SJL strains of inbred mice were immunized with different PVRIG antigens with each cohort being subjected to an immunization strategy comprising a unique combination of PVRIG antigens, dose, injection route, adjuvant and immunization timing. 10 BALB/C strains of inbred mice were immunized with hPVRIG-ECD-mFc (Acrobiosystems, PVG-H5253-1 mg which included amino acids Thr 41-Asp 171

(SEQ ID NO: 154)
(TPEVWVQVRMEATELSSFTIRCGFLGSGSISLVTVSWGGPNGAGGTTLA
VLHPERGIRQWAPARQARWETQSSISLILEGSGASSPCANTTFCCKFASF
PEGSWEACGSLPPSSDPGLSAPPTPAPILRAD).

Five BALB/C strains of inbred mice were immunized with hPVRIG-ECD-his (Acrobiosystems, PVG-H52H4-1 mg (500 g×2)) which included amino acids Thr 41-Asp 171 of PVRIG (SEQ ID NO:154). Five SJL strains of inbred mice were immunized with hPVRIG-ECD-his (Acrobiosystems, PVG-H52H4-1 mg (500 g×2)), which also used Thr 41-Asp 171 (SEQ ID NO:154) as an immunogen. A total of 5 animals in 5 cohorts were immunized. Animals received immunizations over varying periods between 0 and 56 days. To monitor immune responses, titrated serum was screened by ELISA, typically after 21-56 days of 2-4 immunizations. Serum was screened for antibody binding to the PVRIG antigen hPVRIG-ECD-his (Acrobiosystems, PVG-H52H4-1 mg (500 g×2)) used in the immunization (SEQ ID NO:154). PVRIG-specific antibody responses were measured in each animal, and animals with sufficient titers of anti-PVRIG Ig were selected for final boost 4 days.

Lymphoid organs, including spleens and lymph nodes, were isolated from mice immunized as described above. Hybridomas were generated by fusions with immortalized mouse myeloma cells derived from the SP2/0 by PEG based fusion. The resulting cells were plated in 96 well cell culture plates using regular 1640 medium supplemented with HAT for selection of hybridomas. In addition, several antibodies were isolated directly from antigen positive B cells without fusion to myeloma cells, as described in US 2016/0252495A1.

### Example 2. Screening and Selection of Anti-PVRIG Antibodies

In order to generate monoclonal antibodies that bind to PVRIG, mice were immunized with human PVRIG antigens, and hybridomas were generated as described in Example 1. After 10-13 days of culture and growth media replacement, hybridoma culture supernatants were collected from individual wells and screened to identify wells with secreted PVRIG-specific antibodies. All supernatants were initially screened against at least hPVRIG-ECD-hFc (Acrobiosystems, PVG-H5257 and hPVRIG-ECD-his) (Acrobiosystems, PVG-H52H4), containing amino acids Thr 41-Asp 171 (SEQ ID NO:154) was used in antibody generation. Antibodies specifically binding to the human PVRIG recombinant proteins were measured via ELISA. Supernatants from approximately over 23040 culture wells in 5 hybridoma fusions were screened for PVRIG antibody binding. Briefly, 2 μg/mL human PVRIG-His or human PVRIG-hFc was coated in 96 well ELISA plates, and 50 μl of hybridoma culture supernatant was incubated for 30-60 minutes, washed, and detected with anti-mouse IgG Fe or anti-human IgG Fe secondary antibody conjugated to horseradish peroxidase (HRP). After incubation and washing, and the plates were developed with HRP substrate and absorbance was measured.

Hybridomas from positive wells were transferred to 24-well plates with fresh culture media to grow for 2-3 days before screening again by flow cytometry to confirm antibody binding to a PVRIG expressing cell line and Cynomolgus monkey (Cyno) PVRIG cross reactivity. In addition, the hybridoma culture supernatant from 24-well plates was also evaluated for inhibiting the binding between PVRIG and its ligand PVRL2.

Antibody binding on a PVRIG-expressing cell line was measured via FACS. Briefly, 100 μl of hybridoma culture supernatant and PVRIG expressing cells (such as CHO or Jurkat) or control cells (such as parental CHO) were incubated for 30-60 minutes, washed, and incubated with anti-mouse IgG Fe secondary antibody conjugated to allophycocyanin (APC) for detection. After incubation and washing, fluorescence was measured by flow cytometry.

Antibody binding to Cyno PVRIG recombinant protein was measured via ELISA. Briefly, 2 μg/mL human PVRIG-His or human PVRIG-hFc was coated onto 96 well ELISA plates, and 50 μl of hybridoma culture supernatant was co-incubated for 30-60 minutes, washed, and incubated with anti-mouse IgG Fe secondary Ab conjugated to HRP. After incubation and washing, the plates were developed with HRP substrate and absorbance was measured.

The inhibition of binding between PVRIG and its ligand PVRL2 was measured through ELISA. Briefly, 2 μg/mL human PVRIG-His was coated in the 96 well ELISA plates, and 50 μl of hybridoma culture supernatant and 50 μl hPVRL2-hFc (1 g/ml) were co-incubated for 30-60 minutes, washed, and incubated with anti-human IgG Fe secondary antibody conjugated to HRP. After incubation and washing, the plates were developed with HRP substrate and absorbance was measured. The antibodies were gated on this functional ability and the data for chosen antibodies is shown below in Example 7.

### Example 3. Subcloning of Selected Anti-PVRIG Antibodies

Selected anti-PVRIG antibody-secreting hybridomas were subcloned to monoclonality. Briefly, approximately 80-100 viable hybridoma cells were plated in 3 ml of semi-solid methylcellulose medium (Stem Cell Technologies) in a 6 well plate. After 7-10 days, hybridoma colonies arising from single cells as visible clones were transferred to a 96-well plate, cultured with fresh medium, and allowed to proliferate for 2-4 days. Culture supernatant was screened by ELISA and flow cytometry as previously described above to confirm human and Cyno PVRIG binding. Stable hybridoma subclones were cultured in vitro for cryopreservation and also submitted for antibody VH and VL gene cloning and sequencing.

### Example 4. Anti-PVRIG Antibody VH and VL Gene Cloning and Sequencing

After the removal of the supernatant, selected anti-PVRIG secreting hybridomas after subcloning were lysed by 100 ml RLT buffer (Qiagen Inc.) in 96-well plates. The mRNA containing lysates were subsequently transferred to 96-well deep well plates for mRNA isolation, cDNA synthesis and DNA sequencing by standard sequencing techniques (Sanger sequencing and next generation sequencing). In brief, total RNA of the cell lysates was prepared using the Total RNA Isolation Kit™ (New England Biolabs Inc.) according to the manufacturer's instructions. The cDNA was generated by reverse transcription of the mRNA using the Super Script III™ first-strand synthesis SuperMix™ (Invitrogen) according to the manufacturer's instructions. The primary sequences of selected cloned antibodies are provided in Table 5.

### Example 5. EC50 Values for Binding of Chimeric Anti-PVRIG Antibodies to Human and Cyno PVRIG Recombinant Proteins

The antibody VH and VL sequences were cloned into a human IgG expression vector, and transfected into 293T cells for expression, and after 1-14 days in culture, the antibody containing supernatant was harvested with a protein A affinity purification column. After purification, the binding EC50 to human and Cyno PVRIG recombinant proteins was characterized using ELISA assay as shown in Table 6.

TABLE 6

| | Human PVRIG | Cyno PVRIG |
|---|---|---|
| Antibody | binding EC50 (nM) | binding EC50 (nM) |
| BGA38 | 0.5237 | 0.2225 |
| BGA86 | 0.06899 | 0.04485 |
| BGA50 | 0.1051 | 0.02729 |
| BGA12 | 0.1262 | 0.03901 |

### Example 6. EC50 Values for Chimeric Anti-PVRIG Antibodies Binding to PVRIG-Expressing Cells

The antibody VH and VL gene were cloned into a human IgG expression vector, and transfected into 293T cells for expression, and after 1-14 days culture, the antibody containing supernatant was harvested with a protein A affinity purification column. The purified antibodies were then characterized for binding and EC50 to the human PVRIG expressing cells via FACS assay. This data is shown in Table 7.

TABLE 7

| | PVRIG-expressing cells | PVRIG-expressing cells |
|---|---|---|
| Antibody | binding EC50 (nM) | binding Emax (MFI) |
| BGA38 | 0.059 | 1039 |
| BGA86 | 0.038 | 1033 |
| BGA50 | 0.273 | 2611 |
| BGA12 | 0.050 | 1177 |

### Example 7. IC50 Values for Anti-PVRIG Antibodies Reducing PVRL2 Binding to PVRIG

The antibody VH and VL genes were cloned into a human IgG expression vector, and transfected into 293T cells for expression, and after 1-14 days culture, the antibody containing supernatant was harvested with a protein A affinity purification column. After purification, the IC50 values for chimeric anti-PVRIG antibodies reducing the interaction between PVRIG and its ligand PVRL2 were characterized using ELISA assay as shown in Table 8.

TABLE 8

| | PVRIG/PVRL2 blocking | PVRIG/PVRL2 blocking |
|---|---|---|
| Antibody | IC50 (nM) | Emax (% Inhibition) |
| BGA38 | 1.502 | 60.05 |
| BGA86 | 0.885 | 97.16 |
| BGA50 | 1.093 | 95.35 |
| BGA12 | 0.15 | 91.86 |

### Example 8. Affinity Profiles of Chimeric Anti-PVRIG Antibodies by SPR

Chimeric anti-PVRIG antibodies were generated in human IgG1 format and characterized for binding kinetics by surface plasmon resonance (SPR) assays using BIAcore™ T-200 (GE Life Sciences). Briefly, Mouse Anti-human IgG Fc Antibody was immobilized on an activated CM5 biosensor chip (Cat. No. BR100530, GE Life Sciences). Purified chimeric anti-PVRIG antibodies were flowed over the chip surface and captured by anti-human IgG antibody. Then a serial dilution of human or Cyno PVRIG protein was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of chimeric anti-PVRIG antibodies are shown below in Table 9 and Table 10.

TABLE 9
Binding Affinity Profiles Against Human PVRIG

| Antibody | Antigen | ka (1/Ms) | kd (1/s) | KD (M) |
|---|---|---|---|---|
| BGA38 | Human PVRIG | 1.52E⁺06 | 1.70E−04 | 1.12E−10 |
| BGA86 | Human PVRIG | 1.34E⁺07 | 2.15E−03 | 1.61E−10 |
| BGA50 | Human PVRIG | 4.45E⁺6 | 6.52E−4 | 1.47E−10 |
| BGA12 | Human PVRIG | 2.29E⁺07 | 1.57E−03 | 6.84E−11 |

TABLE 10
Binding Affinity Profiles Against Cyno PVRIG

| Antibody | Antigen | ka (1/Ms) | kd (1/s) | KD (M) |
|---|---|---|---|---|
| BGA38 | Cyno PVRIG | 1.44E⁺06 | 1.57E−04 | 1.09E−10 |
| BGA86 | Cyno PVRIG | 1.97E⁺06 | 1.33E−03 | 6.74E−10 |
| BGA50 | Cyno PVRIG | 8.05E⁺6 | 5.88E_3 | 7.30E−10 |
| BGA12 | Cyno PVRIG | 5.61E⁺04 | 3.54E−04 | 6.31E−09 |

### Example 9. Humanization and Engineering of the Anti-PRIG Antibody BGA12

For humanization of BGA12, human germline IgG genes were searched for sequences that share high degrees of homology to the cDNA sequences of BGA12 variable regions by BLAST analysis of the human immunoglobulin gene database in IMGT and the NCBI database. The human IGVH and IGVL genes that are present in human antibody repertoires with high frequencies (Glanville 2009 PNAS 106:20216-20221) and are highly homologous to BGA12 were selected as the templates for humanization.

TABLE 11

| | SEQ | | |
|---|---|---|---|
| Anti- | ID | | |
| body | NO | | SEQUENCE |
| BGA38 | SEQ | Human | QVQLVQSGAEVKKPGASVKVSCKASGYTFT |
| Human | ID | IGVH1- | SYYMHWVRQAPGQGLEWMGIINPSGGSTS |
| frame- | NO: | 46 | YAQKFQGRVTMTRDTSTSTVYMELSSLRSE |
| work | 155 | | DTAVYYCAR |
| | SEQ | Human | EIVLTQSPATLSLSPGERATLSCRASQSVS |
| | ID | IGVK3- | SYLAWYQQKPGQAPRLLIYDASNRATGIPA |
| | NO: | 11 | RFSGSGSGTDFTLTISSLEPEDFAVYYCQQ |
| | 156 | | RSNWP |
| BGA86 | SEQ | Human | EVQLVESGGGLVKPGGSLRLSCAASGFTFS |
| Human | ID | IGVH3- | SYSMNWVRQAPGKGLEWVSSISSSSSYIYY |
| frame- | NO: | 21 | ADSVKGRFTISRDNAKNSLYLQMNSLRAED |
| work | 157 | | TAVYYCAR |
| | SEQ | Human | DIQMTQSPSSLSASVGDRVTITCRASQSIS |
| | ID | IGVH1- | SYLNWYQQKPGKAPKLLIYAASSLQSGVPS |
| | NO: | 39 | RFSGSGSGTDFTLTISSLQPEDFATYYCQQ |
| | 158 | | SYSTP |
| BGA12 | SEQ | Human | QVQLVQSGAEVKKPGASVKVSCKASGYTFT |
| Human | ID | IGVH1- | SYA |
| frame- | NO: | 3 | MHWVRQAPGQRLEWMGWINAGNGNTKYS |
| work | 159 | | QKFQGRVTITRDTSASTAYMELSSLRSEDT |
| | | | AVYYCAR |
| | SEQ | Human | DIVMTQSPDSLAVSLGERATINCKSSQSVL |
| | ID | IGVK4- | YSSNNKNYLAWYQQKPGQPPKLLIYWASTR |
| | NO: | 1 | ESGVPDRFSGSGSGTDFTLTISSLQAEDVA |
| | 160 | | VYYCQQYYSTP |
| BGA50 | SEQ | Human | QVQLVQSGAEVKKPGASVKVSCKASGYTFT |
| Human | ID | IGVH1- | SYGISWVRQAPGQGLEWMGWISAYNGNTN |
| frame- | NO: | 18 | YAQKLQGRVTMTTDTSTSTAYMELRSLRSD |
| work | 161 | | DTAVYYCAR |
| | SEQ | Human | DIVMTQSPDSLAVSLGERATINCKSSQSVL |
| | ID | IGVK4- | YSSNNKNYLAWYQQKPGQPPKLLIYWASTR |
| | NO: | 1 | ESGVPDRFSGSGSGTDFTLTISSLQAEDVA |
| | 162 | | VYYCQQYYSTP |

Humanization was carried out by CDR-grafting (Methods in Molecular Biology, Vol 248: Antibody Engineering, Methods and Protocols, Humana Press) and the humanization antibody variants were engineered into a human IgG1 format using an expression vector developed in-house. In the initial round of humanization, mutations from murine to human amino acid residues in framework regions were guided by a simulated 3D structure, and the murine framework residues of structural importance for maintaining the canonical structures of CDRs were retained in the humanized variants, the rest of the murine framework residues were back mutated to corresponding human framework residues one by one to generate a number of humanized variants, followed by purification and characterization to determine the murine framework residues that can be replaced by human framework residues, while maintaining the desired biophysical and biochemical properties. In the second round of humanization, the critical murine framework residues that were identified during the initial round of humanization were combined to generate the desired versions of humanized BGA12. Specifically, CDRs of BGA12 VL were grafted into the framework of human germline variable gene IGVK 4-1 with 1 murine residue (S₄₃) retained, resulting in the humanized VL sequence of BGA122 (SEQ ID NO:111), or with no murine residue retained, resulting in the humanized VL sequence of BGA121 (SEQ ID NO:101). CDRs of BGA12 VH were grafted into the framework of human germline variable gene IGVH1-3 with 3 murine residue (S₄₄, M₆₉, V₇₁) retained, resulting in the humanized VH sequences of BGA121 (SEQ ID NO:100) and BGA122 (SEQ ID NO:110).

BGA121 and BGA122 were constructed into a humanized full-length antibody format using in-house developed expression vectors that contain constant regions of a human IgG1 and kappa chain respectively, with easy adapting sub-cloning sites. Expression and purification of BGA121 and BGA122 were achieved by co-transfection of the above two constructs into Expi293™ cells and by purification via a protein A column (Cat: 17543801, GE Life Sciences). The purified antibodies were concentrated to 0.5-5 mg/mL in PBS and stored in aliquots in a −80° C. freezer.

Characterization of BGA121 and BGA122 was carried out in detail. The results showed all BGA12 humanized variants were very similar in binding affinity and functional activities such as inhibiting PVRL2-mediated downstream signaling.

### Affinity Determination for Different Versions BGA12

For affinity determination, antibodies were captured by anti-human Fe antibodies, and used in the affinity assay based on surface plasmon resonance (SPR) technology. The results of SPR-determined binding profiles of anti-PVRIG antibodies were summarized in Table 12 and Table 13, BGA121 and BGA122 showed very similar binding profiles to human PVRIG and Cyno PVRIG, and are similar to the parental antibody BGA12. Rmax represents the maximal feasible SPR signal generated by an interaction between a ligand-analyte pair and is represented in response units (RU).

TABLE 12
Binding profiles of anti-PVRIG antibodies to human PVRIG

| Antibody | Ka (M⁻¹ S⁻¹) | Kd S⁻¹) | KD(M) | Rmax |
|---|---|---|---|---|
| BGA121 | 2.33E⁺07 | 1.22E−03 | 5.24E−11 | 32.4 |
| BGA122 | 4.09E⁺07 | 1.99E−03 | 4.88E−11 | 38.1 |
| BGA12 | 2.29E⁺07 | 1.57E−03 | 6.84E−11 | 43.4 |

TABLE 13
Binding profiles of anti-PVRIG antibodies to Cyno PVRIG

| Antibody | Ka (M⁻¹ S⁻¹) | Kd (S⁻¹) | KD(M) | Rmax |
|---|---|---|---|---|
| BGA121 | 3.86E⁺04 | 2.89E−04 | 7.49E−09 | 42 |
| BGA122 | 6.28E⁺04 | 1.16E−04 | 1.85E−09 | 33.1 |
| BGA12 | 5.61E⁺04 | 3.54E−04 | 6.31E−09 | 27.9 |

### Binding Activities of Different Versions of BGA12 to Native Human PVRIG

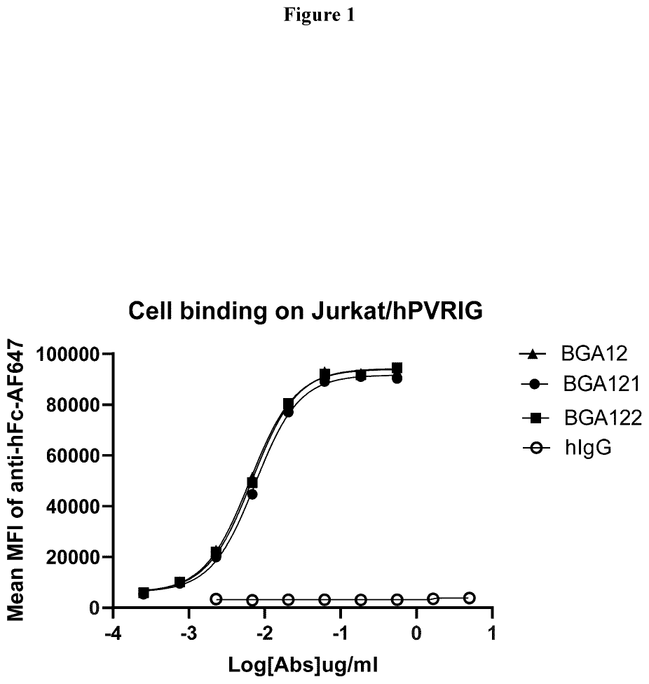

To evaluate the binding activity of anti-PVRIG antibodies to native PVRIG on living cells, Jurkat cells were engineered to express human PVRIG. Jurkat cells expressing human PVRIG were seeded in 96 well plates, and were incubated with serially diluted anti-PVRIG antibodies. Goat anti-Human IgG was used as a secondary antibody to detect antibody binding to the cell surface. EC50 values for dose dependent binding to human native PVRIG were determined by fitting the dose response data to the four-parameter logistic model with GraphPad Prism™. As shown in FIG. 1, all humanized BGA12 antibodies demonstrated high binding affinity to native human PVRIG on living cells.

Anti-PVRIG Antibodies Reduce the Interactions of PVRIG with its Ligand PVRL2

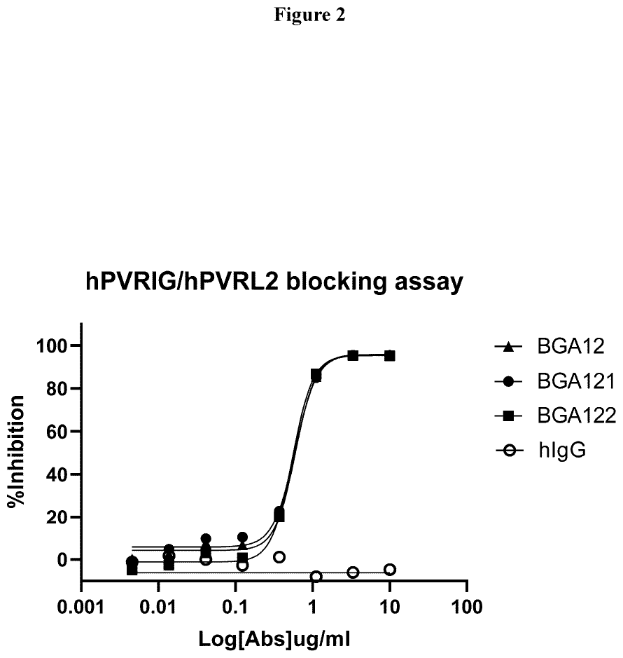

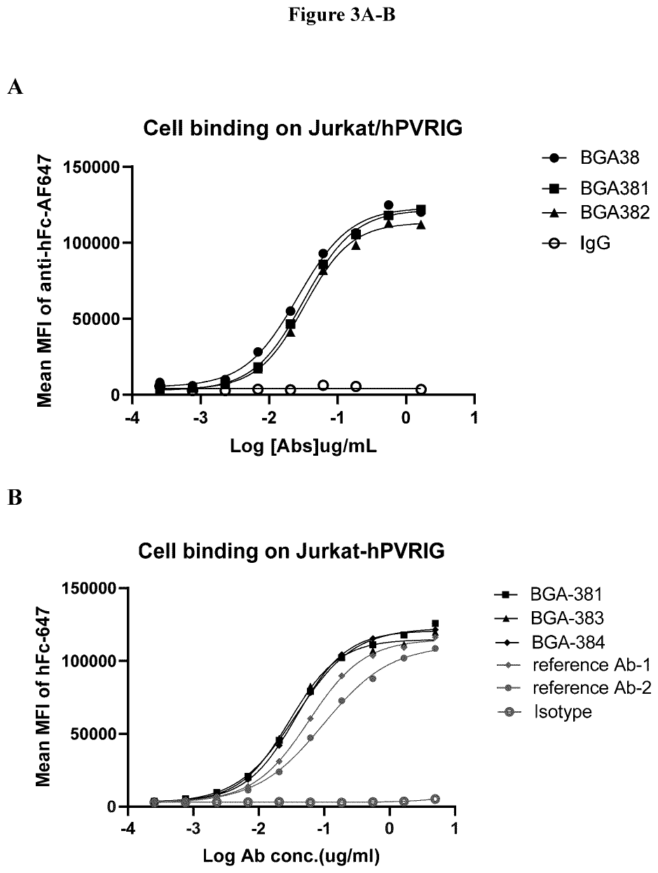

To determine whether anti-PVRIG antibodies could reduce the PVRIG-PVRL2 signaling axis (the binding interaction between receptor and ligand), His-tagged human PVRIG (hPVRIG-His) protein was coated on 96 well ELISA plates. Serial dilutions of PVRIG humanized antibody variants starting from 10 μg/mL were mixed with PVRL2-biotin at a final concentration of 1 μg/mL. As shown in FIG. 2, BGA12, BGA121 and BGA122 antibodies greatly reduced PVRIG binding to PVRL2 in a dose dependent manner with similar IC50.

### Example 10. Humanization and Engineering of the Anti-PVRIG Antibody BGA38

For humanization of the BGA38 antibody, human germline IgG genes were searched for sequences that share high degrees of homology to the cDNA sequences of the BGA38 variable regions by BLAST of the human immunoglobulin gene database in IMGT and the NCBI database. The human IGVH and IGVL genes that are present in human antibody repertoires with high frequencies (Glanville 2009 PNAS 106:20216-20221) and are highly homologous to BGA38 were selected as the templates for humanization.

Humanization was carried out by CDR-grafting (Methods in Molecular Biology, Vol 248: Antibody Engineering, Methods and Protocols, Humana Press) and the humanization antibody variants were engineered into the human IgG1 format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the humanized variants, the rest of the murine framework residues were back mutated to corresponding human framework residue one by one to generate a number of humanized variants, followed by purification and characterization to determine the murine framework residues that can be replaced by human framework residues, while maintaining the desired biophysical and biochemical properties. In the second round of humanization, the critical murine framework residues that were identified during the initial round of humanization were combined to generate the desired versions of humanized BGA38. Specifically, CDRs of BGA38 VL were grafted into the framework of human germline variable gene IGVK 3-11 with 4 murine residues (Q₁ F₃₆ V₅₈ Y₇₁) retained, resulting in the humanized VL sequence of BGA381 (SEQ ID NO:21), or with 5 murine residues (Q₁ N₂ F₃₆ V₅₈ Y₇₁) retained, resulting in the humanized VL sequence of BGA382 (SEQ ID NO:31). CDRs of BGA38 VH were grafted into the framework of human germline variable gene IGVH1-46 with 1 murine residue (Q₁) retained, resulting in the humanized VH sequences of BGA381 (SEQ ID NO:20) and BGA382 (SEQ ID NO:30), BGA383 (SEQ ID NO:40) and BGA384 (SEQ ID NO:50).

BGA381-BGA384 were constructed in human full-length antibody format using in-house developed expression vectors that contain constant regions of a human IgG1 and kappa chain respectively, with easy adapting sub-cloning sites. Expression and preparation of BGA381, BGA382, BGA383 and BGA384, was achieved by co-transfection of the VH and VL constructs into Expi293™ cells and purification via a protein A column (Cat: 17543801, GE Life Sciences). The purified antibodies were concentrated to 0.5-5 mg/mL in PBS and stored in aliquots in a −80° C. freezer.

BGA38 humanized antibody variants were further engineered to remove post translational modification (PTM) site(s) in the CDRs to improve molecular and biophysical properties for therapeutic use in humans. The considerations included amino acid compositions, heat stability (Tm), surface hydrophobicity and isoelectronic points (PIs) while maintaining antibody function.

Taken together, engineered versions of humanized monoclonal antibody BGA381, BGA382, BGA383 and BGA384, were derived from the process described above, and characterized in detail. The results showed all BGA38 humanized variants displayed similarly effective binding affinity and functional activities, such as inhibiting PVRL2-mediated downstream signaling.

### Affinity Determination for Different Versions of BGA38

For affinity determination, anti-PVRIG antibodies were captured by anti-human Fc antibodies, and used in the affinity assay based on surface plasmon resonance (SPR) technology. The results of SPR-determined binding profiles of anti-PVRIG antibodies are summarized in Table 14 and Table 15, BGA381, BGA382, BGA383 and BGA384 showed very similar binding profiles to human PVRIG and Cyno PVRIG, similar to the parental antibody BGA38.

TABLE 14
Binding profiles of anti-PVRIG antibodies to human PVRIG

| Antibody | Ka (M⁻¹ S⁻¹) | Kd (S⁻¹) | KD(M) | Rmax |
|---|---|---|---|---|
| BGA38 | 3.89e⁺6 | 1.75e−4 | 4.50e−11 | 35.5 |
| BGA381 | 4.40e⁺6 | 3.11e−4 | 7.07e−11 | 39.9 |
| BGA382 | 3.26e⁺6 | 2.11e−4 | 6.46e−11 | 42.0 |
| BGA383 | 6.51e⁺6 | 6.49e−4 | 9.98e−11 | 34.4 |
| BGA384 | 1.18e⁺6 | 3.04e−4 | 2.58e−10 | 67.4 |

TABLE 15
Binding profiles of anti-PVRIG antibodies to Cyno PVRIG

| Antibody | Ka (M⁻¹ S⁻¹) | Kd (S⁻¹) | KD(M) | Rmax |
|---|---|---|---|---|
| BGA38 | 4.15e⁺6 | 2.10e−4 | 5.07e−11 | 34.7 |
| BGA381 | 4.98e⁺6 | 3.67e−4 | 7.38e−11 | 42.7 |
| BGA382 | 4.70e⁺6 | 2.43e−4 | 5.18e−11 | 49.4 |
| BGA383 | 5.25e⁺6 | 3.45e−4 | 6.58e−11 | 36.3 |
| BGA384 | 5.99e⁺6 | 3.66e−4 | 6.10e−11 | 34.6 |

### Binding Activities of Different Versions of BGA38 to Native Human PVRIG

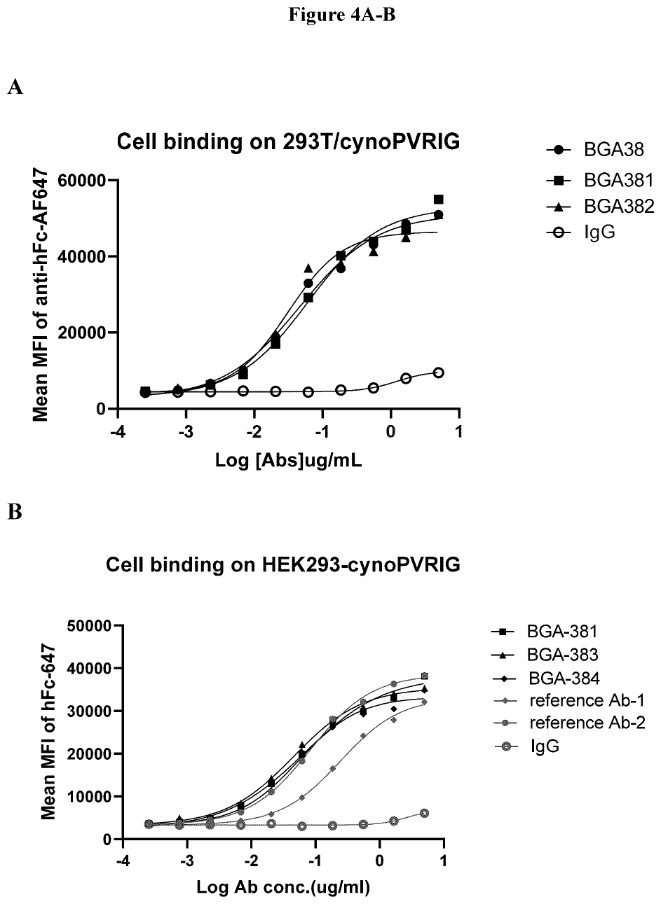

To evaluate the binding activity of anti-PVRIG antibodies to native PVRIG on living cells, Jurkat cells were engineered to express human PVRIG. Jurkat cells expressing PVRIG were seeded in 96 well plates and were incubated with serially diluted anti-PVRIG antibodies. Goat anti-Human IgG was used as secondary antibody to detect antibody binding to the cell surface. EC50 values for dose dependent binding to human native PVRIG were determined by fitting the dose response data to the four-parameter logistic model with GraphPad Prism™. As shown in FIG. 3, all humanized BGA38 antibodies demonstrated high binding affinity to native PVRIG on living cells.

### Binding Activities of Different Versions of BGA38 to Native Cyno PVRIG

To evaluate the binding activity of anti-PVRIG antibodies to native Cyno PVRIG on living cells. HEK293 cells were engineered to express Cyno PVRIG. HEK293 cells transfected with Cyno PVRIG were seeded in 96 well plates and were incubated with a serial dilution of anti-PVRIG antibodies. Goat anti-Human IgG was used as secondary antibody to detect antibody binding to the cell surface. EC50 values for dose dependent binding to Cyno native PVRIG were determined by fitting the dose response data to the four-parameter logistic model with GraphPad Prism™. As shown in FIG. 4, all humanized BGA38 antibodies demonstrated high binding affinity to native Cyno PVRIG on living cells.

Anti-PVRIG Antibodies Reduce the Interactions of PVRIG with its Ligands PVRL2

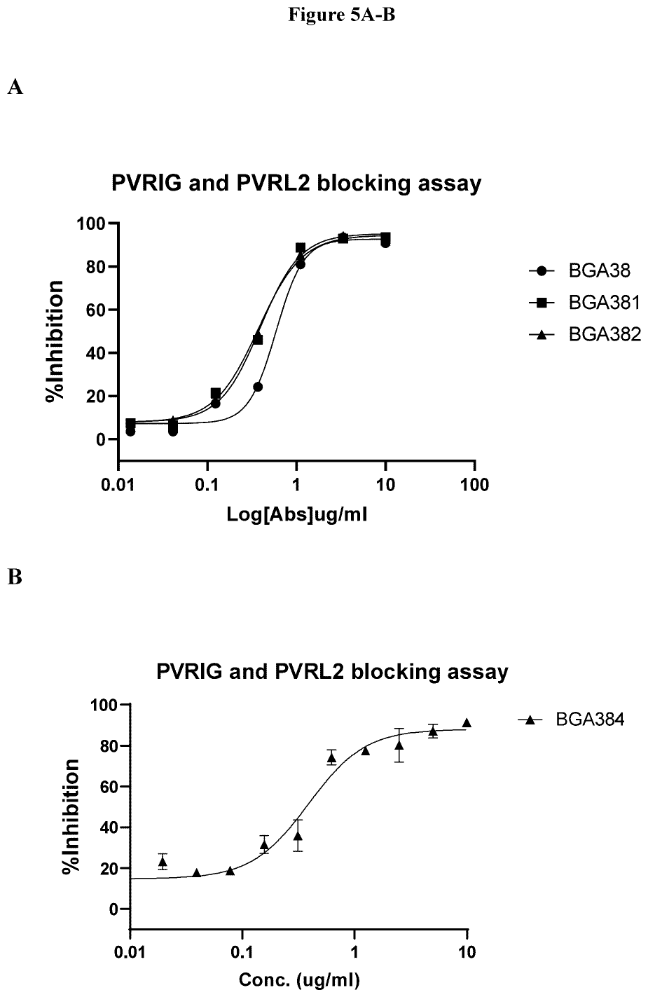

To determine whether anti-PVRIG antibodies could reduce PVRIG-PVRL2 interactions, hPVRIG-His tagged proteins were coated on 96 well ELISA plates. A serial dilution of PVRIG humanized antibody variants starting from 10 μg/mL were mixed with PVRL2-biotin at a final concentration of 1 μg/mL. As shown in FIGS. 5A-B, all humanized BGA38 antibodies greatly reduced PVRIG binding to PVRL2 in a dose dependent manner with similar IC50.

### Example 11. Humanization and Engineering of the Anti-PVRIG Antibody BGA86

For humanization of the BGA86 antibody, human germline IgG genes were searched for sequences that share high degree of homology to the cDNA sequences of BGA86 variable regions by BLAST of the human immunoglobulin gene database in IMGT and the NCBI database. The human IGVH and IGVL genes that are present in human antibody repertoires with high frequencies (Glanville 2009 PNAS 106:20216-20221) and are highly homologous to BGA86 were selected as the templates for humanization.

Humanization was carried out by CDR-grafting (Methods in Molecular Biology, Vol 248: Antibody Engineering. Methods and Protocols, Humana Press) and the humanization antibody variants were engineered into the human IgG1 format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the one of the humanized variants, the rest of the murine framework residues were back mutated to the corresponding human framework residue one by one to generate a number of humanized variants, followed by purification and characterization to determine the murine framework residues that can be replaced by human framework residues, while maintaining the desired biophysical and biochemical properties. In the second round of humanization, the critical murine framework residues that were identified during the initial round of humanization were combined to generate the desired versions of humanized BGA86. Specifically, CDRs of BGA86 VL were grafted into the framework of human germline variable gene IGVK 1-39 with no murine residues retained, resulting in the humanized VL sequence of BGA86 (SEQ ID NO:61). CDRs of BGA86 VH were grafted into the framework of human germline variable gene IGVH3-21 with 2 murine residues (T₇₇, S_{82A}) retained, resulting in the humanized VH sequences of BGA86 (SEQ ID NO:60).

Humanized BGA86 antibody variants were constructed in a human full-length antibody format using in-house developed expression vectors that contain constant regions of a human IgG1 and kappa chain respectively, with easy adapting sub-cloning sites. Expression and preparation of humanized BGA86 variants were achieved by co-transfection of the above two constructs into Expi293™ cells and by purification via protein A column (Cat: 17543801, GE Life Sciences). The purified antibodies were concentrated to 0.5-5 mg/mL in PBS and stored in aliquots in a −80° C. freezer.

BGA86 humanized antibody variants were further engineered to remove the post translational modification (PTM) site(s) in the CDRs to improve molecular and biophysical properties for therapeutic use in humans. The considerations included amino acid compositions, heat stability (Tm), surface hydrophobicity and isoelectronic points (pIs) while maintaining antibody function.

Engineered versions of humanized monoclonal antibody BGA861 and BGA862 were derived from the process described above and characterized in detail. The results showed all BGA86 humanized variants were very similar in binding affinity and functional activities such as inhibiting the PVRIG/PVRL2 signaling axis, and interfering with PVRIG-mediated downstream signaling.

### Affinity Determination for Different Versions of BGA86

For affinity determination, antibodies were captured by anti-human Fc antibodies, and used in the affinity determination based on surface plasmon resonance (SPR) technology. The results of SPR-determined binding profiles of anti-PVRIG antibodies are summarized in Table 16 and Table 17. BGA861 and BGA862 showed very similar binding profiles to human PVRIG and Cyno PVRIG, and are similar to the parental antibody BGA86.

TABLE 16
Binding profiles of anti-PVRIG antibodies to human PVRIG

| Antibody | Ka (M⁻¹ S⁻¹) | Kd (S⁻¹) | KD(M) | Rmax |
|---|---|---|---|---|
| BGA86 | 2.54e⁺6 | 7.72e−4 | 3.04e−10 | 45.4 |
| BGA861 | 1.48e⁺6 | 9.54e−4 | 6.45e−10 | 46.7 |
| BGA862 | 2.41e⁺6 | 2.16e−3 | 8.98e−10 | 48.7 |

TABLE 17
Binding profiles of anti-PVRIG antibodies to Cyno PVRIG

| Antibody | Ka (M⁻¹ S⁻¹) | Kd (S⁻¹) | KD(M) | Rmax |
|---|---|---|---|---|
| BGA86 | 3.08e⁺6 | 1.70e−3 | 5.52e−10 | 38.8 |
| BGA861 | 2.18e⁺6 | 2.21e−3 | 1.01e−9 | 38.1 |
| BGA862 | 2.23e⁺6 | 4.83e−3 | 2.16e−9 | 44.2 |

### Binding Activities of Different Versions of BGA86 Antibodies to Native Human PVRIG

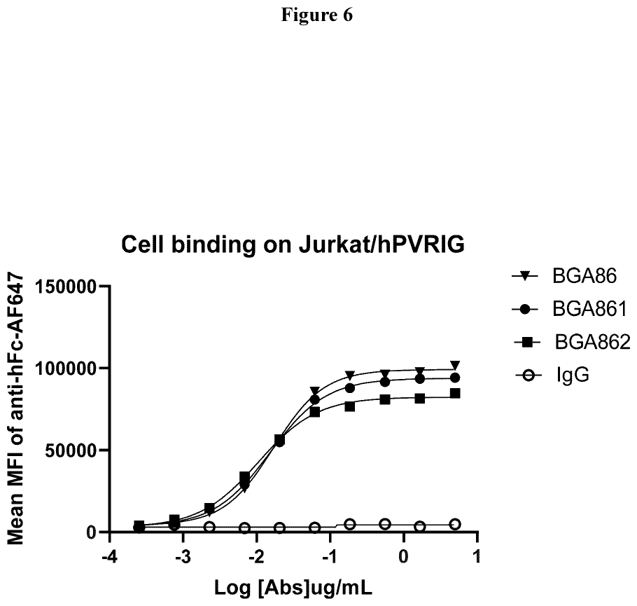

To evaluate the binding activity of anti-PVRIG antibodies to native PVRIG on living cells, Jurkat cells were engineered to express human PVRIG. Jurkat cells expressing hPVRIG were seeded in 96 well plates and were incubated with serially diluted anti-PVRIG antibodies. Goat anti-Human IgG was used as a secondary antibody to detect antibody binding to the cell surface. EC50 values for dose dependent binding to human native PVRIG were determined by fitting the dose response data to the four-parameter logistic model with GraphPad Prism™. As shown in FIG. 6, all humanized BGA86 antibodies demonstrated high binding affinity to native PVRIG on living cells.

### Binding Activities of Different Versions of BGA86 Antibody to Native Cyno PVRIG

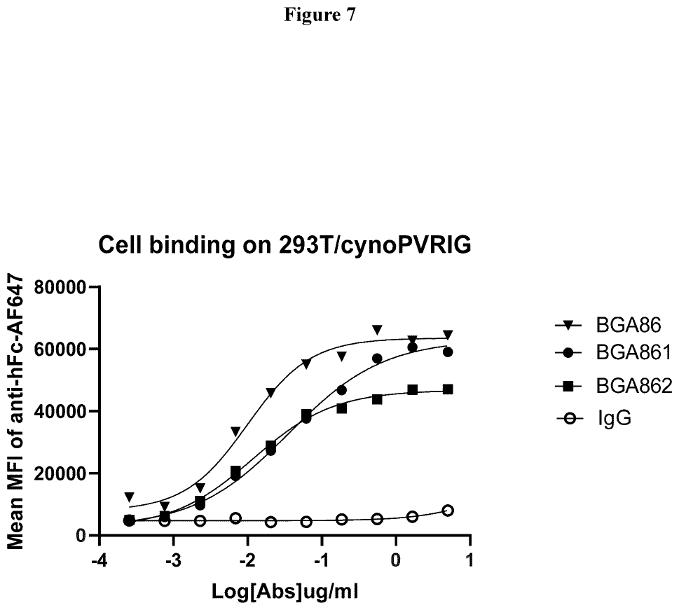

To evaluate the binding activity of anti-PVRIG antibodies to native Cyno PVRIG on living cells. HEK293 cells were engineered to express Cyno PVRIG. HEK293 expressing PVRIG cells were seeded in 96 well plates and were incubated with a serial dilution of anti-PVRIG antibodies. Goat anti-Human IgG was used as secondary antibody to detect antibody binding to the cell surface. EC50 values for dose dependent binding to Cyno native PVRIG were determined by fitting the dose response data to the four-parameter logistic model with GraphPad Prism™. As shown in FIG. 7, all humanized BGA86 antibodies demonstrated high binding affinity to native Cyno PVRIG on living cells.

Anti-PVRIG Antibodies Block the Interactions of PVRIG with its Ligand PVRL2

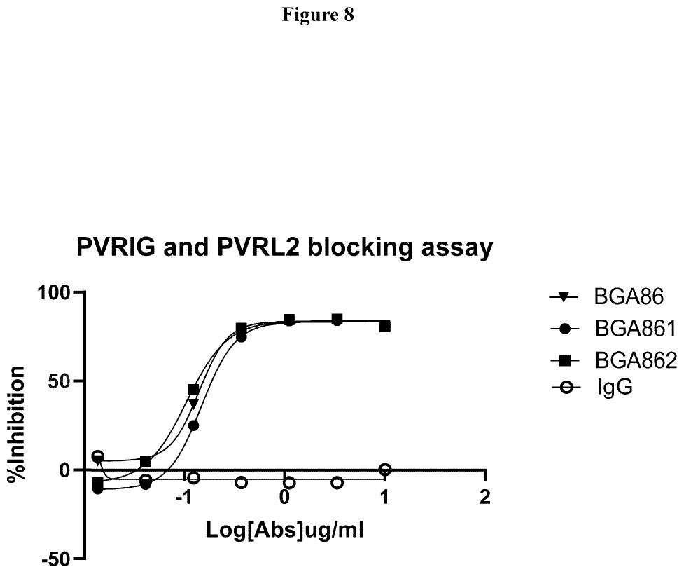

To determine whether anti-PVRIG antibodies could reduce PVRIG-PVRL2 interactions, hPVRIG-His tagged protein was coated on 96 well ELISA plates. A serial dilution of PVRIG humanized antibody variants starting from 10 μg/mL were mixed with PVRL2-biotin at a final concentration of 1 μg/mL. As shown in FIG. 8, all humanized BGA86 antibodies greatly reduced PVRIG binding to PVRL2 in a dose dependent manner with similar IC50.

### Example 12. Humanization and Engineering of the Anti-PVRIG Antibody BGA50

For humanization of BGA50, human germline IgG genes were searched for sequences that share high degrees of homology to the cDNA sequences of BGA50 variable regions by BLAST analysis of the human immunoglobulin gene database in IMGT and the NCBI database. The human IGVH and IGVL genes that are present in human antibody repertoires with high frequencies (Glanville 2009 PNAS 106:20216-20221) and are highly homologous to BGA50 were selected as the templates for humanization.

Humanization was carried out by CDR-grafting (Methods in Molecular Biology. Vol 248: Antibody Engineering. Methods and Protocols. Humana Press) and the humanization antibody variants were engineered into a human IgG1 format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the one of the humanized variants, the rest of the murine framework residues were back mutated to the corresponding human framework residue one by one to generate a number of humanized variants, followed by purification and characterization to determine the murine framework residues that can be replaced by human framework residues, while maintaining their biophysical and biochemical properties. In the second round of humanization, the critical murine framework residues that were identified during the initial round of humanization were combined to generate the desired versions of humanized BGA50. Specifically. CDRs of BGA50 VL were grafted into the framework of human germline variable gene IGVK 4-1 with 1 murine residue (S43) retained, resulting in the humanized VL sequence of BGA501 (SEQ ID NO:131), BGA502 (SEQ ID NO:141), BGA503 (SEQ ID NO:151). CDRs of BGA50 VH were grafted into the framework of human germline variable gene IGVH1-18 with 3 or 4 murine residue (S₂₈, I₄₈, K₇₃, V₇₁) retained, resulting in the humanized VH sequences of BGA501 (SEQ ID NO:130), BGA502 (SEQ ID NO:140) and BGA503 (SEQ ID NO:150).

BGA501, BGA502 and BGA503 were constructed in a human full-length antibody format using in-house developed expression vectors that contain constant regions of a human IgG1 and kappa chain respectively, with easy adapting sub-cloning sites. Expression and preparation of BGA50 variants were achieved by co-transfection of the VH and VL constructs into Expi293™ cells and by purification via a protein A column (Cat: 17543801. GE Life Sciences). The purified antibodies were concentrated to 0.5-5 mg/mL in PBS and stored in aliquots in −80° C. freezer.

BGA50 humanized antibody variants were further engineered by remove the post translational modification (PTM) site(s) in CDRs to improve molecular and biophysical properties for therapeutic use in humans. The considerations include amino acid compositions, heat stability (Tm), surface hydrophobicity and isoelectronic points (PIS) while maintaining antibody function.

Engineered versions of humanized monoclonal antibody BGA501, BGA502 and BGA503 were derived from the mutation process described as above and characterized in detail. The results showed all BGA50 humanized variants were very similar in binding affinity and functional activities such as inhibiting the PVRL2-mediated downstream signaling.

### Affinity Determination for Different Versions of BGA50

For affinity determination, antibodies were captured by anti-human Fc antibodies, and used in the affinity determination based on surface plasmon resonance (SPR) technology. The results of SPR-determined binding profiles of anti-PVRIG antibodies were summarized in Table 18 and Table 19, BGA501. BGA502 and BGA503 showed very similar binding profiles to human PVRIG and Cyno PVRIG and are similar to the parental antibody BGA50.

TABLE 18
Binding profiles of anti-PVRIG antibodies to human PVRIG

| Antibody | Ka (M⁻¹ S⁻¹) | Kd (S⁻¹) | KD(M) | Rmax |
|---|---|---|---|---|
| BGA50 | 5.72e⁺6 | 5.19e−4 | 9.07e−11 | 35.9 |
| BGA501 | 3.21e⁺6 | 4.36e−4 | 1.36e−10 | 46.9 |
| BGA502 | 4.71e⁺6 | 4.86e−4 | 1.03e−10 | 62.6 |
| BGA503 | 3.66e⁺6 | 4.38e−4 | 1.20e−10 | 51.2 |

TABLE 19
Binding profiles of anti-PVRIG antibodies Cyno PVRIG

| Antibody | Ka (M⁻¹ S⁻¹) | Kd (S⁻¹) | KD(M) | Rmax |
|---|---|---|---|---|
| BGA50 | 9.21e⁺6 | 3.20e−3 | 3.47e−10 | 35.4 |
| BGA501 | 6.27e⁺6 | 2.67e−3 | 4.26e−10 | 44.1 |
| BGA502 | 6.16e⁺6 | 2.66e−3 | 4.33e−10 | 58.4 |
| BGA503 | 7.44e⁺6 | 2.94e−3 | 3.96e−10 | 49.8 |

### Binding Activities of Different Versions of BGA50 to Native Human PVRIG

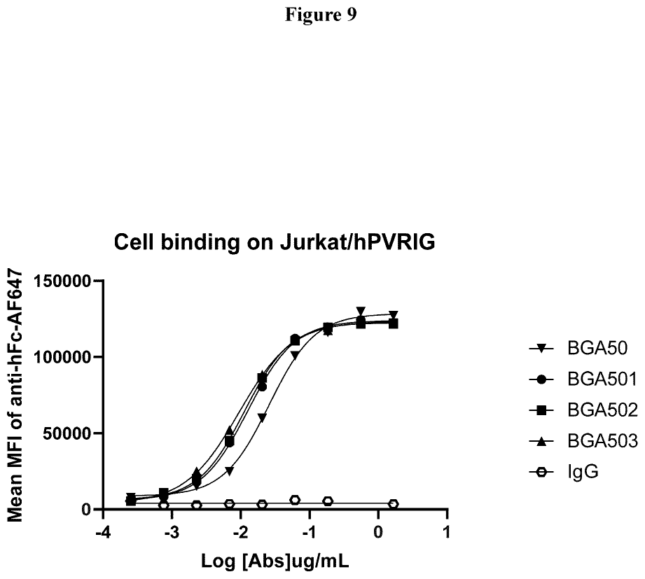

To evaluate the binding activity of anti-PVRIG antibodies to native human PVRIG on living cells, Jurkat cells were engineered to express human PVRIG. Jurkat cells expressing human PVRIG were seeded in 96 well plates, and were incubated with serially diluted anti-PVRIG antibodies. Goat anti-Human IgG was used as secondary antibody to detect antibody binding to the cell surface. EC50 values for dose dependent binding to human native PVRIG were determined by fitting the dose response data to the four-parameter logistic model with GraphPad Prism™. As shown in FIG. 9, all humanized BGA50 antibodies demonstrated high binding affinity to native PVRIG on living cells.

### Binding Activities of Different Versions of BGA50 to Native Cyno PVRIG

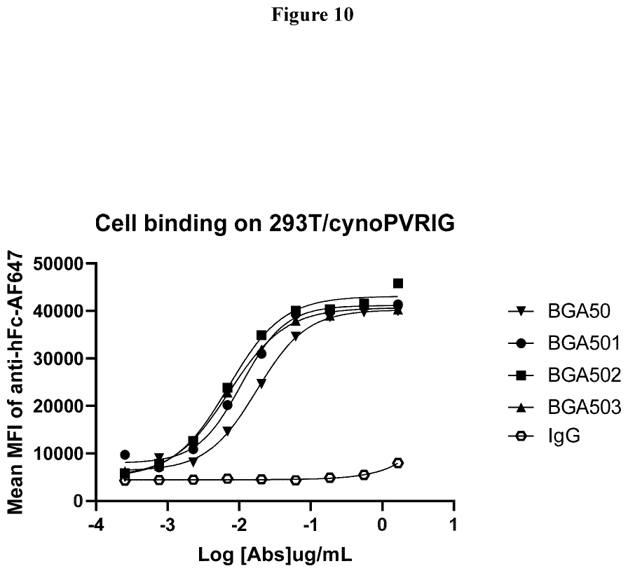

To evaluate the binding activity of anti-PVRIG antibodies to native Cyno PVRIG on living cells. HEK293 cells were engineered to express Cyno PVRIG. HEK293 expressing Cyno PVRIG cells were seeded in 96 well plates and were incubated with serial dilutions of anti-PVRIG antibodies. Goat anti-Human IgG was used as a secondary antibody to detect antibody binding to the cell surface. EC50 values for dose dependent binding to Cyno native PVRIG were determined by fitting the dose response data to the four-parameter logistic model with GraphPad Prism™. As shown in FIG. 10, all humanized BGA50 antibodies demonstrated high binding affinity to native Cyno PVRIG on living cells.

Anti-PVRIG Antibodies Reduce the Interactions of PVRIG with its Ligand PVRL2

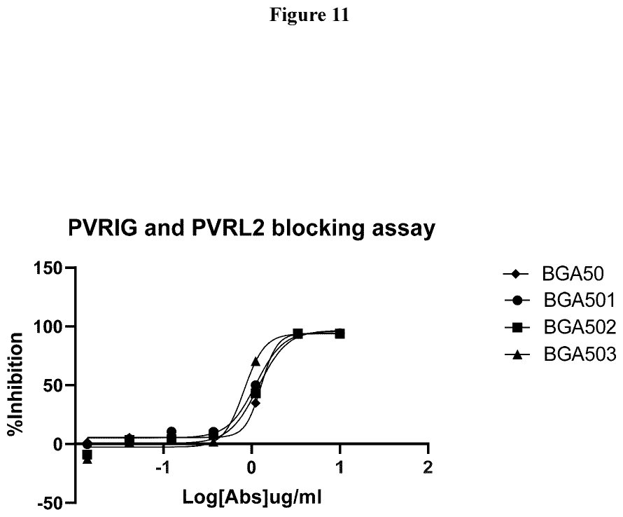

To further determine whether anti-PVRIG antibodies could reduce the PVRIG-PVRL2 binding interaction, hPVRIG-His tagged proteins were coated on 96 well ELISA plates, and a serial dilution of PVRIG humanized antibody variants starting from 10 μg/mL were mixed with PVRL2-biotin at a final concentration of 1 μg/mL. As shown in FIG. 11, all humanized BGA50 antibodies greatly reduced PVRIG binding to PVRL2 in a dose dependent manner with similar IC50.

### Example 13. T Cell Activation Function of PVRIG Antibodies in Jurkat/NFAT-Luciferase Reporter/hPVRIG Assay

The ability of PVRIG antibodies to induce T cell activation though PVIRG/PVRL2 blockade was determined using Jurkat/NFAT Luciferase reporter/hPVRIG assay as described below.

### Jurkat/NFAT-Luciferase Reporter/hPVRIG and A549 Co-Culture Assay

Jurkat/NFAT-Luciferase reporter/hPVRIG were generated from Jurkat cells by stable transfection of expression constructs containing a luciferase reporter and human PVRIG. A549 cells (5×10⁴ cells/well) were co-cultured with Jurkat/NFAT-Luciferase reporter/hPVRIG cells (5×10⁴ cells/well) in the presence of 5 ng/ml of DN001, an internally purified anti-CD3×NKG2D bispecific antibody which binds CD3 on the Jurkat/NFAT-Luciferase reporter/hPVRIG cells and the ligands of NKG2D on A549 and acts to provide a stimulating signal to Jurkat/NFAT-Luciferase reporter/hPVRIG cells. To this mix, serially diluted PVRIG antibodies in a 96 well flat-bottom plate were incubated overnight. NFAT signaling was measured using the One-glo™ luciferase assay system (Promega).

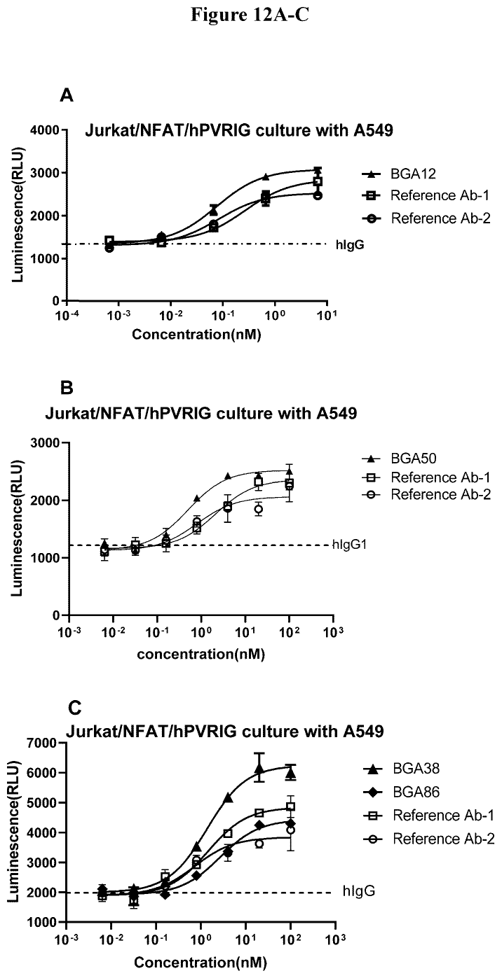

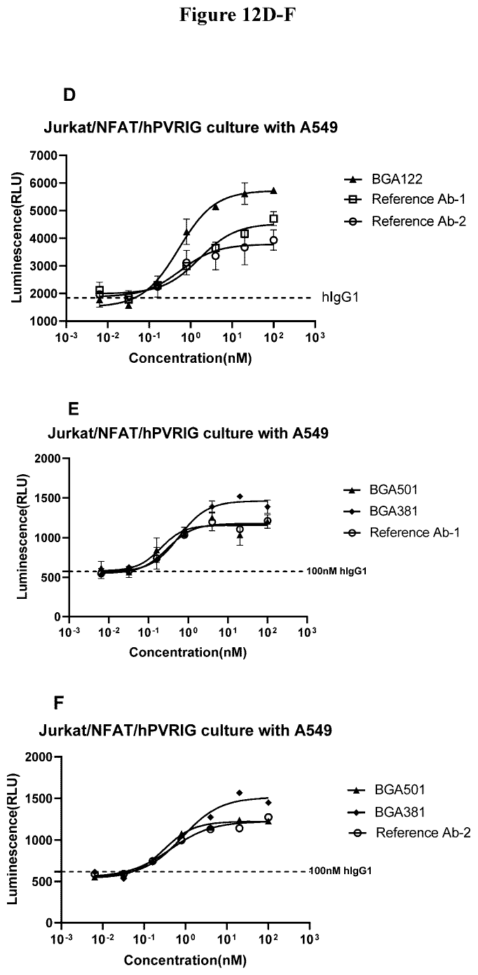

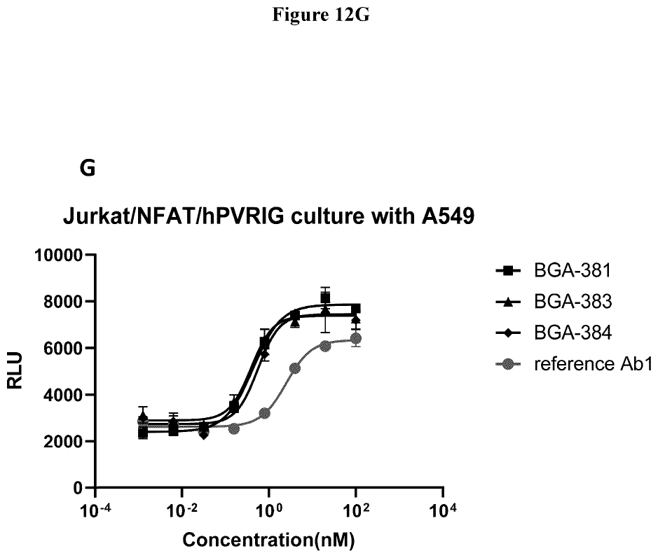

Results: As shown in FIGS. 12A-C. BGA12, BGA50, BGA38, and BGA86 enhanced Jurkat/NFAT-Luciferase reporter/hPVRIG activation in a dose dependent manner. BGA12, BGA50 and BGA38 exhibited superior activity relative to the reference antibodies, and BGA86 showed comparable activity to reference antibodies. After humanization, as shown in FIG. 12D-G. BGA122, BGA501. BGA381. BGA383 and BGA384 activated Jurkat/NFAT-Luciferase reporter/hPVRIG in a dose dependent manner. BGA122. BGA381. BGA383 and BGA384 elicited a stronger maximum response compared to reference antibodies.

Jurkat/NFAT-Luciferase reporter/hPVRIG⁺THP1/OS8 low assay

THP1/OS8^{(low)} cells (5×10⁴ cells/well) were co-cultured with Jurkat/NFAT-Luciferase reporter/hPVIRG (5×10⁴ cells/well) in the presence of serially diluted PVRIG antibodies in a 96 well flat-bottom plate overnight. NFAT signaling was measured using One-glo™ luciferase assay system (Promega). The Fc of the antibodies used was either wild type (WT) with normal function or was an Fc silent version (MT) with reduced ADCC and complement binding.

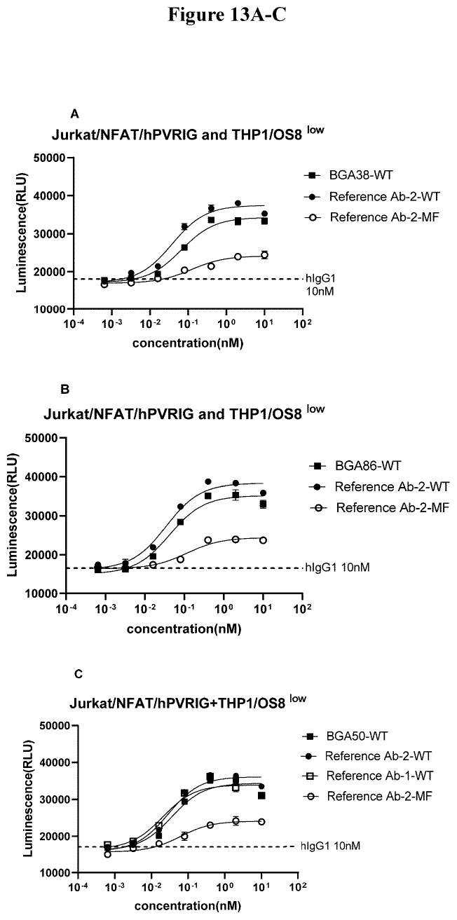

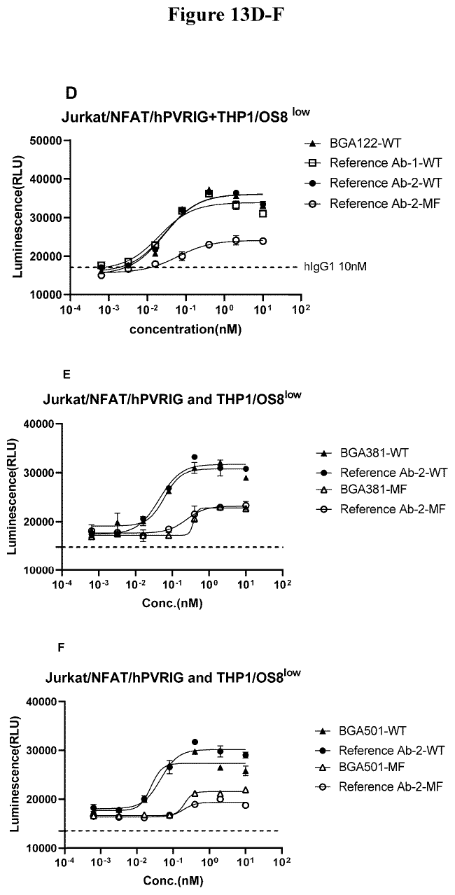

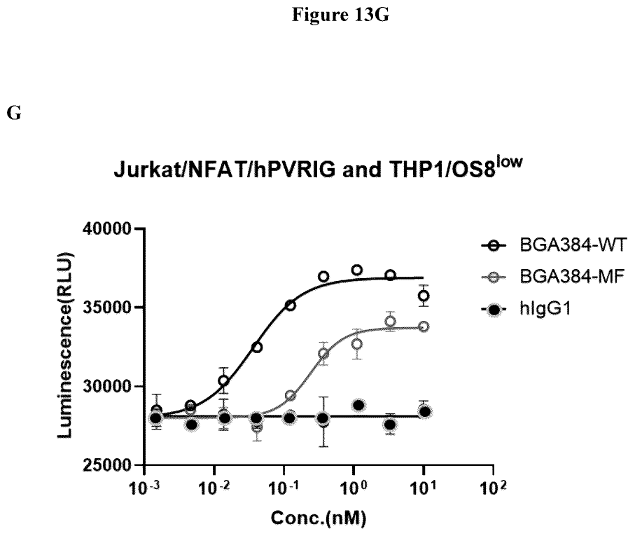

Results: As shown in FIGS. 13A-C. BGA38-WT. BGA86-WT and BGA50-WT enhanced Jurkat/NFAT-Luciferase reporter/hPVRIG activation in a dose dependent manner. After humanization, as shown in FIGS. 13D-G. BGA122-WT. BGA381-WT/MF. BGA501-WT/MF, and BGA384 WT/MF also activated Jurkat/NFAT-Luciferase reporter/hPVRIG dose dependently. Furthermore, it was demonstrated that the Fc function can increase the activity via the comparison of the Fe-competent versions and Fc-silent versions of BGA381. BGA501 and BGA384.

### Example 14. Activation of CMV Antigen Specific Human T Cells by Anti-PVRIG Antibodies

The functional activity of the PVRIG antibodies were further assessed using naturally derived T-cells that recognized human CMV pp65 peptide (NLVPMVATV (SEQ ID NO:185)), amino acids 495-503. HLA-A2.1-restricted) (Boeckh et al., 2011 J Clin Invest. 121:1673-80). Briefly. HLA-A*0201 PBMCs (Donor Z0052) were stimulated by 1 μg/mL CMV pp65 peptide, 2 ng/ml IL-2 and 10 ng/ml IL-7 for 8 days, with IL-2 and IL-7 added on day 3 and day 5. The cells were then rested in media with 500 IU/mL IL-2 for 1 day. HCT116 cells (ATCC CCL-247), a human colorectal carcinoma cell line, were pulsed with 0.01 μg/mL CMV pp65 peptide at 37° C. for 1 hour and washed. Then the PBMCs were co-cultured with CMV pp65 peptide pulsed HCT116 cells in the presence of 0.5 or 5 μg/ml PVRIG antibody overnight. Human IFN-γ was measured by ELISA. 7.518, an anti-PVRIG antibody disclosed in WO2016134333 and Surface 35 an anti-PVRIG antibody disclosed in US20200040081 were used as controls.

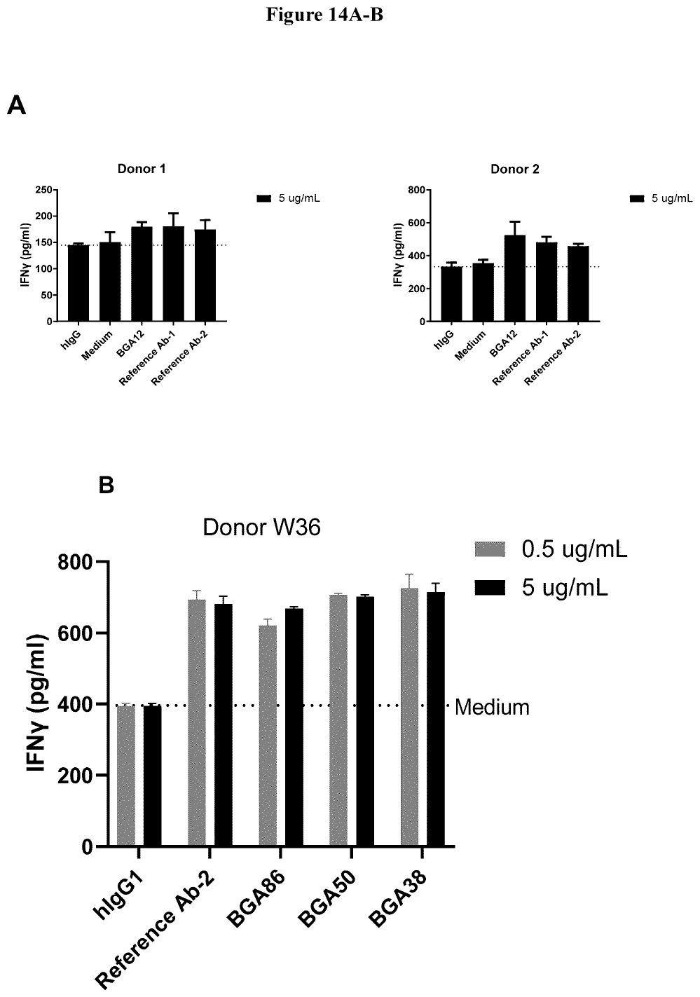

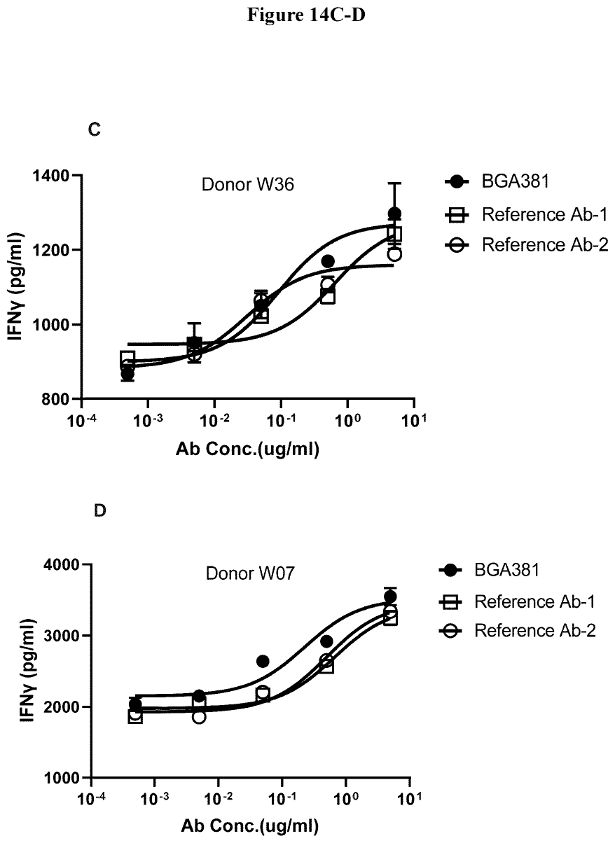

Results: As shown in FIGS. 14A and 14B, BGA12, BGA86, BGA50 and BGA38 promoted IFN-γ production. After humanization, as shown in FIGS. 14C and 14D. BGA381 elicited IFN-γ production in a dose-dependent manner.

### Example 15. PVRIG Antibodies Used in Combination with Other Immune Checkpoint Antibodies

HLA-A*0201 PBMCs were stimulated by 1 μg/mL CMV pp65 peptide NLVPMVATV (SEQ ID NO: 185), 2 ng/ml IL-2 and 10 ng/ml IL-7 for 8 days, with IL-2 and IL-7 added on day 3 and day 5. The cells were then rested in media with 500 IU/mL IL-2 for 1 day. HCT116 cells. (ATCC CCL-247), a human colorectal carcinoma cell line, were pulsed with 0.02 μg/mL CMV pp65 peptide at 37° C. for 1 hour and washed. The PBMCs were then co-cultured with CMV pp65 peptide pulsed HCT116 cells in the presence of 5 μg/ml PVRIG antibody alone or in combination with 5 μg/ml of the anti-PD1 antibody BGB-A317 and/or the anti-TIGIT antibody BGB-A1217 overnight. Human IFN-γ was measured by ELISA.

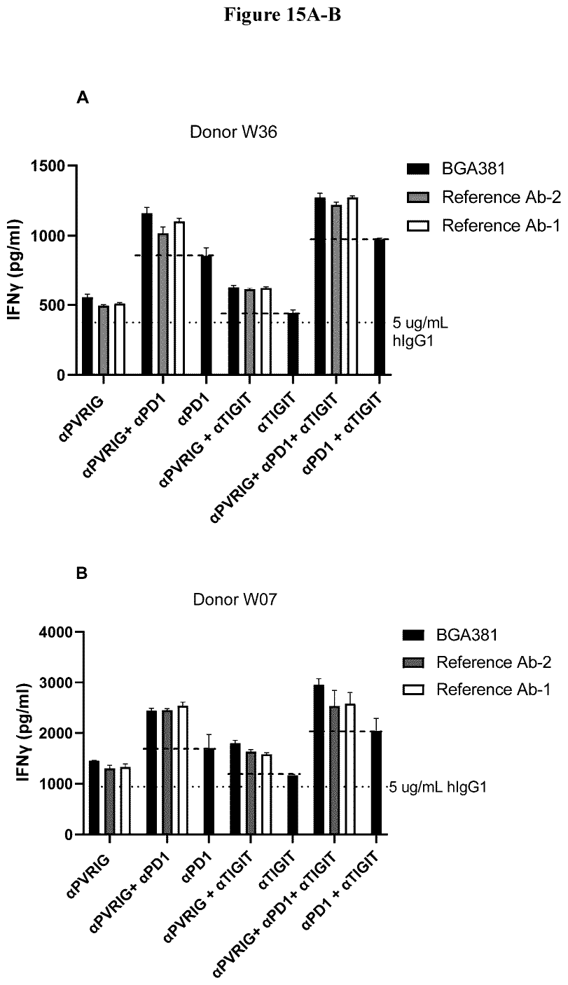

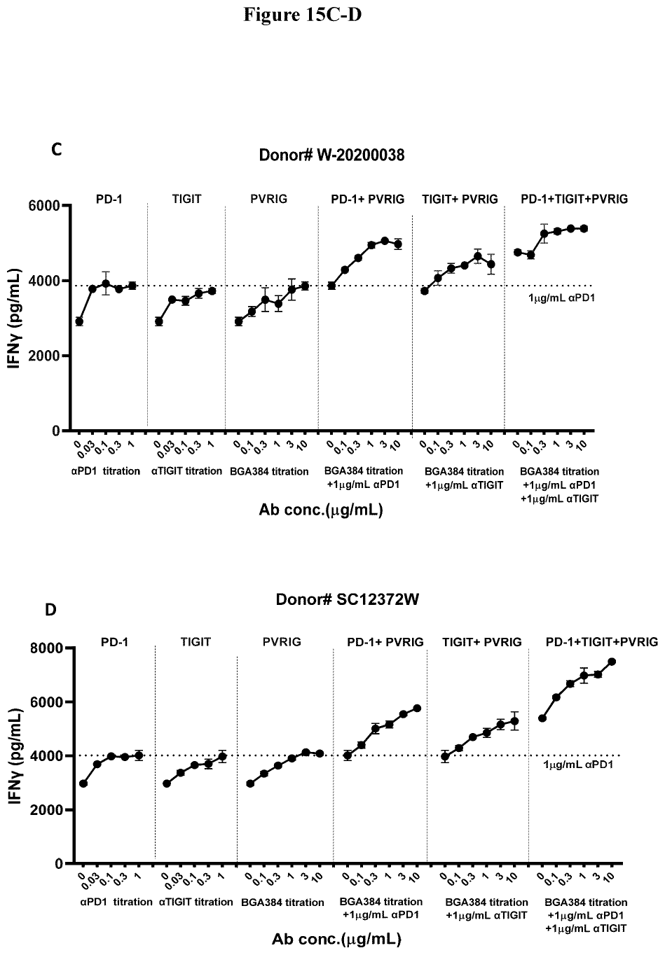

Results: As shown in FIGS. 15A and B, combination of BGA381 with anti-PD-1 antibody BGB-A317 and/or anti-TIGIT antibody BGB-1217 significantly enhanced IFN-γ release compared to BGA381 or BGB-A317 treatment alone, indicating that the combined blockade of PVRIG. TIGIT and PD-1 can mitigate effector cell exhaustion following activation.

HLA-A*0201 PBMCs were stimulated by 1 μg/mL CMV pp65 peptide NLVPMVATV (SEQ ID NO: 185), 2 ng/ml IL-2 and 10 ng/mL IL-7 for 8 days, with IL-2 and IL-7 added on day 4 and day 6. The cells were then rested in media with 500 IU/mL IL-2 for 3 days. HCT116 cells, (ATCC CCL-247), a human colorectal carcinoma cell line, were pre-treated with 0.1 ng/mL IFN-γ for 67 hours to upregulate PD-L1 expression, then pulsed with 0.1 μg/mL CMV pp65 peptide at 37° C. for 1 hour and washed. The PBMCs were then co-cultured with CMV pp65 peptide pulsed HCT116 cells in the presence of PD1. TIGIT or PVRIG antibody as single agent at indicated concentration or in combination with 1 μg/ml of the anti-PD1 antibody BGB-A317 and/or 1 μg/ml of the anti-TIGIT antibody BGB-A1217 overnight. Human IFN-γ was measured by HTRF (Homogeneous Time Resolved Fluorescence).

As shown in FIGS. 15C and 15D. BGA384 exhibited significant dose-dependent single agent activity in CMV antigen specific memory recall assay indicated by increased human IFN-γ release, combo effect was well-demonstrated by dual combination with anti-PD-1 antibody (BGB-A317) or anti-TIGIT antibody (BGB-A1217) and triple combination with anti-PVRIG antibody, BGB-A317 and BGB-A1217, indicating the high potential for co-blockade of PVRIG. TIGIT and PD-1 to maximize T cell immune response and enhance anti-tumor activity.

BGB-A317 (Tislelizumab) is disclosed in U.S. Pat. No. 8,735,553.

TABLE 20
Tislelizumab

| | SEQ | |
|---|---|---|
| | ID | |
| Domain | NO: | Amino Acid Sequence |
| HCDR1 | SEQ | GFSLTSYGVH |
| | ID | |
| | NO: | |
| | 163 | |
| HCDR1 | SEQ | VIYADGSTNYNPSLKS |
| | ID | |
| | NO: | |
| | 164 | |
| HCDR3 | SEQ | ARAYGNYWYIDV |
| | ID | |
| | NO: | |
| | 165 | |
| LCDR1 | SEQ | KSSESVSNDVA |
| | ID | |
| | NO: | |
| | 166 | |
| LCDR2 | SEQ | YAFHRFT |
| | ID | |
| | NO: | |
| | 167 | |
| LCDR3 | SEQ | HQAYSSPYT |
| | ID | |
| | NO: | |
| | 168 | |
| VH | SEQ | QVQLQESGPGLVKPSETLSLTCTVSGFSLTSYG |
| | ID | VHWIRQPPGKGLEWIGVIYADGSTNYNPSLKSR |
| | NO: | VTISKDTSKNQVSLKLSSVTAADTAVYYCARAY |
| | 169 | GNYWYIDVWGQGTTVTVSS |
| VL | SEQ | DIVMTQSPDSLAVSLGERATINCKSSESVSNDV |
| | ID | AWYQQKPGQPPKLLINYAFHRFTGVPDRFSGSG |
| | NO: | YGTDFTLTISSLQAEDVAVYYCHQAYSSPYTFG |
| | 170 | QGTKLEIK |
| IgG4 | SEQ | ASTKGPSVFPLAPCSRSTSESTAALGCLVKDYF |
| constant | ID | PEPVTVSWNSGALTSGVHTFPAVLQSSGLYSLS |
| domain | NO: | SVVTVPSSSLGTKTYTCNVDHKPSNTKVDKRVE |
| | 171 | SKYGPPCPPCPAPPVAGGPSVFLFPPKPKDTLM |
| | | ISRTPEVTCVVVAVSQEDPEVQFNWYVDGVEVH |
| | | NAKTKPREEQFNSTYRVVSVLTVVHQDWLNGKE |
| | | YKCKVSNKGLPSSIEKTISKAKGQPREPQVYTL |
| | | PPSQEEMTKNQVSLTCLVKGFYPSDIAVEWESN |
| | | GQPENNYKTTPPVLDSDGSFFLYSKLTVDKSRW |
| | | QEGNVFSCSVMHEALHNHYTQKSLSLSLGK |
| Heavy | SEQ | QVQLQESGPGLVKPSETLSLTCTVSGFSLTSYG |
| Chain | ID | VHWIRQPPGKGLEWIGVIYADGSTNYNPSLKSR |
| | NO: | VTISKDTSKNQVSLKLSSVTAADTAVYYCARAY |
| | 172 | GNYWYIDVWGQGTTVTVSSASTKGPSVFPLAPC |
| | | SRSTSESTAALGCLVKDYFPEPVTVSWNSGALT |
| | | SGVHTFPAVLQSSGLYSLSSVVTVPSSSLGTKT |
| | | YTCNVDHKPSNTKVDKRVESKYGPPCPPCPAPP |
| | | VAGGPSVFLFPPKPKDTLMISRTPEVTCVVVAV |
| | | SQEDPEVQFNWYVDGVEVHNAKTKPREEQFNST |
| | | YRVVSVLTVVHQDWLNGKEYKCKVSNKGLPSSI |
| | | EKTISKAKGQPREPQVYTLPPSQEEMTKNQVSL |
| | | TCLVKGFYPSDIAVEWESNGQPENNYKTTPPVL |
| | | DSDGSFFLYSKLTVDKSRWQEGNVFSCSVMHEA |
| | | LHNHYTQKSLSLSLGK |
| Light | SEQ | DIVMTQSPDSLAVSLGERATINCKSSESVSNDV |
| chain | ID | AWYQQKPGQPPKLLINYAFHRFTGVPDRFSGSG |
| | NO: | YGTDFTLTISSLQAEDVAVYYCHQAYSSPYTFG |
| | 173 | QGTKLEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |

BGB-A1217 (Ociperlimab) is disclosed in WO2019/129261.

TABLE 21
Ociperlimab

| SEQ | | |
|---|---|---|
| ID | | |
| NO: | Domain | Sequence |
| HCDR1 | SEQ | DYYMY |
| | ID | |
| | NO: | |
| | 174 | |
| HCDR2 | SEQ | YITKGGGSTYYPDSVKG |
| | ID | |
| | NO: | |
| | 175 | |
| HCDR3 | SEQ | QTNYDFTMDY |
| | ID | |
| | NO: | |
| | 176 | |
| LCDR1 | SEQ | KASQDVGTSVA |
| | ID | |
| | NO: | |
| | 177 | |
| LCDR2 | SEQ | WASARHT |
| | ID | |
| | NO: | |
| | 178 | |
| LCDR3 | SEQ | QQYSSYPLT |
| | ID | |
| | NO: | |
| | 179 | |
| VH | SEQ | EVQLVESGGGLVQPGGSLRLSCAASGFTFSDYY |
| | ID | MYWVRQAPGKGLEWVAYITKGGGSTYYPDSVKG |
| | NO: | RFTISRDNAKNTLYLQMNSLRAEDTAVYYCARQ |
| | 180 | TNYDFTMDYWGQGTLVTVSS |
| VL | SEQ | EIVMTQSPATLSVSPGERATLSCKASQDVGTSV |
| | ID | AWYQQKPGQAPRLLIYWASARHTGIPARFSGSG |
| | NO: | SGTEFTLTISSLQSEDFAVYYCQQYSSYPLTFG |
| | 181 | GGTKVEIK |
| Heavy | SEQ | EVQLVESGGGLVQPGGSLRLSCAASGFTFSDYY |
| chain | ID | MYWVRQAPGKGLEWVAYITKGGGSTYYPDSVKG |
| | NO: | RFTISRDNAKNTLYLQMNSLRAEDTAVYYCARQ |
| | 182 | TNYDFTMDYWGQGTLVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPELLGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | PAPIEKTISKAKGQPREPQVYTLPPSREEMTKN |
| | | QVSLTCLVKGFYPSDIAVEWESNGQPENNYKTT |
| | | PPVLDSDGSFFLYSKLTVDKSRWQQGNVFSCSV |
| | | MHEALHNHYTQKSLSLSPGK |
| light | SEQ | EIVMTQSPATLSVSPGERATLSCKASQDVGTSV |
| chain | ID | AWYQQKPGQAPRLLIYWASARHTGIPARFSGSG |
| | NO: | SGTEFTLTISSLQSEDFAVYYCQQYSSYPLTFG |
| | 183 | GGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| IgG1 | SEQ | ASTKGPSVFPLAPSSKSTSGGTAALGCLVKDYF |
| WT | ID | PEPVTVSWNSGALTSGVHTFPAVLQSSGLYSLS |
| | NO: | SVVTVPSSSLGTQTYICNVNHKPSNTKVDKKVE |
| | 184 | PKSCDKTHTCPPCPAPELLGGPSVFLFPPKPKD |
| | | TLMISRTPEVTCVVVDVSHEDPEVKFNWYVDGV |
| | | EVHNAKTKPREEQYNSTYRVVSVLTVLHQDWLN |
| | | GKEYKCKVSNKALPAPIEKTISKAKGQPREPQV |
| | | YTLPPSREEMTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPGK |

### Example 16. Human NK Cell Activation by Anti-PVRIG Antibodies

PVRIG is known to be constitutively expressed on natural killer (NK) cells at relatively higher levels and the interaction between PVRIG and its ligands inhibits NK cell mediated cytotoxicity. To confirm whether PVRIG antibodies could promote NK cell-mediated cytotoxicity. NK activation assay was determined by co-culturing human primary NK cells with PVRL2-expressing SK-BR-3 cells. Briefly, primary NK cells were isolated by negative selection using a human NK cell isolation kit (Mitenyi Biotech, #130-092-657) and stimulated in complete medium with 25 U/ml IL-2 overnight. Pre- stimulated NK cells (5×10⁴ cells/well) and SK-BR-3 cells (5×10⁴ cells/well) were co-cultured in a 96 well plate for 5 hours. Lysosomal membrane protein (CD107a) expression on NK cells was measured by flow cytometry. CD107a is significantly upregulated on the surface of NK cells following stimulation and CD107a expression correlates with both cytokine secretion and NK cell-mediated lysis of target cells.

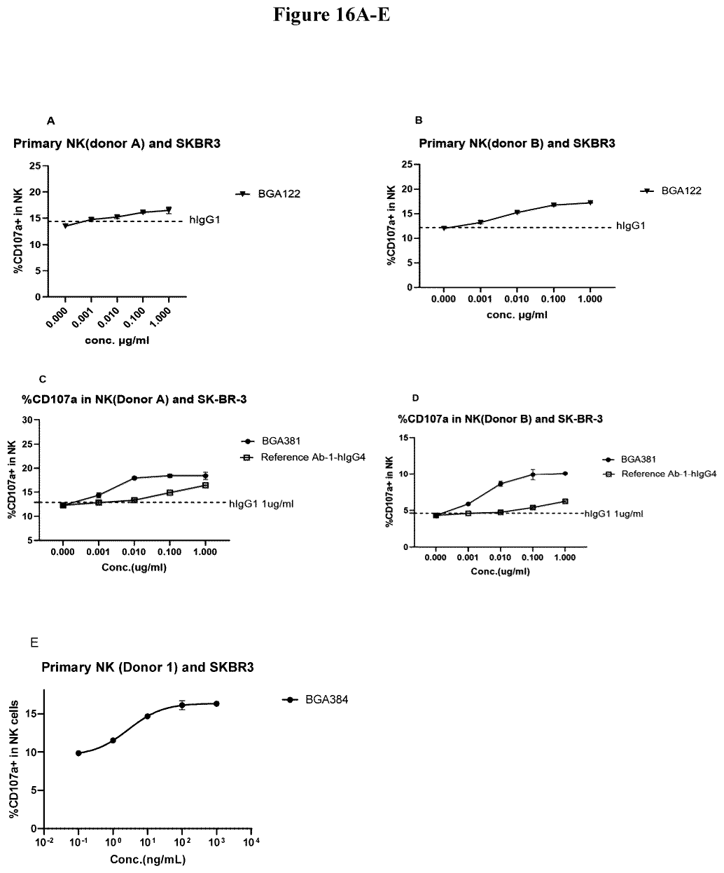

Results: As shown in FIGS. 16A-E, the anti-PVRIG antibodies BGA122, BGA381 and BGA384 significantly enhanced NK activation in a dose dependent manner, shown by the % increase in CD107a expression, as measured by flow cytometry.

### Example 17. Trogocytosis Assay

Trogocytosis is a phenomenon, in which cell surface molecules are transferred from donor cells to acceptor cells (Joly and Hudrisier, 2003 Nat Immunol 4 (9): 815; Beum et al., 2008 J Immunol 181 (11): 8120-8132; Machlenkin et al., 2008 Cancer Res 68 (6): 2006-2013; Rossi et al., 2013 Blood 122 (17): 3020-3029). Antibody-induced trogocytosis via Fey receptors (FcγRs) leads to down-modulation of receptors on the cell surface (Beum, et al., 2008 supra; Beum et al., 2011 supra). Therefore, down-regulation of target receptor by trogocytosis can result in dampened signaling. To address this phenomenon. Jurkat/NFAT-reporter/hPVRIG cells were incubated with CF633 (biotin) labeled BGA381 (10 μg/ml) and washed. Then Jurkat/NFAT-reporter/hPVRIG cells (2×10⁴ cells/well) were co-cultured with biotin labeled HEK cells expressing various FcγRs (including FcγRILAHu1, FcγRIIB and FcγRIIIA) overnight. Change of PVRIG Mean Fluorescent Intensity (MFI) was measured by flow cytometry.

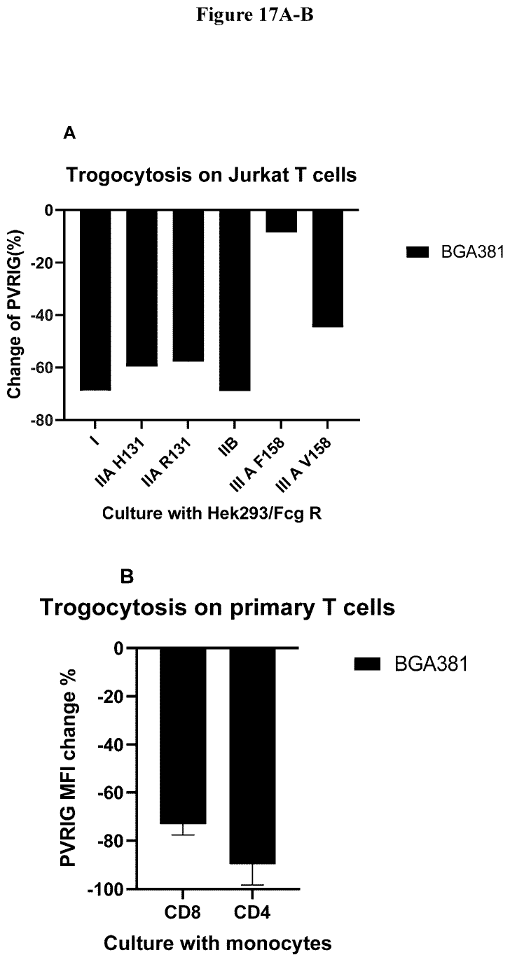

Mutant IgG1 Fc region and WT control antibodies comprising the same VL and VH sequences as BGA381 were compared. As shown in FIG. 17A. BGA381/IgG1 WT but not BGA381/IgG1 mutant caused a significant reduction of PVRIG surface expression compared to the negative control human IgG-treated cells, indicating that the reduction of surface PVRIG on Jurkat/PVRIG cells is FcγR-binding dependent.

In the primary cell-based experiment. T cells (4×10⁴ cells/well) were used as donor cells. Monocytes (8×10⁴ cells/well) isolated from the same donors were used as acceptor cells. T cells and monocytes were purified from human PBMCs using the Pan T cell isolation kit (Miltenyi Biotec, catalog no. 130-096-535) or the monocyte isolation kit (Miltenyi Biotec, catalog no. 130-096-537) respectively. Donor cells were pre-incubated with 10 μg/mL CF633-labeled BGA381 or CF633 biotin-labeled BGA381 mutant Fc for 30 min and washed. The donor cells were then incubated with acceptor cells in 96-well plates overnight. Change of PVRIG MFI was measured by flow cytometry. Results: As shown in FIGS. 17A and 17B. BGA381 significantly induced trogocytosis, removing PVRIG from T cells.

### Example 18. X-Ray Crystal Structure of BGA381 Fab Fragment

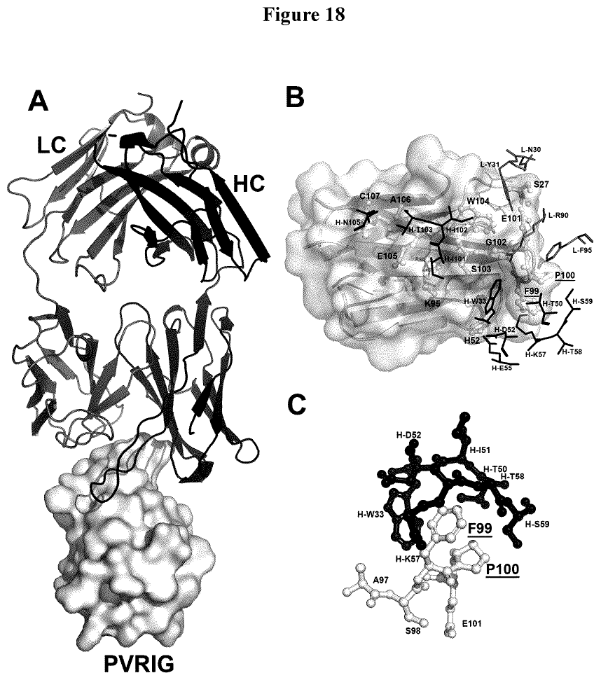

The X-ray crystal structure of the BGA381 Fab fragment was determined at 1.61 angstrom resolution (PDB: 8JBJ). From this structure, it is apparent that Asp55 of BGA381 is located in a loosely ordered structure (FIG. 18A). Panel (A) is the crystal structure of A538-EH Fab bound to human PVRIG, with A538-EH heavy chain (HC) and light chain (LC) regions colored in black and gray cartoon while PVIRG in white surface. Panel (B) shows the atomic interactions on the binding surface of A538-EH/PVRIG complex, identifying certain key residues of A538-EH (paratope residues shown in line) and PVRIG (epitope residues shown in stick). For clarify, non-CDR region of A538-EH are removed with PVRIG colored in white transparent surface. The underlined residues were identified as functionally important epitopic residues by alanine scanning mutagenesis. Panel (C) shows the surrounding residues of F99 PVRIG and P100 PVRIG within A538-EH is shown to emphasize the interaction among these residues which is critical for PVRIG binding. F99PVRIG and P100PVRIG are highlighted with underlined larger text.

Based on the crystal structure of the A538-EH/PVRIG complex, the residues of PVRIG that are contacted by A538-EH (i.e., the epitopic residues of PVRIG bound by A538-EH) and the residues of A538-EH that are contacted by PVRIG (i.e., the paratopic residues of A538-EH contacted by PVRIG) were determined. Table 21.A and Table 21.B below, show the residues of PVRIG and the heavy or light chain residues of A538-EH to which they contact, as assessed using a contact distance stringency of 3.7 Å, a point at which van der Waals (non-polar) interaction forces are highest.

TABLE 21.A
Epitopic residues of PVRIG and their corresponding
paratopic residues on the heavy chain of A538-EH

| | | A538-EH | |
|---|---|---|---|
| PVRIG | | Heavy Chain | |
| His | 52 | Glu | 55 |
| Lys | 95 | Ile | 101 |
| Phe | 99 | Trp | 33 |
| | | Thr | 50 |
| | | Asp | 52 |
| | | Lys | 57 |
| | | Thr | 58 |
| Pro | 100 | Trp | 33 |
| | | Ser | 59 |
| Gly | 102 | Trp | 33 |
| Ser | 103 | Trp | 33 |
| | | Ile | 101 |
| | | Ile | 102 |
| Trp | 104 | Ile | 102 |
| Glu | 105 | Ile | 102 |
| | | Thr | 103 |
| Ala | 106 | Asn | 105 |
| Cys | 107 | Asn | 105 |

TABLE 21.B
Epitopic residues of PVRIG and their corresponding
paratopic residues on the light chain of A538-EH

| | | A538-EH | |
|---|---|---|---|
| PVRIG | | Light Chain | |
| Ser | 27 | Asn | 30 |
| Pro | 100 | Arg | 90 |
| | | Phe | 95 |
| Glu | 101 | Tyr | 31 |
| | | Arg | 90 |

### Example 19. Binding Activity of BGA384

### Target-Binding Activity and Specificity of BGA384

BGA384 was examined for its binding activities by cell-free and cell-based assays. Results from these experiments demonstrate, amongst other things, that BGA384 binds to its target protein, human PVRIG, with high specificity and affinity as described in the following sections.

### Binding to Recombinant and Native PVRIG

Target-binding activity of BGA384 was examined by enzyme-linked immunosorbent assay (ELISA).

Materials and Methods: Each ELISA well was coated with 0.5 μg/mL hPVRIG-mIgG2a, incubated with BGA384 and negative control, HuIgG, and Placebo. EC50 of BGA384 was 8.79 ng/mL. As negative controls, human IgG and placebo had no detectable binding to the hPVRIG-mIgG2a.

For the BGA384 binding analysis by FACS, live HuPVRIG-overexpressed Jurkat cells were seeded in 96-well plates and incubated with BGA384 or negative controls (HuIgG or DS buffer). Fluorescence-labeled anti-human IgG (H+L) was used to detect antibody binding activity to the cell surface. EC50 of BGA384 was 26.9 ng/mL; Human IgG (HuIgG) and DS buffer were completely inactive.

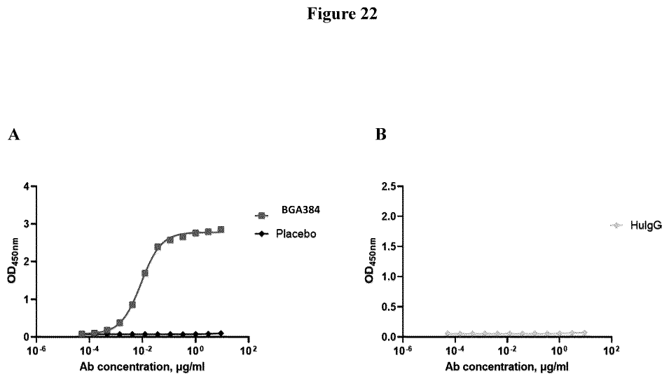

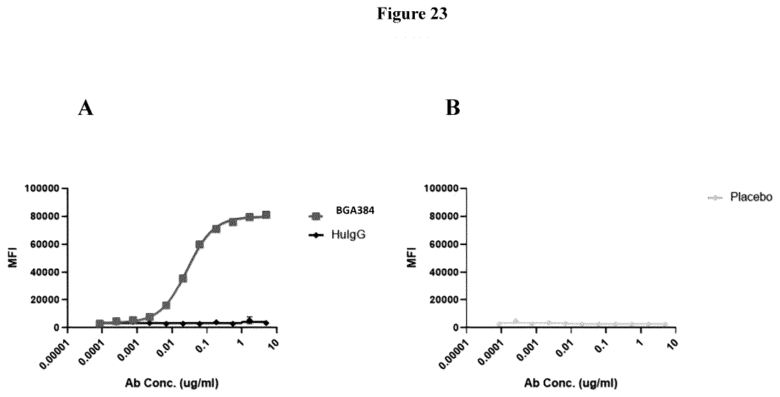

Results: As shown in FIG. 22A, the ELISA results demonstrated significant binding of BGA384 to plates coated with recombinant human antigen PVRIG. The half maximal effective concentration (EC50) of BGA384 was 8.79 ng/mL. Placebo and human immunoglobulin G (HuIgG) served as negative controls and showed no detectable binding activity to recombinant PVRIG (FIG. 22B). In addition, fluorescence-activated cell sorting (FACS) confirmed the binding activity of BGA384 to native huPVRIG overexpressed on the Jurkat cell surface. BGA384 showed strong binding activity to huPVRIG-overexpressed Jurkat cells in dose-dependent manners with an EC50 of 26.9 ng/ml (FIG. 23A), whereas the negative control of placebo and HuIgG did not bind to huPVRIG-overexpressed Jurkat cells (FIG. 23B).

### Binding Specificity

To test for non-specific binding activity of BGA384, FACS was performed to check binding behaviors of BGA384 to Expi293 cells. Expi293 cells are known to be negative for expression of human PVRIG.

Materials and Methods: Live Expi293 cells were seeded in 96-well plates and incubated with BGA384 or negative controls (HuIgG or DS buffer). Fluorescence-labeled anti-human IgG (H+L) was used to detect antibody binding activity to the cell surface. The results showed no binding activity of two batches of BGA384 and the negative control of HuIgG/DS buffer to Expi293 cells.

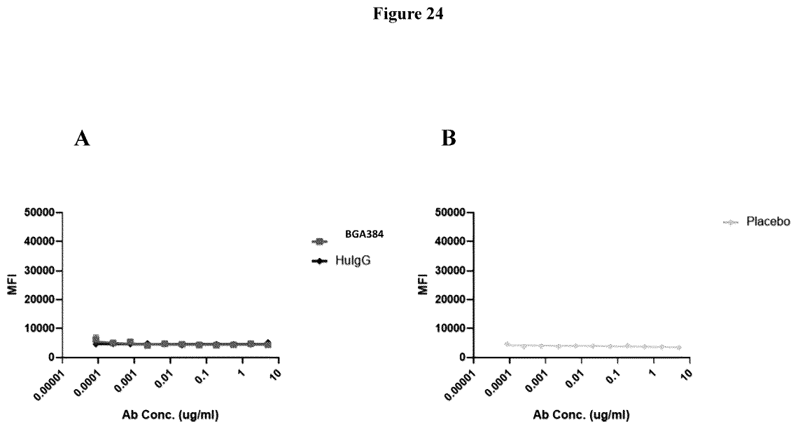

Results: BGA384 and the negative control of placebo/HuIgG showed no binding activity to Expi293 cells, indicating BGA384 binding specificity for PVRIG (FIGS. 24A-B).

### Binding Specificity to Human PVRIG

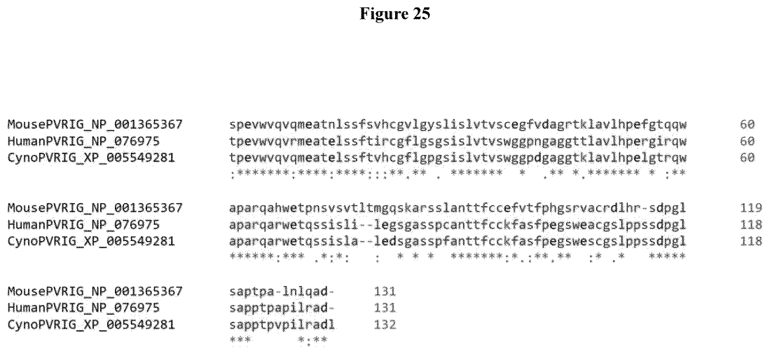

Human PVRIG protein differs from cynomolgus monkey and mouse PVRIG in amino acid sequence of the extracellular domain by 9.9% and 36.8%, respectively (FIG. 25 and Table 22; sources are Human PVRIG, NCBI accession No: NP_076975; Cynomolgus PVRIG, NCBI accession No: XP_005549281; Mouse PVRIG, NP_001365367).

TABLE 22
Homology percentage of Human, Cynomolgus Monkey,
and Murine PVRIG Extracellular Domains

| | Human | Cynomolgus | Mouse |
|---|---|---|---|
| | PVRIG | PVRIG | PVRIG |
| Human PVRIG | 100% | 90.1% | 63.2% |
| Cynomolgus PVRIG | — | 100% | 65.4% |
| Mouse PVRIG | — | — | 100% |

To test species cross-reactivity of BGA384, a Biacore-based SPR assay was performed using human, cynomolgus monkey, or mouse PVRIG-His as test antigens.

Materials and Methods: Binding kinetic sensorgrams of BGA384 to PVRIG were performed. BGA384 was flown over the chip and captured by immobilized anti-human Fc-binding antibody. A series of dilutions of PVRIG of specific species (human/monkey/mouse) were injected, and binding responses to PVRIG were calculated by subtracting response unit (RU) from a reference flow-cell without injection of BGA384. SPR k_{off} and kₒₙ were calculated using the one-to-one Langmuir binding model (BIA Evaluation Software, GE Life Sciences); K_{D} was calculated as the ratio of k_{off}/kₒₙ. BGA384 had no detectable binding signaling to mouse PVRIG in SPR assay. Without being bound by theory, koff represents the time in seconds for the receptor-ligand complex to dissociate to free ligand and free receptor, which translates into a measure of the residence time of a biologic (e.g., an antibody or antigen-binding fragment thereof described herein) in the body with longer residence times being more efficacious (Copeland et al., Nat Rev Drug Discov 5, 730-739, 2006).

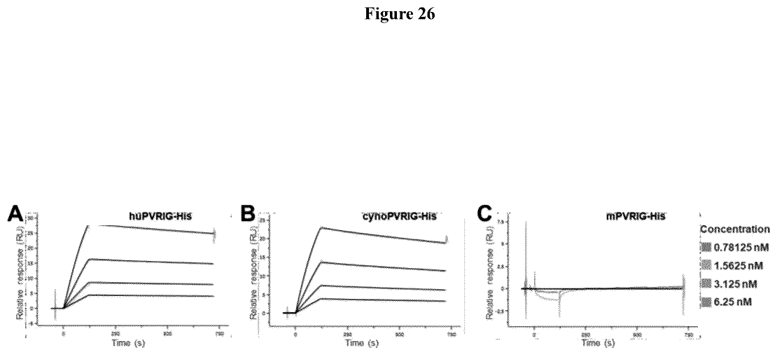

Results: FIGS. 26A-C show sensorgrams of BGA384 binding to human PVRIG (FIG. 26A), cynomolgus PVRIG (FIG. 26B) and mouse PVRIG (FIG. 26C). BGA384 displayed a high binding affinity to human PVRIG with a K_{D} of about 0.0826 nM (Table 23). In comparison, the binding affinity of BGA384 to cynomolgus PVRIG exhibited a similar K_{D} of about 0.228 nM (Table 23). However, BGA384 did not exhibit binding to mouse PVRIG (Table 23).

TABLE 23
Comparative analysis of SPR determined kinetic parameters and affinities
of BGA384 full antibody to different species of PVRIG

| Antigen | Antibody | kₒₙ (1/Ms) | k_{off} (1/s) | K_{D} (M) |
|---|---|---|---|---|
| Human PVRIG | BGA384/Batch | 4.16E+06 | 3.44E−04 | 8.26E−11 |
| Cynomolgus | #A538-230111- | 1.77E+06 | 4.03E−04 | 2.28E−10 |
| PVRIG | BDS- C1 | | | |
| Mouse PVRIG | | ND | ND | ND |

### Critical Binding Epitopes

As a receptor, PVRIG binds to PVRL2 (also known as CD112 or Nectin-2). Blockade of the PVRIG-PVRL2 signaling pathway increases T-cell proliferation, cytokine secretion, and cytotoxicity. BGA384 has been shown to competitively block PVRL2 binding to PVRIG. The crystal structure of human PVRIG in complex with BGA384 Fab has been solved at atomic resolution. From this data, it was suggested that there are 17 residues (E3, G26, S27, S31, L32, H52, K95, A97, F99, P100, E101, G102, S103, W104, E105, A106, C107) of PVRIG directly involved in the epitope-paratope interaction (calculated by the PISA webserver: https://www.ebi.ac.uk/pdbe/pisa/). Based on this structural information, ELISA experiments were performed to further validate whether these amino acids are required or are critical for PVRIG binding by BGA384.

Materials and Methods: The wild type (wt) and mutant PVRIG-Fc were transiently expressed in 293G cells and purified using a Mab Select Sure resin affinity column. Then, these recombinant proteins were analyzed in ELISA-binding assay with BGA384. Relative ELISA readings were derived by normalization to the binding signal of the same antibody to wt PVRIG. All ELISA results were normalized using the mean ELISA readings of wt PVRIG binding signals as the standard.

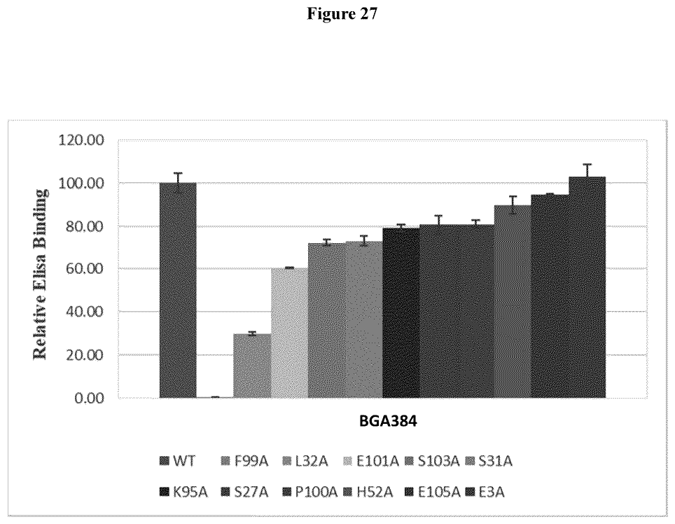

Results: Among these residues in PVRIG, 11 point mutants were successfully prepared individually. The PVRIG mutant proteins along with the wt PVRIG proteins were analyzed for their recognition and binding by BGA384. In the ELISA binding assay using native wt or mutant PVRIG proteins, BGA384 had nearly no binding to F99A single mutant, suggesting that F99A is a critical epitope for BGA384 binding (FIG. 27 and Table 24).

TABLE 24

| Wild type or | |
|---|---|
| mutant PVRIG | BGA384 |
| WT | — |
| F99A | *** |
| L32A | ** |
| E101A | * |
| S103A | * |
| S31A | * |
| K95A | — |
| S27A | — |
| P100A | — |
| H52A | — |
| E105A | — |
| E3A | — |

### Functional Activities of BGA384 in Cell-Based Assays

### BGA384 Competitively Blocks PVRIG Binding to PVRL2

A protein-based ELISA analysis was performed to assess the functional activity of BGA384. Biotinylated human PVRL2 (Fc-Avi-tag) was incubated with a plate coated with human PVRIG in the presence of BGA384, and the ligand bound to human PVRIG was detected by streptavidin-HRP.

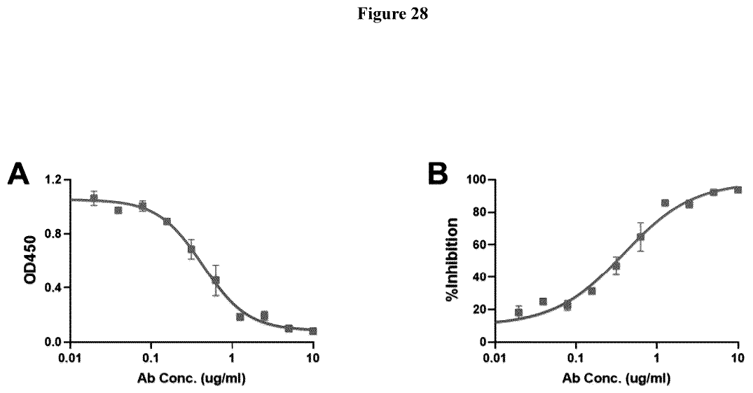

Results: The ligand-binding signal was inversely correlated to BGA384 antibody concentration in this blocking assay, indicating that BGA384 out competes ligand-binding in a dose-dependent manner (FIG. 28A). Inhibition of ligand-binding by BGA384 reached 94% with an IC50 of 0.383 μg/mL (FIG. 28B).

### BGA384 Showed Efficient Blockade Activity Against PVRIG-PVRL2 Binding in a Cell-Based Assay

In addition, to accurately assess the blockade efficiency of BGA384 to PVRIG-PVRL2 binding at a cellular level, a cell-based blockade assay was established, in which the target PVRIG existed as a free fusion protein (human PVRIG-mIgG2a, referred to as “hPVRIG” hereafter) and its ligand PVRL2 was highly overexpressed on Farage cells.

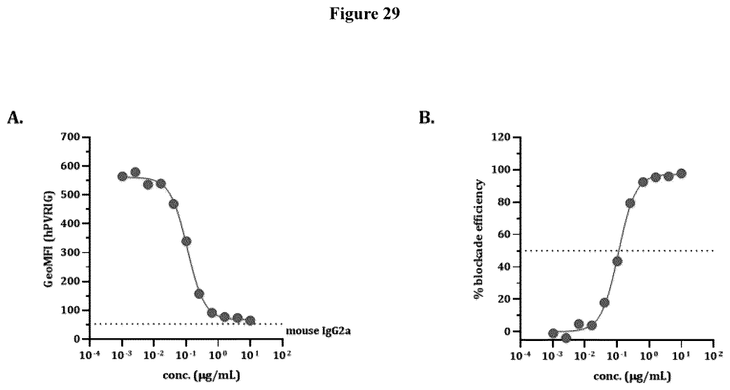

Materials and Methods: The competitive blocking of BGA384 against the PVRIG-PVRL2 interaction was measured by detecting human PVRIG binding to surface PVRL2 on Farage-hPVRL2 cells with the secondary antibody, anti-mIgG2a Fc Ab. Biotinylated human PVRL2 (Fc-Avi-tag) was incubated with a plate coated with human PVRIG in the presence of BGA384. Farage-hPVRL2 cells were blocked with human Fc blocker, and then incubated with the indicated concentration of BGA384 and 300 ng/ml of hPVRIG-mIgG2a for 30 minutes on ice. The competition capacity of BGA384 against PVRIG-PVRL2 binding was determined by ELISA using an absorption value of 450 nm (OD450) as readout. The bound human PVRIG was detected with PE anti-mouse IgG2a antibody by flow cytometry. BGA384 demonstrated blocking function against PVRIG-PVRL2 interaction in a dose-dependent manner (FIG. 29A). Blockade efficiency of BGA384 was calculated, with an IC50 of 109.4 ng/ml (FIG. 29B).

Results: These data provide evidence that BGA384 efficiently blocked PVRIG binding to PVRL2 expressed on the cell surface and in a concentration-dependent manner with an IC50 of 109.4 ng/ml.

### BGA384 Promoted T Cell Activation in PVRIG+ Jurkat T Cells

To determine the function of BGA384 on T cells, Jurkat cells were engineered to express human PVRIG and a reporter-luciferase with a nuclear factor of activated T cells (NFAT) response element, referred to as Jurkat-NFAT-HuPVRIG (JK-NFAT-HuPVRIG). In this assay. JK-NFAT-HuPVRIG cells were further co-cultured with the T-cell engager OS8 (a membrane-bound form of anti-CD3 antibody)-transfected A549 cell line (referred to as A549/OS8-H) in the presence of serially diluted BGA384 or human IgG as the control. After 5 to 6 hours of incubation. Bio-Lite luciferase assay reagent was added into the plate and relative light units (RLUs) were recorded with a microplate reader.

Materials and Methods: JK-NFAT-HuPVRIG (5×104 cells/well) and A549/OS8-H (1×104 cells/well) were co-cultured for 5-6 hours in the presence of serial diluent BGA384. Placebo, and HuIgG. EC50 of BGA384 was 37.74 ng/ml. As negative controls, placebo and HuIgG had no functional activity.

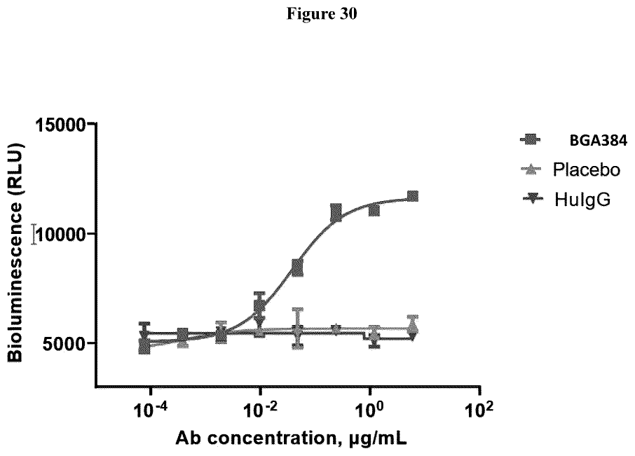

Results: BGA384 blocked inhibitory signals to enhance T-cell activation as demonstrated by increased promoter-driven luminescence in a dose-responsive manner (FIG. 30). The EC50 for BGA384 was 37.74 ng/mL. Placebo and human IgG exhibited no activity.

### BGA384 Promoted Antigen-Specific T Cell Activation in CMV Memory Recall Assay

The functional activity of BGA384 was assessed using naturally derived T cells that recognize human cytomegalovirus (CMV) PP65 peptide (NLVPMVATV, 495-503. HLA-A2.1-restricted; see Boeckh and Geballe et al. 2011. Whelan et al. 2019). In the co-culture system. CMV-specific T cells generated from healthy donors were used as responder cells, whereas CMV antigen pp65-pulsed HCT116/PD-L1 cells (positive for the PVRIG ligand PVRL2) were used as “stimulator” cells. IFN-γ secretion was used as the readout for T-cell activation and biomarker for inhibition of the PVRIG mediated signaling pathway.

Materials and Methods: PP65 peptide-stimulated PBMCs (from HLA-A2.1+ healthy donors) were co-cultured with PP65-pulsed HCT116/PD-L1 cells in the presence of BGA384 or negative control HuIgG overnight. IFN-γ in culture supernatants was determined by HTRF.

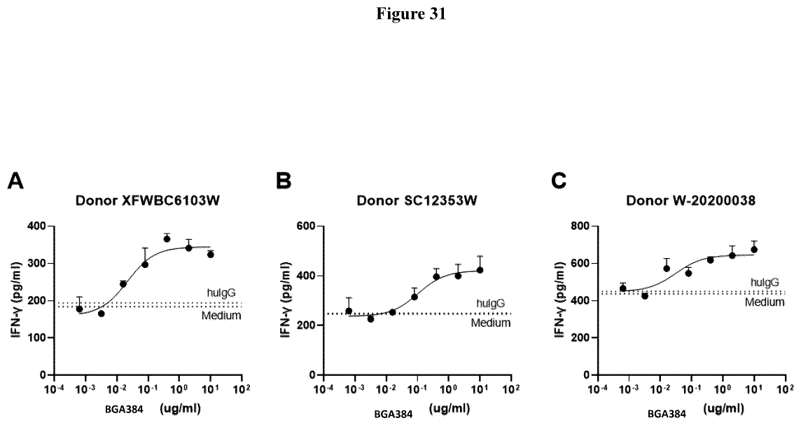

Results: BGA384 enhanced the ability of CMV-specific T cells to produce IFN-γ in a concentration dependent manner (FIGS. 31A-C).

BGA384 Alone or in Combination with BGB-A317 and/or BGB-A1217 Enhanced IFN-γ Secretion by CMV-Specific T Cells

Functional activity of BGA384 alone or in combination with BGB-A317 (an anti-PD-1 antibody) and/or BGB-A1217 (an anti-TIGIT antibody) was assessed using naturally derived T cells that recognize human CMV PP65 peptide (NLVPMVATV, 495-503, HLA-A2.1-restricted). In the co-culture system. CMV-specific T cells generated from healthy donors were used as responder cells, whereas CMV antigen pp65-pulsed HCT116/PD-L1 cells (positive for the PVRIG ligand PVRL2. TIGIT ligands PVR and PVRL2, and PD-1 ligand PD-L1) were used as “stimulator” cells. IFN-γ secretion was used as the readout for T-cell activation and biomarker for inhibition of the PVRIG. TIGIT and PD-1 mediated signaling pathway.

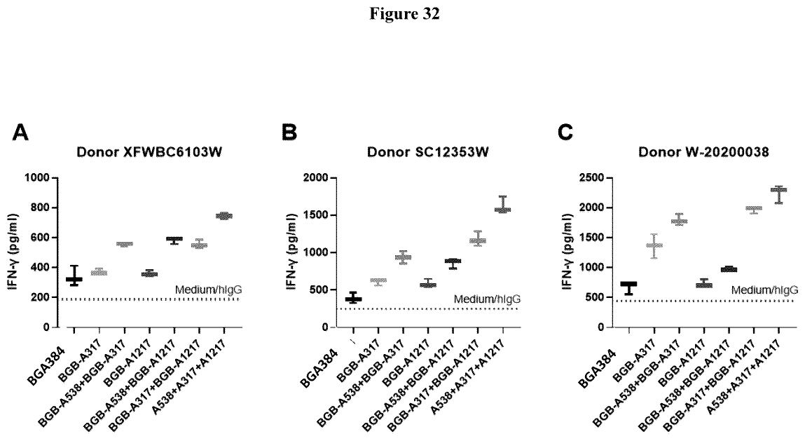

Materials and Methods: PP65 peptide-stimulated PBMCs (from HLA-A2.1+ healthy donors) were co-cultured with PP65-pulsed HCT116/PD-L1 cells in the presence of 5 μg/ml of BGA384 alone or in combination with 5 μg/ml of BGB-A317. BGB-A1217, or negative control HuIgG alone overnight. IFN-γ in the culture supernatants was determined by HTRF. The results of 3 independent experiments are presented in FIGS. 32A to C.

BGA384 alone enhanced the productivity of CMV-specific T cells in IFN-γ production; moreover. BGA384 in combination with BGB-A317 or BGB-A1217 was more efficacious than BGA384. BGB-A317, or BGB-A1217 alone (FIGS. 32A-C). Moreover, BGA384 in combination with BGB-A317 and BGB-A1217 was more efficacious than the dual combinations. Thus, synergistic T cell activation with increased IFN-γ production was observed with triple blockade of PVRIG, TIGIT, and PD-1 in preclinical studies. Without wishing to be bound by theory, such synergistic activity can support blockade of PVRIG in combination with one or both of TIGIT or PD-1 to enhance patient response to treatment.

### Effector Receptor Binding and Effector Functions by BGA384

### BGA384 has Competent Binding Activity to Human FcγRs

Biacore-based assays were performed to assess the binding activity of BGA384 to FcγRs.

Materials and Methods: The placebo (negative control) contained no antibody, but had the same formulation as BGA384. HuIgG (Sigma. Catalog #14506) was a mixture of human IgGs. K¹ (M) values were calculated from the ratio of the kinetic constants as K_{D}=k_{off}/kₒₙ. K2 (M) values were determined by the analyte concentration at which half of the ligands are occupied at equilibrium.

Results: BGA384 had competent binding activity to the human FcγRs and their polymorphic variants similar to human IgG mixture. The placebo had no detectable binding to all FcγRs (Table 25).

TABLE 25

| | | FcγRIIA K_{D}² (M) | | | FcγRIIIA K_{D}² (M) | |
|---|---|---|---|---|---|---|
| Sample | FcγRI K_{D}¹ (M) | H131 | R131 | FcγRIIB K_{D}² (M) | F158 | V158 |
| BGB-A538 | 3.26E−09 | 1.48E−06 | 1.92E−06 | 9.23E−06 | 6.89E−07 | 2.97E−07 |
| HuIgG | 2.70E−09 | 3.79E−07 | 4.81E−07 | 3.12E−06 | 6.91E−07 | 2.84E−07 |
| Placebo | ND | ND | ND | ND | ND | ND |

### BGA384 Bound to C1q, the First Component of Complement Activation Cascade

The classical complement cascade is initiated as an antibody forms a stable interaction with C1q, which may result in complement-dependent cytotoxicity (CDC), i.e., killing of cells with the antibody-target bound by C1q (Arlaud et al 2002). To assess the potential CDC induced by BGA384, direct binding activity between C1q and BGA384 was assayed by ELISA.

Materials and Methods: An ELISA plate was coated with increasing amounts of BGA384. HuIgG (Sigma, Catalog #14506), or placebo. Human C1q (100 uL total: 8 μg/mL) was incubated in the antibody-coated wells. Bound C1q was detected by a specific anti-C1q monoclonal antibody. ELISA readings at 450 nm were plotted on the y-axis.

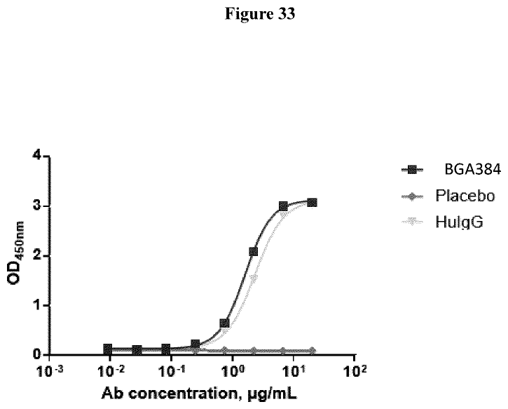

Results: These results indicated that BGA384 had strong binding to C1q similar to human IgG mixture, while the placebo showed no binding to C1q (FIG. 33).

Fc-Competent BGA384 Did not Induce ADCC Against PBMCs from Healthy Donors

As previously shown, BGA384 has competent binding activities to human FcγRs. An antibody can induce a variety of effector functions by binding to FcγRs. One of the important Fc receptors is Fc gamma receptor IIIA (FcγRIIIA), which mediates ADCC (Chen et al 2019). In humans, PVRIG is expressed on T cell and NK cells, but not on B cells, monocytes, or neutrophils (Zhu et al 2016). To assess whether BGA384 can induce ADCC against PVRIG positive cells, a co-culture assay was performed using human PBMC cells from healthy donors as target cells and autologous primary NK as effector cells. After co-culture in the presence of BGA384 for 17 hours, the absolute counts of CD3+CD56-T, CD4+T, CD8+T, Treg, and CD3−CD56+NK cell subsets were quantified by flow cytometry.

Materials and Methods: Healthy donor PBMCs were co-cultured with autologous primary NK cells in the presence of BGA384 for 17 h, the absolute counts of CD3+CD56−T, CD4+T, CD8+T, Treg, CD3−CD56+NK, and CD19+ B cells subsets were quantified by flow cytometry. Rituximab (an anti-human CD20 antibody) was used as a positive control. CD19+ B cells with high CD20 expression were depleted as expected. IgG was a negative control.

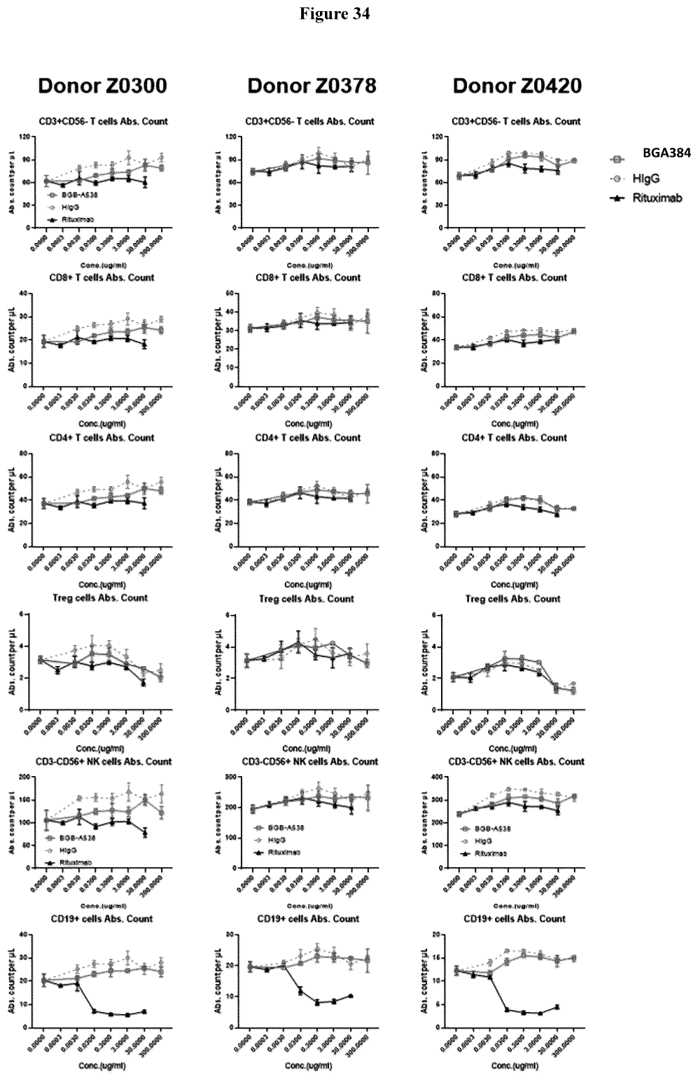

BGA384 did not induce ADCC against PVRIG positive cells in primary human PBMC cells (FIG. 34). As a positive control. Rituximab was shown to significantly reduce the number of CD19+B cells in a dose-dependent manner, while the number remained largely unchanged in the BGA384 or HIgG-treated group. These results indicate that BGA384 does not induce ADCC against PBMCs from healthy donors.

BGA384 does not Induce CDC in Primary Immune Cells

As shown previously. BGA384 has intact binding activity to C1q protein, a subunit of complement C1 complex that initiates the classical complement activation pathway (Kenway et al 2015). However, whether BGA384 will induce the complement activation cascade and complement-dependent cytotoxicity, especially in human primary T cells, has not previously been verified at a cellular level.

Materials and Methods: Pre-activated PBMCs from healthy donors were incubated with autologous scrum (15%) and BGA384 or control antibodies (0.01 to 300 μg/mL) for 3 days. Cell death due to CDC was assayed by the decrease of adenosine triphosphate released from viable cells after cell lysis at the end of the reaction. Anti HLA-A, -B, and -C was used as a positive control. The luminescence was recorded by a multi-detection microplate reader (PHERAstar® FS. BMG LABTECH GmbH, Ortenberg. Germany), and the CDC activities were calculated from the relative luminescence unit (RLU) readout as follows: % CDC activity=[(RLU test−RLU background)/(RLU max−RLU background)]×100.

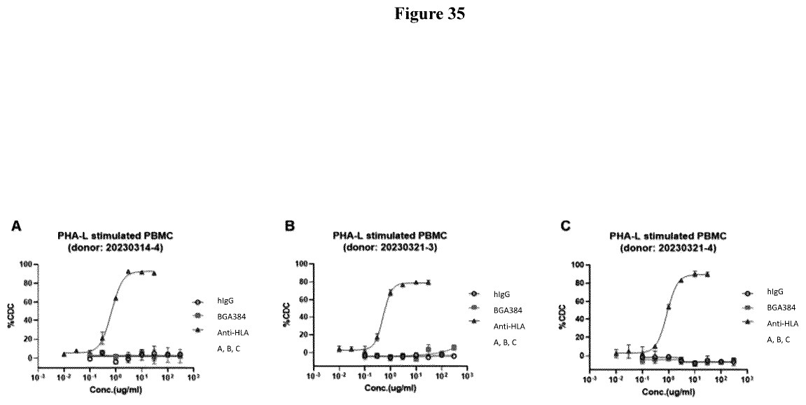

Results: BGA384 had no detectable CDC activity with PBMCs (FIGS. 35A-C). In contrast, the positive control antibody (anti-HLA-A, -B, -C) induced significant CDC activity. It has been reported that the density of target may play a critical role in regulating ADCC and CDC (Van Meerten et al 2006; Nijhof et al 2016). These studies suggest that PVRIG density on immune cell surfaces is not high enough to exert significant ADCC and CDC by BGA384.

### Fc-Competent BGA384 can Further Increase Activity in Addition to Blockade Function in Jurkat T Cells in the Presence of FcγR-Expressing THP1/OS8 Cells

To investigate whether Fc-competent BGA384 can promote stronger human T cell activity than Fc-silent BGA384 (BGA384-MF) in the presence of FcγR-expressing cells, a T cell activation bioluminescent cell-based assay has been established. The assay consists of Jurkat cells, which were genetically engineered to express a luciferase reporter driven by an NFAT-response element (NFAT-RE) and human PVRIG (Jurkat/NFAT/HuPVRIG), and THP1 cells, which were PVRL2 and FcγR positive and engineered to express low levels of T cell engager OS8 (THP1/OS8low). NFAT-RE-mediated luminescence was used as a readout for T-cell activation.

Materials and Methods: Jurkat/NFAT/HuPVRIG cells were co cultured with a mixture of THP1/OS8low in the presence of BGA384 or BGA384-MF at the indicated concentrations for 18 hours, hIgG1 was used as a negative control. NFAT-RE-mediated luminescence was determined by a One-glo luciferase assay system kit (Promega. REF #E6120).

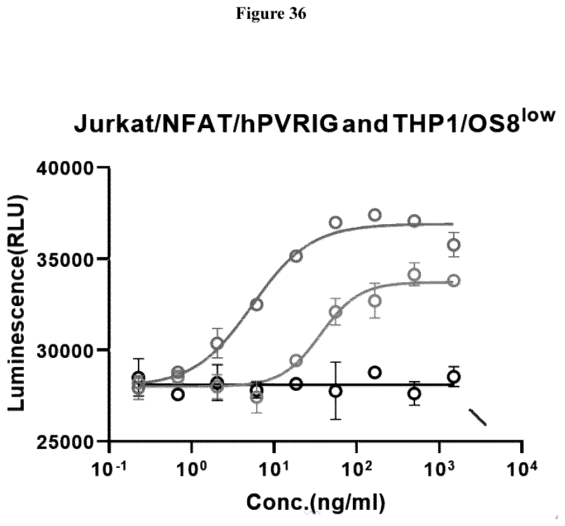

Results: BGA384 activated Jurkat cells to produce NFAT-RE-mediated luminescence dose-dependently, in conjunction with TCR/CD3 complex activation by blocking PVRIG-PVRL2-mediated immune inhibitory signaling (FIG. 36). In comparison to the Fc-silenced BGA384, Fc-competent BGA384 may have further increased activity by binding to FcγR on THP1. In contrast, human IgG1 did not trigger NFAT-RE-mediated luminescence under such assay conditions.

### Fc-Competent BGA384 can Further Increase Activity Besides the Blockade Function in Primary NK Cells

Both PVRIG and FcγRIIIA are expressed on NK cells (Zhu et al 2016, Bournazos et al 2020). To determine whether BGA384 can activate NK cells, purified primary NK cells were co-cultured with SK-BR-3, a PVRL2+ human breast cancer cell line, in the presence of BGA384. NK cell activation was determined by measuring an NK cell degranulation marker CD107a by flow cytometry.

Materials and Methods: In the NK activation assay, primary NK cells were isolated from healthy donor-derived PBMCs and stimulated with 25 U/mL recombinant human IL-2 overnight. Pre-activated NK cells were co-cultured with SK-BR-3 in the presence of anti-PVRIG mAb BGA384 or BGA384-MF for 5 hours. CD107a expression on NK cells was determined by FACS.

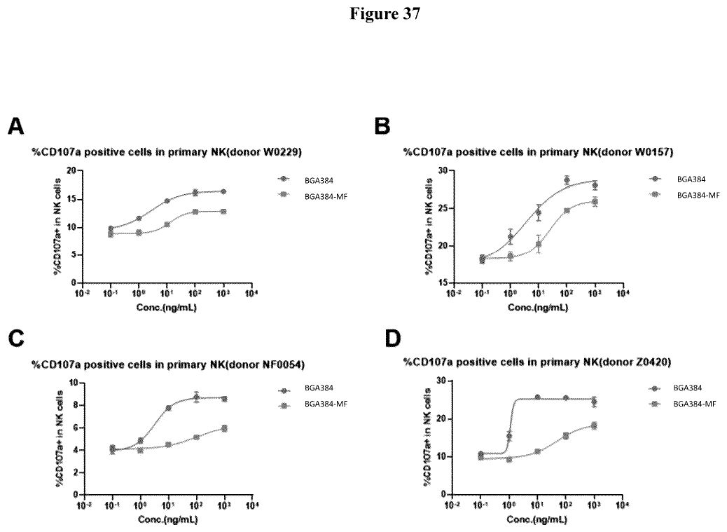

Results: The percentage of CD107a+ cells in total NK cells increased in a dose-dependent manner upon BGA384 treatment (FIGS. 37A-D). Furthermore, Fc-competent BGA384 triggered significantly higher CD107a upregulation compared to Fc-silent BGA384-MF, suggesting that the competent Fc plays a vital role in optimal NK activation.

### Example 20: In Vivo Pharmacology of BGA384 Surrogate Antibody

The in vivo efficacy of an anti-mouse PVRIG surrogate antibody for BGA384 (AB-407) with different Fc formats (WT and N297Q mutation) was examined in the mouse GL261 glioma subcutaneous syngeneic model in C57/B6 mice (Table 26). The surrogate antibody with competent Fc showed significant tumor growth inhibition (TGI) in the GL261 syngeneic model. These data indicate that BGA384 and its competent Fc may further enhance T-cell and NK-cell anti-tumor activities in vivo.

TABLE 26
In Vivo Pharmacology Study Summary

| Report | | Animal | Dose, | Noteworthy |
|---|---|---|---|---|
| No. | Title | Species | Schedule | Findings |
| BGB- A538 | Efficacy of | C57BL/6 | AB-407 WT, 1 | 37% (TGI |
| (surrogate)- | anti-mouse | | mg/kg, i.p., QW | on day 24) |
| IVP-Pha | PVRIG | | AB-407 WT, 5 | 51% (TGI |
| EFC- 0001 | surrogate | | mg/kg, i.p., QW | on day 24) |
| | antibody | | AB-407 WT, 15 | 65% (TGI |
| | AB-407 | | mg/kg, i.p., QW | on day 24) |
| | in GL261 | | AB-407 MF, 1 | 18% (TGI |
| | subcutaneous | | mg/kg, i.p., QW | on day 24) |
| | syngeneic | | AB-407 MF, 5 | 13% (TGI |
| | model | | mg/kg, i.p., QW | on day 24) |
| | | | AB-407 MF, 15 | 38% (TGI |
| | | | mg/kg, i.p., QW | on day 24) |

Abbreviations: AB-407 WT, AB-407 mIgG2a; AB-407 MF, AB-407 mIgG2a-N297Q mutant; i.p., intraperitoneally; QW, once a week; TGI, tumor growth inhibition.
Note:
TGI = 1 − [(treated Tt − treated T0)/(placebo Tt − placebo T0)], in which treated Tt = treated tumor volume at Time t, treated T0 = treated tumor volume at Time 0, placebo Tt = placebo tumor volume at Time t, and placebo T0 = placebo tumor volume at Time 0.

### Efficacy of Anti-Mouse PVRIG Surrogate Antibody AB-407 in GL261 Subcutaneous Syngeneic

The in vivo efficacy of the anti-mouse PVRIG surrogate antibody AB-407 with different mouse IgG2a Fc domains was evaluated in the mouse GL261 glioma subcutaneous syngeneic model in C57BL/6 mice. AB-407 is a chimeric monoclonal antibody against the mouse PVRIG. The Fab sequence of AB-407 was adopted from the antibody BOJ-5G4-F4 (disclosed in U.S. Pat. No. 10,213,505B2, which is hereby incorporated by reference in its entirety), and the Fc was a wild type mouse IgG2a (AB-407 mIgG2a, abbreviated as AB-407 WT) or a mouse IgG2a with N297Q mutation (AB-407 mIgG2a-N297Q, abbreviated as AB-407 MF). The N297Q mutation led to a greatly reduced binding affinity of the antibody to Fcγ receptors (FcγRs) and FcγRs mediated effector functions.

Materials and Methods: GL261 tumor cells (1×10⁷) were implanted subcutaneously on the right back of C57BL/6 mice. On the day of tumor inoculation, mice were randomized according to body weight and inoculation order with 20 mice in each group. From the next day of inoculation, mice were treated intraperitoneally for 32 days with vehicle, AB-407 WT or AB-407 MF (1, 5, and 15 mg/kg, QW), and the data was shown with a cut-off on day 24. Tumor volume was measured twice weekly. Data are presented as mean tumor volume±standard error of the mean (SEM) of 20 animals in each group. A one-way ANOVA with Dunnett multiple comparison method was used to analyze the log transformed tumor volume between treatment groups with the vehicle group (*p<0.05).

GL261 tumor cells (1×107) were implanted subcutaneously on the right back of C57BL/6 mice. On the day of tumor inoculation, mice were randomized according to body weight and inoculation order with 20 mice in each group. From the next day of inoculation, mice were treated intraperitoneally for 32 days with vehicle, AB-407 WT or AB-407 MF (1, 5 and 15 mg/kg, QW), and the data are shown with a cut-off on day 24. The body weight was measured twice weekly. Data are presented as mean body weight±SEM of 20 animals in each group.

In Table 27, all indicated parameters were calculated on day 24. A one-way ANOVA with Dunnett's multiple comparison method was used to analyze the log transformed tumor volume data between treatment groups and vehicle group.

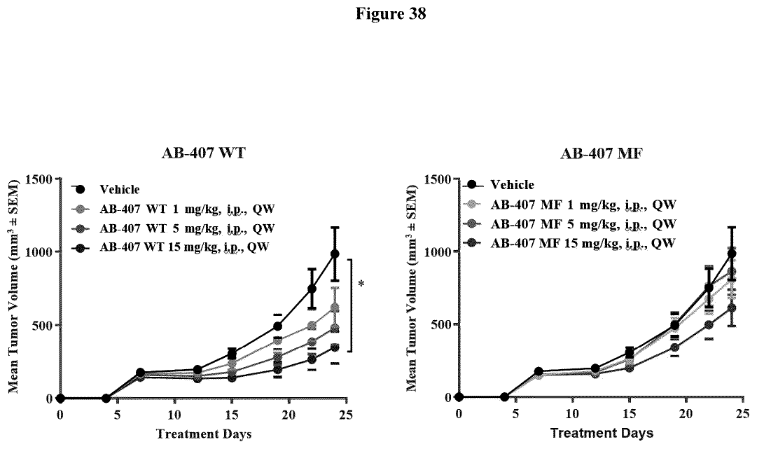

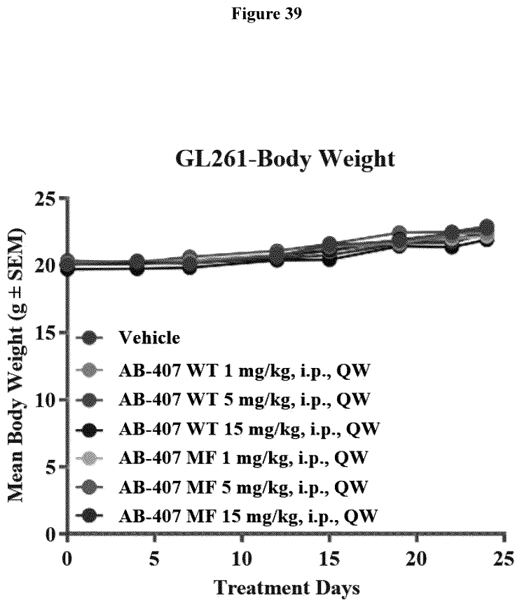

Results: Weekly intraperitoneal administration of 1, 5, and 15 mg/kg AB-407 WT showed tumor growth inhibition (TGI) of 37%, 51%, and 65% on Day 24, and 1, 5 and 15 mg/kg AB-407 MF showed TGI of 18%, 13% and 38% on Day 24, respectively (FIG. 38 and Table 27). The significant anti-tumor effect was found for AB-407 WT treatment at 15 mg/kg, but not for AB-407 MF at any dose, indicating binding of the PVRIG antibody to FcγRs is critical for anti-tumor activity. No significant change of animal body weight (FIG. 39) or abnormal clinical sign were identified in all treatment groups throughout the study.

TABLE 27
The Effect of AB-407 on Tumor Growth in
Mouse GL261 Subcutaneous Syngeneic Model

| | | | | | Mean Tumor | |
|---|---|---|---|---|---|---|
| | | | | | Volume | Adjusted p Value |
| | Dose | | | TGI | (mm³) | versus Vehicle |
| Compound | (mg/kg) | Treatment | N | (Day 24) | (Day 24) | (Day 24) |
| Vehicle | 0 | i.p., QW | 20 | N/A | 984.8 | N/A |
| AB-407 WT | 1 | i.p., QW | 20 | 37% | 621.9 | 0.7000 |
| AB-407 WT | 5 | i.p., QW | 20 | 51% | 481.2 | 0.0525 |
| AB-407 WT | 15 | i.p., QW | 20 | 65% | 347.5 | 0.0353 |
| AB-407 MF | 1 | i.p., QW | 20 | 18% | 807.2 | 1.0000 |
| AB-407 MF | 5 | i.p., QW | 20 | 13% | 861.4 | 1.0000 |
| AB-407 MF | 15 | i.p., QW | 20 | 38% | 612.9 | 0.9954 |

Abbreviations: i.p., intraperitoneally; N, animal number in each group; N/A, not applicable; QW, weekly; TGI, tumor growth inhibition.

### Example 21: Pre-Clinical Studies and Toxicology of BGA384

### Nonclinical Pharmacokinetics

A single-dose intravenous infusion PK study was conducted in cynomolgus monkeys at doses of 1, 5, or 20 mg/kg via intravenous infusion. The repeated-dose toxicokinetic analysis was incorporated in the 4-week repeated-dose toxicity study in cynomolgus monkeys as described below.

Dosing regimen: A total of 18 cynomolgus monkeys were randomly assigned to 3 groups (3/sex/group) and treated with BGA384 at a single dose of 1, 5, or 20 mg/kg via intravenous infusion. Serum drug concentrations were measured using a validated ELISA method.

Results: After a single administration of BGA384 from 1 to 20 mg/kg, no significant sex difference was observed in PK parameters. The serum concentration declined in a bi-exponential manner. The t½ of BGA384 was sustained with the mean values ranging from 168 to 270 hours; the mean Vdₛₛ ranged from 49.8 to 50.6 mL/kg, which was similar to typical monoclonal antibodies; the clearance (CL) was slow with the mean values ranging from 0.156 to 0.191 mL/h/kg; Cmax ranged from 31.7 to 613 μg/mL; and AUC0-840h ranged from 5,090 to 112,000 h·μg/mL. The systemic exposure of BGA384 increased in a dose-proportional manner and exhibited linear PK characteristics over the dose range from 1 to 20 mg/kg. The PK parameters are presented in Table 28.

TABLE 28
Pharmacokinetic Parameters of BGA384 in Monkeys After a Single Administration

| Dose | | | | | | | | | |
|---|---|---|---|---|---|---|---|---|---|
| Level | | Cₘₐₓ | Tₘₐₓ | t½ | AUC_{0-840 h} | AUC₀₋ₗₐₛₜ | AUC_{0-inf} | CL | Vdₛₛ |
| (mg/kg) | Sex | μg/mL | h | h | h · μg/mL | h · μg/mL | h · μg/mL | mL/h/kg | mL/kg |
| 1 | Male | 35.1 ± | 4.0 | 153 | 5100 ± | 5100 ± | 5270 ± | 0.192 ± | 45.5 ± |
| | | 4.94 | (2.0-8.0) | (145-192) | 658 | 658 | 771 | 0.0271 | 1.04 |
| | Female | 28.2 ± | 4.0 | 168 | 5070 ± | 5070 ± | 5300 ± | 0.190 ± | 54.1 ± |
| | | 4.13 | (2.0-4.0) | (168-191) | 548 | 548 | 547 | 0.0185 | 5.86 |
| | Mean | 31.7 ± | 4.0 | 168 | 5090 ± | 5090 ± | 5280 ± | 0.191 ± | 49.8 ± |
| | | 5.53 | (2.0-8.0) | (145-192) | 542 | 542 | 598 | 0.0208 | 6.03 |
| 5 | Male | 149 ± | 2.0 | 229 | 33800 ± | 33800 ± | 38300 ± | 0.134 ± | 48.6 ± |
| | | 11.1 | (0.5-4.0) | (221-329) | 5110 | 5110 | 8230 | 0.0273 | 3.04 |
| | Female | 150 ± | 4.0 | 256 | 26800 ± | 26500 ± | 29300 ± | 0.178 ± | 52.1 ± |
| | | 5.51 | (2.0-4.0) | (131-289) | 5230 | 5680 | 7560 | 0.0428 | 9.33 |
| | Mean | 150 ± | 3.0 | 243 | 30300 ± | 30100 ± | 33800 ± | 0.156 ± | 50.3 ± |
| | | 7.87 | (0.5-4.0) | (131-329) | 6010 | 6300 | 8620 | 0.0400 | 6.50 |
| 20 | Male | 622 ± | 2.0 | 311 | 136000 ± | 136000 ± | 160000 ± | 0.126 ± | 51.8 ± |
| | | 8.08 | (0.5-8.0) | (300-400) | 10200 | 10200 | 20100 | 0.0148 | 2.12 |
| | Female | 604 ± | 4.0 | 215 | 87600 ± | 87400 ± | 92000 ± | 0.226 ± | 49.3 ± |
| | | 78.6 | (0.5-4.0) | (56.8-239) | 17800 | 18100 | 22500 | 0.0523 | 11.3 |
| | Mean | 613 ± | 3.0 | 270 | 112000 ± | 112000 ± | 126000 ± | 0.176 ± | 50.6 ± |
| | | 50.9 | (0.5-8.0) | (56.8-400) | 29600 | 29800 | 41700 | 0.0643 | 7.39 |

Abbreviations: AUC0-inf, area under concentration-time curve from time 0 to infinity; AUC0-last, AUC from time 0 to the last quantifiable concentration; AUC0-840 h, AUC from time 0 to 840 hours post-dose; Cmax, maximum plasma concentration; CL, clearance; t½, half-life; Tmax, time to reach Cmax; Vdss, volume of distribution at steady state.

### Toxicology

### Repeat Dose Studies

A four-week repeat-dose toxicology study in cynomolgus monkeys was performed.

Dosing regimen: A total of 48 cynomolgus monkeys were assigned to 4 groups (6/sex/group) and treated with the control article (BGA384 DS buffer) or BGA384 DS in sodium chloride injection (0.9%) at 10, 30, or 100 mg/kg (once every week on Days 1, 8, 15, 22, and 29; 5 doses in total) via 30-minute intravenous (IV) infusion followed by a 4-week recovery period. The toxicokinetics (TK) profile was evaluated after the first week recovery period and after the first (Day 1) and fourth (Day 22) doses. BGA384 levels were analyzed, and immunotoxicity was evaluated through clinical observations, infusion site observations, clinical pathology, immunoglobins, complements, lymphocyte subsets, NK cells, cytokines, and histopathology evaluations.

Results: No mortality or moribundity was observed in animals throughout the study. No test-article-related changes were noted in clinical observations, administration site observations, body weights, food consumption, body temperature, electrocardiograms (ECGs), ophthalmoscopy, blood pressures, respiratory parameters, blood oxygen saturations, functional observational battery (FOB) for neurobehavioral assessments, clinical pathology (clinical chemistry, hematology, coagulation and urinalysis), TBNK lymphocyte subsets, or cytokines (TNF-α, IFN-γ, IL-2, IL-4, IL-5, complements [C3 and C4], and immunoglobulins [IgM, IgG, and IgA]) at any dose level throughout the study.

The TK profile was characterized after the first (Day 1), and fourth (Day 22) doses. The systemic exposure appeared to increase dose proportionally from 10 to 100 mg/kg. No apparent gender difference or accumulation was noted.

Overall, 100 mg/kg was considered as the highest non-severely toxic dose (HNSTD) under the study conditions, and the corresponding AUC0-last and Cmax on Day 22 were 384 h·mg/mL and 4670 μg/mL in males and 477 h·mg/mL and 4980 μg/mL in females, respectively.

### Local Tolerance Studies

In the repeat-dose toxicity study in cynomolgus monkeys, the infusion sites were observed and collected for microscopic evaluation. No noteworthy test article-related abnormal observations or histopathological changes were noted in the infusion sites.

### Tissue Cross Reactivity (TCR) Study in Normal Human Tissues

The TCR of BGA384 was evaluated in frozen normal human tissues using a validated immunohistochemical method.

Materials and Methods: A total of 35 tissue types, each in triplicate from 3 donors, were used for the evaluation. The study used 0.5 and 1.0 μg/mL Biotin-BGA384. 0.5 and 1.0 g/mL Biotin-Human IgG1-Kappa (IgG1 isotype control), and 0.01 mol/L phosphate-buffered saline (reagent control). The method was established and validated using positive and negative control protein smears of the hPVRIG-mIgG2a and the Human Serum Albumin Protein His Tag, respectively.

Positive Biotin-BGA384 staining was observed on the protein smears of the hPVRIG-mIgG2a, and the staining intensity was strong at 0.5 and 1 μg/mL, but no staining in IgG1 isotype control, PBS, or negative control. These results indicated that the assay was specific and reproducible.

Results: No specific staining of BGA384 was noted in normal human tissues.

### Human PBMC-Based Cytokine Release Assay

To assess the potential risk for BGA384 to induce CRS, an in vitro PBMC-based cytokine release assay was performed.

Materials and Methods: In this assay, antibodies (BGA384. OKT3 (anti-CD3 monoclonal antibody), or human IgG) were coated on polypropylene 96-well plates using an established “air-drying” method (Findlay et al 2010). PBMCs from 32 healthy donors were then cultured in RPMI-1640 media plus 10% fetal bovine serum in the presence of “dry-coated” antibodies for 22 hours. Human IgG was used as an isotype control, placebo as a negative control and Anti-CD3 monoclonal antibody OKT3 as a positive control. Cell-free supernatants were obtained by centrifugation and assayed using a multiplex kit (HCYTOMAG-60K, Millipore) and Luminex 200™. A panel of 15 cytokine/chemokines were analyzed (IL-2, IL-1β, IL-6, IL-8, TNF-α, IFN-γ, IL-4, IL-10, MCP-1/CCL2, MIP-1a/CCL3, IP-10/CXCL10, IL-17A, IL-12p70, IL-13, and GM-CSF).

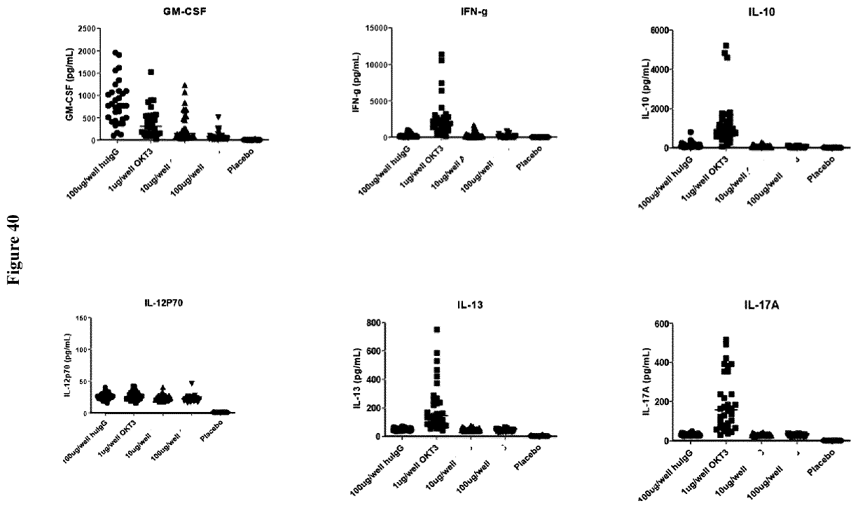

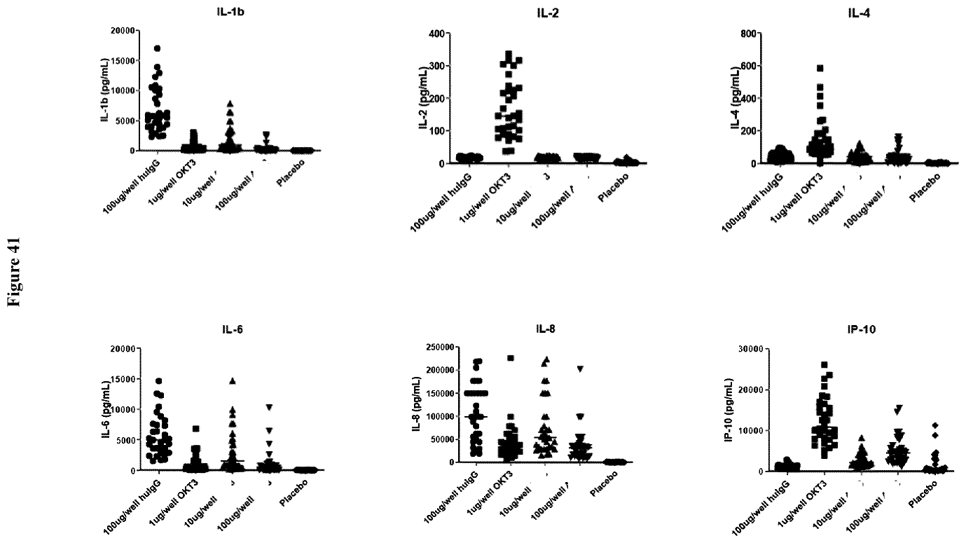

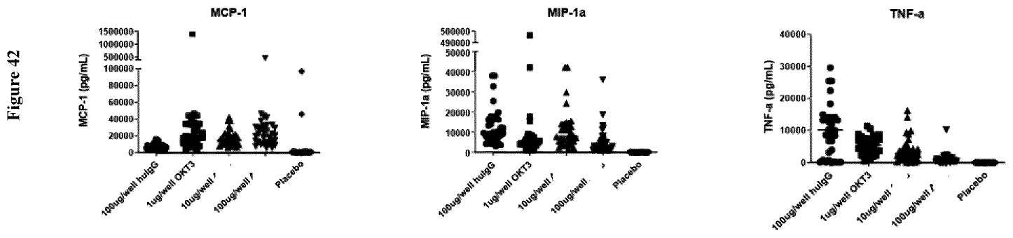

Results: OKT3 (plate-bound) induced significant amounts of cytokine/chemokine release in human PBMC from all 32 donors, whereas the placebo (negative control) induced little or no cytokine release (FIGS. 40-42). Immobilized human IgG induced significant amounts of GM-CSF. IL-1β, IL-6, IL-8, MCP-1, MIP-1a, and TNF-α, probably due to cross-linking of FcγRs on NK cells and monocytes (Roda et al 2006).

For GM-CSF. IL-10, IL-12p70, IL-13, IL-17A, IL-1β, and TNF-α, immobilized BGA384 (100 ug/well) induced a significantly lower release than both human IgG (100 μg/well) and OKT3 (1 μg/well). For some cytokines/chemokines that were involved with T cell activation, like IFN-γ. IL-2, IL-4, and IP-10, immobilized BGA384 (100 μg/well) induced a significantly lower release than OKT3. Also, compared to BGA384 (100 μg/well), human IgG induced a significantly greater release of IL-6, IL-8, and MIP-1A, which may be due to cross-linking of FcγRs on NK cells and monocytes. The selective induction of MCP-1 in PBMCs by BGA384 was observed. MCP-1, also known as CCL2, is a C—C chemokine primarily produced by monocytes and macrophages (Deshmane et al 2009). It is a pleiotropic cytokine that plays important roles in various disease areas, such as infectious diseases, cancer, and autoimmunity macrophages. Robust induction of MCP-1 has been previously observed in a chemokine release assay (CRA) after treatment with elotuzumab, a monoclonal antibody targeting CS1 (SLAM-F7), which is highly expressed on myeloma cells and weakly expressed on NK and T cells (Balasa et al 2008). The response was both dose- and Fc-dependent and correlated significantly with the level of NK cell activation. When dosed in a Phase 1 clinical trial, elotuzumab was found to induce only a transient elevation of the chemokines MCP-1 in the serum of multiple myeloma patients.

In summary, these results suggest that BGA384 has a low risk of causing CRS.

### Estimation of Clinical Doses Based on Nonclinical Data

### Interspecies PK Prediction

Serum concentration-time profiles from 18 cynomolgus monkeys that received single doses of BGA384 at 1, 5, and 20 mg/kg (Section 4.2) were used in a PK analysis with Phoenix® NLME Version 8.3 (Certara USA, Inc., Princeton, New Jersey, USA).

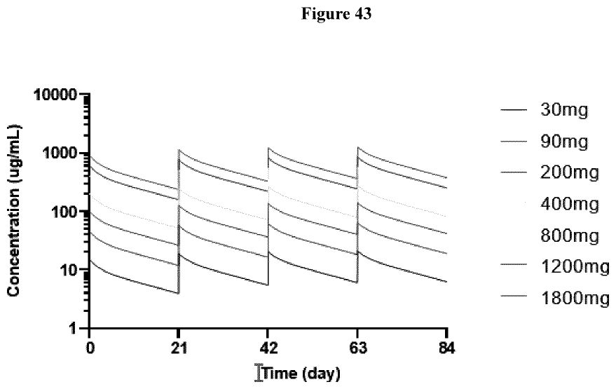

A 2-compartment model with first order-elimination described the data. Human PK parameters were obtained by scaling respective cynomolgus monkey PK parameters multiplied by the ratio of body weight (human/monkey) and taken to the power of 0.75 for CL and 1.0 for the volume of distribution (Mahmood and Balian 1999). The cynomolgus monkey PK parameters and projected human PK parameters are shown in Table 29. Simulated human PK profiles of BGA384 at different dose levels are shown in FIG. 43. The projected human terminal elimination half-life was 18.2 days.

TABLE 29
Population PK Parameter Estimates for Cynomolgus
Monkey and Projected PK Parameters in Humans

| | Monkey | | Human |
|---|---|---|---|
| Parameter | Estimate | % RSE | Projection |
| CL (mL/day/kg) | 3.93 | 5.6 | 2.55 |
| Q (mL/day/kg) | 10.5 | 14 | 6.85 |
| Vc (mL/kg) | 34.5 | 6.5 | 34.5 |
| Vp (mL/kg) | 16.7 | 2.3 | 16.7 |

Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed present disclosure.

1. An antibody that binds to human PVRIG, or antigen-binding fragment thereof, comprising:
(i)
three heavy chain CDRs:
HCDR1 comprising an amino acid sequence as set forth in SEQ ID NO:44,
HCDR2 comprising an amino acid sequence as set forth in SEQ ID NO:45,
HCDR3 comprising an amino acid sequence as set forth in SEQ ID NO:46, and
three light chain CDRs:
LCDR1 comprising an amino acid sequence as set forth in SEQ ID NO:47,
LCDR2 comprising an amino acid sequence as set forth in SEQ ID NO:48,
LCDR3 comprising an amino acid sequence as set forth in SEQ ID NO:49;
(ii)
three heavy chain CDRs:
HCDR1 comprising an amino acid sequence as set forth in SEQ ID NO:34,
HCDR2 comprising an amino acid sequence as set forth in SEQ ID NO:35,
HCDR3 comprising an amino acid sequence as set forth in SEQ ID NO:36, and
three light chain CDRs:
LCDR1 comprising an amino acid sequence as set forth in SEQ ID NO:37,
LCDR2 comprising an amino acid sequence as set forth in SEQ ID NO:38,
LCDR3 comprising an amino acid sequence as set forth in SEQ ID NO:39;
(iii)
three heavy chain CDRs:
HCDR1 comprising an amino acid sequence as set forth in SEQ ID NO:14,
HCDR2 comprising an amino acid sequence as set forth in SEQ ID NO:15,
HCDR3 comprising an amino acid sequence as set forth in SEQ ID NO:16, and
three light chain CDRs:
LCDR1 comprising an amino acid sequence as set forth in SEQ ID NO:17,
LCDR2 comprising an amino acid sequence as set forth in SEQ ID NO:18,
LCDR3 comprising an amino acid sequence as set forth in SEQ ID NO:19;
(iv)
three heavy chain CDRs:
HCDR1 comprising an amino acid sequence as set forth in SEQ ID NO:4,
HCDR2 comprising an amino acid sequence as set forth in SEQ ID NO:5,
HCDR3 comprising an amino acid sequence as set forth in SEQ ID NO:6, and
three light chain CDRs:
LCDR1 comprising an amino acid sequence as set forth in SEQ ID NO:7,
LCDR2 comprising an amino acid sequence as set forth in SEQ ID NO:8,
LCDR3 comprising an amino acid sequence as set forth in SEQ ID NO:9;
(v)
three heavy chain CDRs:
HCDR1 comprising an amino acid sequence as set forth in SEQ ID NO:24,
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:29;
(vi)
three heavy chain CDRs:
HCDR1 comprising an amino acid sequence as set forth in SEQ ID NO:54,
HCDR2 comprising an amino acid sequence as set forth in SEQ ID NO:55,
HCDR3 comprising an amino acid sequence as set forth in SEQ ID NO:56, and
three light chain CDRs:
LCDR1 comprising an amino acid sequence as set forth in SEQ ID NO:57,
LCDR2 comprising an amino acid sequence as set forth in SEQ ID NO:58,
LCDR3 comprising an amino acid sequence as set forth in SEQ ID NO:59;
(vii)
three heavy chain CDRs:
HCDR1 comprising an amino acid sequence as set forth in SEQ ID NO:64,
HCDR2 comprising an amino acid sequence as set forth in SEQ ID NO:65,
HCDR3 comprising an amino acid sequence as set forth in SEQ ID NO:66, and
three light chain CDRs:
LCDR1 comprising an amino acid sequence as set forth in SEQ ID NO:67,
LCDR2 comprising an amino acid sequence as set forth in SEQ ID NO:68,
LCDR3 comprising an amino acid sequence as set forth in SEQ ID NO:69;
(viii)
three heavy chain CDRs:
HCDR1 comprising an amino acid sequence as set forth in SEQ ID NO:74,
HCDR2 comprising an amino acid sequence as set forth in SEQ ID NO:75,
HCDR3 comprising an amino acid sequence as set forth in SEQ ID NO:76, and
three light chain CDRs:
LCDR1 comprising an amino acid sequence as set forth in SEQ ID NO:77,
LCDR2 comprising an amino acid sequence as set forth in SEQ ID NO:78,
LCDR3 comprising an amino acid sequence as set forth in SEQ ID NO:79;
(ix)
three heavy chain CDRs:
HCDR1 comprising an amino acid sequence as set forth in SEQ ID NO:84,
HCDR2 comprising an amino acid sequence as set forth in SEQ ID NO:85,
HCDR3 comprising an amino acid sequence as set forth in SEQ ID NO:86, and
three light chain CDRs:
LCDR1 comprising an amino acid sequence as set forth in SEQ ID NO:87,
LCDR2 comprising an amino acid sequence as set forth in SEQ ID NO:88,
LCDR3 comprising an amino acid sequence as set forth in SEQ ID NO:89;
(x)
three heavy chain CDRs:
HCDR1 comprising an amino acid sequence as set forth in SEQ ID NO:94,
HCDR2 comprising an amino acid sequence as set forth in SEQ ID NO:95,
HCDR3 comprising an amino acid sequence as set forth in SEQ ID NO:96, and
three light chain CDRs:
LCDR1 comprising an amino acid sequence as set forth in SEQ ID NO:97,
LCDR2 comprising an amino acid sequence as set forth in SEQ ID NO:98,
LCDR3 comprising an amino acid sequence as set forth in SEQ ID NO:99;
(xi)
three heavy chain CDRs:
HCDR1 comprising an amino acid sequence as set forth in SEQ ID NO:104,
HCDR2 comprising an amino acid sequence as set forth in SEQ ID NO: 105,
HCDR3 comprising an amino acid sequence as set forth in SEQ ID NO: 106, and
three light chain CDRs:
LCDR1 comprising an amino acid sequence as set forth in SEQ ID NO:107,
LCDR2 comprising an amino acid sequence as set forth in SEQ ID NO:108,
LCDR3 comprising an amino acid sequence as set forth in SEQ ID NO:109;
(xii)
three heavy chain CDRs:
HCDR1 comprising an amino acid sequence as set forth in SEQ ID NO:114,
HCDR2 comprising an amino acid sequence as set forth in SEQ ID NO:115,
HCDR3 comprising an amino acid sequence as set forth in SEQ ID NO:116, and
three light chain CDRs:
LCDR1 comprising an amino acid sequence as set forth in SEQ ID NO:117,
LCDR2 comprising an amino acid sequence as set forth in SEQ ID NO:118,
LCDR3 comprising an amino acid sequence as set forth in SEQ ID NO:119;
(xiii)
three heavy chain CDRs:
HCDR1 comprising an amino acid sequence as set forth in SEQ ID NO:124,
HCDR2 comprising an amino acid sequence as set forth in SEQ ID NO:125,
HCDR3 comprising an amino acid sequence as set forth in SEQ ID NO: 126, and
three light chain CDRs:
LCDR1 comprising an amino acid sequence as set forth in SEQ ID NO:127,
LCDR2 comprising an amino acid sequence as set forth in SEQ ID NO:128,
LCDR3 comprising an amino acid sequence as set forth in SEQ ID NO:129;
(xiv)
three heavy chain CDRs:
HCDR1 comprising an amino acid sequence as set forth in SEQ ID NO: 134,
HCDR2 comprising an amino acid sequence as set forth in SEQ ID NO:135,
HCDR3 comprising an amino acid sequence as set forth in SEQ ID NO:136, and
three light chain CDRs:
LCDR1 comprising an amino acid sequence as set forth in SEQ ID NO:137,
LCDR2 comprising an amino acid sequence as set forth in SEQ ID NO: 138,
LCDR3 comprising an amino acid sequence as set forth in SEQ ID NO:139;
(xv)
three heavy chain CDRs:
HCDR1 comprising an amino acid sequence as set forth in SEQ ID NO:144,
HCDR2 comprising an amino acid sequence as set forth in SEQ ID NO:145,
HCDR3 comprising an amino acid sequence as set forth in SEQ ID NO: 146, and
three light chain CDRs:
LCDR1 comprising an amino acid sequence as set forth in SEQ ID NO:147,
LCDR2 comprising an amino acid sequence as set forth in SEQ ID NO:148,
LCDR3 comprising an amino acid sequence as set forth in SEQ ID NO:149; or
(xvi)
three heavy chain CDRs:
HCDR1 comprising an amino acid sequence as set forth in SEQ ID NO:187,
HCDR2 comprising an amino acid sequence as set forth in SEQ ID NO: 188,
HCDR3 comprising an amino acid sequence as set forth in SEQ ID NO:46, and
three light chain CDRs:
LCDR1 comprising an amino acid sequence as set forth in SEQ ID NO:47,
LCDR2 comprising an amino acid sequence as set forth in SEQ ID NO:48,
LCDR3 comprising an amino acid sequence as set forth in SEQ ID NO:49.

2. The antibody or antigen-binding fragment thereof of claim 1, comprising:
(i) a heavy chain variable region (VH) comprising an amino acid sequence at least 90, 91, 92, 93, 94, 95, 96, 97, 98 or 99% identical to SEQ ID NO:50, and a light chain variable region (VL) comprising an amino acid sequence at least 90, 91, 92, 93, 94, 95, 96, 97, 98 or 99% identical to SEQ ID NO:51;
(ii) a heavy chain variable region (VH) comprising an amino acid sequence at least 90, 91, 92, 93, 94, 95, 96, 97, 98 or 99% identical to SEQ ID NO:40, and a light chain variable region (VL) comprising an amino acid sequence at least 90, 91, 92, 93, 94, 95, 96, 97, 98 or 99% identical to SEQ ID NO:41;
(iii) a heavy chain variable region (VH) comprising an amino acid sequence at least 90, 91, 92, 93, 94, 95, 96, 97, 98 or 99% identical to SEQ ID NO:20, and a light chain variable region (VL) comprising an amino acid sequence at least 90, 91, 92, 93, 94, 95, 96, 97, 98 or 99% identical to SEQ ID NO:21;
(iv) a heavy chain variable region (VH) comprising an amino acid sequence at least 90, 91, 92, 93, 94, 95, 96, 97, 98 or 99% identical to SEQ ID NO:10, and a light chain variable region (VL) comprising an amino acid sequence at least 90, 91, 92, 93, 94, 95, 96, 97, 98 or 99% identical to SEQ ID NO:11;
(v) a heavy chain variable region (VH) comprising an amino acid sequence at least 90, 91, 92, 93, 94, 95, 96, 97, 98 or 99% identical to SEQ ID NO:30, and a light chain variable region (VL) comprising an amino acid sequence at least 90, 91, 92, 93, 94, 95, 96, 97, 98 or 99% identical to SEQ ID NO:31;
(vi) a heavy chain variable region (VH) comprising an amino acid sequence at least 90, 91, 92, 93, 94, 95, 96, 97, 98 or 99% identical to SEQ ID NO:60, and a light chain variable region (VL) comprising an amino acid sequence at least 90, 91, 92, 93, 94, 95, 96, 97, 98 or 99% identical to SEQ ID NO:61;
(vii) a heavy chain variable region (VH) comprising an amino acid sequence at least 90, 91, 92, 93, 94, 95, 96, 97, 98 or 99% identical to SEQ ID NO:70, and a light chain variable region (VL) comprising an amino acid sequence at least 90, 91, 92, 93, 94, 95, 96, 97, 98 or 99% identical to SEQ ID NO:71;
(viii) a heavy chain variable region (VH) comprising an amino acid sequence at least 90, 91, 92, 93, 94, 95, 96, 97, 98 or 99% identical to SEQ ID NO:80, and a light chain variable region (VL) comprising an amino acid sequence at least 90, 91, 92, 93, 94, 95, 96, 97, 98 or 99% identical to SEQ ID NO:81;
(ix) a heavy chain variable region (VH) comprising an amino acid sequence at least 90, 91, 92, 93, 94, 95, 96, 97, 98 or 99% identical to SEQ ID NO:90, and a light chain variable region (VL) comprising an amino acid sequence at least 90, 91, 92, 93, 94, 95, 96, 97, 98 or 99% identical to SEQ ID NO:91;
(x) a heavy chain variable region (VH) comprising an amino acid sequence at least 90, 91, 92, 93, 94, 95, 96, 97, 98 or 99% identical to SEQ ID NO:100, and a light chain variable region (VL) comprising an amino acid sequence at least 90, 91, 92, 93, 94, 95, 96, 97, 98 or 99% identical to SEQ ID NO:101;
(xi) a heavy chain variable region (VH) comprising an amino acid sequence at least 90, 91, 92, 93, 94, 95, 96, 97, 98 or 99% identical to SEQ ID NO:110, and a light chain variable region (VL) comprising an amino acid sequence at least 90, 91, 92, 93, 94, 95, 96, 97, 98 or 99% identical to SEQ ID NO:111;
(xii) a heavy chain variable region (VH) comprising an amino acid sequence at least 90, 91, 92, 93, 94, 95, 96, 97, 98 or 99% identical to SEQ ID NO:120, and a light chain variable region (VL) comprising an amino acid sequence at least 90, 91, 92, 93, 94, 95, 96, 97, 98 or 99% identical to SEQ ID NO:121;
(xiii) a heavy chain variable region (VH) comprising an amino acid sequence at least 90, 91, 92, 93, 94, 95, 96, 97, 98 or 99% identical to SEQ ID NO:130, and a light chain variable region (VL) comprising an amino acid sequence at least 90, 91, 92, 93, 94, 95, 96, 97, 98 or 99% identical to SEQ ID NO:131;
(xiv) a heavy chain variable region (VH) comprising an amino acid sequence at least 90, 91, 92, 93, 94, 95, 96, 97, 98 or 99% identical to SEQ ID NO:140, and a light chain variable region (VL) comprising an amino acid sequence at least 90, 91, 92, 93, 94, 95, 96, 97, 98 or 99% identical to SEQ ID NO:141; or
(xv) a heavy chain variable region (VH) comprising an amino acid sequence at least 90, 91, 92, 93, 94, 95, 96, 97, 98 or 99% identical to SEQ ID NO:150, and a light chain variable region (VL) comprising an amino acid sequence at least 90, 91, 92, 93, 94, 95, 96, 97, 98 or 99% identical to SEQ ID NO:151.

3. The antibody or antigen-binding fragment thereof of claim 2, wherein one, two, three, four, five, six, seven, eight, nine, or ten amino acids within SEQ ID Nos: 50 and 51, SEQ ID Nos: 40 and 41, SEQ ID Nos: 20 and 21, SEQ ID Nos: 10 and 11, SEQ ID Nos: 30 and 31, SEQ ID Nos: 60 and 61, SEQ ID Nos: 70 and 71, SEQ ID Nos: 80 and 81, SEQ ID Nos: 90 and 91, SEQ ID Nos: 100 and 101, SEQ ID Nos: 110 and 111, SEQ ID Nos: 120 and 121, SEQ ID Nos: 130 and 131, SEQ ID Nos: 140 and 141, or SEQ ID Nos: 150 and 151, have been inserted, deleted or substituted.

4. The antibody or antigen-binding fragment thereof of claim 1, that comprises:
(i) a heavy chain variable region (VH) comprising an amino acid sequence as set forth in SEQ ID NO: 50, and a light chain variable region (VL) comprising an amino acid sequence as set forth in SEQ ID NO:51;
(ii) a heavy chain variable region (VH) comprising an amino acid sequence as set forth in SEQ ID NO: 40, and a light chain variable region (VL) comprising an amino acid sequence as set forth in SEQ ID NO:41;
(iii) a heavy chain variable region (VH) comprising an amino acid sequence as set forth in SEQ ID NO: 20, and a light chain variable region (VL) comprising an amino acid sequence as set forth in SEQ ID NO:21;
(iv) a heavy chain variable region (VH) comprising an amino acid sequence as set forth in SEQ ID NO: 10, and a light chain variable region (VL) comprising an amino acid sequence as set forth in SEQ ID NO:11;
(v) a heavy chain variable region (VH) comprising an amino acid sequence as set forth in SEQ ID NO: 30, and a light chain variable region (VL) comprising an amino acid sequence as set forth in SEQ ID NO:31;
(vi) a heavy chain variable region (VH) comprising an amino acid sequence as set forth in SEQ ID NO: 60, and a light chain variable region (VL) comprising an amino acid sequence as set forth in SEQ ID NO:61;
(vii) a heavy chain variable region (VH) comprising an amino acid sequence as set forth in SEQ ID NO: 50, and a light chain variable region (VL) comprising an amino acid sequence as set forth in SEQ ID NO:51;
(viii) a heavy chain variable region (VH) comprising an amino acid sequence as set forth in SEQ ID NO: 70, and a light chain variable region (VL) comprising an amino acid sequence as set forth in SEQ ID NO:71;
(ix) a heavy chain variable region (VH) comprising an amino acid sequence as set forth in SEQ ID NO: 80, and a light chain variable region (VL) comprising an amino acid sequence as set forth in SEQ ID NO:81;
(x) a heavy chain variable region (VH) comprising an amino acid sequence as set forth in SEQ ID NO: 90, and a light chain variable region (VL) comprising an amino acid sequence as set forth in SEQ ID NO:91;
(xi) a heavy chain variable region (VH) comprising an amino acid sequence as set forth in SEQ ID NO: 100, and a light chain variable region (VL) comprising an amino acid sequence as set forth in SEQ ID NO:101;
(xii) a heavy chain variable region (VH) comprising an amino acid sequence as set forth in SEQ ID NO: 110, and a light chain variable region (VL) comprising an amino acid sequence as set forth in SEQ ID NO:111;
(xiii) a heavy chain variable region (VH) comprising an amino acid sequence as set forth in SEQ ID NO: 120, and a light chain variable region (VL) comprising an amino acid sequence as set forth in SEQ ID NO:121;
(xiv) a heavy chain variable region (VH) comprising an amino acid sequence as set forth in SEQ ID NO: 140, and a light chain variable region (VL) comprising an amino acid sequence as set forth in SEQ ID NO:141;
(xv) a heavy chain variable region (VH) comprising an amino acid sequence as set forth in SEQ ID NO: 150, and a light chain variable region (VL) comprising an amino acid sequence as set forth in SEQ ID NO:151.

5. The antibody or antigen-binding fragment thereof of claim 1, further comprising an antigen binding domain that specifically binds to human PVRIG at an epitope comprising S27, S31, L32, H52, F99, K95, P100, E101, S103 and E105 of SEQ ID NO: 154.

6. An isolated human monoclonal antibody, or an antigen-binding portion thereof, wherein the antibody or antigen-binding portion thereof cross-competes for binding to PVRIG with a reference antibody, or a reference antigen-binding portion thereof, comprising the antibody or antigen-binding fragment thereof of claim 1.

7. (canceled)

8. The antibody or antigen-binding portion thereof of claim 6, wherein the antibody binds to a discontinuous epitope on PVRIG, wherein the discontinuous epitope comprises amino acid residues S27, S31 and L32 of human PVRIG comprising SEQ ID NO: 154.

9. The antibody or antigen-binding portion thereof of claim 8, wherein the discontinuous epitope further comprises amino acid residues H52, K95, F99, P100, E101, S103 and E105 of human PVRIG comprising SEQ ID NO:154.

10. The antibody or antigen-binding fragment thereof of claim 1, which is a monoclonal antibody, a chimeric antibody, a humanized antibody, a human engineered antibody, a single chain antibody (scFv), a Fab fragment, a Fab′ fragment, or a F(ab′)₂ fragment.

11. The antibody or antigen-binding fragment thereof of claim 1, wherein the antibody or antigen-binding fragment thereof: (a) has reduced glycosylation or no glycosylation or is hypofucosylated, (b) comprises increased bisecting GlcNac structures, or (c) comprises an IgG1 Fc domain.

12. (canceled)

13. (canceled)

14. A pharmaceutical composition comprising the antibody or antigen-binding fragment thereof of claim 1, and further comprising a pharmaceutically acceptable carrier.

15. An isolated nucleic acid that encodes the antibody or antigen-binding fragment thereof of claim 1.

16. A vector comprising the nucleic acid of claim 15.

17. A host cell comprising the nucleic acid of claim 15.

18. A process for producing an antibody or antigen-binding fragment thereof comprising cultivating the host cell of claim 17 in culture media and recovering the antibody or antigen-binding fragment thereof from the culture media.

19. A purified composition comprising an anti-human PVRIG antibody or antigen binding fragment thereof produced by the process of claim 18.

20. (canceled)

21. (canceled)

22. A method of treating cancer comprising administering to a patient in need thereof an effective amount of the pharmaceutical composition of claim 14.

23.-32. (canceled)

33. A method of stimulating an immune response in a subject comprising administering to the subject the pharmaceutical composition of claim 14.

34. (canceled)

35. A method of inducing trogocytosis of PVRIG from a donor cell to an acceptor cell, comprising contacting the donor cell with an antibody or antigen-binding fragment of claim 1 for a time sufficient to induce trogocytosis from the donor cell to the acceptor cell.

36. A method of activating NK-cells comprising administering to a patient in need thereof an effective amount of the pharmaceutical composition of claim 14.

37.-39. (canceled)
